(19) United States
(12) Patent Application Publication
(10) Pub. No.: US 2013/0131018 A1
(43) Pub. Date: May 23, 2013

(54) SUBSTITUTED ISOQUINOLINES AND THEIR USE AS TUBULIN POLYMERIZATION INHIBITORS

(71) Applicants: Bertrand Leblond, Paris (FR); Thierry Taverne, Saint Martin Les Boulogne S/mer (FR); Eric Beausoleil, Paris (FR); Cédric Chauvignac, Nogent Sur Marne (FR); Anne-Sophie Casagrande, Draveil (FR); Laurent Desire, Paris (FR)

(72) Inventors: Bertrand Leblond, Paris (FR); Thierry Taverne, Saint Martin Les Boulogne S/mer (FR); Eric Beausoleil, Paris (FR); Cédric Chauvignac, Nogent Sur Marne (FR); Anne-Sophie Casagrande, Draveil (FR); Laurent Desire, Paris (FR)

(73) Assignee: Exonhit S.A., Paris (FR)

(21) Appl. No.: 13/701,917

(22) PCT Filed: Jun. 1, 2011
(86) PCT No.: PCT/EP2011/059157

§ 371 Date: Feb. 5, 2013

(30) Foreign Application Priority Data
Jun. 4, 2010 (EP) 10305605.7
Jul. 23, 2010 (EP) 10170684.4

(51) Int. Cl. C07D 217/20; A61K 31/437; A61K 31/472; A61K 45/06; C07D 498/04; C07D 401/10; C07D 405/10; A61K 31/4725; A61K 31/675; C07F 9/62

(52) U.S. Cl. CPC C07D 217/20; A61K 31/675; A61K 31/437; A61K 31/472; C07F 9/62; C07D 498/04; C07D 401/10; C07D 405/10; A61K 31/4725; A61K 45/06
USPC 514/82; 514/293; 514/307; 546/23; 546/83; 546/143; 546/146; 546/149

(57) ABSTRACT

The present invention relates generally to substituted isoquinolines and their use as tubulin polymerization inhibitors. In particular, the invention relates to substituted isoquinolines which possess useful therapeutic activity, use of these compounds in methods of therapy and the manufacture of medicaments as well as compositions containing these compounds.

### FIELD OF THE INVENTION

The present invention relates generally to substituted isoquinolines and their use as tubulin polymerization inhibitors. In particular, the invention relates to substituted isoquinolines which possess useful therapeutic activity, use of these compounds in methods of therapy and the manufacture of medicaments as well as compositions containing these compounds.

### BACKGROUND OF THE INVENTION

Microtubules are dynamic structures that play a crucial role in cellular division and are recognized as an important target for anticancer therapy and inflammation (Jordan, M. A., Wilson, L., Microtubules as a Target for Anticancer, Drugs. Nat. Rev. Cancer 2004, 4, 253-265).

Tubulin is one of several members of a small family of globular proteins. Tubulin is composed of a heterodimer of two related proteins called α-tubulin and β-tubulin. Tubulin polymerizes to form structures called microtubules.

Microtubules, dynamic protein polymers composed of α-tubulin and β-tubulin heterodimers, are a well-established cellular target for anticancer drugs. Dynamic polymerization of tubulin is a necessary and tightly controlled process during mitosis.

Perturbing microtubule dynamics with small molecules blocks the cell cycle in the metaphase/anaphase transition and leads to apoptosis (Jordan, M. A., Mechanism of action of antitumor drugs that interact with microtubules and tubulin, Curr. Med. Chem.: Anti-Cancer Agents 2002, 2, 1-17). Thus, small molecules that target tubulin halt rapid cell division, a characteristic of cancer cells.

Small molecules have been shown to bind at three major binding sites on tubulin: the vinca, taxane, and colchicines binding sites. While the drugs that act on the vinca and taxane sites have well-established roles in the treatment of human cancers, the therapeutic potential of the colchicine site in cancer treatment has yet to be realized.

Tubulin-binding agents are characterized by their binding sites on tubulin and their effects on tubulin polymerization. Vincristine, vinblastine, and vinorelbine (navelbine) bind to the vinca domain and inhibit tubulin assembly (Kruczynski A., Hill B. T., Vinflunine, the latest vinca alkaloid in clinical development. A review of its preclinical anticancer properties, Crit. Rev. Oncol. Hematol., 2001, 40, 159-73; Jordan M. A., Thrower D., Wilson L., Mechanism of inhibition of cell proliferation by Vinca alkaloids, Cancer Res., 1991, 51, 2212-22), whereas paclitaxel, docetaxel, and epothilones bind to the taxane-binding site and stabilize microtubules (Rose W. C., Taxol: a review of its preclinical in vivo antitumor activity, Anticancer Drugs, 1992, 3, 311-21; Choy H., Taxanes in combined modality therapy for solid tumors, Crit. Rev. Oncol. Hematol., 2001, 37, 237-47). These compounds exhibit their anticancer properties by interfering with the dynamics of tubulin polymerization and depolymerization, resulting in mitotic arrest. Their efficacies are well proven in clinic, but a limitation is the development of drug resistance due to the overexpression of the drug efflux pumps (Ark-Otte J., Samelis G., Rubio G., Lopez Saez J. B., Pinedo H. M., Giaccone G., Effects of tubulin-inhibiting agents in human lung and breast cancer cell lines with different multidrug resistance phenotypes, Oncol. Rep. 1998, 5, 249-55) and/or the appearance of mutations in tubulin genes (Berrieman H. K., Lind M. J., Cawkwell L., Do β-tubulin mutations have a role in resistance to chemotherapy? Lancet Oncol., 2004, 5, 158-64).

The tubulin-binding site has been described for its ability to bind a naturally occurring tricyclic alkaloid colchicine, which inhibits tubulin polymerization (Zhou J., Giannakakou P., Targeting microtubules for cancer chemotherapy, Curr. Med. Chem. Anti-Canc. Agents, 2005, 5, 65-71). Colchicine itself is not a useful anticancer agent because of its narrow therapeutic window, but compounds with diverse chemical structures (scheme 1) that bind to this site (or near) are now in clinical or preclinical developments e.g. combretastatin CA-4P (Zybrestat) described in patent U.S. Pat. No. 5,561,122, AVE-8062 (Ombrabulin) described in U.S. Pat. No. 5,674,906, BNC105P described in WO2007087684, MPC-6827 (Azixa) described in WO2005003100, ZD6126 (ANG453) described in US20026423753, Oxi-4503 described in WO01081355, MN029 (Denibulin) described in US20036645950, NPI-2358 (Phenylahistin) described in US20067064201, EPC-2407 (Crinobulin) described in US20056906203, indibulin (ZIO-301), T115 described in WO2006047631, BPR0L075 described in US2003195244 and ABT-751 described in EP472053.

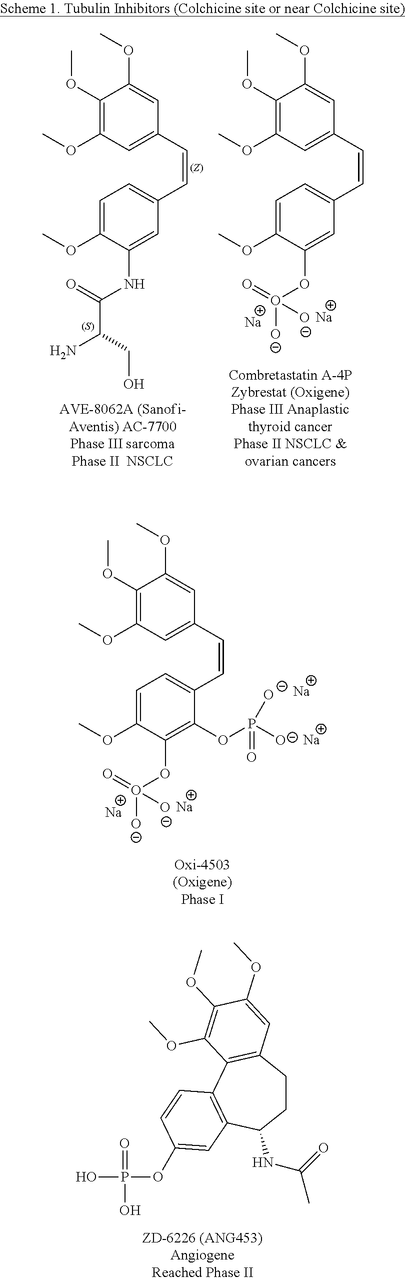

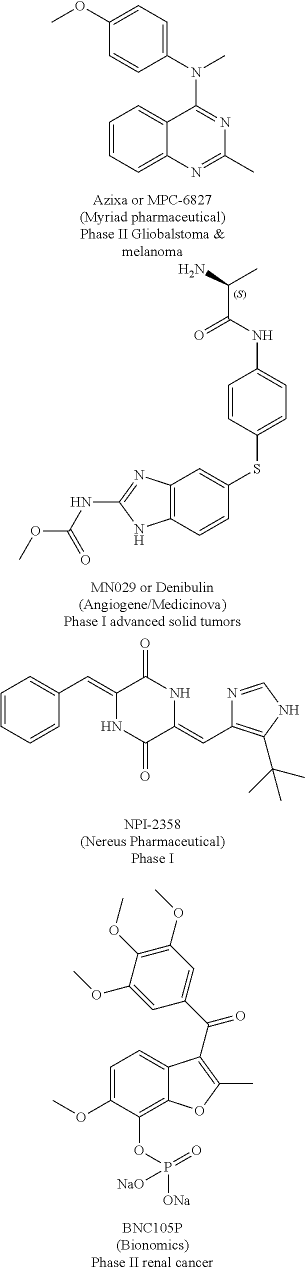

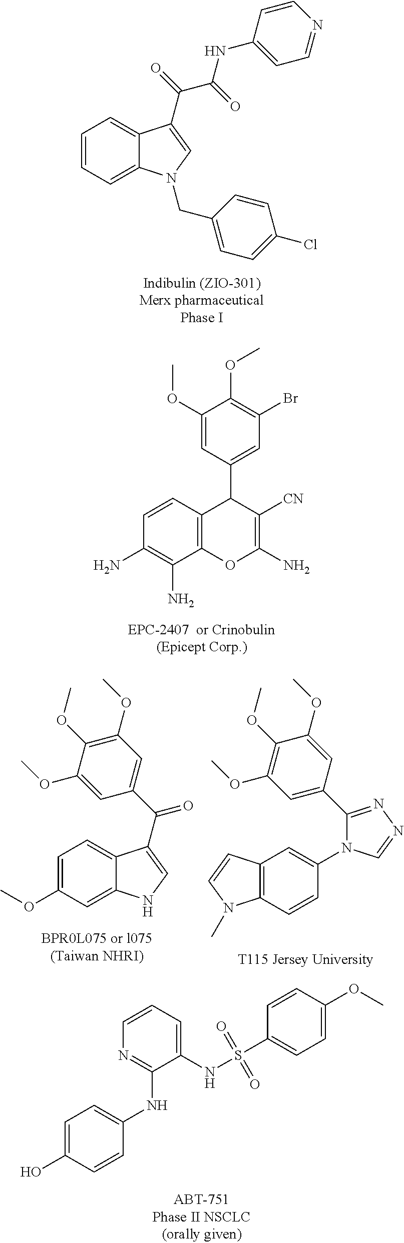

Tumor blood vessels are a promising target for cancer therapy. Compounds that act to deprive tumors of their blood supply fall into two classes: angiogenesis inhibitors and vascular disrupting agents (VDAs). A VDA is a drug designed to damage the vasculature (blood vessels) of cancer tumors causing central necrosis. Angiogenesis inhibitors block the formation of new blood vessels formed in response to tumor growth (Folkman J., Tumor angiogenesis: therapeutic implications, New Engl. J. Med., 1971, 285, 125-134), whereas VDAs disrupt blood flow through existing tumor blood vessels (Denekamp J., Endothelial cell proliferation as a novel approach to targeting tumour therapy, Br. J. Cancer., 1982, 45, 136-139).

Compounds binding to (or near) the colchicine domain are undergoing intensive investigation as VDAs for cancer therapy. For example, clinical candidates of the microtubule inhibitor family, combretastatin CA-4P (Zybrestat), AVE-8062 (Ombrabulin), BNC105P and MPC-6827 (Azixa) act as a vascular disrupting agent (VDA) that rapidly depolymerize microtubules of newly formed vasculatures and subsequently shut down the blood supply to tumors. Neovascular systems are more sensitive to these agents because they are more dependent on microtubule cytoskeletons than normal, healthy, vascular endothelial cells which are also supported by actin based cytoskeletal structures.

These compounds exert highly selective effects in proliferating endothelial cells and, as a consequence, demonstrate strong suppressive activity on tumor blood flow (THF) and induce haemorrhagic necrosis of solid tumors at doses that are well tolerated (Cai S. X., Small molecule Vascular Disrupting Agents: Potential new drugs for cancer treatment, Recent patents on anti-cancer drug discovery, 2007, 2(1), 79-101).

The agents that target the colchicine binding domain are potentially dual mode agents (ie. antimitotic and VDAs). It has a great importance to identify structurally novel VDAs which bind to the colchicine binding site (or near), showing highly potent antiproliferative activity on human tumor cell lines, which are resistant to standard tubulin-binding agents.

CA-4 disodium phosphate (CA-4P, Zybrestat™), a water-soluble prodrug of CA-4 developed by OXiGENE is in phase II/III in patients with metastatic anaplastic thyroid cancer (ATC), in phase II for patients with stage IIIb/IV non-small cell lung cancer (NSCLC) and in phase II in patients with platinum-resistant ovarian cancer. The phosphate of CA-4P has a short plasma half-life and desphosphorylates to CA-4.

In addition several angiogenesis-inhibiting drugs have recently been approved for treatment of wet age-related macular degeneration (ARMD), but require direct injection into the eye (intravitreal injection) on a regular basis and can cause side-effects. A topically-administered anti-vascular drug, such as CA-4P offer significant advantages to patients with ARMD and other ophthalmological diseases in which abnormal blood-vessel pathophysiology plays a role. OXiGENE is developing CA-4P for the treatment of ophthalmological diseases and conditions (preclinical study), with the objective of delivering the drug via a convenient and patient-friendly topical formulation (e.g., eye drops).

To date, the majority of clinical experience has been obtained with CA-4P which is evaluated in a number of clinical trials in combination with either chemotherapy, radiation or anti-angiogenic therapies. Animal studies have shown that CA4P induces a 100-fold blood flow reduction in the tumor, less than 7-fold in spleen, skeletal muscle, and brain, and no significant decrease in heart, kidney, and intestine. The agent is administered intravenously, and three dosing schedules were tested in phase 1 studies. The dose-limiting toxicities included dyspnea, neurological disturbances (syncope, motor neuropathy, ataxia), and cardiac and intestinal ischemia. Responses were seen in thyroid cancer, sarcoma, and adrenocortical carcinoma. The agent was tested in anaplastic thyroid cancer as a single-agent and in combination with carboplatin/paclitaxel and cisplatin/doxorubicin/radiation in phase 2 studies. CA-4P is also being tested with carboplatin, carboplatin/paclitaxel, and bevacizumabin patient with solid tumors.

### SUMMARY OF THE INVENTION

The present invention relates to isoquinoline compounds which can be easily synthesized, which have low toxicity and which have antitumoral properties, and the provision of compositions containing them.

The present invention relates to a compound of formula (I):

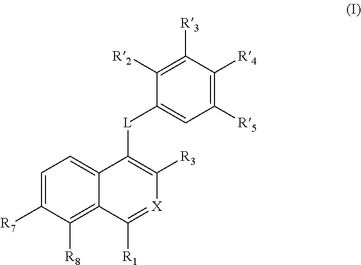

wherein:
X represents N or N⁺—Z, wherein Z is selected in the group consisting of a (C₁-C₆)alkyl, an aryl and an acyl;
R₁ represents H, CN, a CORa or a (C₁-C₅)alkyl;
   wherein Ra represents a NRa′Ra″ or ORa′″;
   wherein Ra′ and Ra″, independently from each other, are selected from the group consisting of H and (C₁-C₅)alkyl;
   wherein Ra′″ represents H or (C₁-C₅)alkyl;
R₃ represents H, CN, OH, a CORb, NH₂ or a (C₁-C₅)alkyl group;
   wherein Rb represents a NRb′Rb″ or ORb′″ group;
   wherein Rb′ and Rb′, independently from each other, are selected from the group consisting of H and (C₁-C₅)alkyl;
   wherein Rb′″ represents H or (C₁-C₅)alkyl;
R₇ represents a (C₁-C₅)alkyl, a (C₁-C₅)alkoxy, a (C₁-C₅)alkylthio, a (C₁-C₅)alkylamino, a (C₁-C₅)dialkylamino;
R₈ represents H, halogen, nitro, CN, N₃, a diarylmethylenehydrazinyl, a di(C₁-C₆)alkyl-N—C═S(O)— group or a di(C₁-C₆)alkyl-N—C═O(S)— group, a (C₁-C₆)alkyl-SO₂, SR, OR or NRR′ group;
   wherein R and R′, independently from each other, are selected from the group consisting of H, a (C₁-C₆)alkyl, a (C₂-C₆)alkenyl, an acyl, an aryl, a heterocyclyl, an amino acid, a Y—SO₂ group, a —P(O)(OG)(OG′) group,
   wherein Y is selected in the group consisting of a hydrogen atom, (C₁-C₆)alkyl, NH₂, (C₁-C₆)alkylamino, di(C₁-C₆)alkylamino and (C₁-C₆)alkoxycarbonylamino;
   wherein G and G′, independently from each other, are selected in the group consisting of H, (C₁-C₆)alkyl and aryl;
R₇ and R₈, taken together with the carbon atom to which they are attached, may form a 5- to 6-membered ring which may contain one or more heteroatom(s) selected from N, O and S;
L represents CH₂, C═O, CF₂, CHF, CHOZ′, O, S, SO, SO₂, C═NZ′, or NR″;
   wherein R″ is selected from H, a (C₁-C₆)alkyl and an acyl group;
   wherein Z′ is H, a (C₁-C₆)alkyl, an aryl, amino or OR′″;
   wherein R′″ is H or a (C₁-C₆)alkyl;
R′₂ represents H, halogen, a (C₁-C₅)alkyl, a (C₁-C₃)alkylthio, a nitro group or OZ″;
   wherein Z″ is selected from H, a (C₁-C₅)alkyl, a (C₂-C₅)alkenyl or a (C₁-C₅)alkylsulfonyl;
R′₃ and R′₅ independently represent halogen, an amino, a nitro group, a (C₁-C₅)alkyl, a (C₁-C₃)alkylthio, a (C₁-C₃)alkylamino, or a di(C₁-C₃)alkylamino, SR* or OR*;
   wherein R* is selected from H, a (C₁-C₆)alkyl, an acyl, a (C₁-C₆)alkenyl or a (C₁-C₅)alkylsulfonyl;
R′₄ represents H, halogen, an amino, (C₁-C₅)alkyl, (C₁-C₃)alkylthio, (C₁-C₃)alkylamino, a di(C₁-C₃)alkylamino, heterocyclyl, aryl, heteroaryl, SR** or OR**;
   wherein R** is selected from H, (C₁-C₆)alkyl, an acyl, a (C₁-C₆)alkenyl, an aryl, a cycloalkenyl, a cycloalkyl, a heterocyclyl, or a (C₁-C₅)alkylsulfonyl
R′₄ and R′₅, taken together with the carbon atom to which they are attached, may form a 5- to 6-membered ring which may contain one or more heteroatom(s) selected from N, O and S.

The alkyl, alkenyl, alkoxy, alkoxycarbonylamino, acyl, alkylamino, alkylsulfonyl, alkylthio, aminoacids, aryl, cycloalkenyl, cycloalkyl, dialkylamino or heterocyclyl groups alone or in combination with other groups, being optionally independently substituted by one or more substituents.

The invention also include pharmaceutically acceptable salts, solvate or prodrugs of compounds of formula (I). The invention further includes tautomers, optical and geometrical isomers of compounds of formula (I), and mixtures thereof. The compounds of the present invention may have one or more asymmetric centers and it is intended that stereoisomers (optical isomers), as separated, pure or partially purified stereoisomers or racemic mixtures thereof are included in the scope of the invention.

The present invention also relates to a compound of formula (I) as a medicament, in particular as an anticancer agent or as a vascular disrupting agent.

The present invention also relates to pharmaceutical compositions comprising a compound as defined above in a pharmaceutically acceptable carrier, optionally in association with another active agent.

The compounds and pharmaceutical compositions of the invention are more particularly intended to treat a disease state by inhibiting tubulin polymerisation.

The present invention thus provides a method for treating a disease state by inhibiting tubulin polymerisation, comprising the step of administering a compound of formula (I) to a patient in need thereof.

The present invention also provides the use of a compound of formula (I) for the manufacture of a medicament for the treatment of a disease state by inhibiting tubulin polymerisation.

Further applications and uses of the compounds of the invention, and methods of preparation thereof, are provided in the following detailed description.

### DETAILED DESCRIPTION OF THE INVENTION

Within the context of the present application, the term “alkyl”, alone or in combination with other groups, denotes linear or branched saturated hydrocarbon radical containing preferably from 1 to 10 carbon atoms, in particular from 1 to 6 carbon atoms, unless otherwise indicated. Examples of alkyl groups having from 1 to 6 carbon atoms inclusive are methyl, ethyl, propyl (e.g., n-propyl, iso-propyl), butyl (e.g., tert-butyl, sec-butyl, n-butyl), pentyl (e.g., neo-pentyl), hexyl (e.g., n-hexyl), 2-methylbutyl, 2-methylpentyl and the other isomeric forms thereof.

The term “halogen” denotes a chlorine, bromine, iodine or fluorine atom.

The term “alkoxy” denotes an alkyl-O— group, with alkyl as defined above. Examples of alkoxy groups are methoxy, ethoxy, n-propyloxy, isopropyloxy and sec-butyloxy.

The term “alkoxycarbonylamino” denotes an alkyl-CO—NH group, with alkyl as defined above.

The term “alkylthio” denotes an alkyl-S group, with alkyl as defined above.

The term “alkylamino” denotes an alkyl-NH group, with alkyl as defined above.

The term “dialkylamino” denotes an (alk₁)(alk₂)—N group, wherein alk₁ and alk₂, which are the same or different, represent an alkyl radical as defined above.

The term “alkenyl” denotes linear or branched hydrocarbon groups containing from 2 to 6 carbon atoms, unless otherwise indicated, and containing at least one double bond. Examples of alkenyl containing from 2 to 6 carbon atoms are vinyl, allyl, 1-propenyl, 2-propenyl, 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl and the isomeric forms thereof.

The term “acyl” denotes a —CO-alkyl group, with alkyl as defined above. As an illustrative acyl group, one can cite the acetyl group.

The term “cycloalkyl” denotes an alkyl group that forms one cycle having preferably from 3 to 14 carbon atoms, and more preferably 3 to 8 carbon atoms, such as cyclopropyl, cyclobutyl, cyclopentyl, cyclohexyl, cycloheptyl, and cyclooctyl.

The term “heterocyclyl” refers to a cycloalkyl as indicated above that further comprises one or several heteroatoms selected among nitrogen, oxygen or sulfur. They generally comprise from four to fourteen carbon atoms, such as morpholinyl, piperidinyl, pyrrolidinyl, tetrahydropyranyl, dithiolanyl.

The term “aryl” includes any aromatic group comprising preferably from 5 to 14 carbon atoms, preferably from 6 to 14 carbon atoms, optionally interrupted by one or several heteroatoms selected from N, O, S or P (termed, more specifically, heteroaryl). Most preferred aryl groups are mono- or bi-cyclic and comprises from 6 to 14 carbon atoms, such as phenyl, α-naphtyl, β-naphtyl, antracenyl.

The term “diarylmethylenehydrazynyl” denotes an (aryl₁)(aryl₂)C═N—NH— group, wherein aryl₁ and aryl₂, which are the same or different, represent an aryl radical as defined above. Preferably, both aryl₁ and aryl₂ represent a phenyl group.

The terms “amino acids” include α-aminoacids, β-aminoacids and γ-amino acids. Preferable examples of amino acids include glycine, alanine, leucine, serine, lysine, glutamic acid, aspartic acid, threonine, valine, isoleucine, ornithine, glutamine, asparagine, tyrosine, phenylalanine, cysteine, methionine, arginine, β-alanine, tryptophan, proline, and histidine. Threonine and serine are especially preferred in terms of pharmaceutical effects and safety. These amino acids may be used as L-isomers or D-isomers or a racemic mixture can be employed. L-isomers are preferable.

The present invention relates to a compound of formula (I):

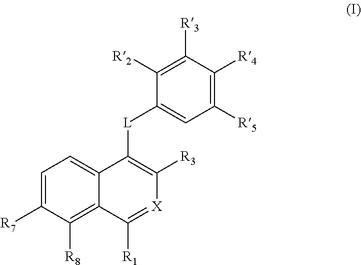

wherein:
X represents N or N⁺—Z, wherein Z is selected in the group consisting of a (C₁-C₆)alkyl, an aryl and an acyl;
R₁ represents H, CN, a CORa or a (C₁-C₅)alkyl;
   wherein Ra represents a NRa′Ra″ or ORa′″;
   wherein Ra′ and Ra″, independently from each other, are selected from the group consisting of H and (C₁-C₅)alkyl;
   wherein Ra′″ represents H or (C₁-C₅)alkyl;
R₃ represents H, CN, OH, a CORb, NH₂ or a (C₁-C₅)alkyl group;
   wherein Rb represents a NRb′Rb″ or ORb′″ group;
   wherein Rb′ and Rb′, independently from each other, are selected from the group consisting of H and (C₁-C₅)alkyl;
   wherein Rb′″ represents H or (C₁-C₅)alkyl;
R₇ represents a (C₁-C₅)alkyl, a (C₁-C₅)alkoxy, a (C₁-C₅)alkylthio, a (C₁-C₅)alkylamino, a (C₁-C₅)dialkylamino;
R₈ represents H, halogen, nitro, CN, N₃, a diarylmethylenehydrazinyl, a di(C₁-C₆)alkyl-N—C═S(O)— group or a di(C₁-C₆)alkyl-N—C═O(S)— group, a (C₁-C₆)alkyl-SO₂, SR, OR or NRR′ group;
   wherein R and R′, independently from each other, are selected from the group consisting of H, a (C₁-C₆)alkyl, a (C₂-C₆)alkenyl, an acyl, an aryl, a heterocyclyl, an amino acid, a Y—SO₂ group, a —P(O)(OG)(OG′) group,
   wherein Y is selected in the group consisting of a hydrogen atom, (C₁-C₆)alkyl, NH₂, (C₁-C₆)alkylamino, di(C₁-C₆)alkylamino and (C₁-C₆)alkoxycarbonylamino;
   wherein G and G′, independently from each other, are selected in the group consisting of H, (C₁-C₆)alkyl and aryl;

R₇ and R₈, taken together with the carbon atom to which they are attached, may form a 5- to 6-membered ring which may contain one or more heteroatom(s) selected from N, O and S;
L represents CH₂, C═O, CF₂, CHF, CHOZ′, O, S, SO, SO₂, C═NZ′, or NR″;
   wherein R″ is selected from H, a (C₁-C₆)alkyl and an acyl group;
   wherein Z′ is H, a (C₁-C₆)alkyl, an aryl, amino or OR′″;
   wherein R′″ is H or a (C₁-C₆)alkyl;
R′₂ represents H, halogen, a (C₁-C₅)alkyl, a (C₁-C₃)alkylthio, a nitro group or OZ″;
   wherein Z″ is selected from H, a (C₁-C₅)alkyl, a (C₂-C₅)alkenyl or a (C₁-C₅)alkylsulfonyl;
R′₃ and R′₅ independently represent halogen, an amino, a nitro group, a (C₁-C₅)alkyl, a (C₁-C₃)alkylthio, a (C₁-C₃)alkylamino, or a di(C₁-C₃)alkylamino, SR* or OR*;
   wherein R* is selected from H, a (C₁-C₆)alkyl, an acyl, a (C₁-C₆)alkenyl or a (C₁-C₅)alkylsulfonyl;
R′₄ represents H, halogen, an amino, (C₁-C₅)alkyl, (C₁-C₃)alkylthio, (C₁-C₃)alkylamino, a di(C₁-C₃)alkylamino, heterocyclyl, aryl, heteroaryl, SR** or OR**;
   wherein R** is selected from H, (C₁-C₆)alkyl, an acyl, a (C₁-C₆)alkenyl, an aryl, a cycloalkenyl, a cycloalkyl, a heterocyclyl, or a (C₁-C₅)alkylsulfonyl
R′₄ and R′₅, taken together with the carbon atom to which they are attached, may form a 5- to 6-membered ring which may contain one or more heteroatom(s) selected from N, O and S.

The alkyl, alkenyl, alkoxy, alkoxycarbonylamino, acyl, alkylamino, alkylsulfonyl, alkylthio, aminoacids, aryl, cycloalkenyl, cycloalkyl, dialkylamino or heterocyclyl groups alone or in combination with other groups, being optionally independently substituted by one or more substituents, which are the same or different, preferably selected in the group consisting of a halogen atom, a (C₁-C₆)alkyl, (C₁-C₆)halogenoalkyl, cycloalkyl, (C₁-C₆)alkenyl, aryl(C₁-C₆)alkyl, aryl, heterocyclyl, heterocyclyl(C₁-C₆)alkyl group, a OH, ═O, NO₂, NH₂, CN, CF₃, CORx, COORy, (C₁-C₆)alkoxy, (di)(C₁-C₆)alkylamino, NHCORx and CONRxRy group, in which Rx and Ry representing a hydrogen atom, a hydroxy, an amino or a (C₁-C₆)alkyl.

In a particular embodiment, preferred substituents of alkyl groups (alone or in combination with another group) are selected in the group consisting of NH₂, halogen (mono or polyhalogenation), cyano, hydroxyl and (C₁-C₆)alkoxy. Illustrative substituted alkyl groups are CH₂NH₂, CF₃, CH₂CN, CH₂OH, CH₂OCH₃.

In a particular embodiment, the present invention relates to a compound of formula (I), wherein:
X represents N or N⁺—Z, wherein Z is selected in the group consisting of a (C₁-C₆)alkyl, an aryl and an acyl;
R₁ represents H, CN, a CORa or a (C₁-C₅)alkyl;
   wherein Ra represents a NRa′Ra″ or ORa′″ group;
   wherein Ra′ and Ra″, independently from each other, are selected from the group consisting of H and (C₁-C₅)alkyl;
   wherein Ra′″ represents H or (C₁-C₅)alkyl;
R₃ represents H, CN, OH, a CORb, NH₂ or a (C₁-C₅)alkyl;
   wherein Rb represents a NRb′Rb″ or ORb′″ group;
   wherein Rb′ and Rb′, independently from each other, are selected from the group consisting of H and (C₁-C₅)alkyl;
   wherein Rb′″ represents H or (C₁-C₅)alkyl;
R₇ represents a (C₁-C₅)alkyl, a (C₁-C₅)alkoxy, a (C₁-C₅)alkylthio, a (C₁-C₅)alkylamino, a (C₁-C₅)dialkylamino;
R₈ represents H, halogen, nitro, a (C₁-C₆)alkyl-SO₂, SR, OR or NRR′ group;
   wherein R and R′, independently from each other, are selected from the group consisting of H, a (C₁-C₆)alkyl, a (C₂-C₆)alkenyl, an acyl, an aryl, a heterocyclyl, an amino acid, a Y—SO₂ group, a P(O)(OG)(OG′) group,
   wherein Y is selected in the group consisting of a hydrogen atom, (C₁-C₆)alkyl, NH₂, (C₁-C₆)alkylamino, di(C₁-C₆)alkylamino and (C₁-C₆)alkoxycarbonylamino;
   wherein G and G′, independently from each other, are selected in the group consisting of H, (C₁-C₆)alkyl and aryl;
L represents CH₂, C═O, CF₂, CHF, CHOZ′, O, S, SO, SO₂, C═NZ′, or NR″;
   wherein R″ is selected from H, a (C₁-C₆)alkyl and an acyl group;
   wherein Z′ is H, a (C₁-C₆)alkyl, an aryl, amino or OR′″;
   wherein R′″ is H or a (C₁-C₆)alkyl;
R′₂ represents H, halogen, a (C₁-C₅)alkyl, a (C₁-C₃)alkylthio, or OZ″;
   wherein Z″ is selected from H, a (C₁-C₅)alkyl, a (C₂-C₅)alkenyl or a (C₁-C₅)alkylsulfonyl;
R′₃ and R′₅ independently represent halogen, an amino, a (C₁-C₅)alkyl, a (C₁-C₃)alkylthio, a (C₁-C₃)alkylamino, or a di(C₁-C₃)alkylamino, SR* or OR*;
   wherein R* is selected from H, a (C₁-C₆)alkyl, an acyl, a (C₁-C₆)alkenyl or a (C₁-C₅)alkylsulfonyl;
R′₄ represents H, halogen, an amino, (C₁-C₅)alkyl, (C₁-C₃)alkylthio, (C₁-C₃)alkylamino, or a di(C₁-C₃)alkylamino, SR** or OR**;
   wherein R** is selected from H, (C₁-C₆)alkyl, an acyl, a (C₁-C₆)alkenyl, an aryl, a cycloalkenyl, a cycloalkyl, a heterocyclyl, or a (C₁-C₅)alkylsulfonyl.

In a particular embodiment, R₈ is a base addition salt of a P(O)(OG)(OG′) group. In particular, R₈ can represent a P(O)O₂Na₂ or a P(O)O₂K₂ group.

Particular compounds of the invention comprise those of formula (I) wherein:
X represents N or N⁺—CH₃; and/or
R₃ represents H; and/or
R₇ represents a hydroxy or an alkoxy, in particular an ethoxy or a methoxy, optionally substituted with a CF₃ group; and/or
R₈ represents H, (C₁-C₆)alkyl-SO₂, OR or NRR′ group; wherein R and R′, independently from each other, are selected from the group consisting of H, a (C₁-C₆)alkyl group, an acyl group, a (C₁-C₆)alkyl-SO₂ group, SO₂NH₂ group, a (C₁-C₆)alkyl-NH—SO₂ group, an alkoxycarbonylamino-SO₂ group, and an amino acid; and/or
R′₄ and R′₅, taken together with the carbon atom to which they are attached, form a 5- or 6-membered ring comprising one or more (e.g. two) oxygen atom(s); and/or
L represents a CH₂ or C═O group.

Other particular compounds of the invention are those of formula (I) wherein:
X represents N; and/or
R₃ represents H; and/or
R₇ represents an alkoxy, in particular an ethoxy or a methoxy; and/or
R₈ represents H, (C₁-C₆)alkyl-SO₂, OR or NRR′ group; wherein R and R′, independently from each other, are selected from the group consisting of H, a (C₁-C₆)alkyl group, an acyl group, a (C₁-C₆)alkyl-SO₂ group, SO₂NH₂ group, a (C₁-C₆)alkyl-NH—SO₂ group, an alkoxycarbonylamino-SO₂ group, and an amino acid; and/or
L represents a CH₂ or C═O group.

In a further particular embodiment, R₇, R′₃, R′₄ and R′₅ independently represent a (C₁-C₆)alkoxy group, in particular a (C₁-C₄)alkoxy group;

In another specific embodiment, R′₅ is a methoxy group.

In a particular embodiment, when R₈ is a OR or NRR′ group, and one of R and R′ represent an amino acid, said amino acid is preferably linked to the O or N atom of the OR or NRR′ group via its carboxyl end to form a O—CO—CH(Res)-NH₂ or a NH—CO—CH(Res)-NH₂ group, respectively, where Res represents the amino acid residue. In a particular embodiment, R or R′ represent a serine, a glycine or a phenylalanine. In a variant of this embodiment, R₈ represents a NRR′ group wherein R is hydrogen and R′ is a serine (see for example compound 26), a glycine (see for example compound 112) or a phenylalanine (see for example compound 114).

In another embodiment, R′₃ and R′₅, which can be the same or different, both represent a (C₁-C₆)alkoxy (i.e., both represent an OR* group wherein R* is a (C₁-C₆)alkyl) or a halogen atom.

In a particular embodiment, advantageous compounds of the invention are those of formula (I) wherein:
X represents N;
R₁ represents H, CN or CNCH₂;
R₃ represents H;
R₇ represents an ethoxy or methoxy;
R₈ represents H, CH₃SO₂, OH, NH₂, CH₃O, CNCH₂O, a CF₃SO₃, NH₂SO₃, CH₃CONH, CH₃SO₂NH, NH₂SO₂NH, serine-NH glycine-NH, phenylalanine-NH (via the carboxyl group of serine, glycine or phenylalanine to form an amide with the NH) or a tert-butyloxycarbonylaminosulfonylamino group;
L represents a CH₂ or C═O group;
R′₂ represents H or bromine;
R′₃ and R′₅ independently from each other represent F, a methoxy or an ethoxy group; and
R′₄ represents H, F, a hydroxyl group, a methoxy, ethoxy, n-propyloxy, sec-butyloxy or CH₃OCH₂CH₂O group.

In a further embodiment, the invention relates to compounds of formula (I) with at least one, preferably all, the following features:
X represents N;
R₁ represents H;
R₃ represents H;
R₇ represents a methoxy or an ethoxy;
R₈ represents H, OH, NH₂, methoxy, NH₂SO₃, NH₂SO₂NH, CH₃COO, OPO₃²⁻, NH₂CH₂CONH;
L represents a CH₂ or C═O group;
R′₂ represents H;
R′₃ represents a methoxy or halogen atom, in particular a bromide or iodine atom;
R′₄ represents a methoxy, ethoxy, n-propyloxy, isobutyloxy, isopentyloxy or allyloxy group;
R′₅ represents a methoxy group.

In another particular embodiment of the invention, the compounds of formula (I) are those wherein:
X represents N;
L represents a CH₂ or CO group;
R₁, R₃ and R′₂ represent H;
R₇, R′₃, R′₄ and R′₅ independently represent a (C₁-C₆)alkoxy group, in particular a (C₁-C₄)alkoxy group;
R₈ represents a NH₂, OH or NH₂SO₃⁻ group.

In another particular embodiment, the invention relates to compounds of formula (I) wherein:
X represents N;
L represents a CH₂ or CO group;
R₁, R₃ and R′₂ represent H;
R₇ represents an ethoxy group;
R′₃ represents a methoxy or ethoxy group;
R′₄ represents a methoxy, ethoxy or n-propyloxy group;
R′₅ represents a methoxy group;
R₈ represents a NH₂, OH or NH₂SO₃⁻ group.

In a further particular embodiment, the invention relates to compounds of formula (I) wherein R′₃ and R′₅ are identical. In one variant of this embodiment, R′₃ and R′₅ are identical and both represent an halogen atom, for example a iodine, a bromine or a fluorine atom, or a methoxy group. In a particular embodiment, both R′₃ and R′₅ represent a methoxy group.

In another embodiment, the invention relates to compounds of formula (I) wherein R′₃ and R′₅ are different. In one variant of this embodiment, R′₃ represents an ethoxy and R′₅ represents a methoxy.

In a further embodiment, the invention relates to a compound of formula (I) wherein R₈ represents a NRR″ group, in particular a NH₂ group.

In a particular embodiment, the compounds of the invention are those of formula (I) wherein R₈ represents an OR group, in particular an OH group.

Other particular compounds of the invention are those of formula (I) wherein R′₄ is an alkoxy group, in particular a methoxy, ethoxy, n-propyloxy, sec-butyloxy or CH₃OCH₂CH₂O group (i.e., an alkoxy group substituted with an alkoxy group).

In a particular embodiment, compounds of the invention are those of formula (I) wherein R′₃, R′₄ and R′₅ represent a halogen atom, preferably all three representing a fluorine atom, or a (C₁-C₆)alkoxy. In a specific variant of this embodiment, R′₃ and R′₅ represent a methoxy group and R′₄ represents a methoxy or ethoxy group, in particular a methoxy group.

Specific examples of compounds of formula (I) which fall within the scope of the present invention include the following compounds:
4-(3,4,5-Trimethoxybenzyl)-7-ethoxyisoquinolin-8-ol hydrochloride 1,
7-ethoxy-4-(3,4,5-trimethoxybenzyl)isoquinolin-8-yl trifluoromethanesulfonate hydrochloride 2,
7-ethoxy-4-(3,4,5-trimethoxybenzyl)isoquinolin-8-amine dihydrochloride 3,
N-(7-ethoxy-4-(3,4,5-trimethoxybenzyl)isoquinolin-8-yl)acetamide hydrochloride 4,
N-(7-ethoxy-4-(3,4,5-trimethoxybenzyl)isoquinolin-8-yl)methanesulfonamide hydrochloride 5,
7-ethoxy-8-(methylsulfonyl)-4-(3,4,5-trimethoxybenzyl)isoquinoline hydrochloride 6,
tert-butyl N-(7-ethoxy-4-(3,4,5-trimethoxybenzyl)isoquinolin-8-yl)sulfamoylcarbamate 7,
7-ethoxy-4-(3,4,5-trimethoxy-benzyl)isoquinolin-8-yl]-sulfamide hydrochloride 8,
4-(4-ethoxy-3,5-dimethoxybenzyl)-7,8-dimethoxyisoquinoline hydrochloride 9,
4-(3,4,5-trimethoxybenzyl)-7,8-dimethoxyisoquinoline hydrochloride 10,
2,6-dimethoxy-4-((7,8-dimethoxyisoquinolin-4-yl)methyl)phenol hydrochloride 11,
7-ethoxy-4-(3,4,5-trifluorobenzyl)isoquinolin-8-ol hydrochloride 12,
7-ethoxy-4-(4-ethoxy-3,5-dimethoxybenzyl)isoquinoline hydrochloride 13,
4-(4-ethoxy-3,5-dimethoxybenzyl)-7-ethoxyisoquinolin-8-ol hydrochloride 14,
4-(3-ethoxy-4,5-dimethoxybenzyl)-7-ethoxyisoquinolin-8-ol hydrochloride 15,
4-(3,4,5-trimethoxybenzyl)-7-ethoxyisoquinoline hydrochloride 16,
4-(2-bromo-3,5-dimethoxybenzyl)-7-ethoxyisoquinolin-8-ol hydrochloride 17,
4-(4-ethoxy-3,5-dimethoxybenzyl)-7-ethoxyisoquinolin-8-amine dihydrochloride 18,
4-(3,5-dimethoxy-4-propoxybenzyl)-7-ethoxyisoquinolin-8-ol hydrochloride 19,
4-(4-isobutoxy-3,5-dimethoxybenzyl)-7-ethoxyisoquinolin-8-ol hydrochloride 20,
4-(3,4,5-trimethoxybenzyl)-7-ethoxyisoquinolin-8-yl sulfamate hydrochloride 21,
4-(4-(2-methoxyethoxy)-3,5-dimethoxybenzyl)-7-ethoxyisoquinolin-8-ol hydrochloride 22,
7-ethoxy-4-(4-ethoxy-3,5-dimethoxybenzyl)isoquinoline-1-carbonitrile 23,
2-(4-(3,4,5-trimethoxybenzyl)-7-ethoxyisoquinolin-8-yloxy)acetonitrile 24,
(4-(4-ethoxy-3,5-dimethoxybenzyl)-7-ethoxyisoquinolin-1-yl)methanamine dihydrochloride 25,
(S)-2-amino-N-(7-ethoxy-4-(3,4,5-trimethoxybenzyl)isoquinolin-8-yl)-3-hydroxypropanamide dihydrochloride 26,
4-(3,4,5-trimethoxybenzyl)-7-methoxyisoquinoline hydrochloride 27,
(7-methoxyisoquinolin-4-yl)(3,4,5-trimethoxyphenyl)methanone hydrochloride 28,
(8-amino-7-ethoxyisoquinolin-4-yl)(3,4,5-trimethoxyphenyl)methanone dihydrochloride 29,
4-(3,5-dimethoxy-4-(2,2,2-trifluoroethoxy)benzyl)-7-ethoxyisoquinolin-8-ol hydrochloride 30,
4-(4-isobutoxy-3,5-dimethoxybenzyl)-7-ethoxyisoquinolin-8-amine dihydrochloride 31,
4-(4-(allyloxy)-3,5-dimethoxybenzyl)-7-ethoxyisoquinolin-8-ol hydrochloride 32,
7-ethoxy-4-(4-(isopentyloxy)-3,5-dimethoxybenzyl)isoquinolin-8-ol hydrochloride 33,
4-(4-(cyclobutylmethoxy)-3,5-dimethoxybenzyl)-7-ethoxyisoquinolin-8-ol hydrochloride 34,
4-(4-(2-cyclohexylethoxy)-3,5-dimethoxybenzyl)-7-ethoxyisoquinolin-8-ol hydrochloride 35,
4-(4-(cyclohexylmethoxy)-3,5-dimethoxybenzyl)-7-ethoxyisoquinolin-8-ol hydrochloride 36,
7-ethoxy-4-(4-(2-ethylbutoxy)-3,5-dimethoxybenzyl)isoquinolin-8-ol hydrochloride 37,
4-(4-butoxy-3,5-dimethoxybenzyl)-7-ethoxyisoquinolin-8-ol hydrochloride 38,
4-(3,5-dimethoxy-4-(neopentyloxy)benzyl)-7-ethoxyisoquinolin-8-ol hydrochloride 39,
(7-ethoxyisoquinolin-4-yl)(3,4,5-trimethoxyphenyl)methanone hydrochloride 40,
(7-ethoxyisoquinolin-4-yl)(3,4,5-trimethoxyphenyl)methanone oxime hydrochloride 41,
(7-ethoxyisoquinolin-4-yl)(3,4,5-trimethoxyphenyl)methanethione hydrochloride 42,
(7-ethoxyisoquinolin-4-yl)(3,4,5-trimethoxyphenyl)methanol hydrochloride hydrochloride 43,
4-(3,5-dimethoxy-4-(3-phenylpropoxy)benzyl)-7-ethoxyisoquinolin-8-ol hydrochloride 44,
4-(3,5-dimethoxy-4-phenethoxybenzyl)-7-ethoxyisoquinolin-8-ol hydrochloride 45,
7-ethoxy-4-(4-ethoxy-3,5-dimethoxybenzyl)-8-methoxyisoquinoline hydrochloride 46,
(4-ethoxy-3,5-dimethoxyphenyl)-(7-ethoxy-8-methoxyisoquinolin-4-yl)-methanone hydrochloride 47,
methyl 2-(4-((7-ethoxy-8-hydroxyisoquinolin-4-yl)methyl)-2,6-dimethoxyphenoxy)acetate hydrochloride 48,
2-(4-((7-ethoxy-8-hydroxyisoquinolin-4-yl)methyl)-2,6-dimethoxyphenoxy)acetamide hydrochloride 49,
2-(4-((7-ethoxy-8-hydroxyisoquinolin-4-yl)methyl)-2,6-dimethoxyphenoxy)acetonitrile hydrochloride 50,
4-(4-(benzylamino)-3,5-dimethoxybenzyl)-7-ethoxyisoquinolin-8-ol hydrochloride 51,
4-(3,5-dimethoxy-4-(phenylamino)benzyl)-7-ethoxyisoquinolin-8-ol hydrochloride 52,
4-((2,6-dimethoxy-[1,1′-biphenyl]-4-yl)methyl)-7-ethoxyisoquinolin-8-ol hydrochloride 53,
(7-methoxyisoquinolin-4-yl)(3,4,5-trimethoxy-2-nitrophenyl)methanone hydrochloride 54,
(8-(benzyloxy)-7-ethoxyisoquinolin-4-yl)(4-ethoxy-3,5-dimethoxyphenyl)methanone 55,
(4-ethoxy-3,5-dimethoxyphenyl)(7-ethoxy-8-hydroxyisoquinolin-4-yl)methanone hydrochloride 56,
7-ethoxy-4-(4-ethoxy-3,5-dimethoxybenzoyl)isoquinolin-8-yl sulfamate 57,
(8-amino-7-ethoxyisoquinolin-4-yl)(4-ethoxy-3,5-dimethoxyphenyl)methanone hydrochloride 58,
7-ethoxy-4-(4-ethoxy-3,5-dimethoxybenzoyl)isoquinolin-8-yl sulfamide 59,
8-(2-(diphenylmethylene)hydrazinyl)-7-ethoxy-4-(4-ethoxy-3,5-dimethoxybenzyl)isoquinoline hydrochloride 60,
7-ethoxy-4-((8-methoxy-2,3-dihydrobenzo[b][1,4]dioxin-6-yl)methyl)isoquinolin-8-ol hydrochloride 61,
7-ethoxy-4-((7-methoxybenzo[d][1,3]dioxol-5-yl)methyl)isoquinolin-8-ol hydrochloride 62,
4-(3,5-dimethoxy-4-(pentyloxy)benzyl)-7-ethoxyisoquinolin-8-ol hydrochloride 63,
4-(3,5-dimethoxy-4-propoxybenzyl)-7-ethoxyisoquinolin-8-amine dihydrochloride 64,
4-(3,5-Dimethoxy-4-propoxybenzoyl)-7-ethoxyisoquinolin-8-yl acetate 65,
(8-amino-7-ethoxyisoquinolin-4-yl)(3,5-dimethoxy-4-propoxyphenyl)methanone hydrochloride 66,
(3,5-dimethoxy-4-propoxyphenyl)(7-ethoxy-8-hydroxyisoquinolin-4-yl)methanone hydrochloride 67,
O-(4-(3,5-dimethoxy-4-propoxybenzyl)-7-ethoxyisoquinolin-8-yl) dimethylcarbamothioate hydrochloride 68,
4-(3,5-dichloro-4-methoxybenzyl)-7-ethoxyisoquinolin-8-ol hydrochloride 69,
4-(3,5-diiodo-4-methoxybenzyl)-7-ethoxyisoquinolin-8-ol hydrochloride 70,
4-(3-bromo-4,5-dimethoxybenzyl)-7-ethoxyisoquinolin-8-ol hydrochloride 71,
7-ethoxy-4-(3-iodo-4,5-dimethoxybenzyl)isoquinolin-8-ol hydrochloride 72,
7-ethoxy-4-(4-ethyl-3,5-dimethoxybenzyl)isoquinolin-8-ol hydrochloride 73,
7-ethoxy-4-(4-ethoxy-3-methoxy-5-nitrobenzyl)isoquinolin-8-ol hydrochloride 74,
4-(3-amino-4-ethoxy-5-methoxybenzyl)-7-ethoxyisoquinolin-8-ol hydrochloride 75,
4-(3,5-dimethoxy-4-(pyrrolidin-1-yl)benzyl)-7-ethoxyisoquinolin-8-ol hydrochloride 76,
4-(3-bromo-4-ethoxy-5-methoxybenzyl)-7-ethoxyisoquinolin-8-ol hydrochloride 77,
7-ethoxy-4-(4-isobutoxy-3,5-dimethoxybenzoyl)isoquinolin-8-yl acetate 78,
(7-ethoxy-8-hydroxyisoquinolin-4-yl)(4-isobutoxy-3,5-dimethoxyphenyl)methanone hydrochloride 79,
6-(3,5-dimethoxy-4-propoxybenzyl)-3-ethyloxazolo[4,5-h]isoquinolin-2(3H)-one hydrochloride 80,
6-(3,5-dimethoxy-4-propoxybenzoyl)-3-ethyloxazolo[4,5-h]isoquinolin-2(3H)-one hydrochloride 81,
S-(4-(3,5-dimethoxy-4-propoxybenzyl)-7-ethoxyisoquinolin-8-yl) dimethylcarbamothioate hydrochloride 82,
S-(4-(3,5-dimethoxy-4-propoxybenzoyl)-7-ethoxyisoquinolin-8-yl) dimethylcarbamothioate hydrochloride 83,
(3,5-dimethoxy-4-propoxyphenyl)(7-ethoxy-8-mercaptoisoquinolin-4-yl)methanone hydrochloride 84,
7-ethoxy-4-(3,4,5-trimethoxybenzyl)isoquinoline-8-carbonitrile hydrochloride 85,
7-ethoxy-4-(3-isobutoxy-4,5-dimethoxybenzyl)isoquinolin-8-ol hydrochloride 86,
4-(3,4-dimethoxy-5-(2,2,2-trifluoroethoxy)benzyl)-7-ethoxyisoquinolin-8-ol hydrochloride 87,
8-amino-7-ethoxy-2-methyl-4-(3,4,5-trimethoxybenzyl)isoquinolinium chloride 88,
7-ethoxy-N-ethyl-4-(3,4,5-trimethoxybenzyl)isoquinolin-8-amine dihydrochloride 89,
7-ethoxy-N-(2,2,2-trifluoroethyl)-4-(3,4,5-trimethoxybenzyl)isoquinolin-8-amine dihydrochloride 90,
7-ethoxy-N,N-dimethyl-4-(3,4,5-trimethoxybenzyl)isoquinolin-8-amine dihydrochloride 91,
7-ethoxy-N-methyl-4-(3,4,5-trimethoxybenzyl)isoquinolin-8-amine dihydrochloride 92,
methyl 2-(7-ethoxy-4-(3,4,5-trimethoxybenzyl)isoquinolin-8-ylamino)acetate dihydrochloride 93,
7-ethoxy-8-iodo-4-(3,4,5-trimethoxybenzyl)isoquinoline hydrochloride 94,
7-ethoxy-4-(1-(3,4,5-trimethoxyphenyl)vinyl)isoquinolin-8-ol hydrochloride 95,
7-ethoxy-4-(3,4,5-trimethoxybenzoyl)isoquinolin-8-yl acetate 96,
(7-ethoxy-8-hydroxyisoquinolin-4-yl)(3,4,5-trimethoxyphenyl)methanone hydrochloride 97,
7-ethoxy-4-(3,4,5-trimethoxybenzoyl)isoquinolin-8-yl disodium phosphate 98,
7-(2,2,2-trifluoroethoxy)-4-(3,4,5-trimethoxybenzyl)isoquinolin-8-ol hydrochloride 99,
4-(3,4,5-trimethoxybenzyl)isoquinoline-7,8-diol hydrochloride 100,
4-(3,4,5-trimethoxybenzyl)isoquinolin-7-ol hydrochloride 101,
7-ethoxy-4-(4-hydroxy-3-methoxy-5-nitrobenzyl)isoquinolin-8-ol hydrochloride 102,
4-(3,4-dimethoxy-5-nitrobenzyl)-7-ethoxyisoquinolin-8-ol hydrochloride 103,
tert-butyl N-(7-ethoxy-4-(3,4,5-trimethoxybenzoyl)isoquinolin-8-yl)sulfamoylcarbamate 104,
N-(7-ethoxy-4-(3,4,5-trimethoxybenzoyl)isoquinolin-8-yl)sulfamide hydrochloride 105,
8-chloro-7-ethoxy-4-(3,4,5-trimethoxybenzyl)isoquinoline hydrochloride 106,
8-azido-7-ethoxy-4-(3,4,5-trimethoxybenzyl)isoquinoline 107,
8-azido-7-ethoxy-4-(3,4,5-trimethoxybenzoyl)isoquinoline 108,
7-ethoxy-4-(3,4,5-trimethoxybenzoyl)isoquinolin-8-yl pivalate 109,
4-(2-chloro-3,4,5-trimethoxybenzyl)-7-ethoxyisoquinolin-8-ol hydrochloride 110,
N-(7-ethoxy-4-(3,4,5-trimethoxybenzoyl)isoquinolin-8-yl)acetamide hydrochloride 111,
2-amino-N-(7-ethoxy-4-(3,4,5-trimethoxybenzoyl)isoquinolin-8-yl)acetamide 112,
isobutyl (7-ethoxy-4-(3,4,5-trimethoxybenzoyl)isoquinolin-8-yl)carbamate hydrochloride 113,
(S)-2-amino-N-(7-ethoxy-4-(3,4,5-trimethoxybenzoyl)isoquinolin-8-yl)-3-phenylpropanamide dihydrochloride 114,
4-(3-bromo-4,5-dimethoxybenzoyl)-7-ethoxyisoquinolin-8-yl acetate 115,
(3-bromo-4,5-dimethoxyphenyl)(7-ethoxy-8-hydroxyisoquinolin-4-yl)methanone hydrochloride 116.

In a particular embodiment, the invention relates to a compound selected in the group consisting of compounds 1, 3, 10, 14, 15, 18, 19, 20, 21, 29, 31, 32, 33, 38, 40, 56, 57, 58, 59, 64, 65, 66, 67, 78, 96, 97, 98, 105 and 112. In particular, the invention relates to a compound selected in the group consisting of compounds 3, 15, 29, 57, 58, 64, 66, 67 and 97.

The compounds according to the present invention may be prepared by various methods known to those skilled in the art. More preferably, the following chemical routes were carried out.

The compounds 1, 10, 16, 21, 24, 27, 28, 40-43 and 54 were obtained following the synthetic route described in scheme 1:

2,3-Disubstituted benzaldehydes were treated in toluene at reflux using a Dean-Stark apparatus by aminoacetaldehyde diethylacetal to obtain in 95 to 100% yields the corresponding E imines (LPO 26046, ECO 33112, LPO 30168 and SAO 33012) that were subsequently reduced using sodium borohydride in methanol at reflux to obtain in 90 to 95% yields the corresponding N-(2,3-disubstituted-benzyl)-2,2-diethoxyethanamines (LPO 26048, ECO 33116, LPO 30170 and SAO 33014 respectively). Reaction of 3,4,5-trimethoxybenzaldehyde with 2,2-diethoxyamines (LPO 26048, ECO 33116, LPO 30170 and SAO 33014) using a 37% HCl solution in ethanol at reflux led to the 7,8-disubstituted-4-(3,4,5-trimethoxybenzyl)isoquinolines hydrochlorides (10, 27, 16, 1 respectively) in 18 to 33% yields after final HCl methanolic treatment. Phenol 1 reacted at room temperature with sulfamoyl chloride in presence of triethylamine to give in 35% yield the corresponding sulphamate hydrochloride 21 after final HCl methanolic treatment. Phenol 1 free base (CCH 34046-2) also reacted with 2-chloroacetonitrile in presence of cesium carbonate in dimethylformamide at 90° C. to afford 2-(7-ethoxy-4-(3,4,5-trimethoxybenzyl)isoquinolin-8-yloxy)acetonitrile hydrochloride 24 in 70% yield after final HCl methanolic treatment. Finally compound 27 can undergo a benzylic oxidation using 70% HNO₃ in presence of acetic anhydride at room temperature to led to (7-methoxyisoquinolin-4-yl)(3,4,5-trimethoxyphenyl)methanone hydrochloride 28 in 45% yield after final HCl methanolic treatment.

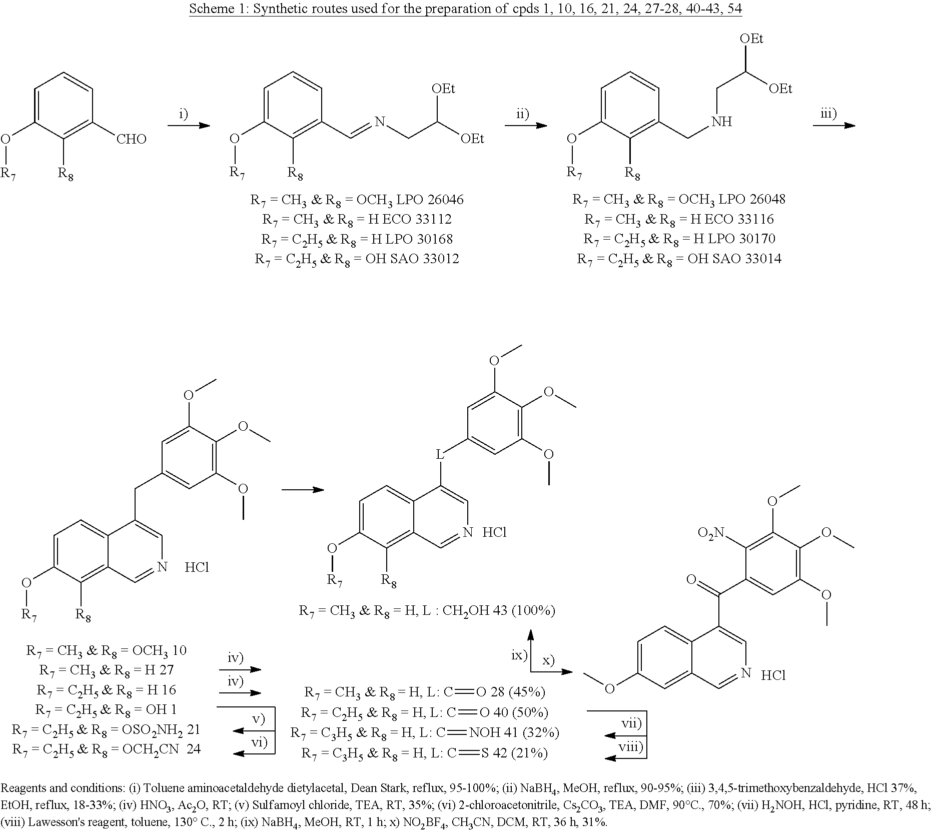

The compounds 40-42 were obtained from compound 16 (scheme 1): Oxidation of compound 16 by nitric acid in presence of acetic anhydride at room temperature gave the keto derivative 40 as a hydrochloride salt in 50% yield after final HCl methanolic treatment. Compound 40 was treated with hydroxylamine hydrochloride in pyridine to yield in 32% yield the corresponding oxime 41 as a hydrochloride salt after final HCl methanolic treatment. Finally keto compound 40 was transformed into its corresponding thio derivative 42 as a hydrochloride salt in 21% yield, by treatment with Lawesson's reagent in refluxing toluene for 2 hours followed by a final HCl methanolic treatment.

The compounds 43 and 54 were obtained from the keto compound 28 (scheme 1): The hydroxy derivative 43 was obtained quantitatively as an hydrochloride salt by reduction using sodium borohydride in methanol followed by a final HCl methanolic treatment. The nitro compound 54 was obtained in 31% yield as an hydrochloride salt by treatment, at room temperature for 36 hours, by nitronium tetrafluoroborate in a mixture of acetonitrile and dichloromethane followed by a final HCl methanolic treatment.

The compounds 2 to 8 were obtained following the synthetic routes described in scheme 2: Reductive amination between 3-ethoxy-2-hydroxybenzaldehyde and 2,2-diethoxyethanamine using sodium cyanoborohydride in MeOH and acetic acid overnight at room temperature gave 2-((2,2-diethoxyethylamino)methyl)-6-ethoxyphenol SAO 33014 in 70% yield. Reaction of 3,4,5-trimethoxybenzaldehyde with SAO 33014, using a 37% HCl solution in ethanol at 100° C. under microwave irradiation, led to 7-ethoxy-4-(3,4,5-trimethoxybenzyl)isoquinolin-8-ol CCH 34046-2 in 30% yield. Phenol CCH 34046-2 reacted for 20 min at room temperature with N-phenyl-bis(trifluoromethanesulfonimide) in presence of triethylamine in DMF to give triflate CCH 34050 that can be converted by methanolic HCl treatment in hydrochloride salt 2. Palladium-catalyzed amination (Buchwald-Hartwig reaction) for 5 hours at 150° C. under microwave irradiation between triflate CCH 34050 and benzophenone imine, using a catalyst consisting of a combination of tris(dibenzylideneacetone)dipalladium(0) and BINAP in presence of cesium carbonate in dry toluene, afforded after 1 N HCl treatment in methanol, to 7-ethoxy-4-(3,4,5-trimethoxybenzyl)isoquinolin-8-amine dihydrochloride 3 in 64% yield.

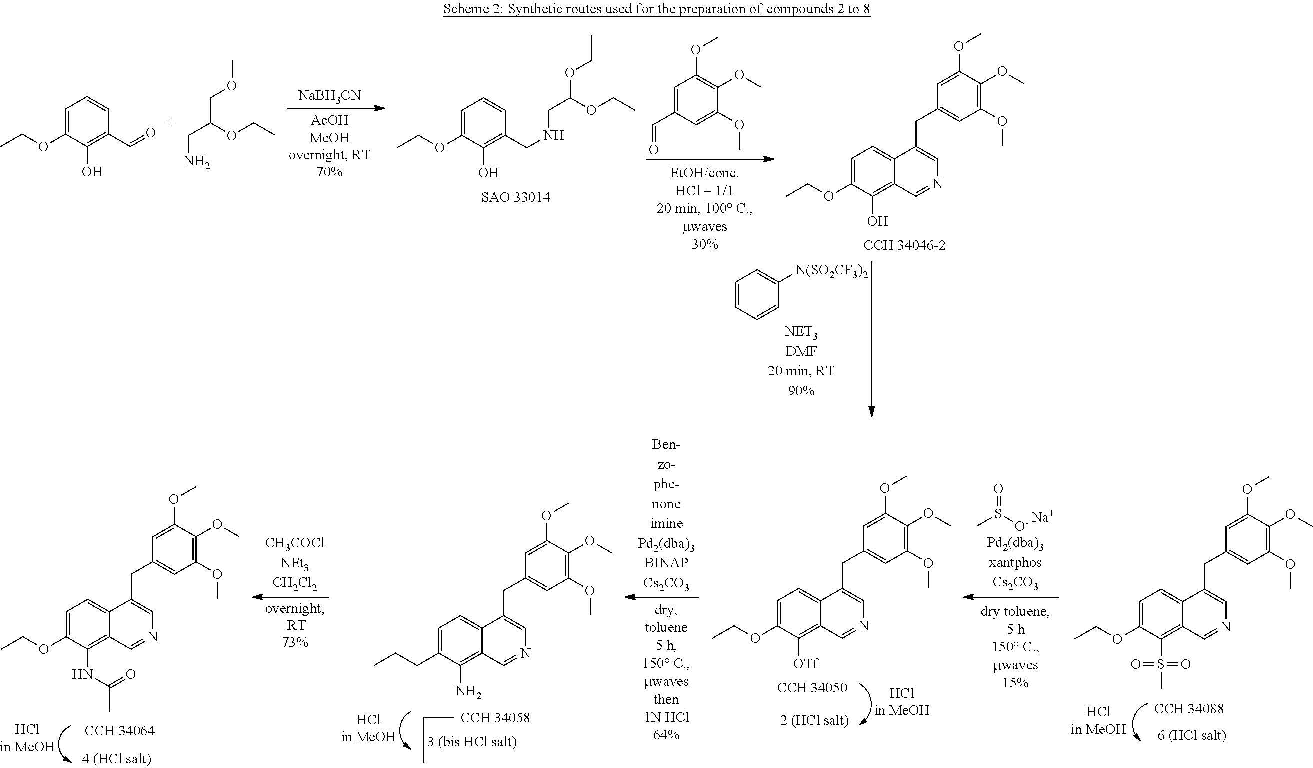

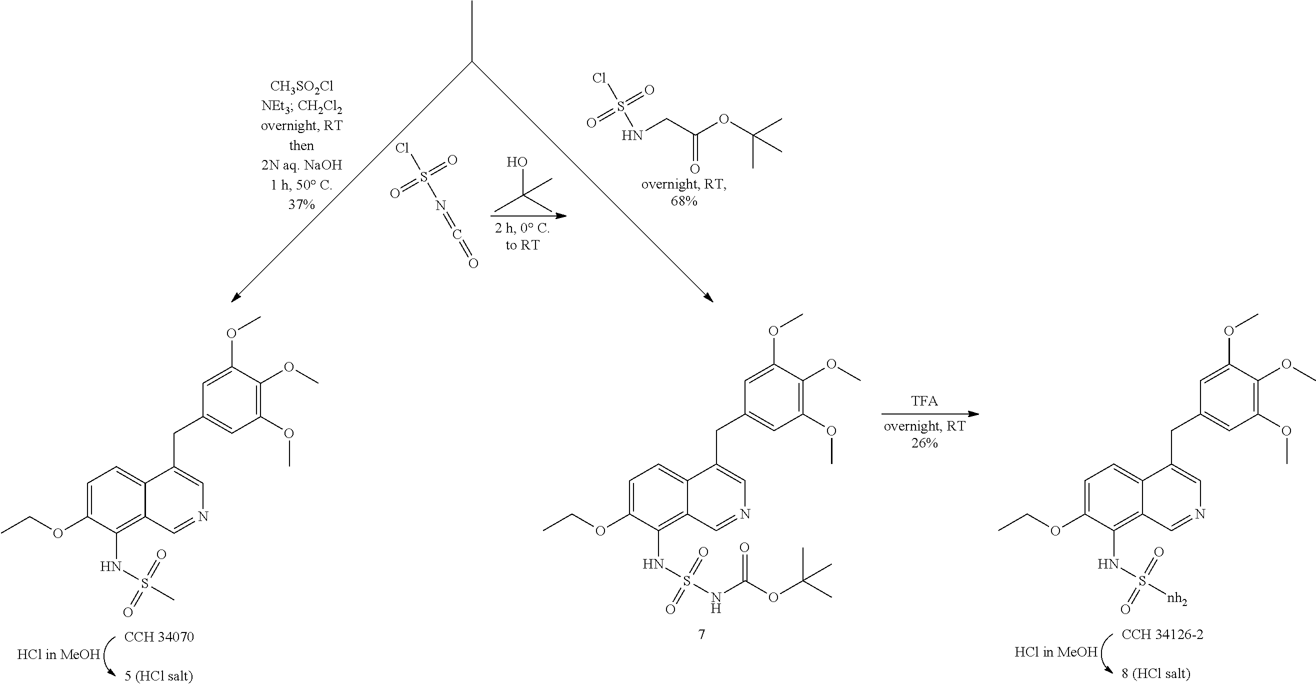

Compound 3 free base (CCH 34058) reacted overnight at room temperature with acetyl chloride in presence of Et₃N in dichloromethane to give N-acetate CCH 34064 that was transformed in 73% yield into its hydrochloride salt 4 upon treatment with a solution of 0.22 N HCl in methanol. Similarly compound 3 free base (CCH 34058) reacted overnight at room temperature with methanesulfonic chloride in presence of Et₃N in dichloromethane to yield, after treatment for 1 hour at 50° C. with an aqueous 2 N NaOH solution, to the N-acetate CCH 34064 that was finally transformed into its hydrochloride salt in 37% yield, upon treatment with a solution of 0.22 N HCl in methanol. Compound 3 free base (CCH 34058) also reacted overnight at room temperature with a solution of tert-butyl chlorosulfonylcarbamate in THF (prepared from chlorosulfonyl isocyanate in dry THF that was reacted at 0° C. with tert-BuOH and the resulting mixture stirred for 2 hours at room temperature) to obtain tert-butyl N-(7-ethoxy-4-(3,4,5-trimethoxybenzyl)isoquinolin-8-yl)sulfamoylcarbamate 7 in 68% yield. tert-Butyl sulfamoylcarbamate 7 was deprotected using trifluoroacetic acid overnight at room temperature to afford to the corresponding N-sulfamide CCH 34126-2 that was transformed into its hydrochloride salt 8 in 26% yield upon treatment with a solution of 0.22 N HCl in methanol.

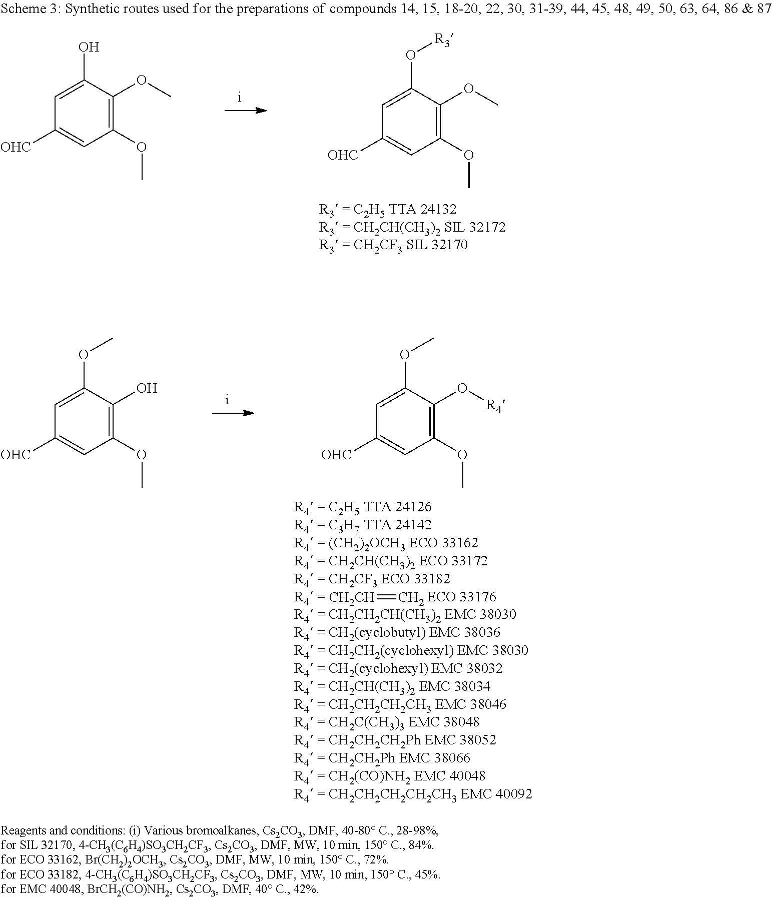

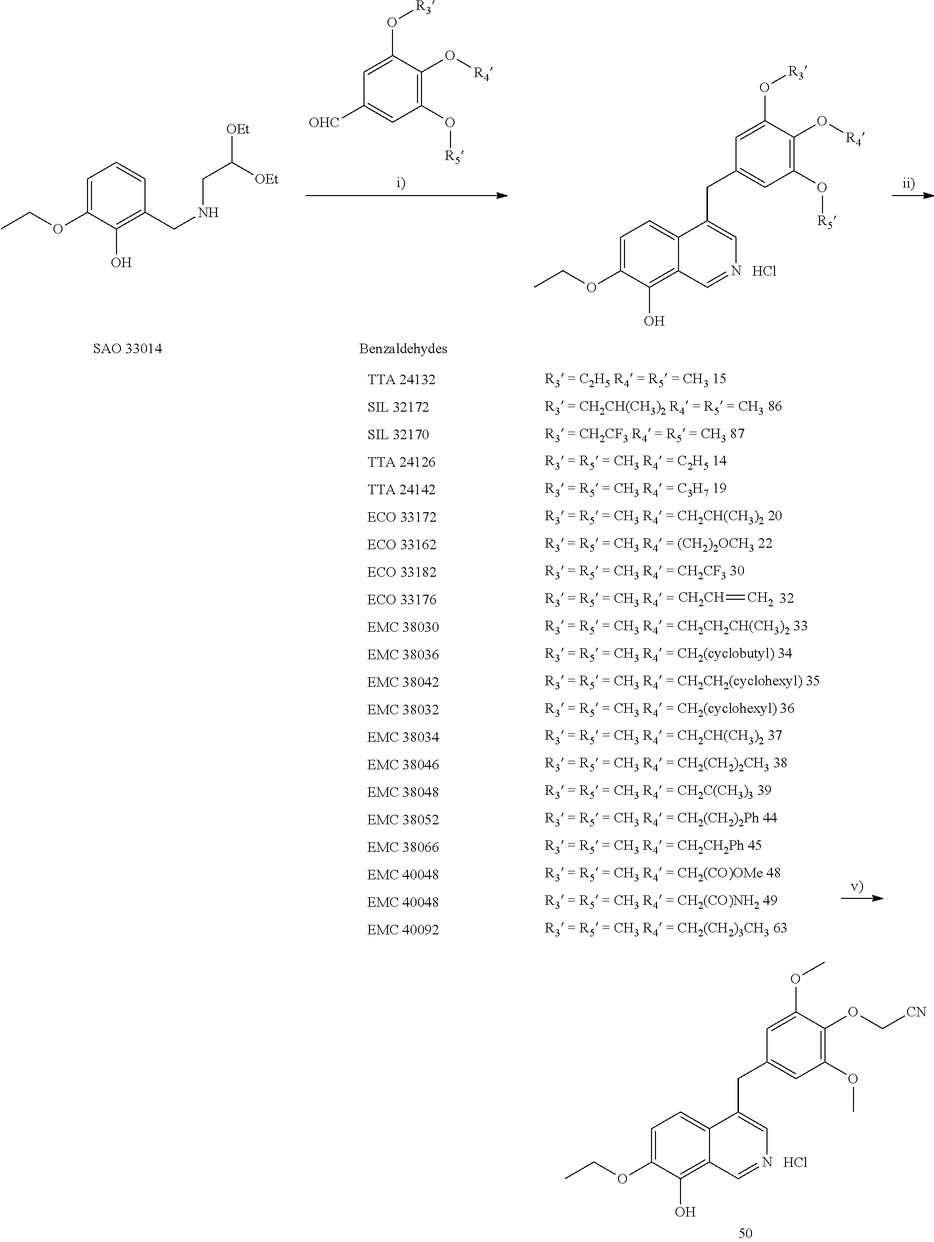

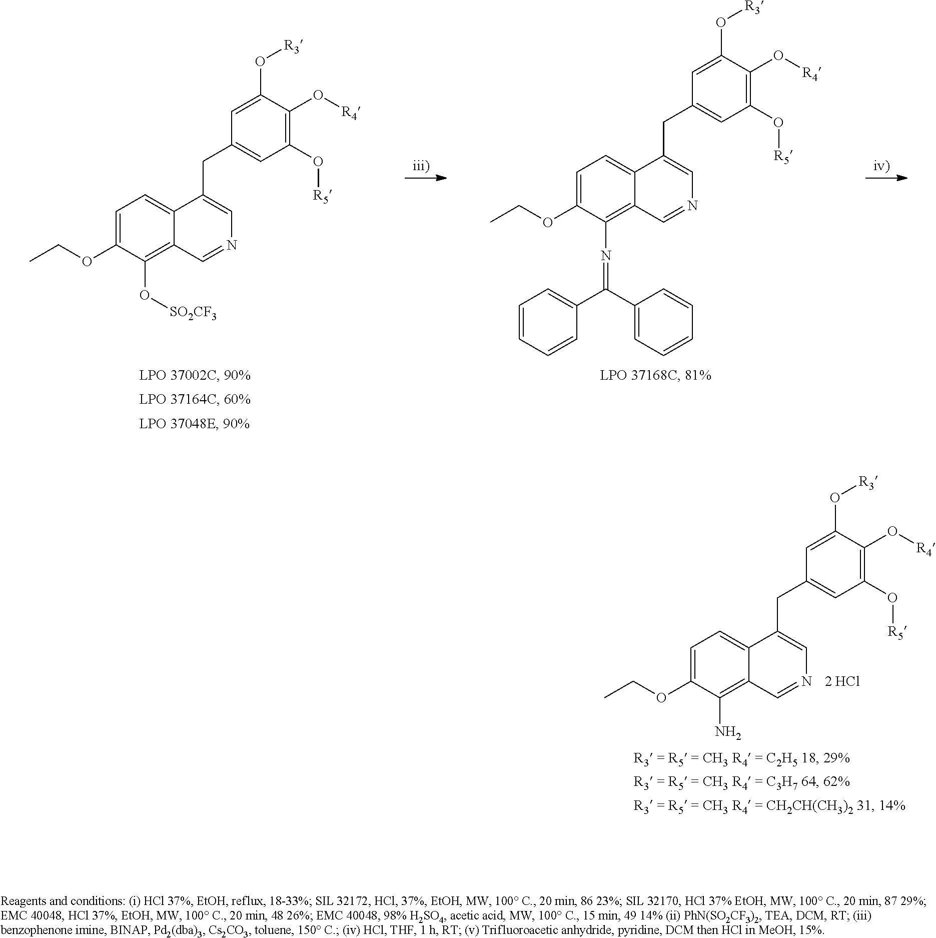

The compounds 14, 15, 18-20, 22, 30, 31-39, 44, 45, 48, 49, 50, 63, 64, 86 & 87 were obtained following the synthetic routes described in scheme 3: 3,4,5-trialkoxybenzaldehydes were prepared as described in scheme 3 by O-alkylation of 3-hydroxy-4,5-dimethoxybenzaldehyde or by O-alkylation of 4-hydroxy-3,5-dimethoxybenzaldehyde by the corresponding bromoalkanes or alkylating agents in DMF using cesium carbonate as base, and were reacted with 2-((2,2-diethoxyethylamino)methyl)-6-ethoxyphenol SAO 33014 in a 37% HCl solution in ethanol at reflux to obtain compounds 15, 14, 19, 20, 22, 30, 32-39, 44, 45, 48, 49, 50, 63, 86 & 87 as hydrochloride salts upon a final HCl methanolic treatment. The free bases of phenols 14, 19 or 20 reacted at room temperature with N-phenyl-bis(trifluoromethanesulfonimide) in presence of triethylamine in DMF to give the corresponding triflates LPO 37002C, LPO 37164C or LPO 37048E in 60-90% yields. Palladium-catalyzed amination (Buchwald-Hartwig reaction) at 150° C. under microwave irradiation between triflates LPO 37002C, LPO 37164C or LPO 37048E and benzophenone imine, using a catalyst consisting of a combination of tris(dibenzylideneacetone)dipalladium(0) and BINAP in presence of cesium carbonate in dry toluene, afforded to imine intermediates that gave, after hydrolysis with HCl in THF and a final 1 N HCl treatment in methanol, the amino derivatives 18, 31 or 64 as dihydrochloride salts in 14%-62% yields.

The cyano compound 50 (hydrochloride salt) was obtained in 15% yield from amide 49 by treatment, at room temperature for 1 hour, by trifluoroacetic anhydride in anhydrous CH₂Cl₂ and pyridine followed by a final HCl methanolic treatment.

The compound 12 was obtained following the synthetic route described in scheme 4: 3,4,5-trifluorobenzaldehyde reacted with 2-((2,2-diethoxyethylamino)methyl)-6-ethoxyphenol SAO 33014 in a 37% HCl solution in ethanol at reflux (100° C.) to obtain the compound 12 in 27% yield as an hydrochloride salt upon a final HCl methanolic treatment.

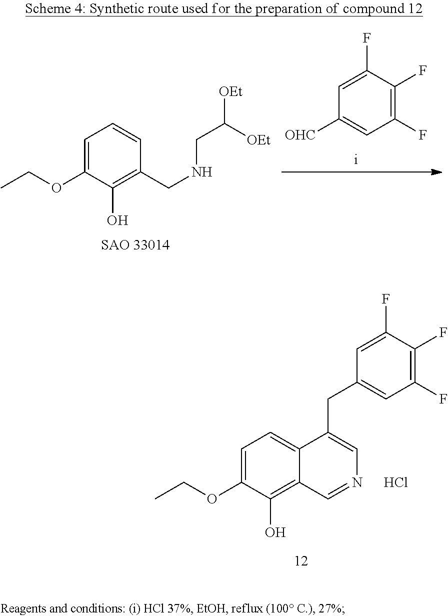 [SMILES: CCOc1cccc(CNCC(OCC)OCC)c1O ; O=Cc1cc(F)c(F)c(F)c1 ; CCOc1ccc2c(Cc3cc(F)c(F)c(F)c3)cncc2c1O ; Cl]

The compounds 9, 11, 13, 17, 46 and 47 were obtained from N-(2,3-disubstituted-benzyl)-2,2-diethoxyethanamines as described in scheme 5: Compound LPO 26048 was treated with 4-hydroxy-3,5-dimethoxybenzaldehyde or 4-ethoxy-3,5-dimethoxybenzaldehyde TTA 24126 using a 37% HCl solution in ethanol at reflux to led respectively to hydrochloride salts 11 or 9 in 25% or 14% yield after final HCl methanolic treatment. The compound LPO 30170 was treated with 4-ethoxy-3,5-dimethoxybenzaldehyde TTA 24126 using a 37% HCl solution in ethanol at reflux to led respectively to the hydrochloride salt 13 in 34% yield after final HCl methanolic treatment. The compound ECO 39026 was treated with 4-ethoxy-3,5-dimethoxybenzaldehyde TTA 24126 using a 37% HCl solution at reflux to led respectively to the hydrochloride salt 46 in 27% yield after final HCl methanolic treatment. The compound 46 was oxidized at room temperature for 5 hours using nitric acid in acetic anhydride to give in 22% yield the compound 47 as an hydrochloride salt after final HCl methanolic treatment. Finally compound LPO 30176 was treated by 2-bromo-3,5-dimethoxybenzaldehyde LPO 30176 using a 37% HCl solution in ethanol at reflux to led respectively to the hydrochloride salt 17 in 27% yield after final HCl methanolic treatment.

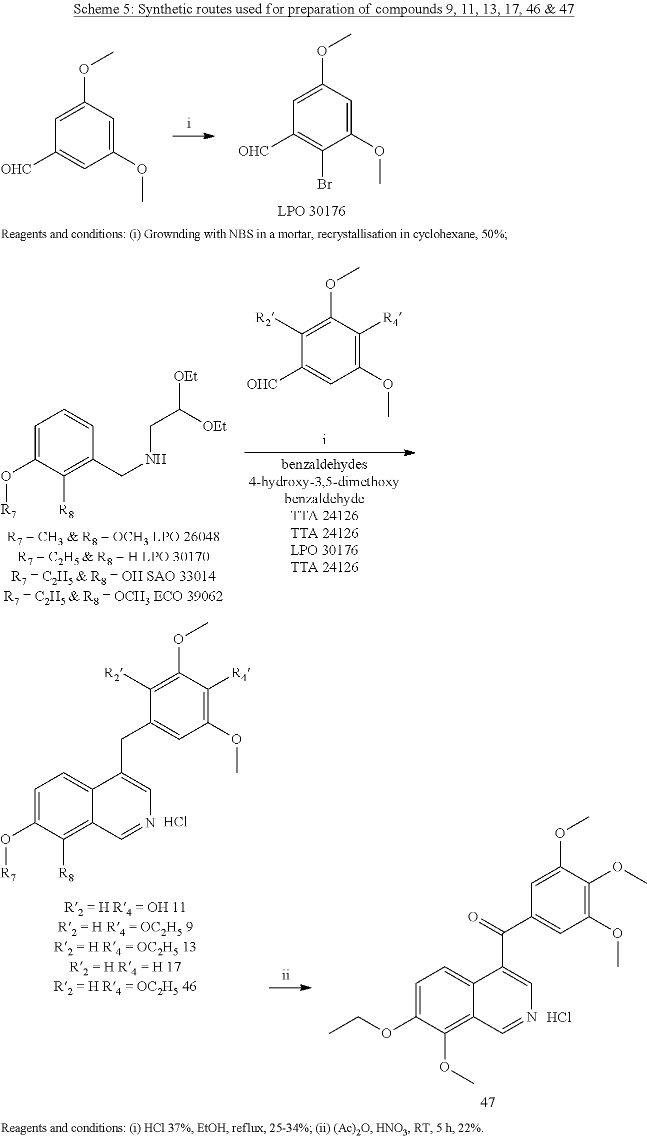

The compounds 23 and 25 were obtained following the synthetic route described in scheme 6:

Compound 13 free base (ANP 31178B) was treated for 15 hours at room temperature with m-chloroperoxybenzoic acid (mCPBA) in dichloromethane at room temperature to afford to the N-oxide TTA 24144A in 86% yield. N-oxide TTA 24144A was refluxed for 4 h in THF with DBU and trimethylsilyl cyanide to give the isoquinoline-1-carbonitrile 23 in 40% yield. Finally the isoquinoline-1-carbonitrile 23 was hydrogenated at room temperature for 3 days using 10% Pd/C in a mixture ethanol/chloroform to lead to the corresponding amine, which was subsequently treated by methanolic HCl to obtain compound 25 as a dihydrochloride salt in 95% yield.

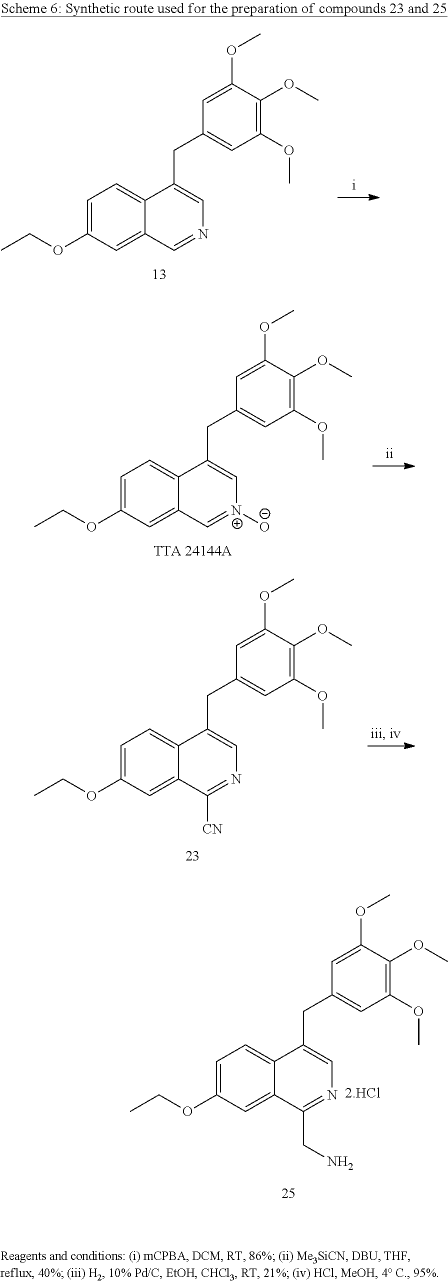

The compound 26 was obtained following the synthetic route described in scheme 7: An overnight coupling reaction at room temperature between amine CCH 34058 and (S)-3-(tert-butoxycarbonyl)-2,2-dimethyloxazolidine-4-carboxylic acid CCH 34168-1 using as coupling agent 1-ethyl-3-(3-dimethylaminopropyl) carbodiimide (EDCI) in dry dichloromethane gave the N-Boc protected compound CCH 34168-2 in quantitative yield. CCH 34168-1 was prepared in 44% yield by overnight saponification from 0° C. to room temperature of the commercially available (S)-3-tert-butyl 4-methyl 2,2-dimethyloxazolidine-3,4-dicarboxylate by using LiOH in a THF:H₂O=1:1 mixture.

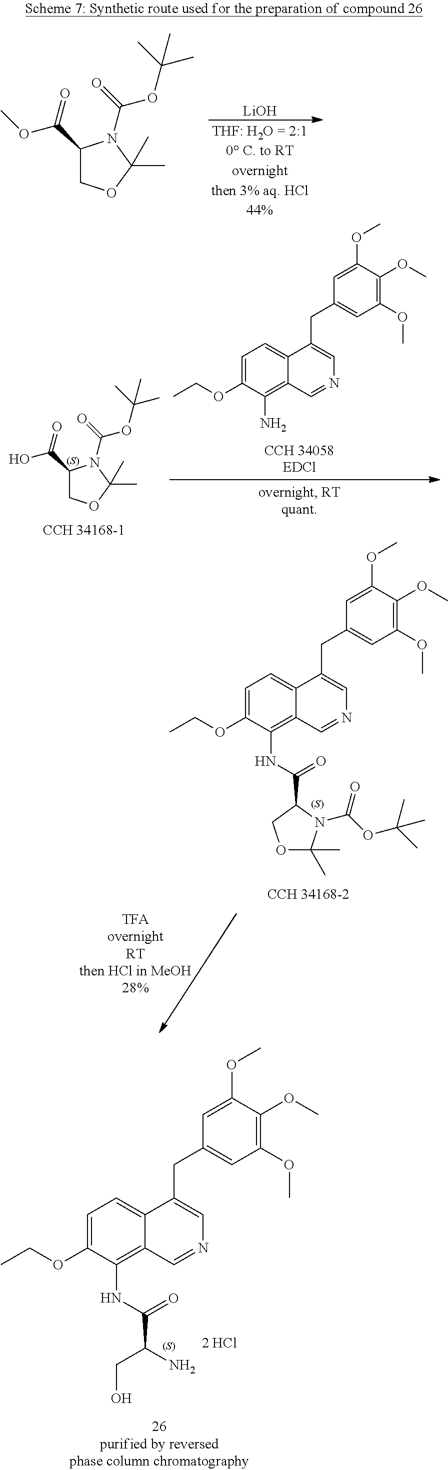

Overnight N-Boc deprotection using trifluoroacetic acid at room temperature of the compound CCH 34168-2 gave, after final treatment with a 0.19 N HCl solution in MeOH, the serinamide dihydrochloride salt 26 in 28% yield (scheme 7).

The compound 29 was obtained following the synthetic route described in scheme 8.

Palladium-catalyzed amination (Buchwald-Hartwig reaction) for 5 hours at 150° C. under microwave irradiation between triflate CCH 34050 and benzophenone imine, using a catalyst consisting of a combination of tris(dibenzylideneacetone)dipalladium(0) and BINAP in presence of cesium carbonate in dry toluene gave N-(diphenylmethylene)-7-ethoxy-4-(3,4,5-trimethoxybenzyl)isoquinolin-8-amine CCH 34090 in 73% yield. Benzylic oxidation of CCH 34090 using sodium chlorite and N-hydroxyphtalimide as catalyst in a mixture of acetonitrile:H₂O=2:1 (modified oxidation conditions described in S. M. Silvestre, J. A. R. Salvador, tetrahedron, 63, 2007, 2439-2445), followed by hydrolysis of the imine with a mixture of 1 N aqueous HCl:THF=1:1 at RT for 30 minutes led to 7-ethoxy-4-(3,4,5-trimethoxybenzoyl)isoquinolin-8-amine that was finally treated by a 0.49 N HCl solution in methanol 15 minutes at RT to obtain 7-ethoxy-4-(3,4,5-trimethoxybenzoyl)isoquinolin-8-amine hydrochloride 29 in 45% overall yield.

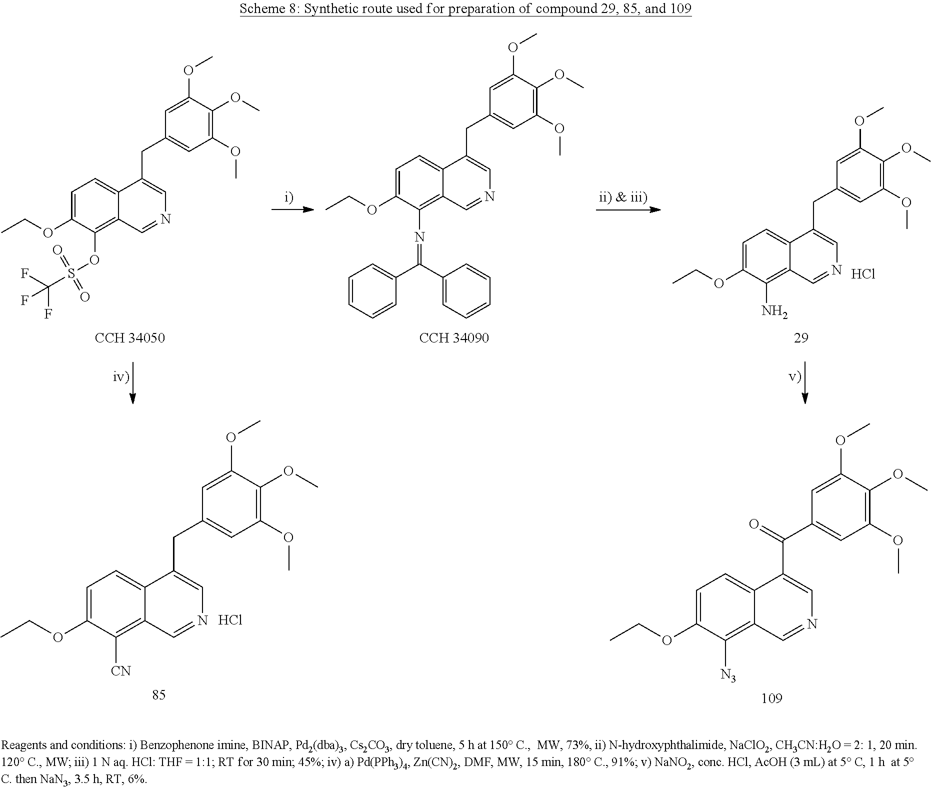

The azido derivative 109 was obtained in 6% yield from compound 29 by diazotation for 1 hour at 5° C. with sodium nitrite in concentrated HCl and acetic acid followed by treatment, for 3.5 hours from 5° C. to room temperature, with sodium azide as nucleophile (Sandmeyer reaction).

The cyano compound 85 hydrochloride was obtained in 91% yield (modified conditions of a similar reaction described in WO2005/66194, p161) from triflate CCH 34050 using zinc cyanide and tetrakis(triphenylphosphine)palladium as a catalyst in DMF at 180° C. for 15 min under microwave irradiation, followed by a final HCl methanolic treatment (scheme 8).

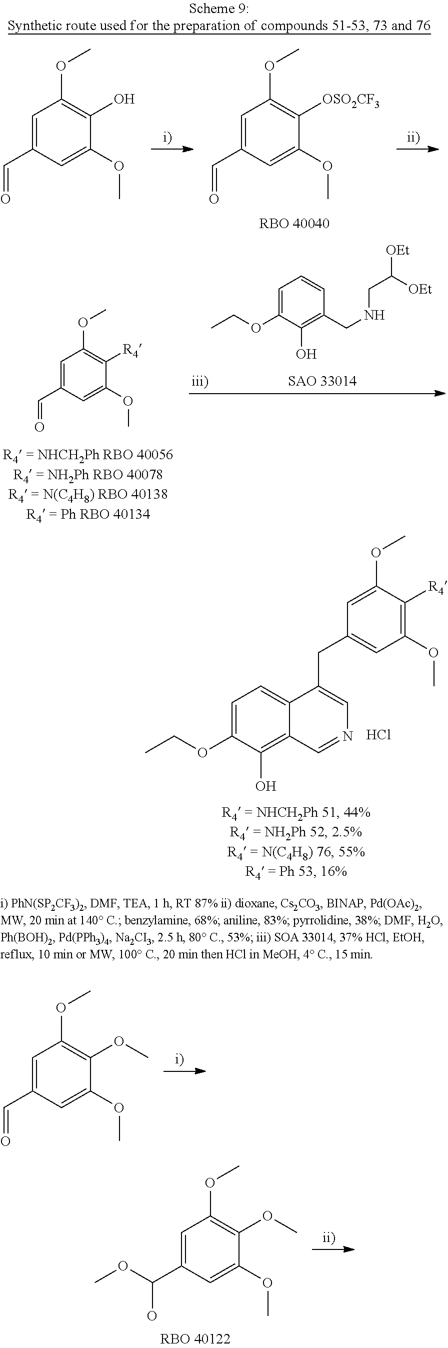

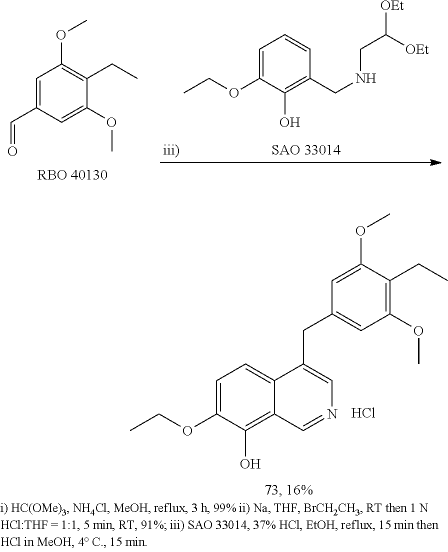 [SMILES: CCc1c(OC)cc(C=O)cc1OC ; CCOc1cccc(CNCC(OCC)OCC)c1O ; CCOc1ccc2c(Cc3cc(OC)c(CC)c(OC)c3)cncc2c1O ; Cl]

The compounds 51-53, 73 and 76 were obtained following the synthetic route described in scheme 9: The conversion of aryl triflate RBO 40040 to the corresponding benzyl, aniline or pyrrolidine derivatives (respectively RBO 40056, RBO 40078, or RBO 40138) was accomplished in moderate to good yields (38-83% yields) using a catalyst consisting of the combination of palladium acetate and BINAP (Buchwald reaction) in dioxane at 140° C. under microwave irradiation and with cesium carbonate as a base.

Suzuki coupling of aryl triflate RBO 40040 with phenyl boronic acid using tetrakis(triphenylphosphine)palladium as a catalyst in a mixture of DMF/H₂O and in presence of sodium carbonate led to the 1,1′-biphenyl derivative RBO 40134 in 53% yield.

The compounds RBO 40056, RBO 40078, RBO 40138 or RBO 40134 were treated with 2-((2,2-diethoxyethylamino)methyl)-6-ethoxyphenol SAO 33014 in a 37% HCl solution in ethanol at reflux to obtain respectively the compounds 51, 52, 76 or 53 as hydrochloride salts upon a final HCl methanolic treatment.

The compound 73 was obtained from 3,4,5-trimethoxybenzaldehyde (scheme 9): 3,4,5-Trimethoxybenzaldehyde reacted with trimethyl orthoformate to obtain the acetal RBO 40122 in nearly quantitative yield. The acetal RBO 40122 was treated with sodium in tetrahydrofuran for 24 hours at room temperature followed by addition of bromoethane at 0° C. and the mixture was stirred for 2 days and hydrolyzed after work-up with a THF:1 N HCl=1:1 solution for 5 min (conditions adapted from Azzena, Ugo et al., Synthetic Communications, 2003, 33, 1309-1318) to give the aldehyde RBO 40130 in 91% yield.

The aldehyde RBO 40130 was treated with 2-((2,2-diethoxyethylamino)methyl)-6-ethoxyphenol SAO 33014 in a 37% HCl solution in ethanol at reflux to give compound 73 as an hydrochloride salt in 16% yield upon a final HCl methanolic treatment.

The compounds 55-60 were obtained following the synthetic routes described in scheme 10:

The compounds 55-57 were obtained from phenol 14. Benzylation of phenol 14 with benzyl bromide in DMF afforded LPO 37138 in 32% yield. Benzylic oxidation of LPO 37138 using sodium chlorite and N-hydroxyphtalimide as catalyst in a mixture of acetonitrile:H₂O=2:1 at 50° C. for 3.5 hours provided the keto compound 55 in 28% yield.

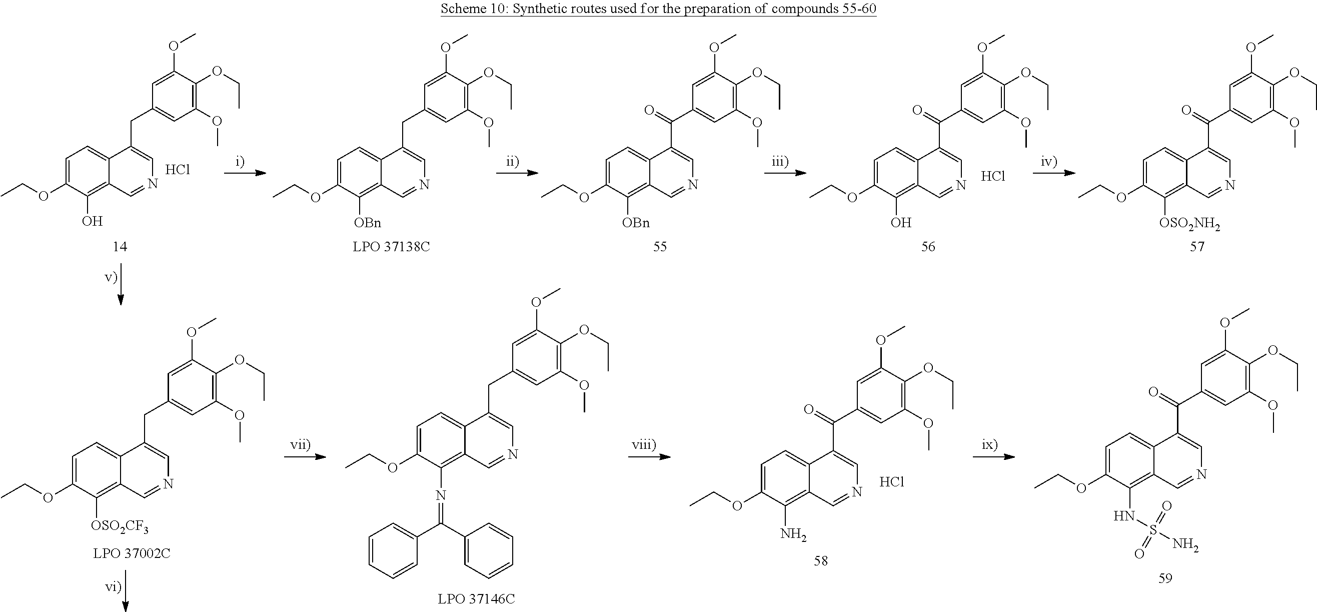

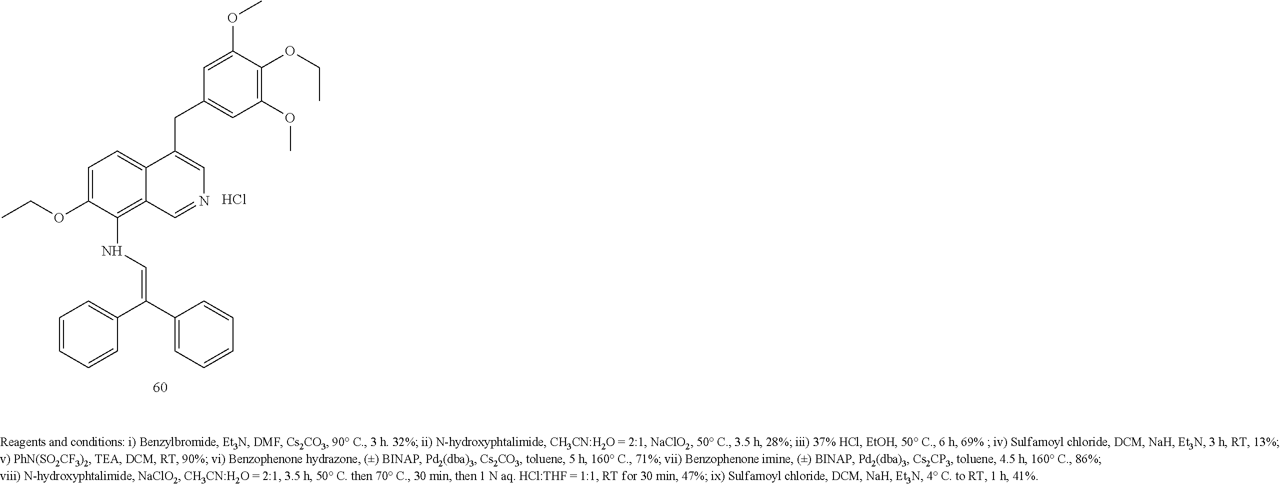 [SMILES: CCOc1ccc2c(Cc3cc(OC)c(OCC)c(OC)c3)cncc2c1NC=C(c1ccccc1)c1ccccc1 ; Cl]

The compound 55 was treated by an HCl ethanolic solution at 50° C. for 6 hours to give in 69% yield the phenol 56 as an hydrochloride salt after a final HCl methanolic treatment. Finally the phenol 56 was treated, for 1 h at 4° C. to room temperature, with sulfamoyl chloride in dichloromethane in presence of triethylamine and NaH to give the corresponding sulfamate derivative 57 in 41% yield (scheme 10).

The compounds 58 and 59 were obtained from the free base of phenol 14 (scheme 10). The phenol 14 (free base) reacted at room temperature with N-phenyl-bis(trifluoromethanesulfonimide) in DMF in presence of triethylamine to give the corresponding triflate LPO 37002C in 90% yield. Palladium-catalyzed amination (Buchwald-Hartwig reaction) at 160° C. for 4.5 hours under microwave irradiation between triflate LPO 37002C and benzophenone imine, using a catalyst consisting of a combination of tris(dibenzylideneacetone)dipalladium(0) and BINAP in presence of cesium carbonate in dry toluene afforded to the imine LPO 37146C in 86% yield. The imine LPO 37146C was oxidized using sodium chlorite and N-hydroxyphtalimide as catalyst in a mixture of acetonitrile:H₂O=2:1 and subsequently hydrolyzed with a 1 N aqueous HCl:THF=1:1 solution at RT for 30 min to give to the keto amino compound 58 as an hydrochloride salt in 47% yield upon final HCl methanolic treatment. The sulfamide compound 59 was obtained in 41% yield from the amino compound 58 by treatment at room temperature for 1 hour with sulfamoyl chloride in DMF in presence of triethylamine and NaH.

Palladium-catalyzed amination (Buchwald-Hartwig reaction) at 160° C. for 5 hours under microwave irradiation between triflate LPO 37002C and benzophenone hydrazone, using a catalyst consisting of a combination of tris(dibenzylideneacetone)dipalladium(0) and BINAP in presence of cesium carbonate in dry toluene afforded to the imine LPO 37146C in 86% yield as an hydrochloride salt upon final HCl methanolic treatment (scheme 10).

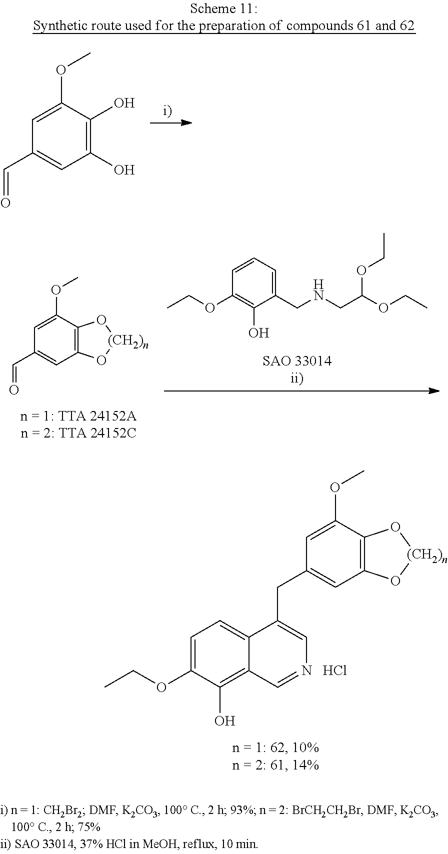

The compounds 61 and 62 were obtained from 3,4-dihydroxybenzaldehyde (scheme 11):

3,4-Dihydroxybenzaldehyde was alkylated using 1,2-dibromomethane or dibromoethane in DMF in presence of potassium carbonate at 100° C. for 2 hours to give, respectively, to dioxane TTA 24152A in 93% yield or dioxolane TTA 24152C in 75% yield. The compounds TTA 24152A or TTA 24152C were treated for 10 minutes with 2-((2,2-diethoxyethylamino)methyl)-6-ethoxyphenol SAO 33014 in HCl 37% in ethanol at reflux to give, respectively, the compounds 62 or 61 as hydrochloride salts upon a final HCl methanolic treatment.

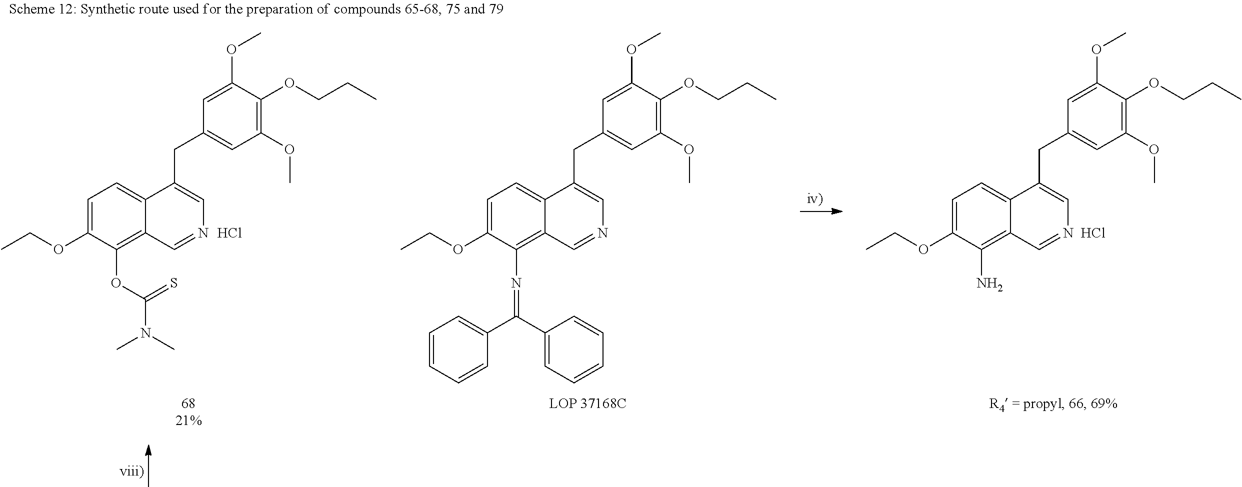 [SMILES: CCCOc1c(OC)cc(Cc2cncc3c(OC(=S)N(C)C)c(OCC)ccc23)cc1OC ; Cl ; CCCOc1c(OC)cc(Cc2cncc3c(N=C(c4ccccc4)c4ccccc4)c(OCC)ccc23)cc1OC ; CCCOc1c(OC)cc(Cc2cncc3c(N)c(OCC)ccc23)cc1OC ; Cl]

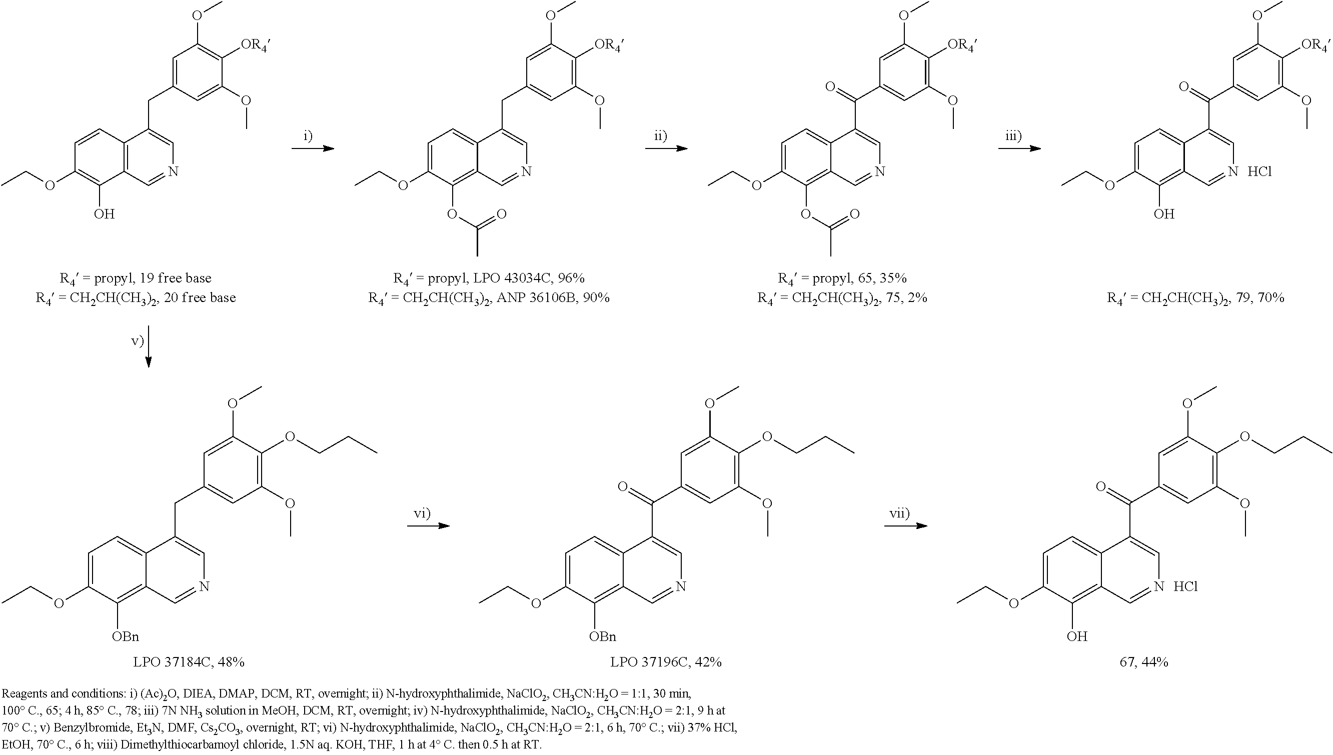

The compounds 65, 66, 67, 68, 75 and 79 were obtained from compounds 19 and 20 (free bases) (scheme 12):

The phenols derivatives 19 or 20 (free bases) were transformed into their corresponding acetates LPO 43034C or ANP 36106B using acetic anhydride in dichloromethane in presence of N,N-diisopropylethylamine and a catalytic amount of 4-dimethylaminopyridine (DMAP). LPO 43034C or ANP 36106B were oxydized using sodium chlorite and N-hydroxyphtalimide as catalyst in a mixture of acetonitrile:H₂O=1:1 to give to the keto compounds 65 or 75. The acetate 75 was hydrolyzed, overnight at room temperature with a 7 N ammonia methanolic solution in dichloromethane, into its corresponding phenol 79 as an hydrochloride salt in 70% yield upon a final HCl methanolic treatment.

The benzophenone imine LPO 37168C was oxidized using sodium chlorite and N-hydroxyphtalimide as catalyst in a mixture of acetonitrile:H₂O=1:1 and subsequently hydrolyzed by a mixture of 1 N aq. HCl:THF=1:1 at room temperature for 40 min to give the keto amino compound 66 as an hydrochloride salt in 69% yield upon a final HCl methanolic treatment. The compound 67 was obtained similarly to compound 79 using a benzyl protected phenol 20 (free base) instead an acetate protection. Finally the compound 68 was obtained in 21% yield as an hydrochloride salt from the compound 19 (free base) by reaction with dimethylthiocarbamoyl chloride in tetrahydrofuran in presence of a 1.5 N aqueous solution of KOH, followed by a final HCl methanolic treatment (scheme 12).

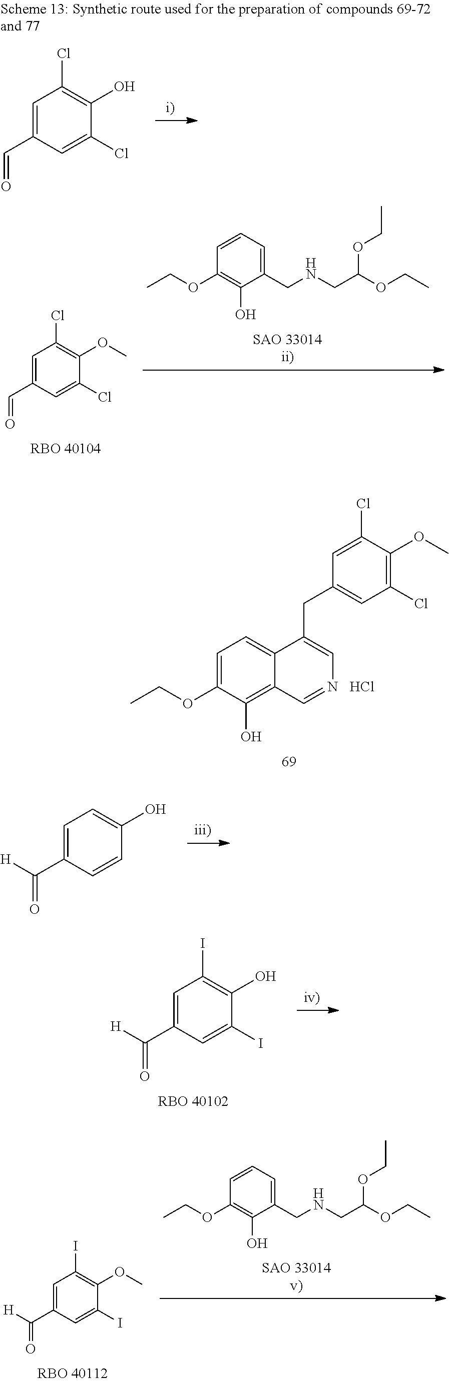

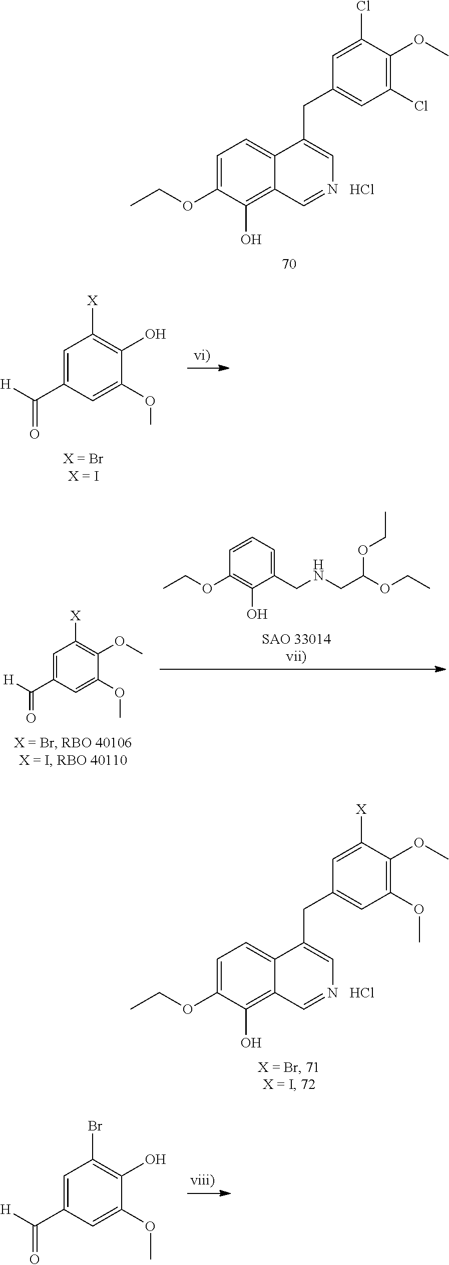

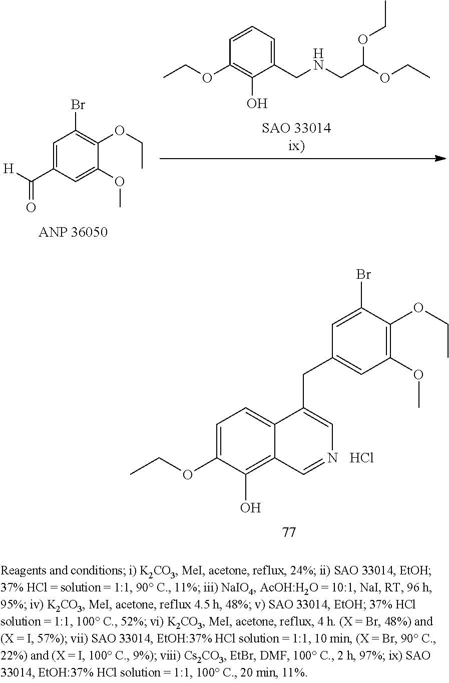

The compounds 69-72 and 77 were obtained as described in scheme 13: The aldehydes RBO 40104, RBO 40112, RBO 40106, RBO 40110 or ANP 36050 reacted with 2-((2,2-diethoxyethylamino)methyl)-6-ethoxyphenol SAO 33014 in a 37% HCl solution in ethanol at reflux (90° C.-100° C.) to give, respectively, the compounds 69, 70, 71, 72 or 77 as hydrochloride salts upon a final HCl methanolic treatment.

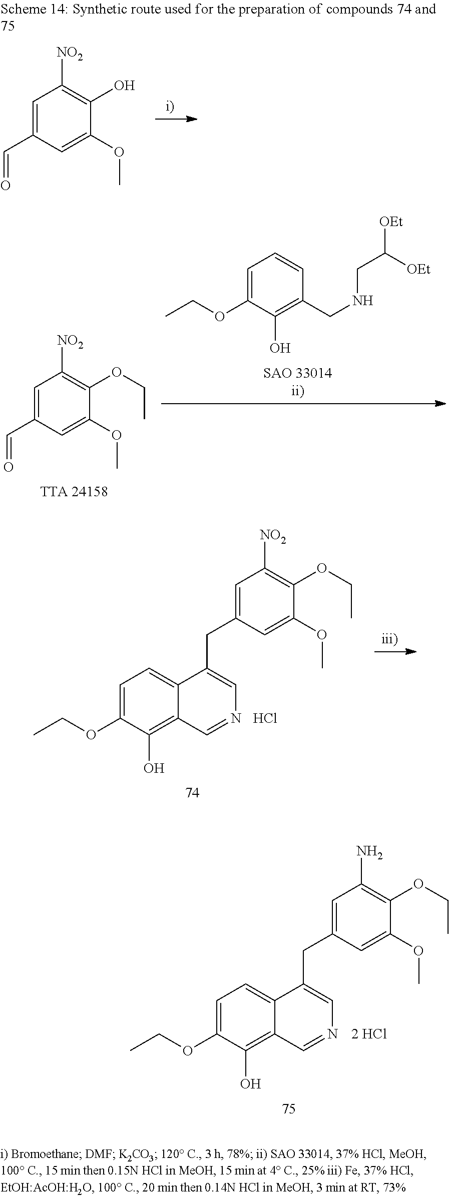

The compounds 74 and 75 were obtained as described in scheme 14: O-Alkylation of 5-nitrovanilline by bromoethane gave aldehyde TTA 24158 in 78% yield. This aldehyde TTA 24158 was treated with SAO 33014 in a 37% HCl solution in ethanol at 100° C. to give the compound 74 as an hydrochloride salt in 25% yield upon a final HCl methanolic treatment. Béchamp reduction of the nitro compound 74 using iron and HCl in acetic acid led to the amino compound 75 as a dihydrochloride salt in 73% yield upon a final HCl methanolic treatment (scheme 14).

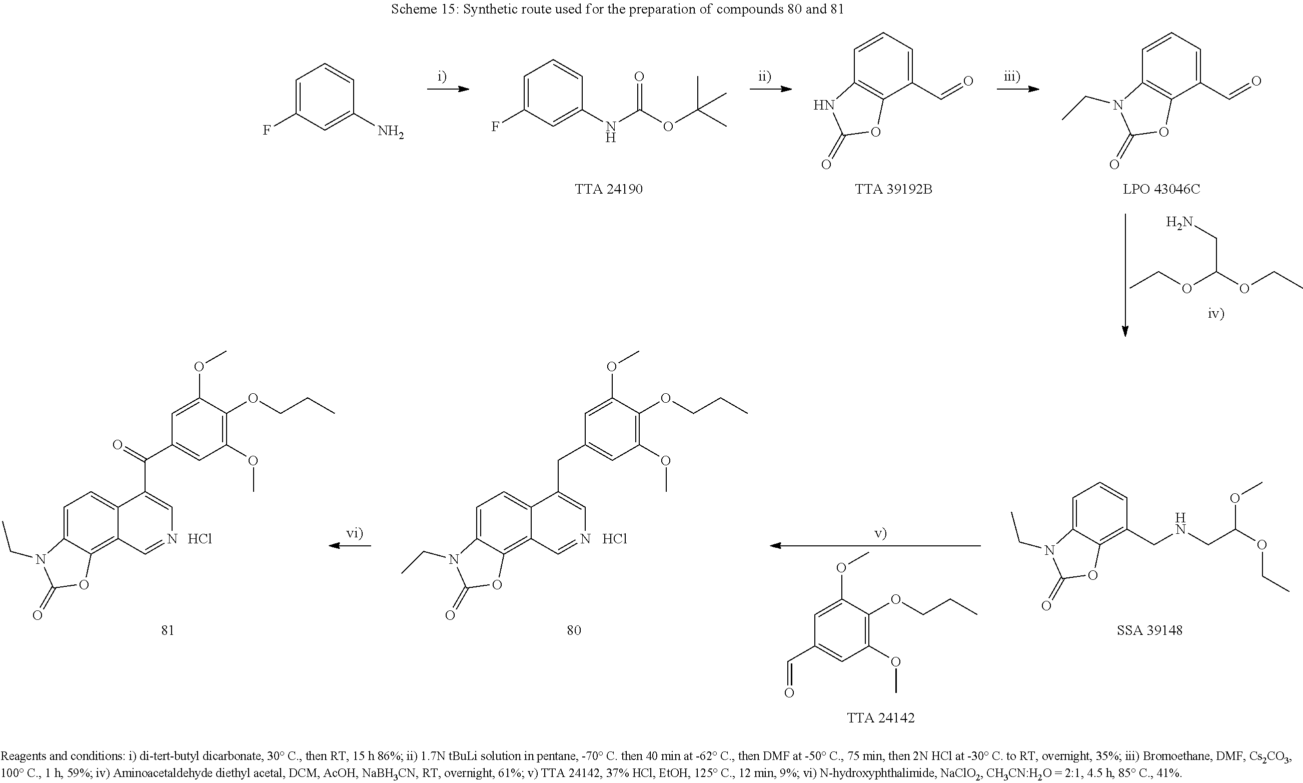

The compounds 80 and 81 were obtained following the synthetic route described in scheme 15: N-Boc protection of 3-fluoroaniline gave TTA 24190 in 86% yield. Metalation of compound TTA 24190 using a 1.7 N tBuli solution in pentane at −70° C. followed by addition of DMF at −50° C. afforded to the aldehyde TTA 39192B in 35% yield. The N-alkylation of compound TTA 39192B by bromoethane in DMF in presence of cesium carbonate led to the N-ethyl derivative LPO 43046C in 59% yield. The reductive amination of the compound LPO 43046C with aminoacetaldehyde diethyl acetal in presence of sodium cyanoborohydride provided compound SSA 39148 in 61% yield. The aldehyde TTA 24142 was treated with SSA 39148 in a 37% HCl solution in ethanol at 125° C. to give the compound 80 as an hydrochloride salt in 9% yield upon a final HCl methanolic treatment. Finally the compound 80 was oxidized using sodium chlorite and N-hydroxyphtalimide as catalyst in a mixture of acetonitrile:H₂O=2:1 at 85° C. for 4.5 hours to obtain the keto compound 81 as an hydrochloride salt in 41% yield after final HCl methanolic treatment (scheme 15).

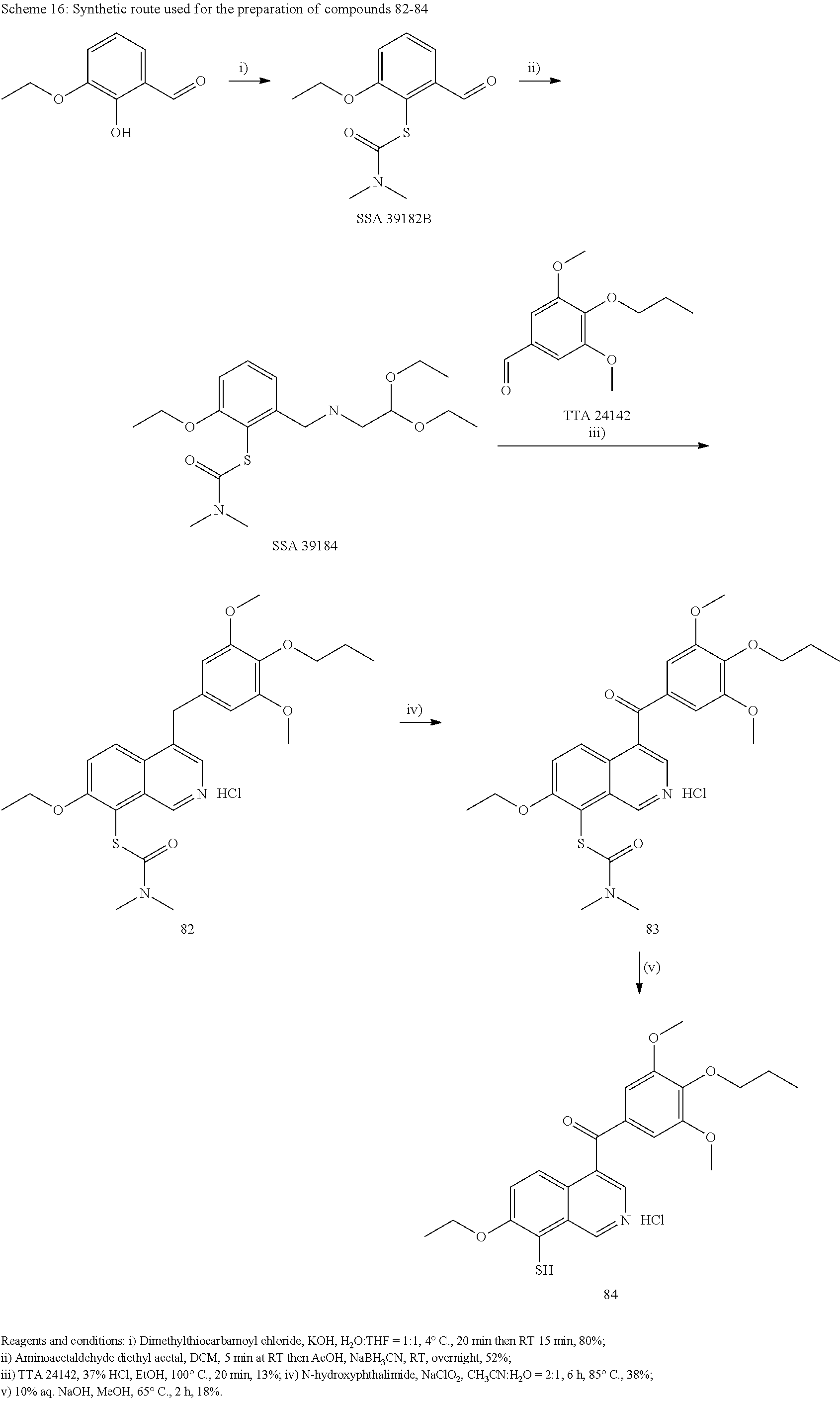

The compounds 82-84 were obtained following the synthetic route described in scheme 16:

3-Ethoxysalicylaldehyde was treated with dimethylthiocarbamoyl chloride in a mixture of THF and water and potassium hydroxide as a base to obtain SSA 381828 in 80% yield. Reductive amination between SSA 381828 and 2,2-diethoxyethanamine using sodium cyanoborohydride in dichloromethane and acetic acid overnight at room temperature gave SSA 39184 in 80% yield. Reaction of aldehyde TTA 24142 with the compound SSA 39184, using a 37% HCl solution in ethanol at 100° C., led to the compound 82 as an hydrochloride salt in 10% yield after a final HCl methanolic treatment. The compound 82 was oxidized using sodium chlorite and N-hydroxyphtalimide as catalyst in a mixture of acetonitrile:H₂O=2:1 at 85° C. for 6 hours to obtain the keto compound 83 as an hydrochloride salt in 38% yield after final HCl methanolic treatment. Finally the compound 83 was treated with a 10% aqueous NaOH solution in methanol at 65° C. for 2 hours to give the thiol derivative 84 as an hydrochloride salt in 18% yield after a final HCl methanolic treatment (scheme 16).

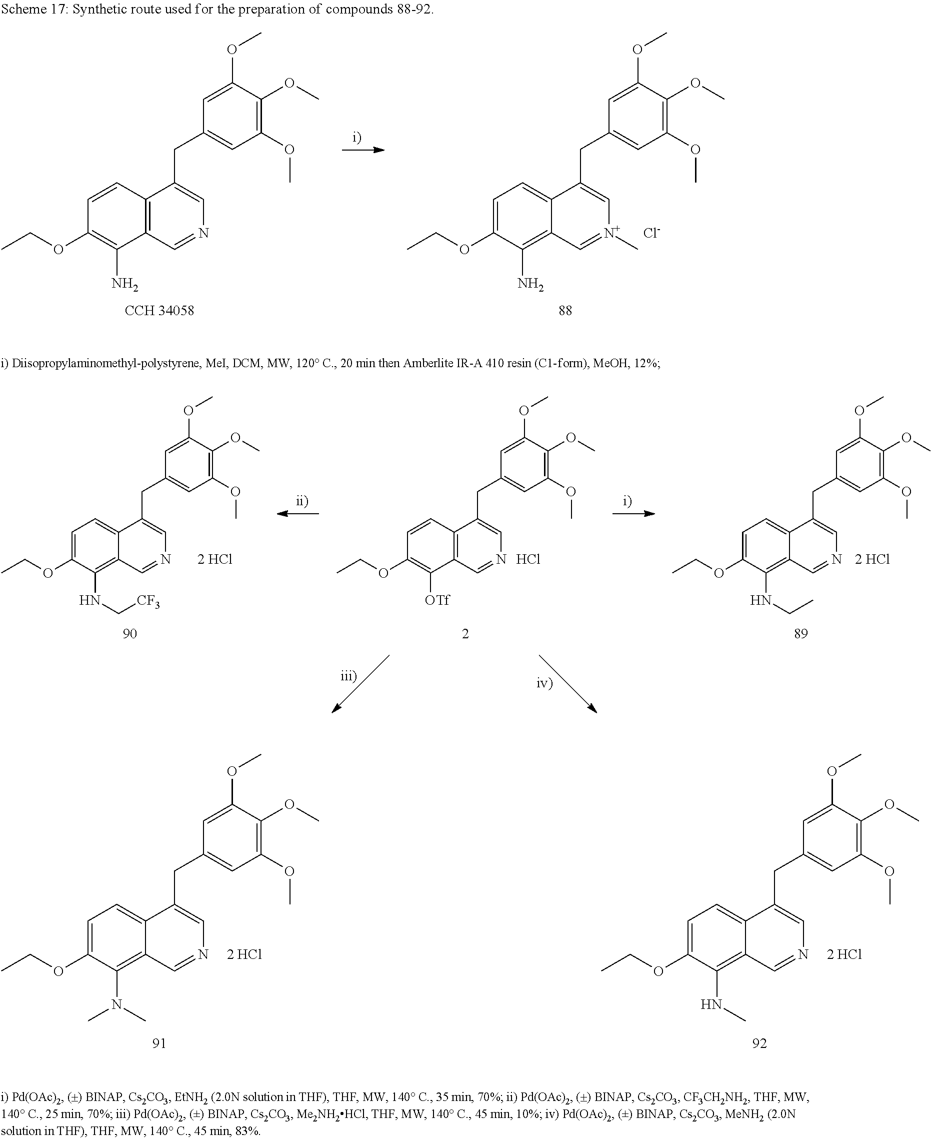

The compounds 88-92 were obtained as described in scheme 17: Compound 88 was obtained from compound CCH 34058. CCH 34058 was treated with methyliodide in dichloromethane at 120° C. for 20 min under microwave irradiation in presence of diisopropylaminomethyl-polystyrene to give, after filtration through Amberlite IR-A 410 resin (Cl₋ form), the compound 88 in 12% yield (scheme 16).

Palladium-catalyzed amination (Buchwald reaction) at 140° C. under microwave irradiation between triflate 2 and diethylamine, 2,2,2-trifluoroethylamine, dimethylamine hydrochloride, or methylamine, using a catalyst consisting of a combination of palladium(II) acetate and BINAP in presence of cesium carbonate in dry tetrahydrofuran afforded, respectively, to the compounds 89, 90, 91, or 92 as dihydrochloride salts in 10-83% yields upon final HCl methanolic treatment (scheme 16).

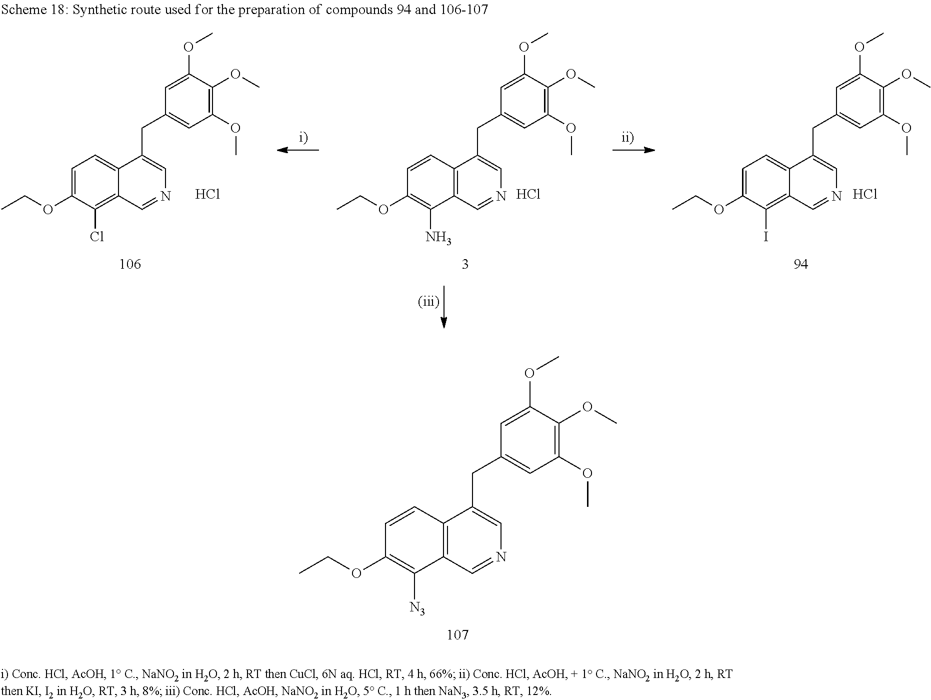

The compounds 94, 106-107 were obtained from the amino compound 3 as described in scheme 18: The substitution of an aromatic amino group is possible via preparation of its diazonium salt and subsequent displacement with a nucleophile. The Sandmeyer reactions of compound 3 using CuCl, KI or NaN₃ (as nucleophile sources) led, respectively, to the chloro derivative 106 or the iodo derivative 94 as hydrochloride salts upon final HCl methanolic treatment, or to the azido derivative 107 (scheme 18).

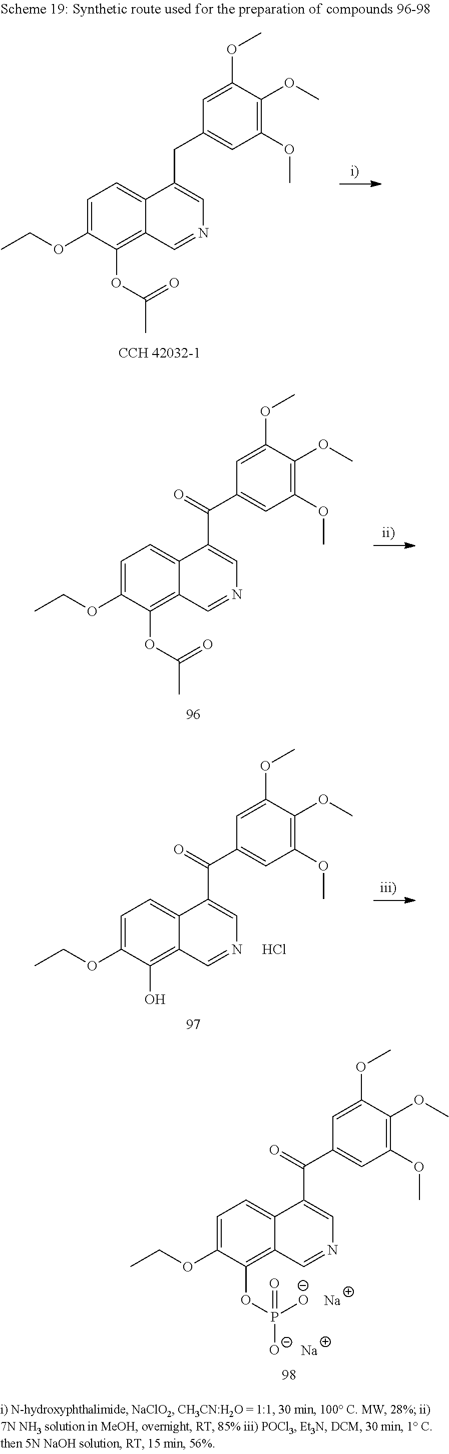

The oxidation of the acetate CCH 42032-1 using sodium chlorite and N-hydroxyphtalimide as catalyst in a mixture of acetonitrile:H₂O=1:1 at 100° C. for 30 min under microwave irradiation led to the corresponding keto compound 96 in 28% yield. The compound 96 was deprotected using a 7 N ammonia solution to give compound 97 as an hydrochloride salt in 85% yield after a final HCl methanolic treatment. Finally the compound 97 was treated for 30 min at 1° C. with phosphoryl chloride in dry CH₂Cl₂ in presence of triethylamine followed by a 5 N aq. NaOH solution, 15 min at room temperature, to give the disodium phosphate salt 98 in 56% yield (scheme 19).

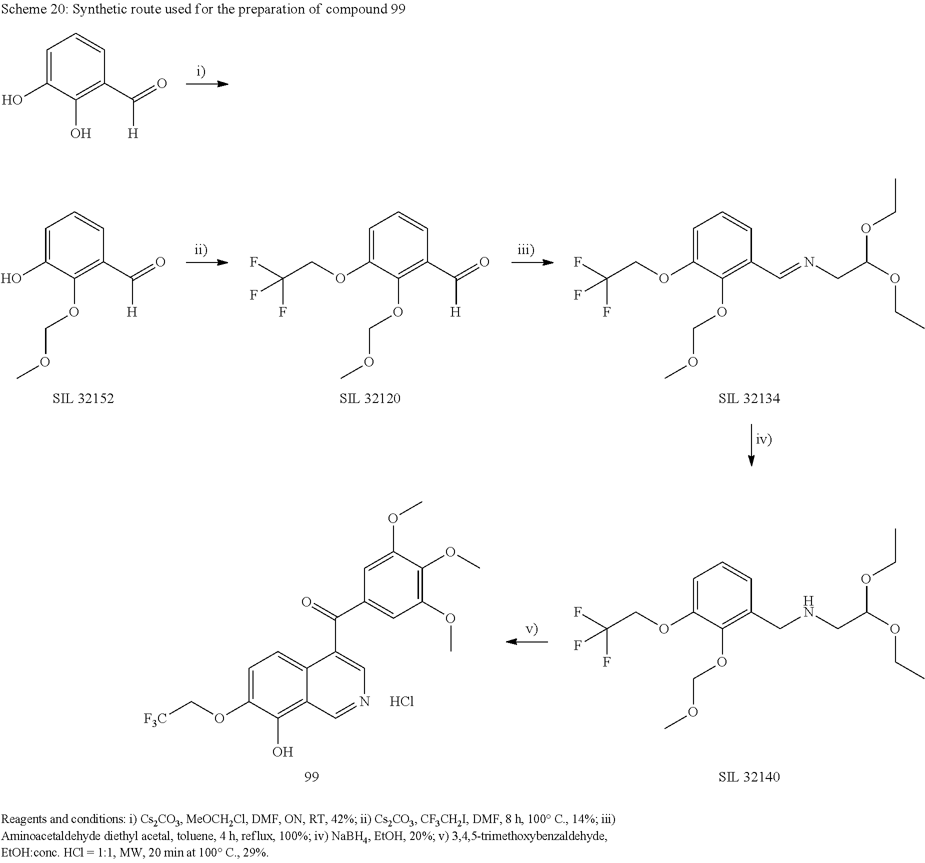

MOM protection of 2,3-dihydroxybenzaldehyde using chloromethyl methyl ether in DMF overnight at room temperature provided compound SIL 32152 in 42% yield. O-Alkylation of phenol SIL 32152 using 2,2,2-trifluoroethyl iodide in DMF for 8 hours at 100° C. in presence of cesium carbonate led to the compound SIL 32120 in 14% yield. The compound SIL 32120 reacted with 2,2-diethoxyethanamine in toluene at reflux for 4 hours to give quantitatively SIL 32134. Reduction of SIL 32134 with sodium borohydride in ethanol led to the compound SIL 32140 in 20% yield. Finally 3,4,5-trimethoxybenzaldehyde was treated with SIL 32140 in a concentrated HCl solution in ethanol, 20 minutes at 100° C. under microwave irradiation, to give the compound 99 as an hydrochloride salt in 29% yield upon a final HCl methanolic treatment (scheme 20).

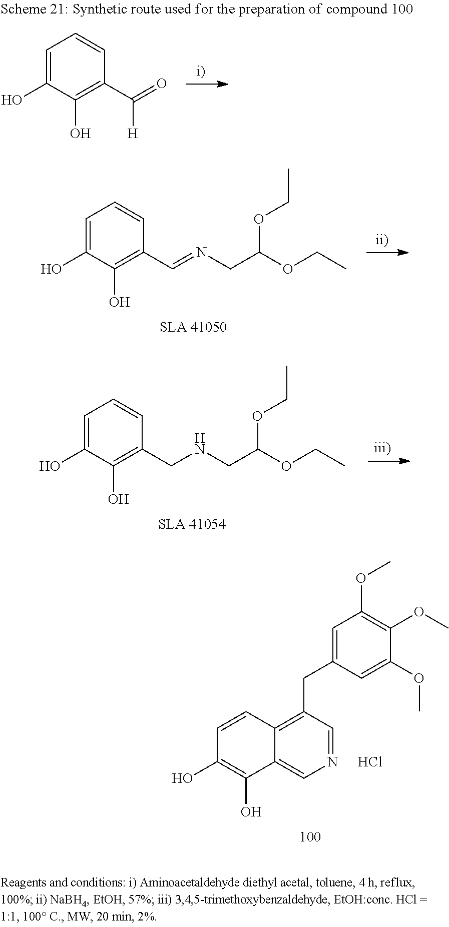 [SMILES: O=Cc1cccc(O)c1O ; CCOC(C/N=C/c1cccc(O)c1O)OCC ; CCOC(CNCc1cccc(O)c1O)OCC ; COc1cc(Cc2cncc3c(O)c(O)ccc23)cc(OC)c1OC ; Cl]

2,3-Dihydroxybenzaldehyde reacted with 2,2-diethoxyethanamine in toluene at reflux for 4 hours to give quantitatively (E)-3-((2,2-diethoxyethylimino)methyl)benzene-1,2-diol SLA 41050. Reduction of the compound SLA 41050 with sodium borohydride in ethanol led to the compound SLA 41054 in 57% yield. Finally 3,4,5-trimethoxybenzaldehyde was treated with SLA 41054 in a concentrated HCl solution in ethanol 20 minutes at 100° C. under microwave irradiation to give the compound 100 as an hydrochloride salt in 2% yield upon a final HCl methanolic treatment (scheme 21).

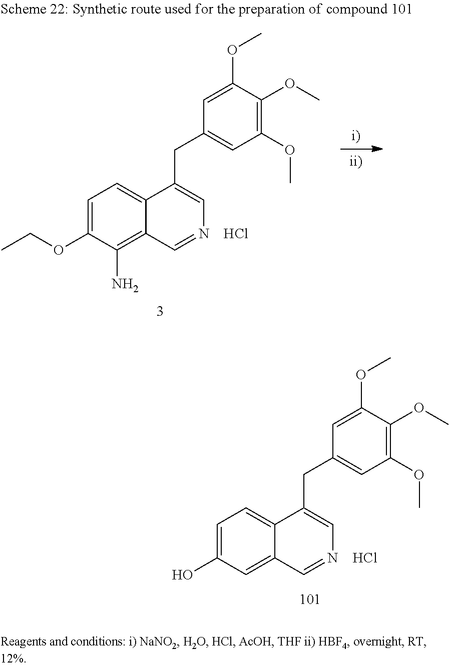 [SMILES: CCOc1ccc2c(Cc3cc(OC)c(OC)c(OC)c3)cncc2c1N ; Cl ; COc1cc(Cc2cncc3cc(O)ccc23)cc(OC)c1OC ; Cl]

The compound 101 was obtained from compound 3 (scheme 22): The compound 3 in concentrated HCl at 5° C. was treated for 1 hour with a solution of sodium nitrite in H₂O before adding AcOH and THF (diazotation). After stirring for 30 min at 5° C., HBF₄ was added and the reaction mixture was stirred overnight at RT. After work-up the phenol 101 was obtained as an hydrochloride salt in 12% upon a final HCl methanolic treatment.

5-Nitrovanilline was treated with SAO 33014 in a 37% HCl solution in ethanol, for 20 minutes at 100° C. under microwave irradiation, to give the compound 102 as an hydrochloride salt in 29% yield upon a final HCl methanolic treatment (scheme 23).

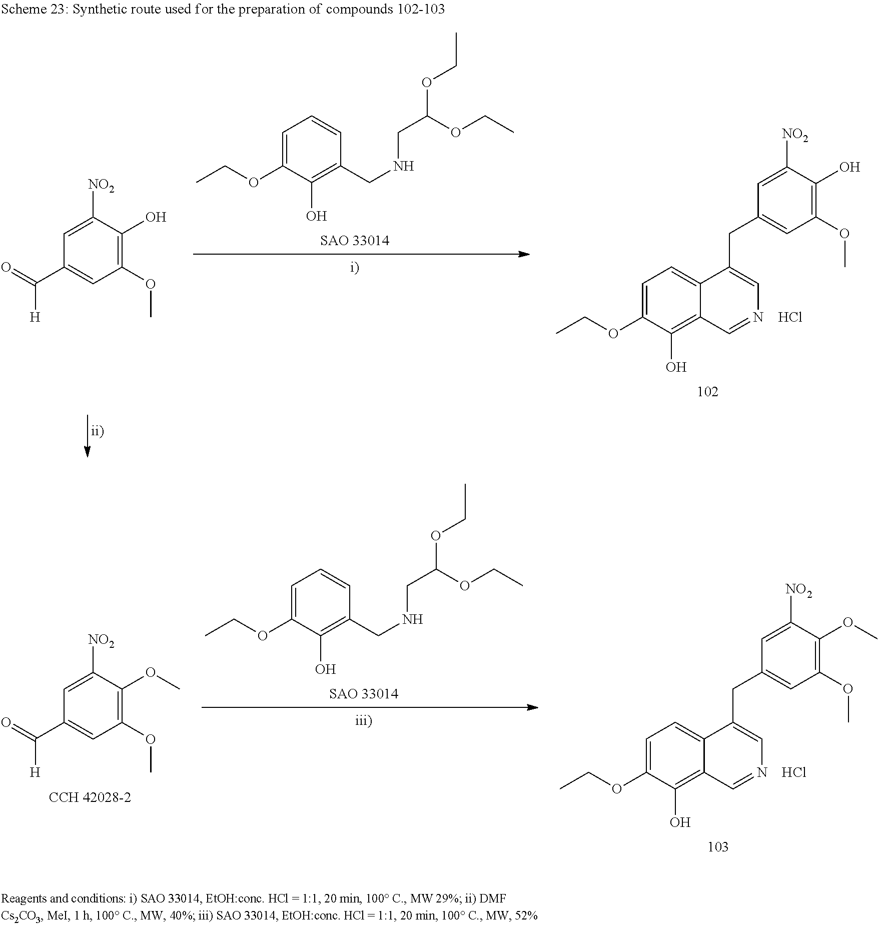

O-Alkylation of 5-nitrovanilline by methyl iodide in DMF, at 100° C. under microwave irradiation and in presence of cesium carbonate, gave aldehyde CCH 42048-2 in 40% yield. This aldehyde CCH 42048-2 was treated with SAO 33014 in a 37% HCl solution in ethanol 20 minutes at 100° C. under microwave irradiation to give the compound 103 as an hydrochloride salt in 52% yield upon a final HCl methanolic treatment (scheme 23).

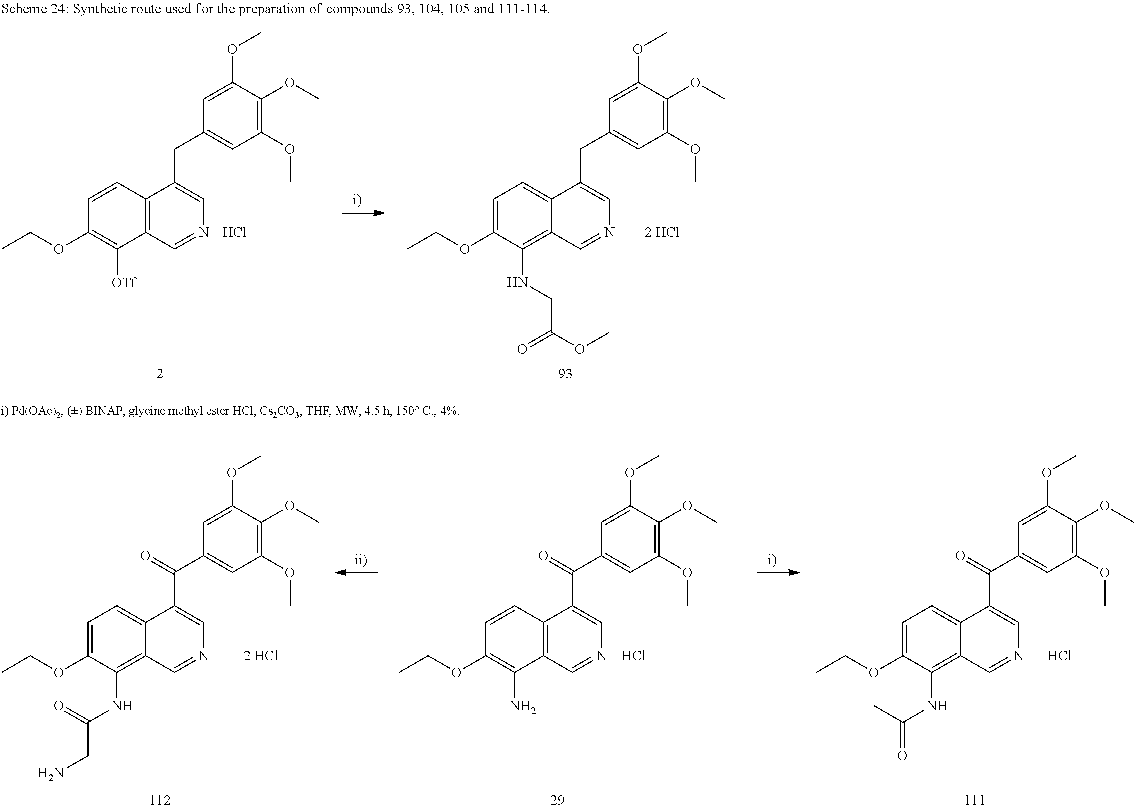

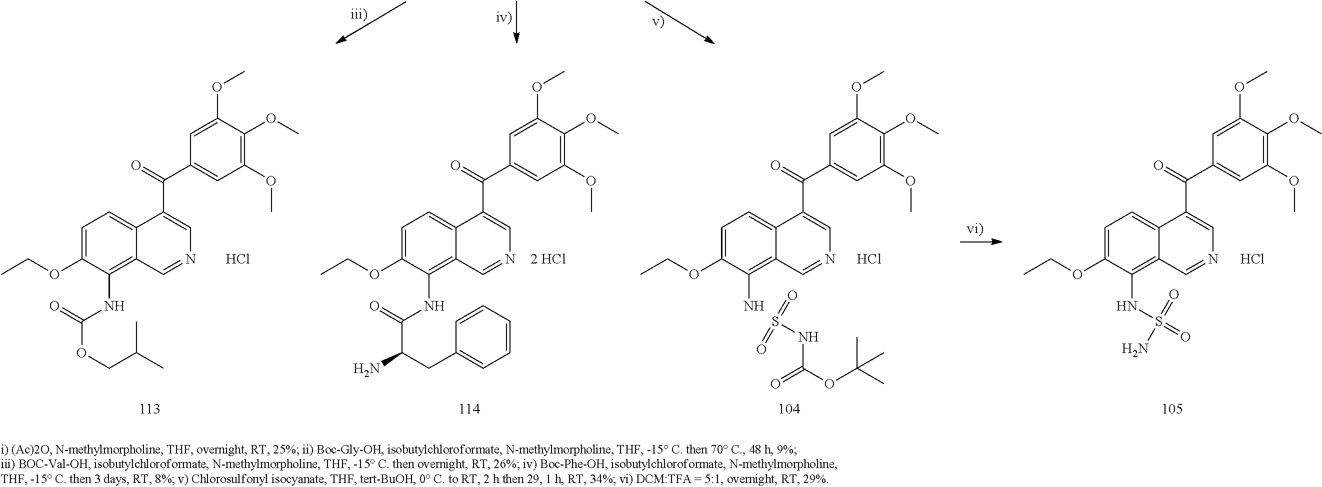

The compounds 93, 104, 105 and 111-114 were obtained following the synthetic route described in scheme 24:

Palladium-catalyzed amination (Buchwald reaction) at 150° C. for 4.5 hours under microwave irradiation between triflate 2 and glycine methyl ester, using a catalyst consisting of a combination of palladium(II) acetate and BINAP in presence of cesium carbonate in dry tetrahydrofuran afforded to the compound 93 as a dihydrochloride salt in 4% yield upon final HCl methanolic treatment (scheme 24).

The compounds 104, 105 and 111-114 were obtained from compound 29. Acetylation of 29 by acetic anhydride in THF in presence of N-methylmorpholine led to the acetate 111 as an hydrochloride salt in 25% yield upon a final HCl methanolic treatment. Coupling reactions using isobutyl chloroformate between amino derivative 29 and Boc-Gly-OH, Boc-Val-OH, or Boc-Phe-OH followed by a final HCl methanolic treatment led to compounds 112, 113 or 114 as hydrochloride salts in 6-26% yields. The compound 104 was obtained in 34% yield after reacting compound 29 with a solution of chlorosulfonyl isocyanate and tert-butanol in THF. The compound 104 was treated overnight at room temperature with a solution of trifluoroacetic acid in dichloromethane to obtain the N-sulfamide 105 as an hydrochloride salt in 29% yield after final HCl methanolic treatment (scheme 24).

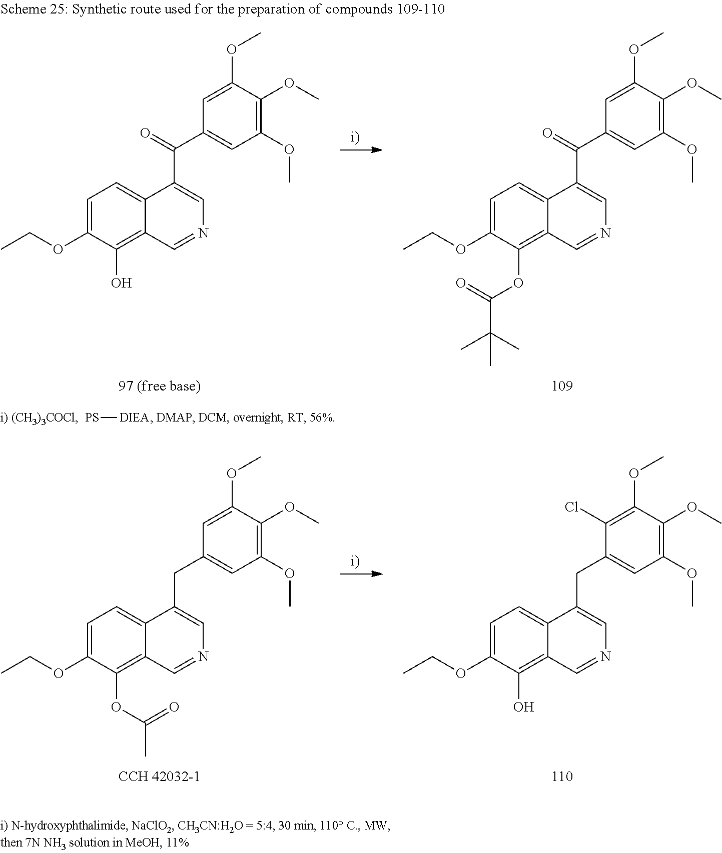

The compound 109 was obtained in 56% yield from compound 97 (free base) by overnight treatment with trimethylacetyl chloride in dichloromethane in presence of diisopropylaminomethyl-polystyrene and a catalytic amount of DMAP (scheme 25).

The reaction of the acetate CCH 42032-1 using sodium chlorite and N-hydroxyphtalimide as catalyst in a mixture of acetonitrile:H₂O=5:4 at 110° C. for 30 min under microwave irradiation provided the corresponding chloro acetate compound that was immediately deprotected using a 7 N ammonia solution to give the chloro compound 110 as an hydrochloride salt in 11% yield after a final HCl methanolic treatment (scheme 25).

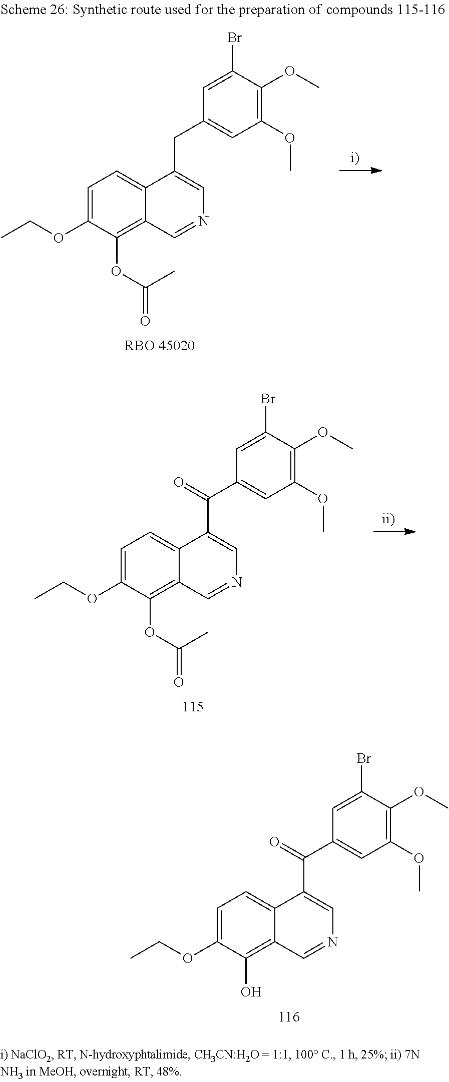

The acetate RBO 45020 was oxidized using sodium chlorite and N-hydroxyphtalimide as catalyst in a mixture of acetonitrile:H₂O=1:1 at 100° C. for 1 hour to obtain the corresponding keto compound 115 in 25% yield. Deprotection of the acetate 115 using a 7 N ammonia solution in methanol overnight at room temperature gave compound 116 as an hydrochloride salt in 48% yield after a final HCl methanolic treatment (scheme 26).

It should be understood that other ways of producing these compounds may be designed by the skilled person, based on common general knowledge and following guidance contained in this application.

Another object of the present invention is the intermediate compounds used for the preparation of compounds of formula (I). In particular, the present invention relates to the intermediate compounds herein below mentioned in the examples.

The compounds according to the invention can be in the form of salts, particularly acid or base salts, preferably compatible with pharmaceutical use (i.e. pharmaceutically acceptable salts of the compounds of the invention). It will be appreciated by those skilled in the art that non-pharmaceutically acceptable salts of compounds of formula (I) are also part of the present invention, since such non-pharmaceutically acceptable salts can be useful as intermediates in the preparation of pharmaceutically acceptable salts.

Salts of compounds of the invention include pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Base salts include, but are not limited to, those formed with pharmaceutically acceptable cations, such as sodium, potassium, lithium, calcium, magnesium, ammonium and alkylammonium. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Other examples of organic bases include lysine, arginine, guanidine, diethanolamine, choline and the like.

The present invention includes in particular cationic salts, for example sodium or potassium salts, or alkyl esters (e.g. methyl or ethyl) of the phosphate group.

The pharmaceutically acceptable salts can in particular be prepared by reacting the compound of formula (I) with acids such as hydrochloric acid, hydrobromic acid, nitric acid, sulfuric acid, phosphoric acid, p-toluenesulphonic acid, methanesulfonic acid, fonic acid, acetic acid, citric acid, maleic acid, salicylic acid, hydroxynaphthoic acid, ascorbic acid, palmitic acid, succinic acid, benzoic acid, benzenesulfonic acid, tartaric acid and the like in solvents like ethyl acetate, ether, alcohols, acetone, THF, dioxane, etc. Mixture of solvents may also be used.

The invention further relates to a prodrug of a compound of formula (I). The term “prodrug” encompasses those derivatives that are converted in vivo to the compounds of the invention. Such derivatives are readilly designed based on the structure of compounds of formula (I) as provided above and include, for example, compounds where a free hydroxy group is converted into an ester, for example an acetate or phosphate ester, or where a free amino group is converted into an amide (for example an α-amino acid amide, in particular a serine, amide). Procedures for esterifying, for example acylating, the compounds of the invention are well known in the art and may include treatment of the compound with an appropriate carboxylic acid, anhydride or chloride in the presence of a suitable catalyst or base. A particularly preferred prodrug is a disodium phosphate ester.

The compounds of the invention can be administered alone, but are generally administered with a pharmaceutical carrier, with respect to standard pharmaceutical practice (such as described in Remington's Pharmaceutical Sciences, Mack Publishing). Accordingly, a further object of this invention relates to a pharmaceutical composition comprising a compound of formula (I), as defined above, and a pharmaceutically acceptable carrier.

The carrier must be pharmaceutically “acceptable” in the sense of being compatible with the other ingredients of the invention, in particular with the compound of formula (I) present in the composition, and not injurious to the subject to be treated. Compositions of the invention include those suitable for oral, rectal, nasal, topical, vaginal or parenteral (e.g., subcutaneous, intramuscular, intravenous, intra-arterial, intradermal, intraperitoneal) administration. They can be presented in unit dosage form and can be prepared by any method well known to those skilled in the art of pharmacy.

The dosages and dosage regimen in which the compounds of formula (I) are administered will vary according to the dosage form, mode of administration, the condition being treated and particulars of the patient being treated. Accordingly, optimal therapeutic concentrations will be best determined at the time and place through routine experimentation.

The compounds according to the invention can be used enterally or parenterally. Orally, the compounds according to the invention are suitably administered in the amount from about 0.1 mg per day to 1,000 mg per day. For parenteral, sublingual, intranasal, or intrathecal administration, the compounds according to the invention are suitably used in the amount from about 0.5 to about 100 mg/day; for depo administration and implants from about 0.5 mg/day to about 50 mg/day; for topical administration from about 0.5 mg/day to about 200 mg/day; for rectal administration from about 0.5 mg to about 500 mg. In a preferred aspect, the therapeutically effective amounts for oral administration is from about 1 mg/day to about 100 mg/day; and for parenteral administration from about 5 to about 50 mg daily. In a more preferred aspect, the therapeutically effective amounts for oral administration are from about 5 mg/day to about 50 mg/day.

Compound of the present invention can be administered orally using any pharmaceutically acceptable dosage form known in the art for such administration. The vehicle may be any solution, suspension, powder, gel, etc., including isotonic solution, buffered and saline solutions, such as syrups or aqueous suspensions, etc. The compounds may be administered by any suitable route, including systemic delivery, intra-venous, intra-arterial, intra-cerebral or intrathecal injections. Repeated injections may be performed, if desired. The dosage can vary within wide limits and will have to be adjusted to the individual requirements in each particular case, depending upon several factors known to those of ordinary skill in the art. Agents determining the dosage of dosage the active compounds can be the pharmacodynamic characteristics of the particular agent and its mode and route of administration; the age, health and weight of the recipient; the nature and extent of the symptoms; the kind of concurrent treatment; the frequency of treatment; and the effect desired. A daily dosage of active ingredient can be expected to be about 0.001 to about 1000 milligrams per kilogram of body weight, with the preferred dose being about 0.1 to about 30 mg/kg. The daily oral dosage can vary from about 0.01 mg to 1000 mg, 0.1 mg to 100 mg, or 10 mg to 500 mg per day of a compound. The daily dose may be administered as single dose or in divided doses and, in addition, the upper limit can also be exceeded when this is found to be indicated.

The compounds of the present invention can be administered in such oral dosage forms as tablets, capsules (each of which can include sustained release or timed release formulations), pills, powders, granules, elixirs, tinctures, suspensions, syrups, and emulsions. Likewise, they may also be administered in intravenous (bolus or infusion), intraperitoneal, subcutaneous, or intramuscular form, all using dosage forms well known to those of ordinary skill in the pharmaceutical arts. An effective but non-toxic amount of the compound desired can be employed to treat a disease state for which tubulin polymerisation plays a crucial role.

Compounds can be administered by any means that produces contact of the active agent with the agent's site of action in the body of a host, such as a human or a mammal. They can be administered by any conventional means available for use in conjunction with pharmaceuticals, either as individual therapeutic agents or in a combination of therapeutic agents, either administered alone, or administered with a pharmaceutical carrier selected on the basis of the chosen route of administration and standard pharmaceutical practice.

The compound for the present invention can be administered in intranasal form via topical use of suitable intranasal vehicles, or via transdermal routes, using those forms of transdermal skin patches wall known to those of ordinary skill in that art.

Oral administration in the form of a tablet or capsule containing the active compound can be combined with an oral, non-toxic, pharmaceutically acceptable, inert carrier such as lactose, starch, sucrose, glucose, methyl cellulose, magnesium stearate, dicalcium phosphate, calcium sulfate, mannitol, sorbitol and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, and coloring agents can also be incorporated into the mixture. Sweetening agents such as those set forth above, and flavoring agents may be added to provide a palatable oral preparation. These compositions may be preserved by the addition of an anti-oxidant such as butylated hydroxyanisol or alpha-tocopherol. Suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methylcellulose, agar, bentonite, xanthan gum, and the like.

Compounds of the invention can also be administered in the form of liposomal particulate delivery systems, such as small unilamellar vesicles, large unilamallar vesicles, and multilamellar vesicles. Liposomes can be formed from a variety of phospholipids, such as cholesterol, stearylamine, or phosphatidylcholines.

Alternatively, compounds of the present invention may also be coupled with soluble polymers as targetable drug carriers, such as polymers made of polyvinylpyrrolidone, pyran copolymer, polyhydroxypropylmethacrylamide—phenol, polyhydroxyethylaspartamide—phenol, or polyethyleneoxide—polylysine substituted with palmitoyl residues. Polymers may also belong to the class of biodegradable polymers useful in achieving controlled release of a drug, for example, polylactic acid, polyglycolic acid, copolymers of polylactic and polyglycolic acid, polyepsilon caprolactone, polycyanoacylates, etc. or block copolymers of hydrogels.

Compounds of the present invention may be formulated into gelatin capsules with the addition of lactose, starch, cellulose derivatives, magnesium stearate, stearic acid, and the like as powdered carriers. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric coated for selective disintegration in the gastrointestinal tract. Liquid dosage forms for oral administration can contain coloring and flavoring to increase patient acceptance. In general, water, a suitable oil, saline, aqueous dextrose (glucose), and related sugar solutions and glycols such as propylene glycol or polyethylene glycols are suitable carriers for parenteral solutions. Solutions for parenteral administration preferably contain a water soluble salt of the active ingredient, suitable stabilizing agents, and if necessary, buffer substances. Antioxidizing agents such as sodium bisulfite, sodium sulfite, or ascorbic acid, either alone or combined, are suitable stabilizing agents. Also used are citric acid and its salts and sodium EDTA. In addition, parenteral solutions can contain preservatives, such as benzalkonium chloride, methyl- or propyl-paraben, and chlorobutanol.

The present invention also relates to a compound of formula (I) as a medicament. The compounds of the invention are particularly useful as anticancer agents or as vascular disrupting agents. The invention thus also relates to a compound of formula (I) as anti-cancer agent or as a angiogenesis and/or vascular disrupting agent.

The compounds and pharmaceutical compositions of the invention are more particularly intended to treat a disease state by inhibiting tubulin polymerisation.

The present invention thus provides a method for treating a disease state by inhibiting tubulin polymerisation, comprising the step of administering a compound of formula (I) to a patient in need thereof.

The present invention also provides the use of a compound of formula (I) or pharmaceutical composition as described above for the manufacture of a medicament for the treatment of a disease state by inhibiting tubulin polymerisation.

In a particular embodiment, the method, compound or pharmaceutical composition of the invention is used for the treatment of cancer, inflammation or a disorder caused by unwanted neovascularisation. Advantageously, the invention relates to the treatment of conditions in which angiogenesis must be inhibited, or established unwanted vascularisation must be disrupted.

“Treatment” or “treating” includes both therapeutic and prophylactic treatments. Accordingly, the compounds may be used at very early stages of a disease, or before early onset, or after significant progression, including metastasis in case of cancer. The term “treatment” or “treating” applied to tumour designates in particular a reduction of the burden in a patient, such as a reduction in cell proliferation rate, a destruction of diseased proliferative cells, a reduction of tumor mass or tumor size, a delaying of tumor progression, as well as a complete tumor suppression.

The term “tumour” or “cancer” is used to define any malignant cancerous growth and may include sarcomas, in particular Kaposi sarcoma, leukemias, melanomas, glioblastomas, oligodendroglioma, astrocytic glioma, thyroid, colon, ovarian, skin, breast, prostate, CNS, renal and lung cancers, and other cancers. In particular treatment of non-small cell lung cancers, liver neoplasms, meningeoma, testis cancer, uterine cancer, cervical neoplasm, bladder cancer, neuroblastoma, retinoblastoma, embryonal carcinoma, Wilm's tumors or Ewing's tumor is intended with compounds of the present invention.

However, it will be understood that compounds of the invention can be used in any disease state for which tubulin polymerisation plays a crucial role. In particular, the present compounds can be used to treat non oncology indications.

Compounds of formula (I) can in particular be used to treat inflammation. Inflammation can be acute or chronic, and inflammatory conditions may include rheumatoid arthritis, inflammatory bowel disease, Crohn's disease, psoriasis and the like.

The compounds of the invention may also be used to treat disorders caused by unwanted neovascularisation. The term “unwated neovascularisation” relates to a disorder involving vascularisation which should not occur, or which is abnormal either in localization or intensity. One can in particular treat angiogenesis disorders such as, but not limited to, age-related macular degeneration (ARMD), neovascular glaucoma, retinal vein obstruction, myopic macular degeneration, retinopathy of prematurity, proliferative diabetic retinopathy, posterior capsular opacification (PCO), pediatric hemangiomas, acne rosacea, Kaposi sarcoma, atopic keratitis, epidemic keratoconjunctivitis, bacterial ulcers, fungal ulcers, Herpes simplex infections, Herpes zoster infections, protozoan infections, Mycobacterium infections, polyarteritis, sarcoidosis, scleritis, flush, Sjogren's disease, systemic lupus, Acquired Immune Deficiency Syndrome (AIDS), syphilis and the like such as infection with Treponema pallidum or related parasites which results in increased angiogenesis.

The invention also relates to a compound of formula (I) or a pharmaceutical composition comprising a compound of formula (I), for inhibiting cancer cell proliferation or inhibiting endothelial cell proliferation.

The compounds of the invention may also be particularly useful in combination therapy, e.g. combining the treatment with other treatment or drugs with different mechanisms, such as chemotherapeutics or radiation treatment. For instance, in order to potentiate the anti-tumor efficacy of treatments using the compounds of the present invention, one or more of the following cytotoxic compounds may also be administered: oxaliplatin 5-fluorouracil, gemcitabine, Interferon alpha, paclitaxel, cisplatin, carboplatin, doxorubicin, caminomycin, daunorubicin, aminopterin, methotrexate, methopterin, mitomycin C, docetaxel, tyrosine kinase inhibitors (eg. Glivec), irinotecan hydrochloride, ecteinascidin 743, or pofiromycin, 6-mercaptopurine, gemcitabine, cytosine arabinoside, podophyllotoxin or podophyllotoxin derivatives such as etoposide, etoposide phosphate or teniposide, melphalan, vinblastine, vincristine, leurosidine, vindesine and leurosine. The combination therapy may also include the addition of an angiogenesis inhibitor (eg. Avastin) or another agent of therapy (eg. radiotherapy). The above examples are provided for illustration only, many other such compounds are known to those skilled in the art.

Compounds that are vascularly active may be preferentially administered with antihypertensive or antihypotensive agents.

The combination partners in such therapies may be administered together, one after the other, separately in one combined unit dosage or in separate unit dosage forms.

The invention thus relates in a particular aspect to a pharmaceutical composition comprising a compound of formula (I) in a pharmaceutically acceptable carrier as defined above, in combination with one or more therapeutic agent, in particular in combination with at least one of the therapeutic agents listed above.

The invention also relates in a further particular aspect to a kit of parts, comprising a first composition comprising a compound of formula (I) and a second composition comprising another therapeutic compound (such as those cited above) for separate, sequential or/and simultaneous administration to a subject.

As provided above, the compounds according to the invention may be administered according to various routes, typically by injection, such as local or systemic injection(s). Intratumoral injections are preferred for treating existing cancers. However, other administration routes may be used as well, such as intramuscular, intravenous, intradermic, subcutaneous, etc. Furthermore, repeated injections may be performed, if needed, although it is believed that limited injections will be needed in view of the efficacy of the compounds.

In view of the advantageous therapeutic potential of tubulin polymerisation inhibitors and vascular disrupting agents, the compound of the present invention, which elicit both activity, are useful in treating the diseases described above and the like. Accordingly, the invention relates to a method for treating a disease state by inhibiting tubulin polymerization and/or unwanted neovascularization, comprising administering to a subject in need thereof a compound of formula (I). The compound of formula (I) is administered in a therapeutically effective amount suitable for the specific disease to be treated.

Specifically, particular embodiments of the invention relate to a compound of formula (I) or a composition as defined above, for use in a method for the treatment of:
a disease state selected in the group consisting of a cancer, inflammation or a disorder caused by unwanted neovascularisation,
a cancer selected in the group consisting of sarcomas, in particular Kaposi sarcoma, leukemias, melanomas, glioblastomas, oligodendroglioma, astrocytic glioma, thyroid, colon, ovarian, skin, breast, prostate, CNS, renal and lung cancers, in particular non-small cell lung cancers, liver neoplasms, meningeoma, testis cancer, uterine cancer, cervical neoplasm, bladder cancer, neuroblastoma, retinoblastoma, embryonal carcinoma, Wilm's tumors or Ewing's tumor,
a cancer by combination therapy with other chemotherapeutic or radiation treatments, or with anti-angiogenic therapies,
a disease caused by abnormal angiogenesis, in particular age-related macular degeneration, neovascular glaucoma, retinal vein obstruction, myopic macular degeneration, retinopathy of prematurity, proliferative diabetic retinopathy, posterior capsular opacification (PCO), or pediatric hemangiomas,
acne rosacea, atopic keratitis, epidemic keratoconjunctivitis, bacterial ulcers, fungal ulcers, Herpes simplex infections, Herpes zoster infections, protozoan infections, Mycobacterium infections, polyarteritis, sarcoidosis, scleritis, flush, Sjogren's disease, systemic lupus, Acquired Immune Deficiency Syndrome (AIDS), syphilis or infection with Treponema pallidum or related parasites.

The invention further relates to a method of inhibiting proliferation of vascular endothelial cells, said method comprising contacting said cells with an effective amount of a compound of formula (I).

The invention also relates to a method of inhibiting proliferation of cancer cells, said method comprising contacting said cells with an effective amount of a compound of formula (I).

The invention further provides a method of inhibiting or disrupting microtubule polymerization in a cell, said method comprising contacting said cell with a compound of formula (I).

In a particular embodiment, the invention relates to the implementation of above-mentioned compound 1, 3, 10, 14, 15, 18, 19, 20, 21, 29, 31, 32, 33, 38, 40, 56, 57, 58, 59, 64, 65, 66, 67, 78, 96, 97, 98, 105 or 112, In particular of compound 3, 15, 29, 57, 58, 64, 66, 67 or 97.

Further aspects and advantages of this invention will be disclosed in the following examples, which should be regarded as illustrative and not limiting the scope of this application.

### EXAMPLES

Herein below are presented the origin, synthesis and physico-chemical properties of compounds 1 to 116 according to formula (I).

### General:

¹H-NMR and ¹³C-NMR spectra were recorded at ambient temperature with an Advance 300 (Bruker) spectrometer.

The compounds were analyzed by reverse phase high performance liquid chromatography (HPLC) using a Waters Autopurification System equipped with a Waters 2525 Pump, a Waters 2696 photodiode array detector. The Method A (10 min) was performed with an XTerra™ column (5 μm, C18, 4.5×50 mm, Model #186000482) or an XBridge™ column (5 μm, C18, 4.5×50 mm, Model #186003113). Solvent A was H₂O with 0.05% TFA and solvent B was CH₃CN with 0.05% TFA. The 10 min gradient run was realized using 1.0 mL min⁻¹ with 5% B in A (0.0-1.0 min), 5% to 100% B in A (1.0-7.0 min), 100% to 5% B in A (7.0-7.5 min), 5 B in A (7.5-10.0 min). The 5 min gradient run (when precised) was realized using 1.0 mL min⁻¹ with 5% B in A (0.0-0.25 min), 5% to 100% B in A (0.25-3.0 min), 100% to 5% B in A (3.0-4.0 min), 5% B in A (4.0-5.0 min).

Melting points were measured with a Büchi B-545 melting point apparatus and were uncorrected. Microwave reactions were performed in a Biotage Initiator 60 EXP microwave reactor.

To isolate reaction products the solvent were removed by evaporation using a vacuum rotatory evaporator, unless otherwise indicated, the water bath temperature did not exceed 40° C.

### Preparation of 4-(3,4,5-trimethoxybenzyl)-7-ethoxyisoquinolin-8-ol hydrochloride 1

### (E)-2-((2,2-Diethoxyethylimino)methyl)-6-ethoxyphenol SAO 33012

Under anhydrous conditions (Dean Stark apparatus), 3-ethoxysalicylaldehyde (5.00 g, 30.1 mmol) was dissolved in toluene (81.8 mL). Aminoacetaldehyde diethyl acetal (6.6 mL, 45.1 mmol) was added via syringe at 20° C. Water (25 mL) and toluene (5 mL) were added in the Dean Stark burette. The reaction mixture was stirred at 150° C. for 4 h under an N₂ atmosphere. The reaction mixture was evaporated under vacuum at 60° C. to yield to (E)-2-((2,2-diethoxyethylimino)methyl)-6-ethoxyphenol SAO 33012 as brown oil (9.14 g, 100% yield). This crude product was used in the next step without further purification.

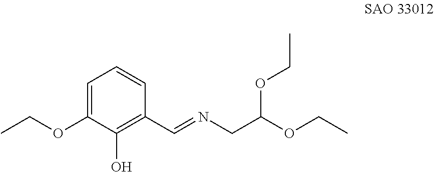 [SMILES: CCOc1cccc(/C=N/CC(OCC)OCC)c1O]

MW: 281.35; Yield: 100%; Brown oil.

¹H-NMR (CDCl₃, δ): 1.20 (t, 6H, J=7.0 Hz, 2×CH₂CH₃), 1.49 (t, 3H, J=7.0 Hz, CH₂CH₃), 3.52-3.62 (m, 2H, CH₃CH₂O), 3.62-3.75 (m, 2H, CH₃CH₂O), 3.75 (d, 2H, J=6.2 Hz, CH₂CH(OEt)₂), 4.13 (q, 2H, J=14.0 Hz, CH₃CH₂O), 4.71 (t, 1H, J=5.4 Hz, CH₂CH(OEt)₂), 6.76-6.95 (m, 3H, 3×ArH), 8.34 (s, 1H, CH═N).

### 2-((2,2-Diethoxyethylamino)methyl)-6-ethoxyphenol SAO 33014

In a 100 mL round bottom flask, the crude product (E)-2-((2,2-diethoxyethylimino)methyl)-6-ethoxyphenol SAO 33012 (9.14 g, 30.10 mmol) and NaBH₄ (2.16 g, 57.19 mmol) were dissolved in absolute ethanol (66.8 mL). The reaction mixture was stirred at 100° C. for 1 h under an N₂ atmosphere. Water (2 mL) was added at RT and the solvents were evaporated at 45° C. Water (80 mL) and CH₂Cl₂ (280 mL) were added to the mixture. The separated organic layer was washed with brine, dried over MgSO₄, filtered and concentrated to dryness under reduced pressure to give 2-((2,2-diethoxyethylamino)methyl)-6-ethoxyphenol SAO 33014 as a pale yellow solid (9.28 g, 100% yield). This crude product was used in the next step without further purification.

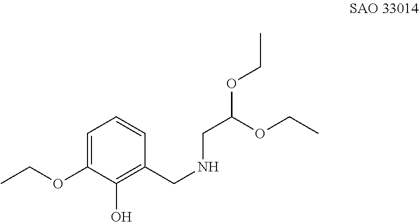 [SMILES: CCOc1cccc(CNCC(OCC)OCC)c1O]

MW: 283.37; Yield: 100%; Pale yellow solid; Mp (° C.): 55.2

¹H-NMR (CDCl₃, δ): 1.20 (t, 6H, J=7.1 Hz, 2×CH₃CH₂O), 1.47 (t, 3H, J=7.0 Hz, OCH₂CH₃), 2.77 (d, 2H, J=5.6 Hz, CH₂CH(OEt)₂), 3.58-3.80 (m, 4H, 2×CH₂CH₃), 4.00 (s, 2H, ArCH₂NH), 4.09 (q, 2H, CH₃CH₂OAr), 4.61 (t, 1H, J=5.6 Hz, OCHO), 6.61-6.83 (m, 3H, 3×ArH), NH not seen.

¹³C-NMR (CDCl₃, δ): 14.9, 15.4 (2×C), 50.8, 52.1, 62.8 (2×C), 64.3, 101.6, 112.4, 118.6, 120.6, 122.9, 147.3, 147.5.

MS-ESI m/z (% rel. Int.): 284.3 ([MH]⁺, 50), 238.2 (100).

HPLC: Method A, detection UV 254 nm, RT=4.67 min, peak area 90%.

### 4-(3,4,5-Trimethoxybenzyl)-7-ethoxyisoquinolin-8-ol hydrochloride 1

In an ace pressure tube (Aldrich, 38 mL), 2-((2,2-diethoxyethylamino)methyl)-6-ethoxyphenol SAO 33014 (722 mg, 2.55 mmol) and 3,4,5-trimethoxybenzaldehyde (500 mg, 2.55 mmol) were dissolved in EtOH (2.8 mL) and a HCl 37% solution (2.8 mL) was added. The reaction was stirred at 90° C. for 30 min, cooled at 4° C. and concentrated. The crude product was transformed into free base with a 1 N NH₄OH aqueous solution (30 mL) and extracted with CH₂Cl₂ (2×80 mL). The combined organic layer were washed with brine (30 mL), dried over MgSO₄, filtered and evaporated to give a yellow solid. This solid was purified by column chromatography (SiO₂, eluent cyclohexane:acetone=8:2 to 7:3) to give, after evaporation and drying, 4-(3,4,5-trimethoxybenzyl)-7-ethoxyisoquinolin-8-ol (320 mg) that was dissolved in MeOH (4 mL) and a 1.51 N HCl solution in MeOH (598 μL, 0.90 mmol) was slowly added at 4° C. The reaction mixture was stirred at 4° C. for 15 min. After evaporation of the solvent and drying under vacuum pump under P₂O₅, 4-(3,4,5-trimethoxybenzyl)-7-ethoxyisoquinolin-8-ol hydrochloride 1 was obtained as a yellow solid (345 mg, 33% yield).

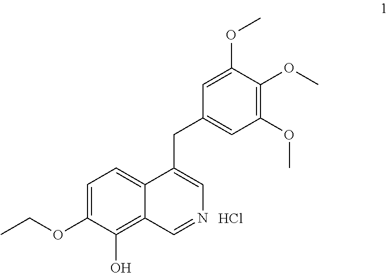 [SMILES: CCOc1ccc2c(Cc3cc(OC)c(OC)c(OC)c3)cncc2c1O ; Cl]

MW: 405.87; Yield: 33%; Yellow solid; Mp (° C.): 235.8

R_{f}: 0.2 (cyclohexane:acetone=7:3, free base).

¹H-NMR (CD₃OD, δ): 1.50 (t, 3H, J=7.0 Hz, CH₂CH₃), 3.73 (s, 3H, OCH₃), 3.77 (s, 6H, 2×OCH₃), 4.34 (q, 2H, J=7.0 Hz, CH₂CH₃), 4.47 (s, 2H, CH₂), 6.61 (s, 2H, 2×ArH), 7.87 (d, 1H, J=9.1 Hz, ArH), 8.00-8.06 (m, 2H, 2×ArH), 9.65 (s, 1H, ArH).

¹³C-NMR (CD₃OD, δ): 15.1, 37.1, 56.7 (2×C), 61.1, 66.9, 107.6 (2×C), 116.6, 120.5, 126.7, 128.6, 132.8, 135.2, 137.9, 138.2, 142.6, 146.4, 146.8, 155.0 (2×C).

MS-ESI m/z (% rel. Int.): 370.3 ([MH]⁺, 100).

HPLC: Method A, detection UV 254 nm, RT=4.44 min, peak area 98.0%.

### Preparation of 7-ethoxy-4-(3,4,5-trimethoxybenzyl)isoquinolin-8-yl trifluoromethanesulfonate hydrochloride 2

### 2-((2,2-Diethoxyethylamino)methyl)-6-ethoxyphenol SAO 33014

To a solution of aminoacetaldehyde diethyl acetal (4.4 mL, 30.26 mmol) in MeOH (100 mL) in a 250 mL round-bottomed flask equipped with a magnetic stirrer was added AcOH (2.9 mL). 3-Ethoxysalicylaldehyde (5.03 g, 30.27 mmol) was added followed by portionwise addition of sodium cyanoborohydride (2.09 g, 33.26 mmol) and the reaction mixture was stirred overnight at RT. An aqueous solution of NaOH (10 N, 5.0 mL, 50.0 mmol) was then added at RT and the mixture was stirred for 30 min before evaporation of the solvent at 40° C. under vacuum. The residue was taken up in CH₂Cl₂ (150 mL) and the organic solution was washed with water (20 mL) then with brine (20 mL), dried over Na₂SO₄, filtered and concentrated at 40° C. under vacuum. The oily residue was purified by column chromatography (SiO₂, eluent: gradient cyclohexane:EtOAc=100:0 to 66:33) to give, after evaporation and drying, a pale yellow solid 2-((2,2-diethoxyethylamino)methyl)-6-ethoxyphenol SAO 33014 (6.0 g 70% yield).

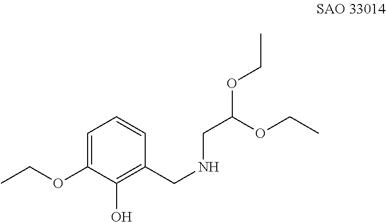 [SMILES: CCOc1cccc(CNCC(OCC)OCC)c1O]

MW: 283.37; Yield: 70%; Pale yellow solid; Mp (° C.): 55

R_{f}: 0.2 (cyclohexane:EtOAc=2:1).

MS-ESI m/z (rel. int.): 284 ([MH]⁺, 25), 238 (100).

### 7-Ethoxy-4-(3,4,5-trimethoxybenzyl)isoquinolin-8-ol CCH 34046-2

A mixture of the above solid 2-((2,2-diethoxyethylamino)methyl)-6-ethoxyphenol SAO 33014 (2.00 g, 7.06 mmol) and 3,4,5-trimethoxybenzaldehyde (1.39 g, 7.08 mmol) in a mixture of EtOH:conc. HCl=1:1 (5 mL) in a 20 mL microwave vial equipped with a magnetic stirrer was stirred for 20 min at 100° C. under microwave irradiation. The volatiles were removed at 40° C. under vacuum and the residue was taken up in CH₂Cl₂ (100 mL) before neutralisation with a saturated aqueous NaHCO₃ solution. The separated organic phase was washed with water (20 mL), with brine (20 mL), dried over Na₂SO₄, filtered and concentrated at 40° C. under vacuum. The obtained crude product was purified by column chromatography (SiO₂, eluent: gradient cyclohexane:acetone=100:0 to 76:24) to give, after evaporation and drying, 7-ethoxy-4-(3,4,5-trimethoxybenzyl)isoquinolin-8-ol CCH 34046-2 as a brown solid (0.77 g, 30% yield).

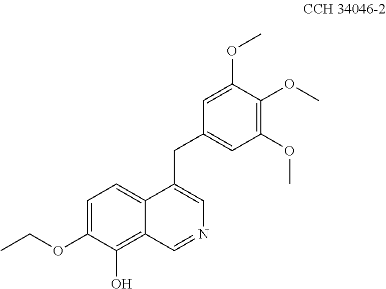 [SMILES: CCOc1ccc2c(Cc3cc(OC)c(OC)c(OC)c3)cncc2c1O]

R_{f}: 0.2 (cyclohexane:acetone=76:24).

MS-ESI m/z (rel. int.): 370 ([MH]⁺, 100).

### 7-Ethoxy-4-(3,4,5-trimethoxybenzyl)isoquinolin-8-yl trifluoromethanesulfonate hydrochloride 2

To a suspension of CCH 34046-2 (168 mg, 455 μmol) in DMF (3 mL) in a 25 mL round-bottomed flask equipped with a magnetic stirrer was added NEt₃ (0.13 mL, 935 μmol) and N-phenyl-bis(trifluoromethanesulfonimide) (0.24 g, 672 μmol). The reaction mixture was stirred for 20 min at RT then diluted with Et₂O (50 mL) and the organic solution was washed with water (10 mL), brine (10 mL), dried over Na₂SO₄, filtered and concentrated at 40° C. under vacuum. Purification by column chromatography (SiO₂, eluent: gradient cyclohexane:EtOAc=100:0 to 75:25) gave, after evaporation and drying, a pale brown solid 7-ethoxy-4-(3,4,5-trimethoxybenzyl)isoquinolin-8-yl trifluoromethanesulfonate CCH 34050 (206 mg, 90% yield). This solid CCH 34050 (43 mg, 86 μmol) was dissolved in MeOH (3 mL) in a 25 mL round-bottomed flask equipped with a magnetic stirrer and the solution was cooled to 0° C. in an ice bath and a 0.26N HCl solution in MeOH (0.5 mL) was added. The solution was stirred for 15 min at 0° C. and concentrated to dryness at RT under vacuum to afford 7-ethoxy-4-(3,4,5-trimethoxybenzyl)isoquinolin-8-yl trifluoromethanesulfonate hydrochloride 2 as a pale brown solid (46 mg, 100% yield).

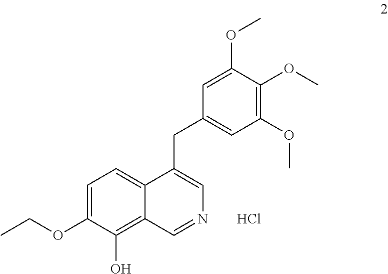 [SMILES: CCOc1ccc2c(Cc3cc(OC)c(OC)c(OC)c3)cncc2c1O ; Cl]

MW: 537.93; Yield: 90%; Pale brown solid; Mp (° C.): 182.2

R_{f}: 0.2 (cyclohexane:EtOAc=3:1, free base).

¹H-NMR (CD₃OD, δ): 1.54 (t, 3H, J=6.8 Hz, CH₂CH₃), 3.73 (s, 3H, OCH₃), 3.77 (s, 3H, OCH₃), 3.78 (s, 3H, OCH₃), 4.51 (q, 2H, J=6.8 Hz, CH₂CH₃), 4.61 (s, 2H, CH₂), 6.64 (s, 2H, 2×ArH), 8.33 (d, 1H, J=9.4 Hz), 8.40 (s, 1H, ArH), 8.59 (d, 1H, J=9.4 Hz), 9.47 (s, 1H, ArH).

¹³C-NMR (CD₃OD, δ): 14.8, 37.0, 56.8 (2×C), 61.1, 68.0, 107.7 (2×C), 120.1 (q, J=319.9 Hz), 124.4, 127.2, 128.1, 131.8, 133.2, 133.9, 134.7, 138.4, 139.6, 140.0, 152.8 (2×C), 155.1.

MS-ESI m/z (rel. int.): 502 ([MH]⁺, 100), 369 (50).

HPLC: Method A, detection UV 254 nm, RT=5.86 min, peak area 97.8%.

### Preparation of 7-ethoxy-4-(3,4,5-trimethoxybenzyl)isoquinolin-8-amine dihydrochloride 3

### 7-Ethoxy-4-(3,4,5-trimethoxybenzyl)isoquinolin-8-amine dihydrochloride 3

A mixture of (±) BINAP (100 mg, 0.16 mmol), Pd₂(dba)₃ (40 mg, 0.04 mmol), Cs₂CO₃ (0.50 g, 1.53 mmol), 7-ethoxy-4-(3,4,5-trimethoxybenzyl)isoquinolin-8-yl trifluoromethane-sulfonate CCH 34050 (377 mg, 0.75 mmol) and benzophenone imine (300 mg, 1.66 mmol) in dry toluene (15 mL) in a 20 mL microwave vial equipped with a magnetic stirrer was stirred for 5 h at 150° C. under microwave irradiation. After cooling to RT the reaction mixture was diluted with THF (50 mL) and filtered through celite. The filtrate was poured in a 250 mL round-bottomed flask equipped with a magnetic stirrer before addition of 1 N aqueous HCl solution (15 mL) and the mixture was stirred for 40 min at RT. The volatiles were then removed at 40° C. under vacuum and the residue was taken up in CH₂Cl₂ (100 mL), neutralized with saturated aqueous NaHCO₃ solution, washed with H₂O (20 mL), brine (10 mL), dried over Na₂SO₄, filtered and concentrated at 40° C. under vacuum. Purification by column chromatography (SiO₂, eluent: gradient CH₂Cl₂:EtOAc=100:0 to 33:66 then 0:100) gave, after evaporation and drying, 7-ethoxy-4-(3,4,5-trimethoxybenzyl)isoquinolin-8-amine CCH 34058 (177 mg, 64%). CCH 34058 (22 mg, 60 μmol) was then dissolved in MeOH (3 mL) in a 25 mL round-bottomed flask equipped with a magnetic stirrer and the solution was cooled to 0° C. in an ice bath before adding 0.7 mL of a 0.26N HCl solution in MeOH. The solution was stirred for 15 min at 0° C. and concentrated to dryness at RT under vacuum to afford 3 as a red solid (26 mg, 100%).

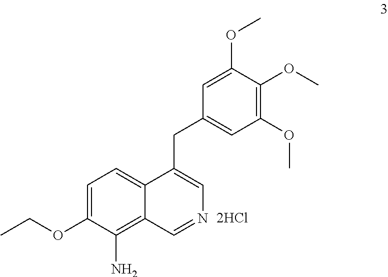 [SMILES: CCOc1ccc2c(Cc3cc(OC)c(OC)c(OC)c3)cncc2c1N]

MW: 441.35; Yield: 64%; Red solid; Mp (° C.)>250 (dec.)

R_{f}: 0.2 (EtOAc, free base).

¹H-NMR (CD₃OD, δ): 1.54 (t, 3H, J=6.9 Hz, CH₂CH₃), 3.82 (s, 9H, 3×OCH₃), 4.30 (q, 2H, J=6.9 Hz, CH₂CH₃), 4.37 (s, 2H, CH₂), 6.50 (s, 2H, 2×ArH), 7.49 (d, 1H, J=8.8 Hz), 7.73 (d, 1H, J=8.8 Hz), 7.76 (s, 1H, ArH), 9.74 (s, 1H, ArH).

¹³C-NMR (CD₃OD, δ): 15.0, 37.0, 56.5 (2×C), 61.1, 65.7, 106.6 (2×C), 111.7, 116.0, 123.0, 126.0, 131.3, 133.7, 136.4, 137.4, 138.9, 141.7, 144.7, 154.1 (2×C).

MS-ESI m/z (rel. int.): 369 ([MH]⁺, 100).

HPLC: Method A, detection UV 254 nm, RT=4.69 min, peak area 96.6%.

### Preparation of N-(7-ethoxy-4-(3,4,5-trimethoxybenzyl)isoquinolin-8-yl)acetamide hydrochloride 4

### N-(7-Ethoxy-4-(3,4,5-trimethoxybenzyl)isoquinolin-8-yl)acetamide hydrochloride 4

To a solution of 7-ethoxy-4-(3,4,5-trimethoxybenzyl)isoquinolin-8-amine CCH 34058 (43 mg, 117 μmol) and NEt₃ (21 μL, 151 μmol) in dry CH₂Cl₂ (5 mL) in a 25 mL round-bottomed flask equipped with a magnetic stirrer was added acetyl chloride (12 μL, 168 μmol) and the mixture was stirred overnight at RT, quenched by adding a 2 N aqueous NaOH solution (3 mL). The organic phase was diluted with CH₂Cl₂ (20 mL), washed with brine (5 mL), dried over Na₂SO₄, filtered and concentrated at 40° C. under vacuum. Purification by column chromatography (SiO₂, eluent: gradient CH₂Cl₂:MeOH=100:0 to 96:4) gave, after evaporation and drying, N-(7-ethoxy-4-(3,4,5-trimethoxybenzyl)isoquinolin-8-yl)acetamide as a pale yellow solid CCH 34064 (35 mg, 73% yield). CCH 34064 (35 mg, 85 μmol) was dissolved in MeOH (3 mL) in a 25 mL round-bottomed flask equipped with a magnetic stirrer and the solution was cooled to 0° C. in an ice bath before adding 0.6 mL of a 0.22 N HCl solution in MeOH. The solution was stirred for 15 min at 0° C. before concentration to dryness at RT under vacuum to afford a pale yellow solid 4 (38 mg, 100% yield).

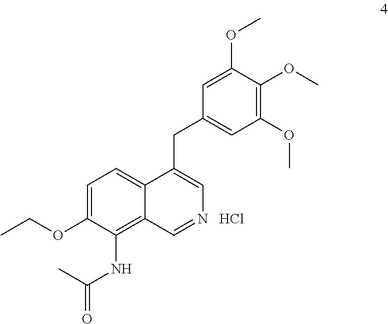 [SMILES: CCOc1ccc2c(Cc3cc(OC)c(OC)c(OC)c3)cncc2c1NC(C)=O ; Cl]

MW: 446.92; Yield: 73%; Pale yellow solid; Mp (° C.): 226.1 (dec.)

R_{f}: 0.2 (CH₂Cl₂:MeOH=96:4, free base).

¹H-NMR (CD₃OD, δ): 1.49 (t, 3H, J=7.0 Hz, CH₂CH₃), 2.34 (s, 3H, CH₃), 3.71 (s, 3H, OCH₃), 3.72 (s, 3H, OCH₃), 3.77 (s, 3H, OCH₃), 4.30 (q, 2H, J=7.0 Hz, CH₂CH₃), 4.55 (s, 2H, CH₂), 6.62 (s, 2H, 2×ArH), 8.16 (d, 1H, J=9.4 Hz), 8.22 (s, 1H, ArH), 8.43 (d, 1H, J=9.4 Hz), 9.43 (s, 1H, ArH).

¹³C-NMR (CD₃OD, δ): 15.1, 23.0, 37.0, 56.7 (2×C), 61.1, 66.9, 107.6 (2×C), 123.7, 126.3, 126.4, 127.3, 129.3, 133.7, 135.1, 138.2, 139.0, 143.6, 155.0 (2×C), 155.8, 173.8.

MS-ESI m/z (rel. int.): 411 ([MH]⁺, 100).

HPLC: Method A, detection UV 254 nm, RT=4.37 min, peak area 97.8%.

### Preparation of N-(7-ethoxy-4-(3,4,5-trimethoxybenzyl)isoquinolin-8-yl)methanesulfonamide hydrochloride 5

### N-(7-Ethoxy-4-(3,4,5-trimethoxybenzyl)isoquinolin-8-yl)methanesulfonamide hydrochloride 5

To a solution of 7-ethoxy-4-(3,4,5-trimethoxybenzyl)isoquinolin-8-amine CCH 34058 (40 mg, 109 μmol) and NEt₃ (30 μL, 216 μmol) in dry CH₂Cl₂ (5 mL) in a 25 mL round-bottomed flask equipped with a magnetic stirrer was added methanesulfonyl chloride (10 μL, 129 μmol) and the mixture was stirred overnight at RT. Another portion of methanesulfonyl chloride (10 μL, 129 μmol) and NEt₃ (30 μL, 216 μmol) was then added and stirring was continued for another day at RT. The volatiles were removed at 40° C. under vacuum and the residue was taken up in MeOH (5 mL) before addition of 2 N aqueous NaOH (3 mL). The mixture was stirred for 1 h at 50° C. then concentrated at 40° C. under vacuum and the resulting material was taken up in CH₂Cl₂ (25 mL) and H₂O (5 mL). The organic phase was isolated, washed with brine (5 mL), dried over Na₂SO₄, filtered and concentrated at 40° C. under vacuum. Purification by column chromatography (SiO₂, eluent: gradient cyclohexane:EtOAc=100:0 to 0:100) gave, after evaporation and drying, N-(7-ethoxy-4-(3,4,5-trimethoxybenzyl)isoquinolin-8-yl)methanesulfonamide CCH 34070 (18 mg, 37% yield). CCH 34070 (18 mg, 40 μmol) was dissolved in MeOH (3 mL) in a 25 mL round-bottomed flask equipped with a magnetic stirrer and the solution was cooled to 0° C. in an ice bath before adding a 0.22 N HCl solution in MeOH (0.3 mL). The solution was stirred for 15 min at 0° C. before concentration to dryness at RT under vacuum to afford N-(7-ethoxy-4-(3,4,5-trimethoxybenzyl)isoquinolin-8-yl)methanesulfonamide hydrochloride 5 as a pale brown solid (19 mg, 100% yield).

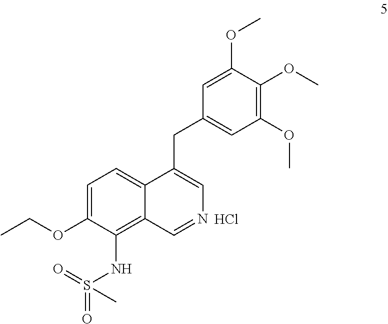 [SMILES: CCOc1ccc2c(Cc3cc(OC)c(OC)c(OC)c3)cncc2c1NS(C)(=O)=O ; Cl]

MW: 482.98; Yield: 37%; Pale brown solid; Mp (° C.): 238.3 (dec.)

R_{f}(free base): 0.2 (EtOAc).

¹H-NMR (CDCl₃ exchanged with CD₃OD, δ): 1.63 (t, 3H, J=5.9 Hz, CH₂CH₃), 3.18 (s, 3H, CH₃), 3.81 (s, 3H, OCH₃), 3.83 (s, 3H, OCH₃), 3.84 (s, 3H, OCH₃), 4.38-4.43 (m, 2H, CH₂CH₃), 4.43 (s, 2H, CH₂), 6.42 (s, 2H, 2×ArH), 7.90 (d, 1H, J=8.9 Hz), 8.12 (s, 1H, ArH), 8.22 (d, 1H, J=8.9 Hz), 9.79 (s, 1H, ArH).

¹³C-NMR (CDCl₃ exchanged with CD₃OD, δ): 14.8, 36.6, 41.5, 56.2 (2×C), 60.8, 66.0, 106.0 (2×C), 122.1, 123.8, 125.0, 128.2, 132.2, 132.6, 135.9, 137.2, 142.9, 153.8 (2×C), 154.5.

MS-ESI m/z (rel. int.): 447 ([MH]⁺, 100).

HPLC: Method A, detection UV 254 nm, RT=4.41 min, peak area 95.4%.

### Preparation of 7-ethoxy-8-(methylsulfonyl)-4-(3,4,5-trimethoxybenzyl)isoquinoline hydrochloride 6

### 7-Ethoxy-8-(methylsulfonyl)-4-(3,4,5-trimethoxybenzyl)isoquinoline hydrochloride 6

A mixture of 7-ethoxy-4-(3,4,5-trimethoxybenzyl)isoquinolin-8-yl trifluoromethanesulfonate CCH 34050 (95 mg, 189 μmol), methanesulfinic acid sodium salt (85%, 25 mg, 208 μmol), Pd₂(dba)₃ (20 mg, 22 μmol), 9,9-dimethyl-4,5-bis(diphenylphosphino)xanthene (22 mg, 38 μmol) and Cs₂CO₃ (0.11 g, 338 μmol) in toluene (5 mL) in a 20 mL microwave vial equipped with a magnetic stirrer was stirred for 5 h at 150° C. under microwave irradiation. After cooling to RT, the mixture was filtered through celite and the flask was rinsed several times with EtOAc (4×10 mL) and the washings were filtered through a celite plug. The filtrate was washed with brine (10 mL), dried over Na₂SO₄, filtered and concentrated at 40° C. under vacuum. Purification by column chromatography (SiO₂, gradient cyclohexane:EtOAc=100:0 to 0:100) afforded, after evaporation and drying, 7-ethoxy-8-(methylsulfonyl)-4-(3,4,5-trimethoxybenzyl)isoquinoline CCH 34088 (12.5 mg, 15%). CCH 34088 (12.5 mg, 29 μmol) was then dissolved in MeOH (3 mL) in a 25 mL round-bottomed flask equipped with a magnetic stirrer and the solution was cooled to 0° C. in an ice bath before adding a 0.22 N HCl solution in MeOH (0.2 mL). The solution was stirred for 15 min at 0° C. before concentration to dryness at RT under vacuum to afford 7-ethoxy-8-(methylsulfonyl)-4-(3,4,5-trimethoxybenzyl)isoquinoline hydrochloride 6 as a pale brown solid (13.5 mg, 100% yield).

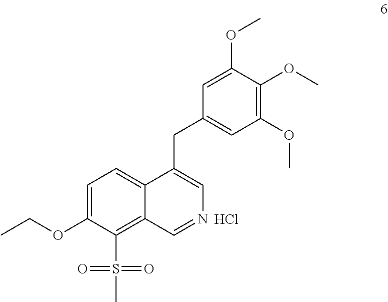 [SMILES: CCOc1ccc2c(Cc3cc(OC)c(OC)c(OC)c3)cncc2c1S(C)(=O)=O ; Cl]

MW: 467.96; Yield: 15%; Pale brown solid; Mp (° C.): 191.4 (dec.)

R_{f}: 0.2 (EtOAc, free base).

¹H-NMR (CDCl₃, δ): 1.63 (t, 3H, J=6.6 Hz, CH₂CH₃), 3.49 (s, 3H, CH₃), 3.79 (s, 3H, OCH₃), 3.80 (s, 3H, OCH₃), 3.82 (s, 3H, OCH₃), 4.49 (s, 2H, CH₂), 4.53 (q, 2H, J=6.6 Hz, CH₂CH₃), 6.37 (s, 2H, 2×ArH), 8.00 (d, 1H, J=7.3 Hz), 8.29 (s, 1H, ArH), 8.58 (d, 1H, J=7.3 Hz), 10.76 (s, 1H, ArH).

¹³C-NMR (CDCl₃, δ): 14.7, 37.1, 45.6, 56.3 (2×OCH₃), 60.9, 67.7, 106.0 (2×CH), 123.7, 124.4 (CH), 126.1, 129.9 (CH), 131.9 (CH), 132.0, 133.1, 137.0, 137.4, 141.1 (CH), 153.9 (2×C).

MS-ESI m/z (rel. int.): 432 ([MH]⁺, 100), 370 (13).

HPLC: Method A, detection UV 254 nm, RT=4.75 min, peak area 97.5%.

### Preparation of tert-butyl N-(7-ethoxy-4-(3,4,5-trimethoxybenzyl)isoquinolin-8-yl)sulfamoylcarbamate 7

### tert-butyl N-(7-ethoxy-4-(3,4,5-trimethoxybenzyl)isoquinolin-8-yl)sulfamoylcarbamate 7

To a solution of chlorosulfonyl isocyanate (16 μl, 184 μmol) in dry THF (5 mL) at 0° C. in a 25 mL round-bottomed flask equipped with a magnetic stirrer was added dropwise tert-BuOH (13.6 mg, 17.4 μl, 183 μmol) and the mixture was stirred for 2 h at RT. 7-Ethoxy-4-(3,4,5-trimethoxybenzyl)isoquinolin-8-amine CCH 34058 (60 mg, 163 μmol) was added to the reaction mixture and stirring was continued overnight at RT. The volatiles were removed at 40° C. under vacuum and the residue was taken up in CH₂Cl₂ (20 mL). The organic solution was washed with water (5 mL), brine (5 mL), dried over Na₂SO₄, filtered and concentrated at 40° C. under vacuum. Purification by column chromatography (SiO₂, eluent: gradient CH₂Cl₂:MeOH=100:0 to 96:4) gave, after evaporation and drying, N-(7-ethoxy-4-(3,4,5-trimethoxybenzyl)isoquinolin-8-yl)sulfamoylcarbamate 7 as a brown solid (61 mg, 68% yield).

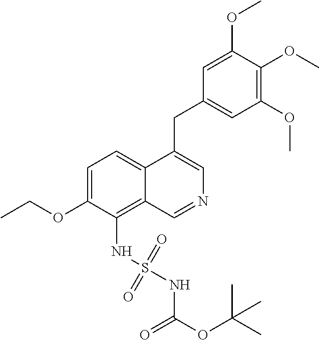 [SMILES: CCOc1ccc2c(Cc3cc(OC)c(OC)c(OC)c3)cncc2c1NS(=O)(=O)NC(=O)OC(C)(C)C]

MW: 547.62; Yield: 68%; Brown solid; Mp (° C.): 190.9 (dec.)

R_{f}: 0.3 (CH₂Cl₂:MeOH=95:5).

¹H-NMR (CDCl₃, δ): 1.44-1.52 (m, 3H, CH₂CH₃), 1.48 (s, 9H, C(CH₃)₃), 3.73 (s, 6H, 2×OCH₃), 3.80 (s, 3H, OCH₃), 4.21 (s, 2H, CH₂), 4.29 (q, 2H, J=6.7 Hz, CH₂CH₃), 6.36 (s, 2H, 2×ArH), 7.50 (d, 1H, J=9.1 Hz), 7.88 (d, 1H, J=9.1 Hz), 8.17 (s, 1H, ArH), 9.72 (s, 1H, ArH).

¹³C-NMR (CDCl₃, δ): 14.8, 28.1, 36.7, 56.1 (2×C), 60.8, 65.6, 82.8, 105.7 (2×C), 119.1, 120.1, 124.6, 127.0, 129.7, 130.6, 134.9, 136.6, 139.2, 147.9, 151.6, 152.0, 153.4 (2×C).

MS-ESI m/z (rel. int.): 548 ([MH]⁺, 100), 448 (28).

### Preparation of 7-ethoxy-4-(3,4,5-trimethoxy-benzyl)isoquinolin-8-yl]-sulfamide hydrochloride 8

### 7-Ethoxy-4-(3,4,5-trimethoxy-benzyl)isoquinolin-8-yl]-sulfamide hydrochloride 8

A solution of N-(7-ethoxy-4-(3,4,5-trimethoxybenzyl)isoquinolin-8-yl)sulfamoylcarbamate 7 (42 mg, 77 μmol) in TFA (2 mL) in a 10 mL round-bottomed flask equipped with a magnetic stirrer was stirred overnight at RT. The volatiles were removed at 40° C. under vacuum and the residue was neutralized with a saturated aqueous NaHCO₃ solution (20 mL) and extracted with CH₂Cl₂ (2×10 mL). The combined organic layers were washed with brine (5 mL), dried over Na₂SO₄, filtered and concentrated at 40° C. under vacuum. Purification by column chromatography (SiO₂, eluent: CH₂Cl₂:MeOH=100:0 to 93:7) gave, after evaporation and drying, CCH 34126 as a brown oil (9 mg, 26% yield). The oil CCH 34126 (9 mg, 20 μmol) was dissolved in MeOH (3 mL) in a 25 mL round-bottomed flask equipped with a magnetic stirrer and the solution was cooled to 0° C. in an ice bath before adding a 0.22 N HCl solution in MeOH (0.15 mL). The solution was stirred for 15 min at 0° C. before concentration to dryness at RT under vacuum to afford 7-ethoxy-4-(3,4,5-trimethoxy-benzyl)isoquinolin-8-yl]-sulfamide hydrochloride 8 as a brown solid (10 mg, 100% yield).

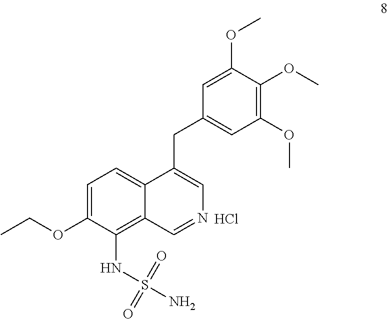 [SMILES: CCOc1ccc2c(Cc3cc(OC)c(OC)c(OC)c3)cncc2c1NS(N)(=O)=O ; Cl]

MW: 483.97; Yield: 26%; Brown solid; Mp (° C.): 139.1 (dec.)

R_{f}: 0.2 (CH₂Cl₂:MeOH=93:7, free base).

¹H-NMR (CD₃OD, δ): 1.53 (t, 3H, J=6.8 Hz, CH₂CH₃), 3.71 (s, 3H, OCH₃), 3.76 (s, 6H, 2×OCH₃), 4.41 (q, 2H, J=6.8 Hz, CH₂CH₃), 4.54 (s, 2H, CH₂), 6.60 (s, 2H, 2×ArH), 8.16 (d, 1H, J=9.4 Hz), 8.22 (s, 1H, ArH), 8.42 (d, 1H, J=9.4 Hz), 9.73 (s, 1H, ArH).

¹³C-NMR (CD₃OD, δ): 14.9, 37.0, 56.7 (2×OCH₃), 61.1, 66.9, 107.5 (2×CH), 123.9, 126.0, 126.5, 128.9, 129.1, 133.9, 135.1, 138.2, 138.5, 144.9, 155.0 (2×C), 156.5.

MS-ESI m/z (rel. int.): 448 ([MH]⁺, 100).

HPLC: Method A, detection UV 254 nm, RT=4.37 min, peak area 95.1%.

### Preparation of 4-(4-ethoxy-3,5-dimethoxybenzyl)-7,8-dimethoxyisoquinoline hydrochloride 9

### (E)-N-(2,2-Diethoxy-ethyl)-(2,3-dimethoxy-benzylidene)-amine LPO 26046

In a Dean Stark apparatus, 2,3-dimethoxybenzaldehyde (8.7 g, 52.36 mmol) was dissolved in toluene (140 mL) and aminoacetaldehyde diethyl acetal (11.40 mL, 78.53 mmol, 1.5 eq) was added at 20° C. via syringe at 20° C. under an N₂ atmosphere. Water (25 mL) and toluene (5 mL) were added in the Dean Stark burette and the reaction was stirred at 150° C. for 4 h. The solvent was evaporated at 60° C. to give after drying, (E)-N-(2,2-diethoxy-ethyl)-(2,3-dimethoxy-benzylidene)amine LPO 26046 as a yellow oil (16.47 g, 100% crude yield). This crude product was used in the next step without further purification.

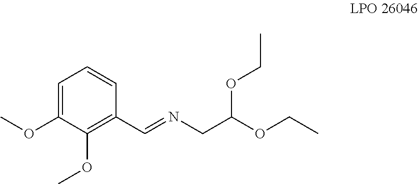 [SMILES: CCOC(C/N=C/c1cccc(OC)c1OC)OCC]

MW: 281.35; Yield: 100% (crude); Yellow oil.

¹H-NMR (CDCl₃, δ): 1.21 (t, 6H, J=7 Hz, 2×CH₂CH₃), 3.52-3.64 (m, 2H, OCH₂), 3.69-3.79 (m, 2H, OCH₂), 3.82 (dd, 2H, J=1.28 Hz, J=4.09 Hz, NCH₂CH), 3.88 (s, 6H, 2×OCH₃), 4.81 (t, 1H, J=5.37 Hz, CHO), 6.97 (dd, 1H, J=1.55 Hz, J=8.11 Hz, ArH), 7.08 (t, 1H, J=7.93 Hz, ArH), 7.53 (dd, 1H, =1.56 Hz, J=9.39 Hz, ArH), 8.66 (s, 1H, ArH).

MS-ESI m/z (% rel. Int.): 167.1 ([MH]⁺, 68), 168.1 (6), 139.1 (100).

HPLC: Method A, detection UV 254 nm, RT=4.93 min, peak area 99.9%.

### N-(2,3-Dimethoxybenzyl)-2,2-diethoxyethanamine LPO 26048

(E)-N-(2,2-Diethoxy-ethyl)-(2,3-dimethoxy-benzylidene)-amine LPO 26046 (16.47 g, 58.54 mmol) was dissolved in EtOH (130 mL). NaBH₄ (4.21 g, 111.22 mmol, 1.9 eq) was added at 20° C. under a N₂ atmosphere for 45 min. The reaction mixture was evaporated at 45° C. to give an oily residue and CHCl₃ (600 mL) and H₂O (150 mL) were added. The separated organic layer was washed with brine (50 mL), dried over MgSO₄, filtered and concentrated to dryness under reduced pressure to give N-(2,3-dimethoxybenzyl)-2,2-diethoxyethanamine LPO 26048 as a yellow oil (14.1 g, 85% crude yield). This crude product was used in the next step without further purification.

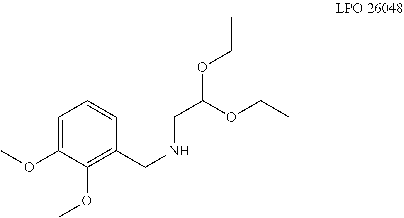 [SMILES: CCOC(CNCc1cccc(OC)c1OC)OCC]

MW: 283.36; Yield=85% (crude); Yellow oil.

¹H-NMR (CDCl₃, δ): 1.20 (t, 6H, J=7.05 Hz, 2×CH₂CH₃), 2.74 (d, 2H, J=5.62 Hz, CH₂NH), 3.49-3.57 (m, 2H, OCH₂), 3.63-3.70 (m, 2H, OCH₂), 3.82 (s, 2H, ArCH₂), 3.85 (s, 3H, OCH₃), 3.86 (s, 3H, OCH₃), 4.62 (t, 1H, J=5.6 Hz, CHO), 6.84 (dd, 1H, J=1.41 Hz, J=8.04 Hz, ArH), 6.89 (dd, 1H, J=1.31 Hz, J=7.6 Hz, ArH), 7.01 (t, 1H, J=7.85 Hz, ArH), 7.27 (s, 2H, 2×ArH), NH not seen.

MS-ESI m/z (% rel. Int.): 285.2 (6), 284.2 ([MH]⁺, 38), 238.2 (100).

HPLC: Method A, detection UV 254 nm, RT=4.14 min, peak area 99.9%.

### Preparation of 4-(4-ethoxy-3,5-dimethoxybenzyl)-7,8-dimethoxyisoquinoline hydrochloride 9

### 4-(4-Ethoxy-3,5-dimethoxybenzyl)-7,8-dimethoxyisoquinoline hydrochloride 9

To solution of N-(2,3-dimethoxybenzyl)-2,2-diethoxyethanamine LPO 26048 (500 mg, 1.76 mmol) in EtOH (3 mL) was added TTA 24126 (370 mg, 1.76 mmol) and a HCl 37% solution (3 mL) at room temperature in an ace pressure tube (Aldrich, 100 mL). The solution was stirred at 100° C. for 20 min and cooled to 4° C. (water-ice bath). The solvent was evaporated and the residue was dried to give ANP 31118A as an orange solid (761 mg). The solid was transformed into freebase with a 1 M K₂CO₃ solution (50 mL) and extracted with EtOAc (120 mL). The organic layer was washed with brine, dried over MgSO₄, filtered and the solvent was evaporated to give after drying ANP 31118B as a brown oil (643 mg). ANP 31118B was purified by column chromatography (SiO₂; gradient elution cyclohexane:EtOAc 100:0 to 6:4) to yield, after evaporation and drying, ANP 31118D as a yellow solid (product of cyclisation, 66.8 mg) and ANP 31118E (100 mg, yellow oil). ANP 31118E (100 mg, 0.26 mmol) was dissolved in MeOH with HCl 1.51N in MeOH (181 μL, 0.27 mmol). The solution was stirred at 4° C. for 10 min, the solvent was evaporated to give 4-(4-ethoxy-3,5-dimethoxybenzyl)-7,8-dimethoxyisoquinoline hydrochloride 9 as a yellow solid (107 mg, 14% yield).

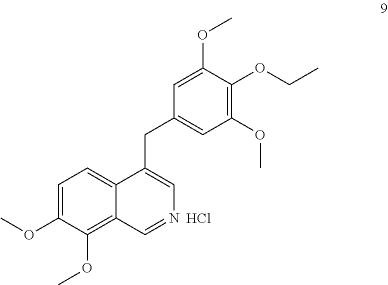 [SMILES: CCOc1c(OC)cc(Cc2cncc3c(OC)c(OC)ccc23)cc1OC ; Cl]

MW: 419.90; Yield: 14%; Yellow solid; Mp (° C.): 151.5

R_{f}: 0.25 (cyclohexane:EtOAc=6:4, free base).

¹H-NMR (CD₃OD, δ): 1.28 (t, 3H, CH₃, J=7.04 Hz), 3.77 (s, 6H, 2×CH₃), 3.94 (q, 2H, CH₂, J=7.08 Hz), 4.11 (s, 3H, CH₃), 4.20 (s, 3H, CH₃), 4.53 (s, 2H, CH₂), 6.61 (s, 2H, 2×ArH), 8.17 (m, 3H, 3×ArH), 9.65 (s, 1H, ArH).

¹³C-NMR (CD₃OD, δ): 15.7, 30.7, 37.0, 56.7 (2×C), 57.5, 62.6, 69.9, 107.5 (2×C), 121.6, 124.8, 127.4, 129.3, 133.6, 134.9, 137.0, 138.5, 142.5, 146.8, 152.3, 155.2.

MS-ESI m/z (% rel. Int.): 384.3 ([MH]⁺, 100), 385.3 (26), 386.3 (4).

HPLC: Method A, detection UV 254 nm, RT=4.71 min, peak area 99.9%.

### Preparation of 4-(3,4,5-trimethoxybenzyl)-7,8-dimethoxyisoquinoline hydrochloride 10

### 4-(3,4,5-Trimethoxybenzyl)-7,8-dimethoxyisoquinoline hydrochloride 10

In an ace pressure tube (Aldrich, 38 mL), N-(2,3-dimethoxybenzyl)-2,2-diethoxyethanamine LPO 26048 (722.1 mg, 2.548 mmol) and 3,4,5-trimethoxybenzaldehyde (500 mg, 2.548 mmol) were dissolved in EtOH (2.8 mL) and a 37% HCl solution (2.8 mL) was added. The reaction mixture was stirred at 90° C. for 30 min, cooled to 4° C. and concentrated. The crude product was transformed into free base with a 1 M K₂CO₃ aqueous solution (30 mL) and extracted with EtOAc (2×150 mL). The combined organic layers were washed with brine (30 mL), dried over MgSO₄, filtered and evaporated to give a yellow oil. This crude oil was purified by column chromatography (SiO₂, eluent: cyclohexane:acetone=7:3 to 6:4) to give, after evaporation and drying, 4-(3,4,5-trimethoxybenzyl)-7,8-dimethoxyisoquinoline (171.6 mg) that was dissolved in MeOH (4 mL) and a 1.51 N HCl solution in MeOH (323.0 μL, 0.488 mmol) was slowly added at 4° C. The reaction mixture was stirred at 4° C. for 15 min. After evaporation of solvent and drying under vacuum pump under P₂O₅, 4-(3,4,5-trimethoxybenzyl)-7,8-dimethoxyisoquinoline hydrochloride 10 was obtained as a yellow solid (188 mg, 18% yield).

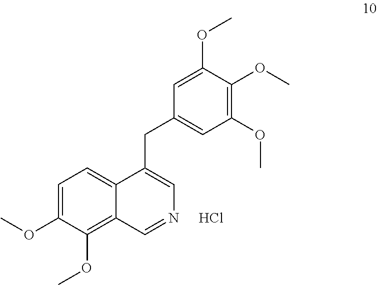

MW: 405.87; Yield: 18%; Yellow Solid; Mp (° C.): 178.8

R_{f}: 0.2 (cyclohexane:acetone=6:4, free base).

¹H-NMR (CD₃OD, δ): 3.72 (s, 3H, OCH₃), 3.76 (s, 6H, 2×OCH₃), 4.09 (s, 3H, OCH₃), 4.17 (s, 3H, OCH₃), 4.50 (s, 2H, CH₂), 6.60 (s, 2H, 2×ArH), 8.10-8.18 (m, 3H, 3×ArH), 9.60 (s, 1H, ArH).

¹³C-NMR (CD₃OD, δ): 37.0, 56.7 (2×C), 57.6, 61.1, 62.6, 107.6 (2×C), 121.6, 124.9, 126.7, 131.0, 133.4, 135.4, 137.6, 138.2, 143.1, 146.6, 152.0, 155.0 (2×C).

MS-ESI m/z (% rel. Int.): 370.4 ([MH]⁺, 100).

HPLC: Method A, detection UV 254 nm, RT=4.24 min, peak area 98.0%.

### Preparation of 2,6-dimethoxy-4-((7,8-dimethoxyisoquinolin-4-yl)methyl)phenol hydrochloride 11

### 2,6-Dimethoxy-4-((7,8-dimethoxyisoquinolin-4-yl)methyl)phenol hydrochloride 11

In an ace pressure tube (Aldrich, 38 mL), N-(2,3-dimethoxybenzyl)-2,2-diethoxyethanamine LPO 26048 (778 mg, 2.74 mmol) and 4-hydroxy-3,5-dimethoxybenzaldehyde (500 mg, 2.74 mmol) were dissolved in EtOH (2.8 mL) and a 37% HCl solution (2.8 mL) was added. The reaction mixture was stirred at 90° C. for 30 min, cooled to 4° C. and concentrated to dryness. The crude product was transformed into free base with a 1 M K₂CO₃ solution (30 mL) and extracted with EtOAc (2×150 mL). The combined organic layers were washed with brine (30 mL), dried over MgSO₄, filtered and evaporated to give a yellow solid. This solid was purified by column chromatography (SiO₂, eluent: CH₂Cl₂:MeOH=98:2) to give, after evaporation and drying, 2,6-dimethoxy-4-((7,8-dimethoxyisoquinolin-4-yl)methyl)phenol freebase (278.5 mg) that was dissolved in MeOH (4 mL) and a 1.51 N HCl solution in MeOH (545 μL, 0.823 mmol) was slowly added at 4° C. The reaction mixture was stirred at 4° C. for 15 min. After evaporation of the solvent and drying under vacuum pump under P₂O₅, 2,6-dimethoxy-4-((7,8-dimethoxyisoquinolin-4-yl)methyl)phenol hydrochloride 11 was obtained as a yellow solid (265.5 mg, 25% yield).

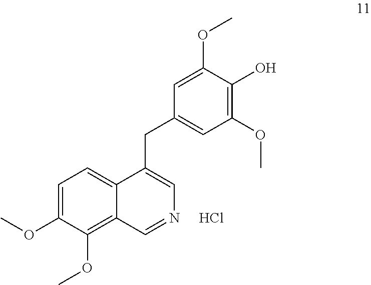

MW: 391.85; Yield: 25%; Yellow Solid; Mp (° C.): 270.9

R_{f}: 0.2 (CH₂Cl₂: MeOH=98:2, free base).

¹H-NMR (CD₃OD, δ): 3.77 (s, 6H, OCH₃), 4.10 (s, 3H, OCH₃), 4.19 (s, 3H, OCH₃), 4.48 (s, 2H, CH₂), 6.58 (s, 2H, 2×ArH), 8.12-8.18 (m, 3H, 3×ArH), 9.63 (s, 1H, ArH).

¹³C-NMR (CD₃OD, δ): 36.9, 56.9 (2×C), 57.6, 62.6, 107.6 (2×C), 121.7, 124.8, 127.4, 129.2, 129.5, 133.7, 135.9, 139.1, 142.3, 146.8, 149.7 (2×C), 152.3.

MS-ESI m/z (% rel. Int.): 356.3 ([MH]⁺, 100).

HPLC: Method A, detection UV 254 nm, RT=4.14 min, peak area 97%.

### Preparation of 7-ethoxy-4-(3,4,5-trifluorobenzyl)isoquinolin-8-ol hydrochloride 12

### 7-Ethoxy-4-(3,4,5-trifluorobenzyl)isoquinolin-8-ol hydrochloride 12

A solution of 2-((2,2-diethoxyethylamino)methyl)-6-ethoxyphenol SAO 33014 (500 mg, 1.76 mmol) in EtOH (3 mL) was added 3,4,5-trifluorobenzaldehyde (289 mg, 1.76 mmol) and a 37% HCl solution (3 mL) at RT. The solution was stirred at 100° C. for 25 min and cooled to RT (water-ice bath). The solvent was evaporated and the obtained residue was dried to give a yellow solid. This solid was dissolved in water (20 mL) and an 18 N NH₄OH solution (60 μL, 1.08 mmol, 0.6 eq) was added (pH=7) and a precipitate was observed. The aqueous layer was extracted with EtOAc (60 mL). The combined organic layers were washed with brine, dried over MgSO₄, filtered and evaporated to give after drying ANP 31152A as a brown solid (330 mg). ANP 31152A was purified by column chromatography (SiO₂, eluent cyclohexane:EtOAc=10:0 to 6:4) to give, after evaporation and drying, ANP 31152B as a yellow solid (162 mg). ANP 31152B was dissolved in a mixture of MeOH:CH₂Cl₂ (3:2, 5 mL) and a 1.47 N HCl solution in MeOH (347 μL, 0.51 mmol, 1.05 eq) was slowly added. The solution was stirred at 4° C. for 10 min and the solvents were evaporated and the obtained residue was dried to give 7-ethoxy-4-(3,4,5-trifluorobenzyl)isoquinolin-8-ol hydrochloride 12 as a yellow solid (177.7 mg, 27% yield).

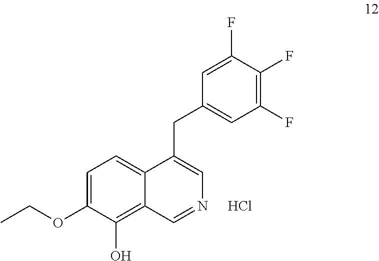 [SMILES: CCOc1ccc2c(Cc3cc(F)c(F)c(F)c3)cncc2c1O ; Cl]

MW: 369.77; Yield: 27%; Yellow solid; Mp (° C.): 233.1

R_{f}: 0.30 (cyclohexane:EtOAc=6:4, free base).

¹H-NMR (CD₃OD, δ): 1.50 (t, 3H, CH₃, J=6.53 Hz), 4.32-4.36 (q, 2H, CH₂, J=4.6 Hz), 4.53 (s, 2H, CH₂), 7.08-7.13 (m, 2H, ArH), 7.75 (d, 1H, ArH, J=9.08 Hz), 8.02 (d, 1H, ArH, J=9.05 Hz), 8.19 (s, 1H, ArH), 9.70 (s, 1H, ArH).

¹³C-NMR (CD₃OD, δ): 15.0, 35.8, 66.9, 114.1-114.4 (m, 2×C, CH_{Ar}CF), 116.3, 120.5, 126.8, 128.8, 132.4, 136.1, 136.4-136.7 (m, 1×C, CH₂CCHCF), 139.8 (dt, CF(CF)₂, J=249.1 Hz), 143.2, 146.5, 147.1, 152.5 (ddd, 2×C, CHCFCF, J₁=234.8 Hz, J₂=6 Hz, J₃=4 Hz).

MS-ESI m/z (% rel. Int.): 334.2 ([MH]⁺, 100).

HPLC: Method A, detection UV 254 nm, RT=5.18 min, peak area 99.9%.

### Preparation of 7-ethoxy-4-(4-ethoxy-3,5-dimethoxybenzyl)isoquinoline hydrochloride 13

### (E)-N-(3-Ethoxybenzylidene)-2,2-diethoxyethanamine LPO 30168

In a Dean Stark apparatus, 3-ethoxybenzaldehyde (4 g, 26.64 mmol) was dissolved in toluene (65 mL) and aminoacetaldehyde diethyl acetal (5.81 mL, 39.95 mmol) was added at 20° C. via a syringe at 20° C. under an N₂ atmosphere. Water (25 mL) and toluene (5 mL) were added in the Dean Stark burette and the reaction mixture was stirred at 150° C. for 4 h. After cooling, the solvent was evaporated at 60° C. to give (E)-N-(3-ethoxybenzylidene)-2,2-diethoxyethanamine LPO 30168 as a yellow oil (7.1 g, 100% yield). The crude product was used in the next step without further purification.

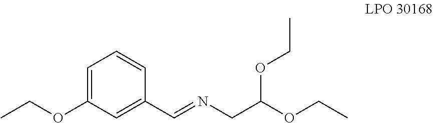 [SMILES: CCOc1cccc(/C=N/CC(OCC)OCC)c1]

MW: 265.35; Yield: 100% (crude); Yellow Oil.

¹H-NMR (CDCl₃, δ): 1.20 (t, 6H, J=7.1 Hz, 2×CH₂CH₃), 1.42 (t, 3H, J=7.0 Hz, CH₂CH₃), 3.52-3.64 (m, 2H, OCH₂), 3.69-3.79 (m, 4H, OCH₂& NCH₂), 4.08 (q, 2H, J=7.0 Hz, OCH₂), 4.80 (t, 1H, J=5.4 Hz, CHO), 6.95-6.99 (m, 1H, ArH), 7.23-7.33 (m, 3H, 3×ArH), 8.25 (s, 1H, ArH).

MS-ESI m/z (% rel. Int.): 439.3 (100).

HPLC: Method A, detection UV 254 nm, RT=6.07 min, peak area 98%.

### 2,2-Diethoxy-N-(3-ethoxybenzyl)ethanamine LPO 30170

(E)-N-(3-Ethoxybenzylidene)-2,2-diethoxyethanamine LPO 30168 (7.1 g, 26.76 mmol) was dissolved in EtOH (60 mL). NaBH₄ (1.9 g, 50.84 mmol) was added at 20° C. under an N₂ atmosphere for 30 min. The reaction mixture was stirred at 100° C. for 1 h and evaporated at 45° C. to give an oily residue. Dichloromethane (500 mL) and water (100 mL) were added. The organic layer was washed with brine (50 mL), dried over MgSO₄, filtered and concentrated to dryness under reduced pressure to give 2,2-diethoxy-N-(3-ethoxybenzyl)ethanamine LPO 30170 as a yellow oil (6.8 g, 95% crude yield). This crude product was used in the next step without further purification.

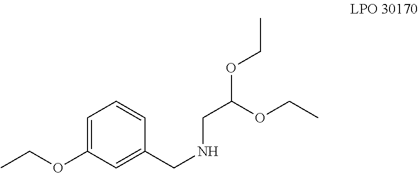 [SMILES: CCOc1cccc(CNCC(OCC)OCC)c1]

MW: 267.37; Yield=95% (crude); Yellow oil.

¹H-NMR (CDCl₃, δ): 1.20 (t, 6H, J=7.0 Hz, 2×CH₂CH₃), 1.40 (t, 3H, J=6.9 Hz, CH₂CH₃), 1.62 (s, 1H, NH), 2.74 (d, 2H, J=5.3 Hz, CH₂NH), 3.50-3.58 (m, 2H, OCH₂), 3.63-3.73 (m, 2H, OCH₂), 3.78 (s, 2H, ArCH₂), 4.03 (q, 2H, J=6.9 Hz, OCH₂), 4.62 (t, 1H, J=5.3 Hz, CHO), 6.77 (d, 1H, J=8.1 Hz, ArH), 6.87 (s, 2H, 2×ArH), 7.21 (t, 1H, J=7.9 Hz, ArH).

¹³C-NMR (CDCl₃, δ): 14.9, 15.4 (2×C), 51.6, 53.8, 62.3 (2×C), 63.3, 102.2, 113.0, 114.2, 120.3, 129.3, 141.9, 159.1.

MS-ESI m/z (% rel. Int.): 268.3 ([MH]⁺, 10), 176.2 (100).

HPLC: Method A, detection UV 254 nm, RT=4.50 min, peak area 95%.

### 4-Ethoxy-3,5-dimethoxybenzaldehyde TTA 24126

4-Hydroxy-3,5-dimethoxybenzaldehyde (800 mg, 4.30 mmol) was dissolved in DMF (15 mL) and Cs₂CO₃ (1.4 g, 4.30 mmol) was added at 20° C. Bromoethane (352 μL, 4.70 mmol) was added and the reaction mixture was stirred at 40° C. for 15 h under an N₂ atmosphere. The reaction mixture was poured into water (150 mL) and extracted with EtOAc (100 mL). The organic layer was washed with water (2×50 mL), brine (50 mL), dried over MgSO₄, filtered and concentrated to dryness under reduced pressure to give a viscous oil. After drying 24 h under vacuum, 4-ethoxy-3,5-dimethoxybenzaldehyde TTA 24126 (700 mg, 78% crude yield) was obtained as an off-white solid. This crude product was used in the next step without further purification.

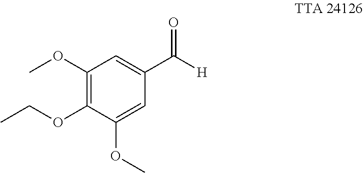

MW: 210.23; Yield: 78% (crude); Off-white solid; Mp (° C.): 50.9

R_{f}: 0.8 (CH₂Cl₂:EtOAc=8:2).

¹H-NMR (CDCl₃, δ): 1.38 (t, 3H, J=7.1 Hz, CH₂CH₃), 3.92 (s, 6H, 2×OCH₃), 4.17 (q, 2H, J=7.1 Hz, OCH₂), 7.13 (s, 2H, 2×ArH), 9.87 (s, 1H, CHO).

¹³C-NMR (CDCl₃, δ): 15.5, 56.2 (2×C), 69.2, 106.7 (2×C), 131.6, 142.7, 154.0 (2×C), 191.1

MS-ESI m/z (% rel. Int.): 211.2 ([MH]⁺, 40), 183.2 (100).

HPLC: Method A, detection UV 254 nm, RT=5.50 min, peak area 98%.

### 7-Ethoxy-4-(4-ethoxy-3,5-dimethoxybenzyl)isoquinoline hydrochloride 13

A solution of 2,2-diethoxy-N-(3-ethoxybenzyl)ethanamine LPO 30170 (382 mg, 1.43 mmol) in EtOH (2.5 mL) was added 4-ethoxy-3,5-dimethoxybenzaldehyde TTA 24126 (300 mg, 1.43 mmol, 1 eq) and a 37% HCl solution (2.5 mL) at RT. The reaction mixture was stirred at 90° C. for 20 min and cooled to 4° C. (ice-water bath). The solvent was evaporated and residue was dried to give an orange solid. This crude product was transformed into freebase with a 1 M K₂CO₃ aqueous solution (20 mL) and extracted with EtOAc (60 mL). The organic layer was washed with brine, dried over MgSO₄, filtered and the solvent was evaporated to give after drying ANP 31178A as a yellow oil (554 mg). ANP 31178A was purified by column chromatography (SiO₂, eluent cyclohexane:EtOAc=9:1 to 4:6) to yield, after evaporation and drying, to 7-ethoxy-4-(4-ethoxy-3,5-dimethoxybenzyl)isoquinoline ANP 31178B as yellow solid (178 mg). ANP 31178B (46 mg, 0.125 mmol) was dissolved in MeOH and a 0.41 N HCl solution in MeOH (320 μL, 0.131 mmol) was added at 4° C. The solution was stirred at 4° C. for 10 min then the solvent was evaporated and the residue was dried under vacuum to give 7-ethoxy-4-(4-ethoxy-3,5-dimethoxybenzyl)isoquinoline hydrochloride 13 as a yellow solid (52.6 mg, 34% yield).

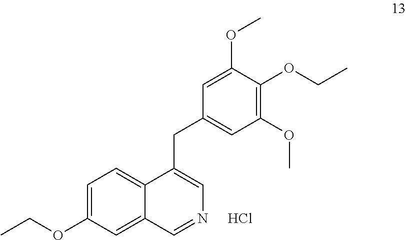 [SMILES: CCOc1ccc2c(Cc3cc(OC)c(OCC)c(OC)c3)cncc2c1 ; Cl]

MW: 403.90; Yield: 34%; Yellow solid; Mp (° C.): 67.1

R_{f}: 0.25 (cyclohexane:EtOAc=4:6, free base).

¹H-NMR (CD₃OD, δ): 1.25-1.31 (t, 3H, CH₂CH₃, J=7.04 Hz); 1.49-1.54 (t, 3H, CH₂CH₃, J=6.95 Hz); 3.76 (s, 6H, 2×OCH₃); 3.92-3.99 (q, 2H, OCH₂, J=7.07 Hz); 4.27-4.34 (q, 2H, OCH₂, J=6.96 Hz); 4.55 (s, 2H, CH₂), 6.61 (s, 2H, 2×ArH), 7.82-7.86 (m, 2H, 2×ArH), 8.23 (s, 1H, ArH), 8.40 (d, 1H, ArH, J=9.99 Hz), 9.50 (s, 1H, ArH).

¹³C-NMR (CD₃OD, δ): 16.4 (CH₂CH₃), 17.3 (CH₂CH₃), 38.5 (CH₂), 58.3 (2×OCH₃), 67.5 (OCH₂CH₃), 71.5 (OCH₂CH₃), 109.1 (2×C), 111.1, 128.9, 131.5, 132.7, 133.0, 136.6 (2×C), 138.6, 140.4, 146.5, 156.9 (2×C), 163.3.

MS-ESI m/z (% rel. Int.): 368.2 ([MH]⁺, 100).

HPLC: Method A, detection UV 254 nm, RT=5.20 min, peak area 99.9%.

### Preparation of 4-(4-ethoxy-3,5-dimethoxybenzyl)-7-ethoxyisoquinolin-8-ol hydrochloride 14

### 4-(4-Ethoxy-3,5-dimethoxybenzyl)-7-ethoxyisoquinolin-8-ol hydrochloride 14

In an ace pressure tube (Aldrich, 38 mL), 2-((2,2-diethoxyethylamino)methyl)-6-ethoxyphenol SAO 33014 (324 mg, 1.14 mmol) and 4-ethoxy-3,5-dimethoxybenzaldehyde TTA 24126 (240 mg, 1.14 mmol) were dissolved in EtOH (1.4 mL) and a 37% HCl solution (1.4 mL) was added. The reaction mixture was stirred at 90° C. for 20 min, cooled to 4° C. (ice-water bath) and concentrated. The crude product was transformed into free base with a 1M K₂CO₃ solution (30 mL) and extracted with EtOAc (2×150 mL). The combined organic layers were washed with brine (30 mL), dried over MgSO₄, filtered and evaporated to give a yellow solid. This solid was purified by flash chromatography, (SiO₂, eluent cyclohexane:acetone=8:2 to 7:3) to give, after evaporation and drying, 4-(4-ethoxy-3,5-dimethoxybenzyl)-7-ethoxyisoquinolin-8-ol (152 mg). 4-(4-Ethoxy-3,5-dimethoxybenzyl)-7-ethoxyisoquinolin-8-ol was dissolved in MeOH (2 mL) and a 1.47 N HCl solution in MeOH (284 μL, 0.42 mmol) was slowly added. The reaction mixture was stirred at 4° C. for 15 min. After evaporation and drying under vacuum pump under P₂O₅, 4-(4-ethoxy-3,5-dimethoxybenzyl)-7-ethoxyisoquinolin-8-ol hydrochloride 14 was obtained as a yellow solid (144 mg, 30% yield).

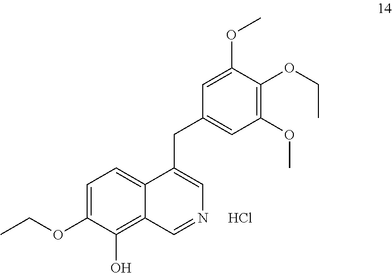 [SMILES: CCOc1ccc2c(Cc3cc(OC)c(OCC)c(OC)c3)cncc2c1O ; Cl]

MW: 419.9; Yield: 30%; Yellow Solid; Mp (° C.): 235.0

R_{f}: 0.2 (cyclohexane:acetone=7:3, free base).

¹H-NMR (CD₃OD, δ): 1.29 (t, 3H, J=7.1 Hz, CH₂CH₃), 1.50 (t, 3H, J=7.0 Hz, CH₂CH₃), 3.76 (s, 6H, 2×OCH₃), 3.95 (q, 2H, J=7.1 Hz, CH₂CH₃), 4.37 (q, 2H, J=7.0 Hz, CH₂CH₃), 4.48 (s, 2H, CCH₂), 6.61 (s, 2H, 2×ArH), 7.87 (d, 1H, J=9.1 Hz, ArH), 8.02-8.06 (m, 2H, 2×ArH), 9.65 (s, 1H, ArH).

¹³C-NMR (CD₃OD, δ): 15.0, 15.7, 37.0, 56.7 (2×C), 66.9, 69.9, 107.4 (2×C), 116.5, 120.5, 126.7, 128.4, 132.8, 135.0, 136.9, 137.9, 142.5, 146.4, 146.8, 155.2 (2×C).

MS-ESI m/z (% rel. Int.): 384.3 ([MH]⁺, 100).

HPLC: Method A, detection UV 254 nm, RT=5.08 min, peak area 98%.

### Preparation of 4-(3-ethoxy-4,5-dimethoxybenzyl)-7-ethoxyisoquinolin-8-ol hydrochloride 15

### 3-Ethoxy-4,5-dimethoxybenzaldehyde TTA 24132

3-Hydroxy-4,5-dimethoxybenzaldehyde (1.2 g, 6.60 mmol) was dissolved in DMF (20 mL) and Cs₂CO₃ (2.2 g, 6.60 mmol) was added at 20° C. Bromoethane (540 μL, 7.20 mmol) was added and the reaction mixture was stirred at 40° C. for 15 h under an N₂ atmosphere. The reaction mixture was poured into water (150 mL) and extracted with EtOAc (100 mL). The organic layer was washed with water (2×50 mL), brine (50 mL), dried over MgSO₄, filtered and concentrated to dryness under reduced pressure to give a viscous oil. After drying 24 h under vacuum, 3-ethoxy-4,5-dimethoxybenzaldehyde TTA 24132 (1.0 g, 72% crude yield) was obtained as a beige solid. This crude product was used in the next step without further purification.

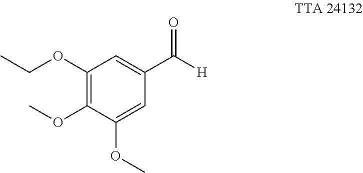

MW: 210.23; Yield: 72% (crude); Beige solid; Mp (° C.): 51.5

R_{f}: 0.8 (CH₂Cl₂:EtOAc=8:2).

¹H-NMR (CDCl₃, δ): 1.48 (t, 3H, J=7.0 Hz, CH₂CH₃), 3.93 (s, 3H, OCH₃), 3.95 (s, 3H, OCH₃), 4.16 (q, 2H, J=7.0 Hz, OCH₂), 7.12 (s, 2H, 2×ArH), 9.86 (s, 1H, CHO).

MS-ESI m/z (% rel. Int.): 211.0 ([MH]⁺, 52), 183.0 (100).

HPLC: Method A, detection UV 254 nm, RT=4.75 min, peak area 98%.

### Preparation of 4-(3-ethoxy-4,5-dimethoxybenzyl)-7-ethoxyisoquinolin-8-ol hydrochloride 15

### 4-(3-Ethoxy-4,5-dimethoxybenzyl)-7-ethoxyisoquinolin-8-ol hydrochloride 15

In ace pressure tube (Aldrich 100 mL), 2-((2,2-diethoxyethylamino)methyl)-6-ethoxyphenol SAO 33014 (674 mg, 2.38 mmol) and 3-ethoxy-4,5-dimethoxybenzaldehyde TTA 24132 (500 mg, 2.38 mmol) were dissolved in EtOH (2.8 mL) and a 37% HCl solution (2.8 mL) was added. The reaction mixture was stirred at 90° C. for 20 min, cooled to 4° C. (ice-water bath) and concentrated. The crude product was transformed into free base with a 18 N NH₄OH solution (2×500 μL) and extracted with EtOAc (2×150 mL). The combined organic layers were washed with brine (30 mL), dried over MgSO₄, filtered and evaporated to give a yellow solid. This solid was purified by column chromatography (SiO₂, eluent cyclohexane: acetone=8:2 to 6:4) to give after evaporation and drying 4-(3-ethoxy-4,5-dimethoxybenzyl)-7-ethoxyisoquinolin-8-ol (312 mg). 4-(3-ethoxy-4,5-dimethoxybenzyl)-7-ethoxyisoquinolin-8-ol was dissolved in MeOH (4 mL) and a 1.47 N HCl solution in MeOH (581 μL, 0.85 mmol) was slowly added. The reaction mixture was stirred at 4° C. for 15 min. After evaporation and drying under vacuum pump under P₂O₅, 4-(3-ethoxy-4,5-dimethoxybenzyl)-7-ethoxyisoquinolin-8-ol hydrochloride 15 was obtained as a yellow solid (242.5 mg, 24% yield).

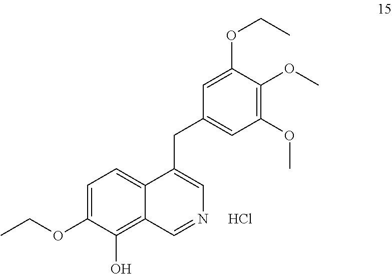 [SMILES: CCOc1cc(Cc2cncc3c(O)c(OCC)ccc23)cc(OC)c1OC ; Cl]

MW: 419.9; Yield: 24%; Yellow Solid; Mp (° C.): 225.6

R_{f}: 0.2 (cyclohexane:acetone=8:2, free base).

¹H-NMR (CD₃OD, δ): 1.35 (t, 3H, J=7.0 Hz, CH₂CH₃), 1.51 (t, 3H, J=7.0 Hz, CH₂CH₃), 3.76 (d, 3H, J=1.8 Hz, OCH₃), 3.78 (d, 3H, J=1.8 Hz, OCH₃), 4.01 (q, 2H, J=7.0 Hz, CH₂CH₃), 4.37 (q, 2H, J=7.0 Hz, CH₂CH₃), 4.48 (s, 2H, CCH₂), 6.60 (m, 2H, 2×ArH), 7.88 (d, 1H, J=9.0 Hz, ArH), 8.02-8.07 (m, 2H, 2×ArH), 9.67 (s, 1H, ArH).

¹³C-NMR (CD₃OD, δ): 15.0, 15.2, 37.0, 56.7, 61.1, 65.8, 66.9, 107.4, 108.7, 116.6, 120.5, 126.7, 128.3, 132.8, 135.0, 138.0, 138.4, 142.5, 146.4, 146.8, 154.2, 155.0.

MS-ESI m/z (% rel. Int.): 384.2 ([MH]⁺, 100).

HPLC: Method A, detection UV 254 nm, RT=5.14 min, peak area 98%.

### Preparation of 4-(3,4,5-trimethoxybenzyl)-7-ethoxyisoquinoline hydrochloride 16

### 4-(3,4,5-Trimethoxybenzyl)-7-ethoxyisoquinoline hydrochloride 16

In an ace pressure tube (Aldrich, 38 mL), 2,2-diethoxy-N-(3-ethoxybenzyl)ethanamine LPO 30170 (681 mg, 2.55 mmol) and 3,4,5-trimethoxybenzaldehyde (500 mg, 2.55 mmol) were dissolved in EtOH (2.8 mL) and a 37% HCl solution (2.8 mL) was added. The reaction mixture was stirred at 90° C. for 20 min, cooled to 4° C. and concentrated. The crude product was transformed into freebase with a 1 M K₂CO₃ solution (50 mL) and extracted with EtOAc (250 mL). The organic layer was washed with brine (30 mL), dried over MgSO₄, filtered and evaporated to give a yellow solid. This solid was purified by column chromatography, (SiO₂, eluent: cyclohexane:acetone=7:3 to 6:4) to give, after evaporation and drying, 4-(3,4,5-trimethoxybenzyl)-7-ethoxyisoquinoline (318.3 mg) that was dissolved in MeOH (4 mL) and a 1.47 N HCl solution in MeOH (643 μL, 0.946 mmol) was slowly added at 4° C. The reaction mixture was stirred at 4° C. for 15 min. After evaporation of solvent and drying under vacuum pump under P₂O₅, 4-(3,4,5-trimethoxybenzyl)-7-ethoxyisoquinoline hydrochloride 16 was obtained as a yellow solid (325 mg, 33% yield).

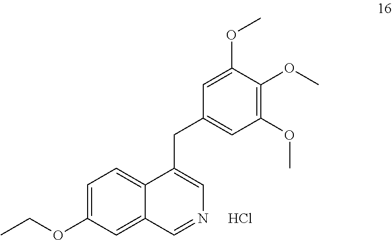 [SMILES: CCOc1ccc2c(Cc3cc(OC)c(OC)c(OC)c3)cncc2c1 ; Cl]

MW: 389.87; Yield: 33%; Yellow solid; Mp (° C.): 205.0

R_{f}: 0.2 (cyclohexane:acetone=7:3, free base).

¹H-NMR (CD₃OD, δ): 1.52 (t, 3H, J=6.9 Hz, CH₂CH₃), 3.74 (s, 3H, OCH₃), 3.78 (s, 6H, 2×OCH₃), 4.32 (q, 2H, J=7.0 Hz, CH₂CH₃), 4.55 (s, 2H, CCH₂), 6.63 (s, 2H, 2×ArH), 7.81-7.85 (m, 2H, 2×ArH), 8.24 (s, 1H, ArH), 8.38 (d, 1H, J=8.8 Hz, ArH), 9.50 (s, 1H, ArH).

¹³C-NMR (CD₃OD, δ): 14.8, 36.8, 56.7 (2×C), 61.1, 65.8, 107.5 (2×C), 109.5, 127.3, 130.4, 131.1, 131.2, 134.8, 135.2, 138.2, 138.4, 145.2, 155.0 (2×C), 161.5.

MS-ESI m/z (% rel. Int.): 354.3 ([MH]⁺, 100).

HPLC: Method A, detection UV 254 nm, RT=4.76 min, peak area 98%.

### Preparation of 4-(2-bromo-3,5-dimethoxybenzyl)-7-ethoxyisoquinolin-8-ol hydrochloride 17

### 2-Bromo-3,5-dimethoxybenzaldehyde LPO 30176

3,5-Dihydroxybenzaldehyde (1.0 g, 6.02 mmol) and N-bromosuccinimide (1.3 g, 7.3 mmol) were ground in a mortar for 2-5 min to obtain a pasty mixture. EtOAc (30 mL) and water (20 mL) were added and the separated organic layer was washed with water (20 mL), brine (20 mL), dried over MgSO₄, filtered and concentrated to dryness under reduced pressure to give a white solid. After recrystallization in cyclohexane, 2-bromo-3,5-dimethoxybenzaldehyde LPO 30176 (524 mg, 50% yield) was obtained as a white solid.

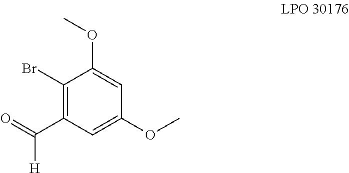

MW: 245.07; Yield=50%; White solid; Mp (° C.)=100.6

¹H-NMR (CDCl₃, δ): 3.86 (s, 3H, OCH₃), 3.92 (s, 3H, OCH₃), 6.72 (d, 1H, J=2.78

Hz, ArH), 7.05 (d, 1H, J=2.81 Hz, ArH), 10.42 (s, 1H, CHO).

¹³C-NMR (CDCl₃, δ): 55.8 (OCH₃), 56.6 (OCH₃), 103.4, 105.9, 109.1, 134.7, 157.1, 160.0, 192.1 (CHO).

MS-ESI m/z (% rel. Int.): 247.0/245.0 ([MH]⁺, 18/20), 166.1 (50), 138.1 (100).

HPLC: Method A, detection UV 254 nm, RT=6.03 min, peak area 99.9%.

### 4-(2-Bromo-3,5-dimethoxybenzyl)-7-ethoxyisoquinolin-8-ol hydrochloride 17

In an ace pressure tube (Aldrich, 100 mL), 2-((2,2-diethoxyethylamino)methyl)-6-ethoxyphenol SAO 33014 (578 mg, 2.04 mmol) and 2-bromo-3,5-dimethoxybenzaldehyde LPO 30176 (500 mg, 2.04 mmol) were dissolved in EtOH (2.8 mL) and a 37% HCl solution (2.8 mL) was added. The reaction mixture was stirred at 90° C. for 20 min, cooled at 4° C. (ice-water bath) and concentrated. The crude product was transformed into free base with a 1 M K₂CO₃ solution (50 mL) and extracted with EtOAc (250 mL). The separated organic layer was washed with brine (30 mL), dried over MgSO₄, filtered and evaporated to give a yellow solid. This solid was purified by column chromatography (SiO₂, eluent: CH₂Cl₂:MeOH=98:2) to give, after evaporation and drying, 4-(4-bromo-3,5-dimethoxybenzyl)-7-ethoxyisoquinolin-8-ol (241 mg). 4-(4-Bromo-3,5-dimethoxybenzyl)-7-ethoxyisoquinolin-8-ol was dissolved in MeOH (4 mL) and a 1.47 N HCl solution in MeOH (411 μL, 0.605 mmol) was slowly added. The reaction mixture was stirred at 4° C. for 15 min. After evaporation and drying under vacuum pump and P₂O₅, 4-(4-bromo-3,5-dimethoxybenzyl)-7-ethoxyisoquinolin-8-ol hydrochloride 17 was obtained as a yellow solid (247 mg, 27% yield).

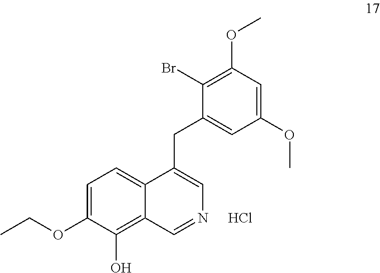 [SMILES: CCOc1ccc2c(Cc3cc(OC)cc(OC)c3Br)cncc2c1O ; Cl]

MW: 454.74; Yield: 27%; Yellow Solid; Mp (° C.): 255.4

R_{f}: 0.2 (CH₂Cl₂:MeOH=98:2, free base).

¹H-NMR (CD₃OD, δ): 1.50 (t, 3H, J=6.9 Hz, CH₂CH₃), 3.74 (s, 3H, OCH₃), 3.89 (s, 3H, OCH₃), 4.35 (q, 2H, J=7.0 Hz, CH₂CH₃), 4.58 (s, 2H, CCH₂), 6.40 (s, 1H, ArH), 6.59 (s, 1H, ArH), 7.74-7.77 (m, 2H, ArH), 7.99 (dd, 1H, J=9.1 Hz, J=2.4 Hz, ArH), 9.62 (s, 1H, ArH).

¹³C-NMR (CD₃OD, δ): 15.1, 37.6, 56.0, 56.9, 66.9, 99.3, 105.8, 109.2, 115.9, 120.4, 126.2, 129.2, 132.5, 136.1, 139.9, 143.1, 146.2, 146.7, 158.8, 161.8.

MS-ESI m/z (% rel. Int.): 418/420 ([MH]⁺, 50/50).

HPLC: Method A, detection UV 254 nm, RT=5.36 min, peak area 98.0%.

### Preparation of 4-(4-ethoxy-3,5-dimethoxybenzyl)-7-ethoxyisoquinolin-8-amine dihydrochloride 18

### 4-(4-Ethoxy-3,5-dimethoxybenzyl)-7-ethoxyisoquinolin-8-yl trifluoromethanesulfonate LPO 37002C

To a suspension of 4-(4-ethoxy-3,5-dimethoxybenzyl)-7-ethoxyisoquinolin-8-ol (free base of hydrochloride 14, 310 mg, 0.81 mmol) in CH₂Cl₂ (5 mL) in a 25 mL round-bottomed flask equipped with a magnetic stirrer was added NEt₃ (226 μL, 1.618 mmol) and N-phenyl-bis(trifluoromethanesulfonimide) (433.7 mg, 1.214 mmol). The reaction mixture was stirred for 20 min at RT then diluted with CH₂Cl₂ (50 mL) and the organic solution was washed with water (10 mL), brine (10 mL), dried over MgSO₄, filtered and concentrated at 40° C. under vacuum. The crude product was purified by column chromatography (SiO₂, eluent cyclohexane:EtOAc=10:0 to 7:3) to give, after evaporation and drying, 4-(4-ethoxy-3,5-dimethoxybenzyl)-7-ethoxyisoquinolin-8-yl trifluoromethanesulfonate LPO 37002C as an off-white solid (135.7 mg, 33% yield).

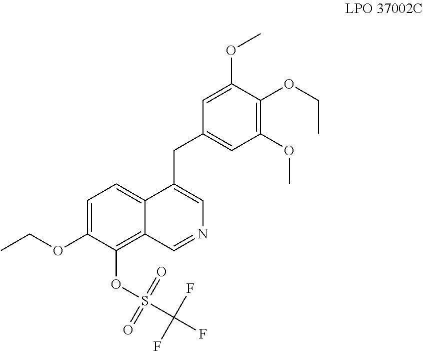 [SMILES: CCOc1ccc2c(Cc3cc(OC)c(OCC)c(OC)c3)cncc2c1OS(=O)(=O)C(F)(F)F]

MW: 515.5; Yield: 33%; Off-White Solid.

R_{f}: 0.2 (cyclohexane:EtOAc=7:3, free base).

¹H-NMR (CD₃OD, δ): 1.34 (t, 3H, J=7.1 Hz, CH₂CH₃), 1.52 (t, 3H, J=7.0 Hz, CH₂CH₃), 3.75 (s, 6H, 2×OCH₃), 4.02 (q, 2H, J=7.1 Hz, CH₂CH₃), 4.28 (q, 2H, J=7.0 Hz, CH₂CH₃), 4.30 (s, 2H, CH₂), 6.37 (s, 2H, 2×ArH), 7.53 (d, 2H, J=9.4 Hz, 2×ArH), 7.94 (d, 2H, J=9.3 Hz, 2×ArH), 8.40 (s, 1H, ArH), 9.36 (s, 1H, ArH).

¹³C-NMR (CD₃OD, δ): 14.4, 15.5, 36.6, 56.1 (2×C), 65.8, 68.8, 105.7 (2×C), 118.7 (q, CF₃, J=317.0 Hz), 119.0, 122.6, 125.1, 129.3, 130.0, 132.2, 134.5, 135.6, 142.7, 144.4, 148.2, 153.7 (2×C).

MS-ESI m/z (% rel. Int.): 516.0 ([MH]⁺, 100).

HPLC: Method A, detection UV 254 nm, RT=5.47 min, peak area 98%.

### 4-(4-Ethoxy-3,5-dimethoxybenzyl)-7-ethoxyisoquinolin-8-amine dihydrochloride 18

A mixture of (±) BINAP (32.5 mg, 0.052 mmol), Pd₂(dba)₃ (14.4 mg, 0.016 mmol), Cs₂CO₃ (170.1 mg, 0.52 mmol), 4-(4-ethoxy-3,5-dimethoxybenzyl)-7-ethoxyisoquinolin-8-yl trifluoromethanesulfonate 19 (134.7 mg, 0.261 mmol) and benzophenone imine (104 mg, 0.57 mmol) in dry toluene (5 mL) in a 20 mL microwave vial equipped with a magnetic stirrer was stirred for 5 h at 150° C. under microwave irradiation (150 W). After cooling to RT, the reaction mixture was diluted with THF (20 mL) and filtered through celite. The filtrate was poured in a 250 mL round-bottomed flask equipped with a magnetic stirrer before addition of a 1 N aqueous HCl solution (5 mL) and the mixture was stirred for 1 h at RT. The volatiles were then removed at 40° C. under vacuum and the residue was taken up in CH₂Cl₂ (50 mL), washed with K₂CO₃ (10 mL), with brine (10 mL), dried over MgSO₄, filtered and concentrated at 40° C. under vacuum. Purification by column chromatography (SiO₂, eluent cyclohexane:EtOAc=5:5 to 0:10) gave, after evaporation and drying, 4-(4-ethoxy-3,5-dimethoxybenzyl)-7-ethoxyisoquinolin-8-amine (32.5 mg). This product was dissolved in MeOH (2 mL) and a 1.35 N HCl solution in MeOH (129 μL, 0.174 mmol) was slowly added. The reaction mixture was stirred at 4° C. for 15 min. After evaporation and drying under P₂O₅, 4-(4-ethoxy-3,5-dimethoxybenzyl)-7-ethoxyisoquinolin-8-amine dihydrochloride 18 was obtained as a red solid (35 mg, 29% yield).

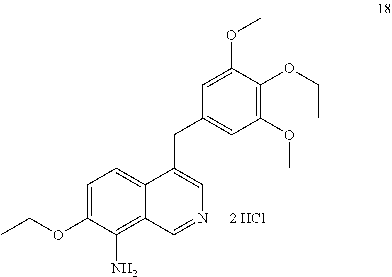 [SMILES: CCOc1ccc2c(Cc3cc(OC)c(OCC)c(OC)c3)cncc2c1N ; "2 HCl"]

MW: 455.37; Yield: 29%; Red solid; Mp (° C.): 235.0

R_{f}: 0.2 (cyclohexane:EtOAc=5:5, free base).

¹H-NMR (CD₃OD, δ): 1.29 (t, 3H, J=7.1 Hz, CH₂CH₃), 1.52 (t, 3H, J=6.9 Hz, CH₂CH₃), 3.78 (s, 6H, 2×OCH₃), 3.96 (q, 2H, J=7.0 Hz, CH₂CH₃), 4.31 (q, 2H, J=7.0 Hz, CH₂CH₃), 4.41 (s, 2H, CCH₂), 6.61 (s, 2H, 2×ArH), 7.58 (d, 2H, J=8.8 Hz, ArH), 7.83 (d, 2H, J=8.8 Hz, 2×ArH), 7.92 (s, 1H, ArH), 9.67 (s, 1H, ArH).

¹³C-NMR (CD₃OD, δ): 15.1, 15.7, 37.2, 56.7 (2×C), 66.4, 69.9, 107.4 (2×C), 112.9, 116.9, 124.0, 127.2, 132.2, 135.1, 136.8, 137.4, 139.2, 142.3, 145.7, 155.1 (2×C).

MS-ESI m/z (% rel. Int.): 383.2 ([MH]⁺, 100).

HPLC: Method A, detection UV 254 nm, RT=4.02 min, peak area 98%.

### Preparation of 4-(3,5-dimethoxy-4-propoxybenzyl)-7-ethoxyisoquinolin-8-ol hydrochloride 19

### 3,5-Dimethoxy-4-propoxybenzaldehyde TTA 24142

4-Hydroxy-3,5-dimethoxybenzaldehyde (1.0 g, 5.50 mmol) was dissolved in DMF (15 mL) and Cs₂CO₃ (1.8 g, 5.50 mmol) was added at 20° C. 1-Bromopropane (550 μL, 6.00 mmol) was added and the reaction mixture stirred at 40° C. for 10 h under an N₂ atmosphere. The reaction mixture was poured into water (150 mL) and extracted with EtOAc (100 mL). The organic layer was washed with water (2×50 mL), brine (50 mL), dried over MgSO₄, filtered and concentrated to dryness under reduced pressure to give a brown oil. After drying 24 h under vacuum, 3,5-dimethoxy-4-propoxybenzaldehyde TTA 24142 (900 mg, 73% crude yield) was obtained as brown oil. This crude product was used in the next step without further purification.

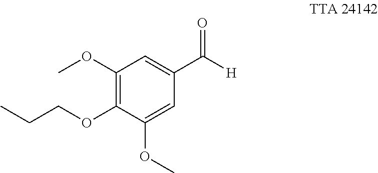 [SMILES: CCCOc1c(OC)cc(C=O)cc1OC]

MW: 224.25; Yield: 73% (crude); Brown oil.

R_{f}: 0.8 (CH₂Cl₂:EtOAc=8:2).

¹H-NMR (CDCl₃, δ): 1.02 (t, 3H, J=7.4 Hz, CH₂CH₃), 1.79 (sext., 2H, J=7.2 Hz, CH₂CH₂CH₃), 3.92 (s, 6H, 2×OCH₃), 4.05 (t, 2H, J=6.9 Hz, OCH₂), 7.13 (s, 2H, 2×ArH), 9.87 (s, 1H, CHO).

¹³C-NMR (CDCl₃, δ): 10.3, 23.4, 56.3 (2×C), 75.3, 106.7 (2×C), 131.5, 143.1, 153.9 (2×C), 191.1.

MS-ESI m/z (% rel. Int.): 225.1 ([MH]⁺, 63), 155.0 (100).

HPLC: Method A, detection UV 254 nm, RT=5.26 min, peak area 98%.

### Preparation of 4-(3,5-dimethoxy-4-propoxybenzyl)-7-ethoxyisoquinolin-8-ol hydrochloride 19

### 4-(3,5-Dimethoxy-4-propoxybenzyl)-7-ethoxyisoquinolin-8-ol hydrochloride 19

In an ace pressure tube (Aldrich, 100 mL), 2-((2,2-diethoxyethylamino)methyl)-6-ethoxyphenol SAO 33014 (632 mg, 2.23 mmol) and 3,5-dimethoxy-4-propoxybenzaldehyde TTA 24142 (500 mg, 2.23 mmol) were dissolved in EtOH (2.8 mL) and a 37% HCl solution (2.8 mL) was added. The reaction mixture was stirred at 90° C. for 25 min, cooled at 4° C. (ice-water bath) and concentrated. The crude product was transformed into free base with 1 M K₂CO₃ solution (50 mL) and extracted with EtOAc (250 mL). The separated organic layer was washed with brine (30 mL), dried over MgSO₄, filtered and evaporated to give a yellow solid. This solid was purified by column chromatography (SiO₂, eluent: gradient cyclohexane:EtOAc=10:0 to 5:5) to give, after evaporation and drying, 4-(3,5-dimethoxy-4-propoxybenzyl)-7-ethoxyisoquinolin-8-ol (247 mg). This compound was dissolved in MeOH (3 mL) and a 1.35 N HCl solution in MeOH (482 μL, 0.651 mmol) was slowly added. The reaction mixture was stirred at 4° C. for 15 min. After evaporation of solvent and drying under vacuum pump and under P₂O₅, 4-(3,5-dimethoxy-4-propoxybenzyl)-7-ethoxyisoquinolin-8-ol hydrochloride 19 was obtained as a yellow solid (271 mg, 28% yield).

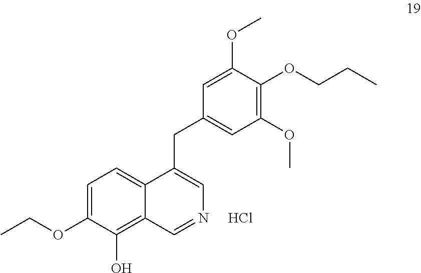 [SMILES: CCCOc1c(OC)cc(Cc2cncc3c(O)c(OCC)ccc23)cc1OC ; Cl]

MW: 433.93; Yield: 28%; Yellow Solid; Mp (° C.): 235.3

R_{f}: 0.2 (cyclohexane:EtOAc=5:5, free base).

¹H-NMR (CD₃OD, δ): 1.01 (t, 3H, J=4.4 Hz, CH₂CH₃), 1.52 (t, 3H, J=4.0 Hz, CH₂CH₃), 1.67-1.74 (m, 2H, CH₂CH₂CH₃), 3.77 (s, 6H, 2×OCH₃), 3.85 (m, 2H, OCH₂), 4.37 (q, 2H, J=6.9 Hz, OCH₂), 4.49 (s, 2H, CCH₂), 6.61 (s, 2H, 2×ArH), 7.89 (d, 1H, J=9.1 Hz, ArH), 8.02-8.08 (m, 2H, 2×ArH), 9.66 (s, 1H, ArH).

¹³C-NMR (CD₃OD, δ): 10.8, 15.1, 24.3, 37.0, 56.7 (2×C), 66.9, 76.2, 107.5 (2×C), 116.5, 120.5, 126.7, 128.3, 132.8, 134.9, 137.3, 138.0, 142.4, 146.4, 146.8, 155.2 (2×C).

MS-ESI m/z (% rel. Int.): 398.0 ([MH]⁺, 100).

HPLC: Method A, detection UV 254 nm, RT=4.20 min, peak area 98.0%.

### Preparation of 4-(4-Isobutoxy-3,5-dimethoxybenzyl)-7-ethoxyisoquinolin-8-ol hydrochloride 20

### 4-Isobutoxy-3,5-dimethoxybenzaldehyde ECO 33172

4-Hydroxy-3,5-dimethoxybenzaldehyde (500 mg, 2.75 mmol) was dissolved in DMF (8 mL) and Cs₂CO₃ (900 mg, 2.75 mmol, 1 eq) was added at 20° C. 1-Bromo-2-methylpropane (329 μL, 3.03 mmol, 1.1 eq) was added and the reaction mixture was stirred at 80° C. for 2 h under an N₂ atmosphere. After cooling, the reaction mixture was poured into water (40 mL) and extracted by Et₂O (300 mL). The organic layer was washed with water (2×50 mL), brine (50 mL), dried over MgSO₄, filtered and concentrated to dryness under reduced pressure to give an orange oil. The crude oil was purified by column chromatography (SiO₂, eluent cyclohexane:EtOAc=10:0 to 9:1) to give, after evaporation and drying, 4-isobutoxy-3,5-dimethoxybenzaldehyde ECO 33172 (218 mg, 33% yield) as yellow oil.

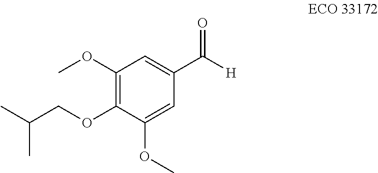 [SMILES: COc1cc(C=O)cc(OC)c1OCC(C)C]

MW: 238.28; Yield: 33%; Yellow oil.

R_{f}: 0.7 (cyclohexane:EtOAc=5:5).

¹H-NMR (CDCl₃, δ): 1.01 (s, 3H, CHCH₃), 1.04 (s, 3H, CHCH₃), 2.02-2.11 (m, 1H, J=6.69 Hz, CHCH₃), 3.84 (d, 2H, J=6.7 Hz, OCH₂), 3.91 (s, 6H, 2×OCH₃), 7.12 (s, 2H, 2×ArH), 9.86 (s, 1H, CHO).

MS-ESI m/z (% rel. Int.): 239.1 ([MH]⁺, 15), 183.0 (100).

HPLC: Method A, detection UV 254 nm, RT=5.69 min, peak area 99.9%.

### Preparation of 4-(4-isobutoxy-3,5-dimethoxybenzyl)-7-ethoxyisoquinolin-8-ol hydrochloride 20

### 4-(4-Isobutoxy-3,5-dimethoxybenzyl)-7-ethoxyisoquinolin-8-ol hydrochloride 20

In an ace pressure tube (Aldrich, 100 mL), 2-((2,2-diethoxyethylamino)methyl)-6-ethoxyphenol SAO 33014 (259 mg, 0.91 mmol) and 4-isobutoxy-3,5-dimethoxybenzaldehyde ECO 33172 (217.5 mg, 0.91 mmol) were dissolved in EtOH (1.6 mL) and a 37% HCl solution (1.6 mL) was added. The reaction mixture was stirred at 90° C. for 25 min, cooled to 4° C. (ice-water bath) and concentrated. The crude product was transformed into free base with a 18 N NH₄OH solution (500 μL) and extracted with CH₂Cl₂ (250 mL). The separated organic layer was washed with brine (30 mL), dried over MgSO₄, filtered and evaporated to give a yellow solid. This solid was purified by column chromatography (SiO₂, eluent cyclohexane:EtOAc=10:0 to 5:5) to give, after evaporation and drying, 4-(4-isobutoxy-3,5-dimethoxybenzyl)-7-ethoxyisoquinolin-8-ol (168 mg). This compound was dissolved in MeOH (2 mL) and a 1.35 N HCl solution in MeOH (316 μL, 0.43 mmol) was slowly added. The reaction mixture was stirred at 4° C. for 15 min. After evaporation and drying under vacuum pump and under P₂O₅, 4-(4-isobutoxy-3,5-dimethoxybenzyl)-7-ethoxyisoquinolin-8-ol hydrochloride 20 was obtained as a yellow solid (178 mg, 43% yield).

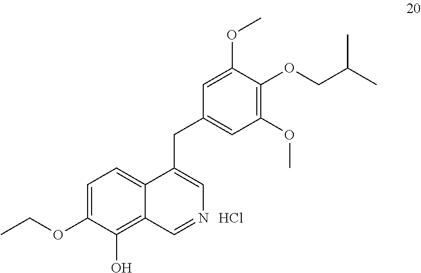 [SMILES: CCOc1ccc2c(Cc3cc(OC)c(OCC(C)C)c(OC)c3)cncc2c1O ; Cl]

MW: 447.95; Yield: 43%; Yellow solid; Mp (° C.): 241.3

R_{f}: 0.3 (cyclohexane:EtOAc=5:5, free base).

¹H-NMR (CD₃OD, δ): 1.00 (dd, 6H, J=6.7 Hz, J=2.7 Hz, 2×CH₃), 1.51 (t, 3H, J=7.0 Hz, CH₂CH₃), 1.92-2.02 (m, 1H, CH(CH₃)₂), 3.66 (dd, 2H, J=6.6 Hz, J=2.7 Hz, OCH₂CH₃), 3.77 (s, 6H, 2×OCH₃), 4.36 (t, 2H, J=7.0 Hz, OCH₂CH), 4.49 (s, 2H, CH₂), 6.61 (s, 2H, 2×ArH), 7.89 (dd, 1H, J=9.1 Hz, J=2.2 Hz, ArH), 8.06 (dd, 2H, J=9.1 Hz, J=2.6 Hz, 2×ArH), 9.67 (s, 1H, ArH).

¹³C-NMR (CD₃OD, δ): 15.0, 19.6 (2×C), 30.2, 37.0, 56.7 (2×C), 66.9, 81.2, 107.6 (2×C), 116.5, 120.5, 126.7, 128.3, 132.8, 134.7, 137.7, 138.0, 142.4, 146.4, 146.8, 155.1 (2×C).

MS-ESI m/z (% rel. Int.): 412.0 ([MH]⁺, 100).

HPLC: Method A, detection UV 254 nm, RT=4.53 min, peak area 98%.

### Preparation of 4-(3,4,5-trimethoxybenzyl)-7-ethoxyisoquinolin-8-yl sulfamate hydrochloride 21

### Preparation of a Solution of Sulfamoyl Chloride in Toluene:

Formic acid (266 μL, 7.07 mmol) was added to chlorosulfonyl isocyanate (615 μL, 7.07 mmol) at 4° C. for 1 h and anhydrous toluene (5 mL) was added. The mixture was stirred at RT overnight, the precipitate was filtered off and the obtained filtrate was used without further purification.

### 4-(3,4,5-Trimethoxybenzyl)-7-ethoxyisoquinolin-8-yl sulfamate hydrochloride 21

4-(3,4,5-Trimethoxybenzyl)-7-ethoxyisoquinolin-8-ol hydrochloride 1 (536 mg, 1.45 mmol) was dissolved in DMA (2 mL) and anhydrous Et₃N (365 μL, 2.61 mmol) and NaH 60% (69.6 mg, 1.74 mmol) were added at 4° C. under N₂. A solution of sulfamoyl chloride in toluene (3.11 mL, 4.35 mmol) was slowly added dropwise. The reaction mixture was stirred overnight at 4° C. to RT under an N₂ atmosphere and was poured into mixture of CH₂Cl₂ (100 mL) and water (30 mL). The organic layer was washed with brine (30 mL), dried over MgSO₄, filtered and evaporated. The obtained crude product was purified by column chromatography, (SiO₂, eluent CH₂Cl₂:MeOH=100:0 to 98:2) to give, after evaporation and drying, 2-(3,4,5-trimethoxybenzyl)-7-ethoxyisoquinolin-8-yl sulfamate (125 mg, 19% yield). A fraction of 4-(3,4,5-trimethoxybenzyl)-7-ethoxyisoquinolin-8-yl sulfamate (54.7 mg, 0.122 mmol) was dissolved in MeOH (2 mL) at 4° C. with 1.35 N HCl solution in MeOH (95 μL, 0.128 mmol) to give after evaporation of solvent and drying in vacuo under P₂O₅, 4-(3,4,5-trimethoxybenzyl)-7-ethoxyisoquinolin-8-yl sulfamate hydrochloride 21 as a yellow solid (49.6 mg, 16% yield).

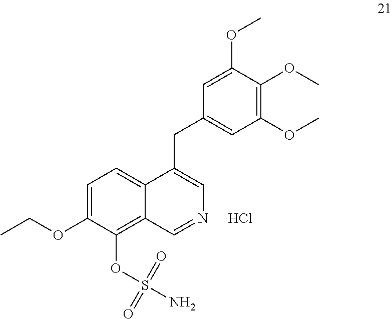 [SMILES: CCOc1ccc2c(Cc3cc(OC)c(OC)c(OC)c3)cncc2c1OS(N)(=O)=O ; Cl]

MW: 484.95; Yield: 16%; Yellow solid; Mp (° C.): 192.9

R_{f}: 0.25 (CH₂Cl₂:MeOH=98:2, free base).

¹H-NMR (CD₃OD, δ): 1.53 (t, 3H, J=5.5 Hz, CH₂CH₃), 3.74 (s, 3H, OCH₃), 3.79 (s, 6H, 2×OCH₃), 4.21 (q, 2H, J=6.9 Hz, OCH₂CH₃), 4.58 (s, 2H, CH₂), 6.64 (s, 2H, 2×ArH), 8.23-8.29 (m, 2H, 2×ArH), 8.44 (d, 1H, J=9.2 Hz, ArH), 9.60 (s, 1H, ArH).

¹³C-NMR (CD₃OD, δ): 15.0, 37.0, 56.7 (2×C), 61.1, 67.2, 107.5 (2×C), 125.8, 125.9, 127.7, 130.1, 133.6, 135.0, 135.9, 138.2, 138.8, 142.6, 153.7, 155.0 (2×C).

MS-ESI m/z (% rel. Int.): 449.2 ([MH]⁺, 100).

HPLC: Method A, detection UV 254 nm, RT=4.64 min, peak area 98%.

### Preparation of 4-(4-(2-methoxyethoxy)-3,5-dimethoxybenzyl)-7-ethoxyisoquinolin-8-ol hydrochloride 22

### 3,5-Dimethoxy-4-(2-methoxyethoxy)benzaldehyde ECO 33162

4-Hydroxy-3,5-dimethoxybenzaldehyde (500 mg, 2.75 mmol) was dissolved in DMF (8 mL) and Cs₂CO₃ (900 mg, 2.75 mmol, 1 eq) was added at 20° C. 2-Bromoethyl-methyl ether (550 μL, 5.8 mmol, 2.1 eq) was added and the reaction mixture was stirred at 150° C. for 10 min under microwave irradiation (150 W). After cooling, the reaction mixture was poured into water (150 mL) and extracted by Et₂O (200 mL). The organic layer was washed with water (2×50 mL), brine (50 mL), dried over MgSO₄, filtered and concentrated to dryness under reduced pressure to give an orange solid (595 mg). Purification by column chromatography (SiO₂, eluent cyclohexane:EtOAc=7:3) gave, after evaporation and drying, 3,5-dimethoxy-4-(2-methoxyethoxy)benzaldehyde ECO 33162 as an off-white solid (477 mg, 72% yield).

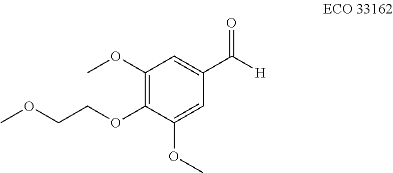 [SMILES: COCCOc1c(OC)cc(C=O)cc1OC]

MW: 240.25; Yield: 72%; Off-white solid; Mp (° C.): 66.2

R_{f}: 0.7 (cyclohexane:EtOAc=5:5).

¹H-NMR (CDCl₃, δ): 3.43 (s, 3H, OCH₃), 3.72 (t, 2H, J=3.5 Hz, CH₂), 3.93 (s, 6H, 2×OCH₃), 4.23 (t, 2H, J=4.77 Hz, CH₂), 7.13 (s, 2H, 2×ArH), 9.87 (s, 1H, CHO).

MS-ESI m/z (% rel. Int.): 241.1 ([MH]⁺, 72), 183.0 (100).

HPLC: Method A, detection UV 254 nm, RT=4.21 min, peak area 98.0%.

### Preparation of 4-(4-(2-methoxyethoxy)-3,5-dimethoxybenzyl)-7-ethoxyisoquinolin-8-ol hydrochloride 22

### 4-(4-(2-Methoxyethoxy)-3,5-dimethoxybenzyl)-7-ethoxyisoquinolin-8-ol hydrochloride 22

In an ace pressure tube (Aldrich, 38 mL), 2-((2,2-diethoxyethylamino)methyl)-6-ethoxyphenol SAO 33014 (294.9 mg, 1.04 mmol) and 3,5-dimethoxy-4-(2-methoxyethoxy)benzaldehyde ECO 33162 (250 mg, 1.04 mmol) were dissolved in EtOH (1.6 mL) and a HCl 37% solution (1.6 mL) was added. The reaction mixture was stirred at 90° C. for 25 min, cooled to 4° C. (ice-water bath) and concentrated. The obtained crude product was transformed into free base using a 18 N NH₄OH solution (500 μL) and extracted with CH₂Cl₂ (250 mL). The organic layer was washed with brine (30 mL), dried over MgSO₄, filtered and evaporated to give a yellow oil. This crude oil was purified by column chromatography (SiO₂, eluent cyclohexane:EtOAc=100:0 to 0:100) to give, after evaporation and drying, 4-(4-(2-methoxyethoxy)-3,5-dimethoxybenzyl)-7-ethoxyisoquinolin-8-ol (84.1 mg). This solid was dissolved in MeOH (2 mL) and a 1.35 N HCl solution in MeOH (158 μL, 0.214 mmol) was slowly added. The reaction mixture was stirred at 4° C. for 15 min. After evaporation and drying in vacuo under P₂O₅, 4-(4-(2-methoxyethoxy)-3,5-dimethoxybenzyl)-7-ethoxyisoquinolin-8-ol hydrochloride 22 was obtained as a yellow solid (89.5 mg, 19% yield).

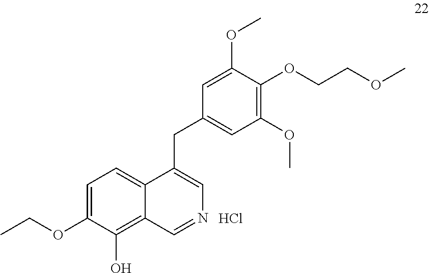 [SMILES: CCOc1ccc2c(Cc3cc(OC)c(OCCOC)c(OC)c3)cncc2c1O ; Cl]

MW: 449.92; Yield: 19%; Yellow Solid; Mp (° C.): 210.2

R_{f}: 0.25 (EtOAc, free base).

¹H-NMR (CD₃OD, δ): 1.51 (t, 3H, J=7.0 Hz, CH₂CH₃), 3.41 (s, 3H, OCH₃), 3.66 (t, 2H, J=4.6 Hz, OCH₂), 3.78 (s, 6H, 2×OCH₃), 4.03 (t, 2H, J=4.6 Hz, OCH₂), 4.36 (q, 2H, J=7.0 Hz, OCH₂CH₃), 4.49 (s, 2H, CH₂), 6.62 (s, 2H, 2×ArH), 7.88 (d, 1H, J=9.1 Hz, ArH), 8.04 (d, 1H, J=9.1 Hz, ArH), 8.08 (s, 1H, ArH), 9.67 (s, 1H, ArH).

¹³C-NMR (CD₃OD, δ): 15.0, 37.0, 56.7 (2×C), 59.0, 66.9, 72.9, 73.1, 107.5 (2×C), 116.5, 120.5, 126.7, 128.3, 132.8, 135.2, 137.1, 137.9, 142.5, 146.4, 146.9, 155.1 (2×C).

MS-ESI m/z (% rel. Int.): 414.1 ([MH]⁺, 100).

HPLC: Method A, detection UV 254 nm, RT=4.04 min, peak area 96.0%.

### Preparation of 7-ethoxy-4-(4-ethoxy-3,5-dimethoxybenzyl)isoquinoline-1-carbonitrile 23

### 7-Ethoxy-4-(4-ethoxy-3,5-dimethoxybenzyl)isoquinoline-1-carbonitrile 23

7-Ethoxy-4-(4-ethoxy-3,5-dimethoxybenzyl)isoquinoline ANP 311788 (300 mg, 0.82 mmol) was dissolved in CH₂Cl₂ (15 mL) and mCPBA (303 mg, 1.22 mmol) was added at RT. The reaction mixture was stirred at RT for 15 h and the reaction mixture was poured into a 1 N NaHCO₃ solution (30 mL) and extracted with CH₂Cl₂ (100 mL). The organic layer was washed with brine (30 mL), dried over MgSO₄, filtered and concentrated to dryness to give 7-ethoxy-4-(4-ethoxy-3,5-dimethoxybenzyl)isoquinoline 2-oxide TTA 24144A as yellow solid (271 mg, 86% yield). TTA 24144A (271 mg, 0.71 mmol) was refluxed in THF (15 mL) with DBU (240 μL, 1.55 mmol) and trimethylsilyl cyanide (165 μL, 1.25 mmol) for 4 h under an N₂ atmosphere. The reaction mixture was evaporated at 45° C. to give 310 mg of a brown residue. Purification by column chromatography (SiO₂, eluent cyclohexane:EtOAc=10:0 to 8:2) gave, after evaporation and drying, 7-ethoxy-4-(4-ethoxy-3,5-dimethoxybenzyl)isoquinoline-1-carbonitrile 23 as a white solid (105 mg, 33% yield).

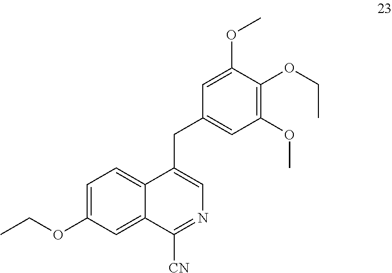 [SMILES: CCOc1ccc2c(Cc3cc(OC)c(OCC)c(OC)c3)cnc(C#N)c2c1]

MW: 392.45; Yield: 33%; White Solid; Mp (° C.): 141.0

R_{f}: 0.2 (cyclohexane:acetone=8:2).

¹H-NMR (CD₃OD, δ): 1.33 (t, 3H, J=7.0 Hz, CH₂CH₃), 1.52 (t, 3H, J=6.9 Hz, CH₂CH₃), 3.75 (s, 6H, OCH₃), 4.01 (q, 2H, J=7.0 Hz, CH₂CH₃), 4.24 (q, 2H, J=6.9 Hz, CH₂CH₃), 4.35 (s, 2H, CH₂), 6.35 (s, 2H, 2×ArH), 7.41 (dd, 1H, J=9.2 Hz, J=2.5 Hz, ArH), 7.51 (d, 1H, J=2.4 Hz, ArH), 7.96 (d, 1H, J=9.2 Hz, ArH), 8.37 (s, 2H, 2×ArH).

¹³C-NMR (CD₃OD, δ): 14.5, 15.5, 36.7, 56.1 (2×C), 64.3, 68.8, 103.1, 105.8 (2×C), 116.4, 125.5, 125.8, 130.5, 131.1, 131.9, 133.9, 134.6, 135.8, 142.4, 153.8 (2×C), 159.4.

MS-ESI m/z (% rel. Int.): 393.3 ([MH]⁺, 100).

HPLC: Method A, detection UV 254 nm, RT=5.7 min, peak area 98.0%.

### Preparation of 2-(4-(3,4,5-trimethoxybenzyl)-7-ethoxyisoquinolin-8-yloxy)acetonitrile 24

### 2-(4-(3,4,5-Trimethoxybenzyl)-7-ethoxyisoquinolin-8-yloxy)acetonitrile 24

4-(3,4,5-Trimethoxybenzyl)-7-ethoxyisoquinolin-8-ol CCH 34046-2 (compound 1 freebase, 200 mg, 0.541 mmol) was dissolved in DMF (3 mL) and Cs₂CO₃ (265 mg, 0.81 mmol) and triethylamine (79.4 μL, 0.57 mmol) were added. 2-Chloroacetonitrile (37.8 μL, 0.595 mmol) was slowly added dropwise. The reaction mixture was stirred for 2 h at 90° C. under a nitrogen atmosphere. The reaction mixture was evaporated at 65° C. and the obtained crude product was added in H₂O (50 mL) and extracted with Et₂O (2×100 mL). The combined organic layers were washed with brine (30 mL), dried over MgSO₄, filtered and evaporated to give a yellow oil. This crude oil was purified by column chromatography (SiO₂, eluent: CH₂Cl₂:MeOH=100:0 to 97:3) to give, after evaporation and drying, 2-(4-(3,4,5-trimethoxybenzyl)-7-ethoxyisoquinolin-8-yloxy)acetonitrile 24 as a brown oil (155 mg, 70% yield).

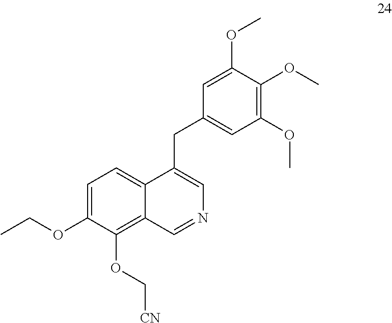 [SMILES: CCOc1ccc2c(Cc3cc(OC)c(OC)c(OC)c3)cncc2c1OCC#N]

MW: 408.45; Yield: 70%; Brown oil.

R_{f}: 0.3 (CH₂Cl₂:MeOH).

¹H-NMR (CDCl₃, δ): 1.51 (t, 3H, J=7.0 Hz, CH₂CH₃), 3.75 (s, 6H, 2×OCH₃), 3.81 (s, 3H, OCH₃), 4.24 (q, 2H, J=7.0 Hz, OCH₂CH₃), 4.28 (s, 2H, OCH₂CN), 5.10 (s, 2H, CH₂), 6.39 (s, 2H, 2×ArH), 7.72 (d, 1H, J=9.2 Hz, ArH), 7.46 (d, 1H, J=9.3 Hz, ArH), 8.33 (s, 1H, ArH), 9.51 (s, 1H, ArH).

¹³C-NMR (CDCl₃, δ): 15.0, 36.7, 56.1 (2×C), 57.6, 60.8, 65.5, 105.6 (2×C), 115.5, 119.7, 121.5, 123.9, 128.9, 130.3, 135.3, 136.6, 140.2, 142.4, 146.2, 147.3, 153.3 (2×C).

MS-ESI m/z (% rel. Int.): 409.1 ([MH]⁺, 100).

HPLC: Method A, detection UV 254 nm, RT=4.30 min, peak area 98%.

### Preparation of (4-(4-ethoxy-3,5-dimethoxybenzyl)-7-ethoxyisoquinolin-1-yl)methanamine dihydrochloride 25

### (4-(4-Ethoxy-3,5-dimethoxybenzyl)-7-ethoxyisoquinolin-1-yl)methanamine dihydrochloride 25

7-Ethoxy-4-(4-ethoxy-3,5-dimethoxybenzyl)isoquinoline 2-oxide TTA 24144A (34.4 mg, 0.088 mmol) was dissolved in EtOH (5 mL) and CHCl₃ (1 mL) and Pd/C (10%, 18 mg, 0.169 mmol) was added. The reaction mixture was stirred at RT for 3 days under an H₂ atmosphere. The reaction mixture was filtered under celite, evaporated and the obtained crude product was purified by column chromatography (SiO₂, eluent: CH₂Cl₂:MeOH=95:5 to 9:1) to give, after evaporation and drying, (4-(4-ethoxy-3,5-dimethoxybenzyl)-7-ethoxyisoquinolin-1-yl)methanamine (8.2 mg). (4-(4-Ethoxy-3,5-dimethoxybenzyl)-7-ethoxyisoquinolin-1-yl)methanamine was dissolved in MeOH (2 mL) and a 1.35 N HCl solution in MeOH (32 μL, 0.042 mmol) was slowly added. The reaction mixture was stirred at 4° C. for 15 min. After evaporation and drying in vacuo under P₂O₅, (4-(4-ethoxy-3,5-dimethoxybenzyl)-7-ethoxyisoquinolin-1-yl)methanamine dihydrochloride 25 was obtained as a green solid (8.7 mg, 21% yield).

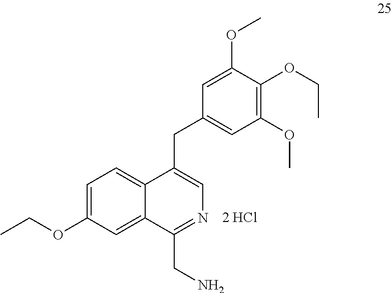 [SMILES: CCOc1ccc2c(Cc3cc(OC)c(OCC)c(OC)c3)cnc(CN)c2c1]

MW: 469.4; Yield: 21%; Green Solid; Mp (° C.): 135.2

R_{f}: 0.25 (CH₂Cl₂:MeOH=95:5, free base).

¹H-NMR (CD₃OD, δ): 1.29 (t, 3H, J=7.1 Hz, CH₂CH₃), 1.50 (t, 3H, J=6.9 Hz, CH₂CH₃), 3.73 (s, 6H, 2×OCH₃), 3.93 (q, 2H, J=7.1 Hz, OCH₂CH₃), 4.33 (q, 2H, J=7.0 Hz, OCH₂CH₃), 4.47 (s, 2H, CH₂), 4.93 (s, 2H, CH₂N), 6.56 (s, 2H, 2×ArH), 7.60 (d, 1H, J=2.1 Hz, ArH), 7.67 (dd, 1H, J=9.3 Hz, J=2.4 Hz, ArH), 8.28 (d, 1H, J=9.2 Hz, ArH), 8.34 (s, 1H, ArH).

¹³C-NMR (CD₃OD, δ): 13.2, 14.0, 35.3, 38.7, 54.9 (2×C), 64.0, 68.2, 103.3, 105.6 (2×C), 126.1, 126.2, 127.3, 131.8, 134.1, 134.3, 134.5, 135.0, 146.9, 153.4 (2×C), 159.3.

MS-ESI m/z (% rel. Int.): 397.2 ([MH]⁺, 100).

HPLC: Method A, detection UV 254 nm, RT=4.50 min, peak area 96.0%.

### Preparation of (S)-2-amino-N-(7-ethoxy-4-(3,4,5-trimethoxybenzyl)isoquinolin-8-yl)-3-hydroxypropanamide dihydrochloride 26

### (S)-3-(tert-Butoxycarbonyl)-2,2-dimethyloxazolidine-4-carboxylic acid

To a solution of methyl (S)-3-tert-butyl 4-methyl 2,2-dimethyloxazolidine-3,4-dicarboxylate (0.35 g, 1.35 mmol) in THF (10 mL) in a 50 mL round-bottomed flask equipped with a magnetic stirrer was added a solution of LiOH monohydrate (62 mg, 1.48 mmol) in H₂O (5 mL) and the mixture was stirred overnight at RT. THF was evaporated at 40° C. under vacuum and the residue was diluted with H₂O (25 mL) before acidification with 3% aqueous HCl to pH=4. The solution was extracted with Et₂O (3×50 mL) and the organic layers were combined, washed with brine (20 mL), dried over Na₂SO₄, filtered and concentrated at 40° C. under vacuum to give (S)-3-(tert-butoxycarbonyl)-2,2-dimethyloxazolidine-4-carboxylic acid CCH 34168-1 as a colorless oil (147 mg, 44% yield).

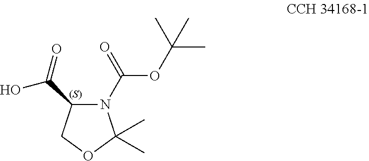

MW: 469.4; Yield: 21%; Colorless oil.

### (S)-2-Amino-N-(7-ethoxy-4-(3,4,5-trimethoxybenzyl)isoquinolin-8-yl)-3-hydroxypropanamide dihydrochloride 26

To a solution of (S)-3-(tert-butoxycarbonyl)-2,2-dimethyloxazolidine-4-carboxylic acid CCH 34168-1 (136 mg, 554 μmol) in dry CH₂Cl₂ (10 mL) under N₂ in a 50 mL round-bottomed flask equipped with a magnetic stirrer was added a solution of 7-ethoxy-4-(3,4,5-trimethoxybenzyl)isoquinolin-8-amine CCH 34058 (143 mg, 388 μmol) in dry CH₂Cl₂ (10 mL) and the mixture was cooled to 0° C. before portionwise addition of EDCI (149 mg, 777 μmol). The reaction mixture was then allowed to warm up to RT and stirring was continued overnight before dilution with CH₂Cl₂ to a volume of 50 mL. Water (10 mL) was then added and stirring was continued at RT for 3 h, after which the organic phase was isolated, washed with 0.1 N aqueous NaOH (2×10 mL), H₂O (10 mL), dried (Na₂SO₄) and concentrated at 40° C. under vacuum to give 230 mg of CCH 34168-2 as a brown oil. The oil was immediately dissolved in TFA (5 mL) in a 25 mL round-bottomed flask equipped with a magnetic stirrer and the mixture was stirred overnight at RT. After evaporation of TFA at 40° C. under vacuum, the residue was purified by reversed phase column chromatography, elution from H₂O to H₂O:CH₃CN=7:3, lyophilised, taken up in a 0.19 N HCl solution in MeOH (10 mL) and concentrated to dryness to give (S)-2-amino-N-(7-ethoxy-4-(3,4,5-trimethoxybenzyl)isoquinolin-8-yl)-3-hydroxypropanamide dihydrochloride 26 as a pale brown solid (81 mg, 28% yield).

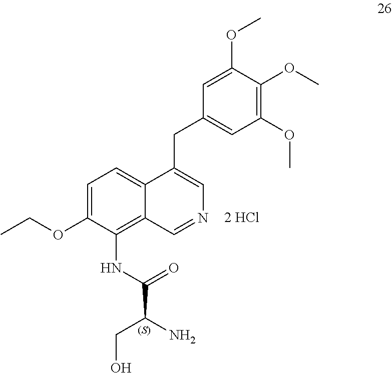

MW: 528.43; Yield: 28%; Pale brown solid; Mp (° C.): 206.3 (dec.)

¹H-NMR (CD₃OD, δ): 1.52 (t, 3H, J=6.9 Hz, CH₂CH₃), 3.75 (s, 3H, OCH₃), 3.79 (s, 6H, 2×OCH₃), 4.23 (d, 2H, J=4.6 Hz, CHCH₂O), 4.41 (q, 2H, J=6.9 Hz, CH₂CH₃), 4.47 (t, 1H, J=4.6 Hz, CHCH₂O), 4.60 (s, 2H, CH₂), 6.64 (s, 2H, 2×ArH), 8.21 (d, 1H, J=9.4 Hz), 8.28 (s, 1H, ArH), 8.51 (d, 1H, J=9.4 Hz), 9.52 (s, 1H, ArH).

¹³C-NMR (CD₃OD, δ): 15.1, 37.0, 56.7, 61.1, 62.0, 67.0, 107.6 (2×C), 122.2, 126.2, 127.0, 127.2, 129.5, 133.8, 135.0, 138.2, 139.1, 143.3, 155.0 (2×C), 156.0, 169.1.

MS-ESI m/z (rel. int.): 456 ([MH]⁺, 100), 369 (40).

HPLC: Method A, detection UV 254 nm, RT=3.31 min, peak area 99.2%.

### Preparation of (4-(3,4,5-trimethoxybenzyl)-7-methoxyisoquinoline hydrochloride 27

### (E)-2,2-Diethoxy-N-(3-methoxybenzylidene)ethanamine ECO 33112

Under anhydrous conditions (Dean Stark apparatus), m-anisaldehyde (5 mL, 41.09 mmol) was dissolved in toluene (100 mL). Aminoacetaldehyde diethyl acetal (9 mL, 61.64 mmol) was added via syringe at RT. Water (25 mL) and toluene (5 mL) were added in the Dean Stark burette. The reaction mixture was stirred at 150° C. for 4 h under an N₂ atmosphere. The reaction mixture was evaporated under vacuum at 60° C. to yield to (E)-2,2-diethoxy-N-(3-methoxybenzylidene)ethanamine ECO 33112 as yellow oil (9.60 g, 92% yield). This crude product was used in the next step without further purification.

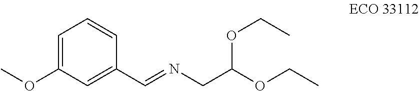 [SMILES: CCOC(C/N=C/c1cccc(OC)c1)OCC]

MW: 251.33; Yield: 92%; Yellow oil.

¹H-NMR (CDCl₃, δ): 1.21 (t, 6H, J=7.0 Hz, 2×CH₂CH₃), 3.54-3.62 (m, 2H, CH₂N), 3.69-3.80 (m, 4H, 2×CH₃CH₂O), 3.85 (s, 3H, CH₃O), 4.81 (t, 1H, J=5.3 Hz, CH₂CH(OEt)₂), 6.97-7.00, (m, 1H, ArH), 7.25-7.35 (m, 3H, 3×ArH), 8.26 (s, 1H, CH═N).

### 2,2-Diethoxy-N-(3-methoxybenzyl)ethanamine ECO 33116

In a 100 mL round bottom flask, the crude product (E)-2,2-diethoxy-N-(3-methoxybenzylidene)ethanamine ECO 33112 (9.60 g, 38.2 mmol) and NaBH₄ (2.75 g, 72.60 mmol) were dissolved in absolute ethanol (80 mL). The reaction mixture was stirred at 100° C. for 1 h under an N₂ atmosphere. H₂O (3.5 mL) was added at RT and the solvents were evaporated at 45° C. H₂O (100 mL) and CH₂Cl₂ (200 mL) were added to the mixture. The separated organic layer was washed with brine, dried over MgSO₄, filtered and concentrated to dryness under reduced pressure to give 2,2-diethoxy-N-(3-methoxybenzyl)ethanamine ECO 33116 as a yellow oil (8.91 g, 92% yield). This crude product was used in the next step without further purification.

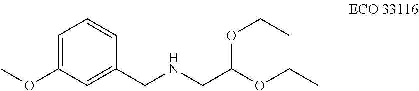 [SMILES: CCOC(CNCc1cccc(OC)c1)OCC]

MW: 253.34; Yield: 92%; Yellow oil.

¹H-NMR (CDCl₃, δ): 1.22 (t, 6H J=7.0 Hz, 2×CH₂CH₃), 1.95 (s, 1H, NH); 2.77 (d, 2H J=5.5 Hz, NCH₂), 3.52-3.60 (m, 4H, 2×CH₂O), 3.79 (s, 2H, CH₂N), 3.81 (s, 3H, CH₃O), 4.08 (m, 2H, CH₂O), 4.61 (t, 1H, J=5.6 Hz, CHO), 6.77-6.91 (m, 3H, 3×ArH).

¹³C-NMR (CDCl₃, δ): 15.4 (2×C), 51.6, 53.8, 55.2, 62.4 (2×C), 102.2, 112.5, 113.5, 120.4, 129.4, 141.9, 159.7.

MS-ESI m/z (% rel. Int.): 209.2 (7), 208.3 (49), 162.2 (100).

HPLC: Method A, detection UV 254 nm, RT=4.22 min, peak area 99.9%.

### 4-(3,4,5-Trimethoxybenzyl)-7-methoxyisoquinoline hydrochloride 27

In an ace pressure tube (Aldrich, 38 mL), ECO 33116 (3.0 g, 11.84 mmol) and 3,4,5-trimethoxybenzaldehyde (2.3 g, 11.84 mmol) were dissolved in EtOH (15 mL) and a 37% HCl solution (15 mL) was added. The reaction mixture was stirred at 90° C. for 30 min, cooled at 4° C. and concentrated. The obtained crude product was transformed into free base using a 18 N NH₄OH solution (2 mL) and extracted with CH₂Cl₂ (500 mL). The organic layer was washed with brine (50 mL), dried over MgSO₄, filtered and evaporated to give a yellow solid. This solid was purified by column chromatography (SiO₂, eluent cyclohexane:EtOAc=100:0 to 0:100) to give, after evaporation and drying, 4-(3,4,5-trimethoxybenzyl)-7-methoxyisoquinoline LPO 37024C (864 mg). 4-(3,4,5-Trimethoxybenzyl)-7-methoxyisoquinoline (30 mg, 0.088 mmol) was dissolved in MeOH (2 mL) and a 1.35 N HCl solution in MeOH (69 μL, 0.093 mmol) was slowly added. The reaction mixture was stirred at 4° C. for 15 min. After evaporation and drying under P₂O₅, 4-(3,4,5-trimethoxybenzyl)-7-methoxyisoquinoline hydrochloride 27 was obtained as a yellow solid (32 mg, 22% yield).

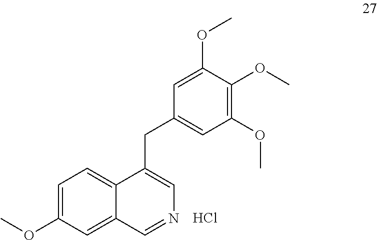

MW: 375.85; Yield: 22%; Yellow solid; Mp (° C.): 229.2

R_{f}: 0.25 (cyclohexane:EtOAc=4:6, free base).

¹H-NMR (CD₃OD, δ): 3.74 (s, 3H, OCH₃), 3.78 (s, 6H, 2×OCH₃), 4.06 (s, 3H, OCH₃), 4.55 (s, 2H, CH₂), 6.62 (s, 2H, 2×ArH), 7.82-7.85 (m, 2H, 2×ArH), 8.25 (s, 1H, ArH), 8.38 (d, 1H, J=8.9 Hz, ArH), 9.67 (s, 1H, ArH).

¹³C-NMR (CD₃OD, δ): 36.8, 56.7 (2×C), 56.7, 61.1, 107.5 (2×C), 108.8, 127.2, 130.7, 130.8, 131.1, 134.8, 135.2, 138.2, 138.3, 145.4, 155.0 (2×C), 162.2.

MS-ESI m/z (% rel. Int.): 340.1 ([MH]⁺, 100).

HPLC: Method A, detection UV 254 nm, RT=4.03 min, peak area 98.0%.

### Preparation of (7-methoxyisoquinolin-4-yl)(3,4,5-trimethoxyphenyl)methanone hydrochloride 28

### (7-Methoxyisoquinolin-4-yl)(3,4,5-trimethoxyphenyl)methanone hydrochloride 28

4-(3,4,5-Trimethoxybenzyl)-7-methoxyisoquinoline LPO 37024C (520 mg, 1.53 mmol) was dissolved in acetic anhydride (5.2 mL) at 4° C. under N₂ and a 70% HNO₃ solution (746 μL, 16.70 mmol) was added dropwise. The reaction mixture was stirred overnight from 4° C. to RT. The precipitate was filtered to give (7-methoxyisoquinolin-4-yl)(3,4,5-trimethoxyphenyl)methanone nitrate (260 mg). This solid was transformed into its freebase using a 1 M K₂CO₃ aqueous solution (30 mL) and the resulting mixture was extracted by EtOAc (150 mL). The separated organic layer was washed with brine, dried over MgSO₄, filtered and evaporated to give (7-methoxyisoquinolin-4-yl)(3,4,5-trimethoxyphenyl)methanone (251 mg). This compound (50 mg, 0.141 mmol) was dissolved in MeOH (2 mL) and a 1.35 N HCl solution in MeOH (110 μL, 0.149 mmol) was slowly added. The reaction mixture was stirred at 4° C. for 15 min. After evaporation and drying under P₂O₅, (7-methoxyisoquinolin-4-yl)(3,4,5-trimethoxyphenyl)methanone hydrochloride 28 was obtained as a yellow solid (55 mg, 47% yield).

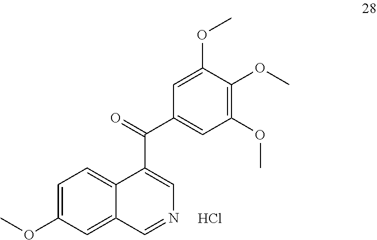

MW: 389.83; Yield: 47%; Yellow solid; Mp (° C.): 178.5

R_{f}: 0.4 (EtOAc, free base).

¹H-NMR (CD₃OD, δ): 3.82 (s, 6H, 2×OCH₃), 3.89 (s, 3H, OCH₃), 4.09 (s, 3H, OCH₃), 7.21 (s, 2H, 2×ArH), 7.85 (d, 1H, J=9.3 Hz, ArH), 7.97 (s, 1H, ArH), 8.14 (d, 1H, J=9.3 Hz, ArH), 8.62 (s, 1H, ArH), 9.74 (s, 1H, ArH).

¹³C-NMR (CD₃OD, δ): 55.4, 55.5 (2×C), 59.8, 107.6, 107.9, 126.7, 129.8, 130.2, 130.4 (2×C), 131.4, 131.9, 134.2, 146.9, 153.4, 161.2, 190.9.

MS-ESI m/z (% rel. Int.): 354.1 ([MH]⁺, 100).

HPLC: Method A, detection UV 254 nm, RT=4.37 min, peak area 98.0%.

### Preparation of (8-amino-7-ethoxyisoquinolin-4-yl)(3,4,5-trimethoxyphenyl)methanone hydrochloride 29

### N-(Diphenylmethylene)-7-ethoxy-4-(3,4,5-trimethoxybenzyl)isoquinolin-8-amine CCH 34090

A mixture of (±) BINAP (15 mg, 24 μmol), Pd₂(dba)₃ (6 mg, 7 μmol), Cs₂CO₃ (70 mg, 215 μmol), 7-ethoxy-4-(3,4,5-trimethoxybenzyl)isoquinolin-8-yl trifluoromethanesulfonate CCH 34050 (free base, 50 mg, 100 μmol) and benzophenone imine (41 mg, 226 μmol) in dry toluene (5 mL) in a 10 mL microwave vial equipped with a magnetic stirrer was stirred for 5 h at 150° C. under microwave irradiation. After cooling to RT, the reaction mixture was diluted with EtOAc (20 mL) and filtered through celite. The filtrate was washed with brine (5 mL), dried over Na₂SO₄, filtered and concentrated at 40° C. under vacuum. Purification by column chromatography (SiO₂, eluent CH₂Cl₂ to EtOAc) gave, after evaporation and drying, N-(diphenylmethylene)-7-ethoxy-4-(3,4,5-trimethoxybenzyl)-isoquinolin-8-amine CCH 34090 as a yellow oil (39 mg, 73% yield).

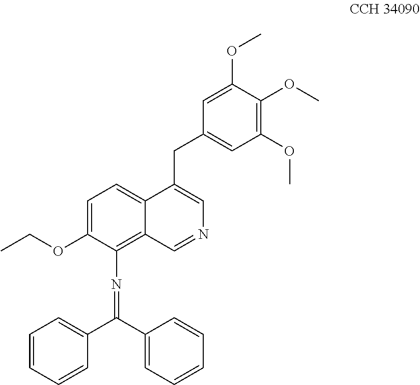 [SMILES: CCOc1ccc2c(Cc3cc(OC)c(OC)c(OC)c3)cncc2c1N=C(c1ccccc1)c1ccccc1]

MW: 532.63; Yield: 73%; Yellow oil.

R_{f}: 0.15 (CH₂Cl₂:EtOAc=7:1).

¹H-NMR (CDCl₃, δ): 1.30 (t, 3H, J=7.0 Hz, CH₂CH₃), 3.71 (s, 6H, 2×OCH₃), 3.81 (s, 3H, OCH₃), 3.85-4.02 (m, 2H, CH₂CH₃), 4.26 (s, 2H, CH₂), 6.32 (s, 2H, 2×ArH), 7.07-7.27 (m, 6H, 6×ArH), 7.44-7.54 (m, 4H, 4×ArH), 7.88 (d, 2H, J=7.4 Hz, 2×ArH), 8.23 (s, 1H, ArH), 9.27 (s, 1H, ArH).

¹³C-NMR (CDCl₃, δ): 14.8, 36.4, 55.7 (2×C), 60.6, 64.4, 105.2 (2×C), 119.1, 119.5, 122.2, 127.2, 127.7, 128.0, 128.5, 128.6, 129.4, 129.8, 130.9, 135.3, 136.1, 136.7, 138.6, 141.4, 142.5, 147.9, 153.0, 171.5.

MS-ESI m/z (rel. int.): 533.4 ([M+H]⁺, 100).

HPLC: Method A, detection UV 254 nm, RT=5.90 min.

### (8-Amino-7-ethoxyisoquinolin-4-yl)(3,4,5-trimethoxyphenyl)methanone hydrochloride 29

A mixture of N-(diphenylmethylene)-7-ethoxy-4-(3,4,5-trimethoxybenzyl)-isoquinolin-8-amine CCH 34090 (68 mg, 128 μmol), N-hydroxyphthalimide (4 mg, 24 μmol) and NaClO₂ (80% pure, 20 mg, 177 μmol) in a CH₃CN:H₂O=2:1 solution (6 mL) in a 10 mL microwave vial equipped with a magnetic stirrer was stirred for 20 min at 120° C. under microwave irradiation. After cooling to RT, the mixture was diluted with Et₂O (50 mL), washed with 10% aq. sodium sulfite solution (10 mL), washed with brine (10 mL), dried over Na₂SO₄, filtered and concentrated at 40° C. under vacuum. The residue was then taken up in a solution of 1 N aq. HCl:THF=1:1 (15 mL) and the mixture was stirred at RT for 30 min after which THF was removed at 40° C. under vacuum. The residue was neutralized with saturated aqueous NaHCO₃ before extraction with CH₂Cl₂ (50 mL) and the organic solution was washed with brine (10 mL), dried over Na₂SO₄, filtered and concentrated at 40° C. under vacuum. Purification by column chromatography (SiO₂, eluent from cyclohexane to EtOAc) gave, after evaporation and drying, 22 mg of a pale yellow oil (45% yield). This product (22 mg, 58 μmol) was dissolved in MeOH (3 mL) in a 10 mL round-bottomed flask equipped with a magnetic stirrer and the solution was cooled to 0° C. in an ice bath before adding 0.4 mL of a 0.49 N HCl solution in MeOH. The solution was stirred for 15 min at 0° C. before concentration to dryness at RT under vacuum, affording (8-amino-7-ethoxyisoquinolin-4-yl)(3,4,5-trimethoxyphenyl)methanone hydrochloride 29 as a red solid (26 mg, 100% yield).

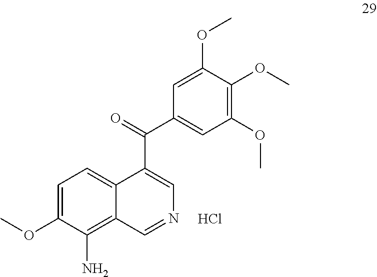

MW: 455.33; Yield: 45%; Red solid; Mp (° C.): 234.1 (dec.)

R_{f}: 0.25 (EtOAc, free base).

¹H-NMR (CD₃OD, δ): 1.41 (t, 3H, J=7.0 Hz, CH₂CH₃), 3.69 (s, 3H, OCH₃), 3.69 (s, 3H, OCH₃), 3.77 (s, 3H, OCH₃), 4.19 (q, 2H, J=7.0 Hz, CH₂CH₃), 7.08 (s, 2H, 2×ArH), 7.12 (d, 1H, J=8.8 Hz), 7.69 (d, 1H, J=8.8 Hz), 8.13 (s, 1H, ArH), 9.74 (s, 1H, ArH).

¹³C-NMR (CD₃OD, δ): 15.1, 56.9 (2×C), 61.3, 66.3, 109.1 (2×C), 113.0, 116.8, 124.3, 127.8, 130.0, 133.0, 135.1, 141.0, 145.4, 145.5, 154.8 (2×C), 193.0.

MS-ESI m/z (rel. int.): 383 ([M+H]⁺, 100).

HPLC: Method A, detection UV 254 nm, RT=4.39 min, peak area 96.0%.

### Preparation of 4-(3,5-dimethoxy-4-(2,2,2-trifluoroethoxy)benzyl)-7-ethoxyisoquinolin-8-ol hydrochloride 30

### 3,5-Dimethoxy-4-(2,2,2-trifluoroethoxy)-benzaldehyde ECO 33182

4-Hydroxy-3,5-dimethoxybenzaldehyde (500 mg, 2.75 mmol, 1.0 eq) was dissolved in DMF (8 mL) and Cs₂CO₃ (900 mg, 2.75 mmol, 1.0 eq) was added at 20° C. 2,2,2-Trifluoroethyl-p-toluene sulfonate (768 mg, 3.02 mmol, 1.1 eq) was added and the reaction mixture was stirred at 150° C. for 10 min under microwave irradiation. After cooling to RT, The reaction mixture was poured into water (80 mL) and extracted by Et₂O (300 mL). The separated organic layer was washed with brine (50 mL), dried over MgSO₄, filtered and concentrated to dryness to give a yellow solid (692 mg). Purification by column chromatography (SiO₂, eluent cyclohexane:EtOAc=100:0 to 8:2) gave, after evaporation and drying, 3,5-dimethoxy-4-(2,2,2-trifluoroethoxy)-benzaldehyde ECO 33182 (327 mg, 45% yield) as an off-white solid.

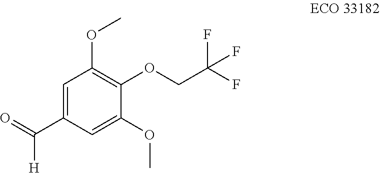 [SMILES: COc1cc(C=O)cc(OC)c1OCC(F)(F)F]

MW: 264.20; Yield: 45%; Off-white solid; Mp (° C.): 86-87

R_{f}: 0.25 (cyclohexane:EtOAc=8:2).

¹H-NMR (CDCl₃, δ): 3.94 (s, 6H, 2×OCH₃), 4.42 (q, 2H, OCH₂, J=8.48 Hz), 7.14 (s, 2H, 2×ArH), 9.88 (s, 1H, CHO).

¹³C-NMR (CDCl₃, δ): 56.4 (2×OCH₃), 69.5 (q, CF₃CH₂, J=35.1 Hz), 106.6, 123.4 (q, CF₃, J=279 Hz), 132.7, 141.1, 153.3 (2×C), 190.9.

MS-ESI m/z (% rel. Int.): 265.1 ([MH]⁺, 36), 237.1 (100).

HPLC: Method A, XBridge™ column, detection UV 254 nm, RT=5.44 min, peak area 99.9%.

### 4-(3,5-Dimethoxy-4-(2,2,2-trifluoroethoxy)benzyl)-7-ethoxyisoquinolin-8-ol hydrochloride 30

In an ace pressure tube, 2-((2,2-diethoxyethylamino)methyl)-6-ethoxyphenol SAO 33014 (268.1 mg, 0.946 mmol) and 3,5-dimethoxy-4-(2,2,2-trifluoro-ethoxy)-benzaldehyde ECO 33182 (250 mg, 0.946 mmol) were dissolved in EtOH (1.6 mL) and a 37% HCl solution (1.6 mL) was added. The reaction mixture was stirred at 90° C. for 25 min, cooled to 4° C. and concentrated. The crude product was transformed into its freebase with a 18 N NH₄OH solution (500 μL) and CH₂Cl₂ (250 mL) was added. The separated organic layer was washed with brine (30 mL), dried over MgSO₄, filtered and evaporated to give a yellow oil. The crude product was purified by column chromatography (SiO₂, eluent CH₂Cl₂:MeOH=98:2 to 96:4) to give, after evaporation and drying, 4-(3,5-dimethoxy-4-(2,2,2-trifluoroethoxy)benzyl)-7-ethoxyisoquinolin-8-ol (29.3 mg). This solid was dissolved in MeOH (2 mL) and a 1.35 N HCl solution in MeOH (52 μL, 0.070 mmol) was slowly added. The reaction mixture was stirred at 4° C. for 15 min. After evaporation of solvent and drying under vacuum pump under P₂O₅, 4-(3,5-dimethoxy-4-(2,2,2-trifluoroethoxy)benzyl)-7-ethoxyisoquinolin-8-ol hydrochloride 30 was obtained as a yellow solid (30.4 mg, 7% yield).

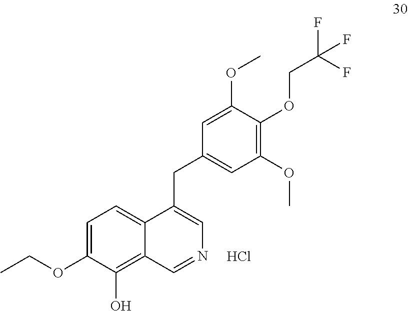 [SMILES: CCOc1ccc2c(Cc3cc(OC)c(OCC(F)(F)F)c(OC)c3)cncc2c1O ; Cl]

MW: 473.87; Yield: 7%; Yellow solid; Mp (° C.): 234.8

R_{f}: 0.20 (CH₂Cl₂:MeOH=98:2, free base).

¹H-NMR (CD₃OD, δ): 1.46 (t, 3H, J=7.0 Hz, CH₂CH₃), 3.74 (s, 6H, 2×OCH₃), 4.23-4.33 (m, 4H, 2×OCH₂), 4.43 (s, 2H, CH₂), 6.59 (s, 2H, ArH), 7.77 (d, 1H, J=9.0 Hz, ArH), 7.90 (d, 1H, J=9.1 Hz, ArH), 8.05 (s, 1H, ArH), 9.58 (s, 1H, ArH).

¹³C-NMR (CD₃OD, δ): 13.5, 35.5, 55.2 (2×C), 65.3, 68.7 (q), 105.8 (2×C), 114.8, 119.2, 124.2, 125.5, 129.4, 131.0, 134.6, 135.0 (2×C), 142.0, 144.3, 144.8, 152.9 (2×C).

MS-ESI m/z (% rel. Int.): 438.1 ([MH]⁺, 100).

HPLC: Method A, XBridge™ column, detection UV 254 nm, RT=4.39 min, peak area 97.0%.

### Preparation of 4-(4-isobutoxy-3,5-dimethoxybenzyl)-7-ethoxyisoquinolin-8-amine dihydrochloride 31

### 7-Ethoxy-4-(4-isobutoxy-3,5-dimethoxybenzyl)isoquinolin-8-yl trifluoromethanesulfonate LPO 37048E

7-Ethoxy-4-(4-isobutoxy-3,5-dimethoxybenzyl)isoquinolin-8-ol hydrochloride 20 (600 mg) was transformed into its freebase with a 18 N NH₄OH solution in MeOH and CH₂Cl₂ (200 mL) was added. The separated organic layer was washed with brine, dried over MgSO₄, filtered and solvent was evaporated to give LPO 37048B (560 mg). This crude product was purified by column chromatography (SiO₂, eluent: cyclohexane:EtOAc=100:0 to 5:5) to give, after evaporation and drying, 7-ethoxy-4-(4-isobutoxy-3,5-dimethoxybenzyl)isoquinolin-8-ol LPO 37048C (197.5 mg). To a suspension of LPO 37048C in CH₂Cl₂ (3 mL) in a 25 mL round-bottom flask equipped with a magnetic stirrer, was added anhydrous Et₃N (134.2 μL, 0.96 mmol, 2.0 eq) and N-phenyl-bis(trifluoromethanesulfonimide) (257.2 mg, 0.72 mmol, 1.5 eq). The reaction mixture was stirred for 30 min at RT under nitrogen then diluted with CH₂Cl₂ (50 mL) and the organic solution was washed with water (10 mL), brine (10 mL), dried over MgSO₄, filtered and concentrated at 40° C. under vacuum. Purification by column chromatography (SiO₂, eluent cyclohexane:EtOAc=100:0 to 7:3) gave, after evaporation and drying, 7-ethoxy-4-(4-isobutoxy-3,5-dimethoxybenzyl)isoquinolin-8-yl trifluoromethanesulfonate LPO 37048E (234 mg, 90% yield) as a yellow oil.

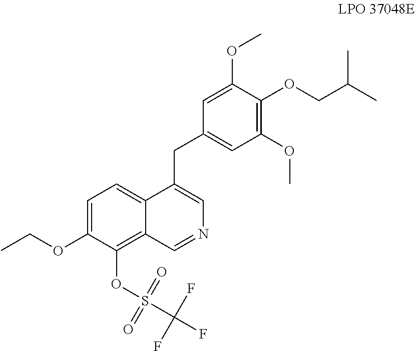 [SMILES: CCOc1ccc2c(Cc3cc(OC)c(OCC(C)C)c(OC)c3)cncc2c1OS(=O)(=O)C(F)(F)F]

MW: 543.55; Yield: 90%; Yellow oil.

R_{f}: 0.2 (cyclohexane:EtOAc=7:3, free base).

¹H-NMR (CD₃OD, δ): 1.00 (d, 6H, J=6.68 Hz, 2×CH₃), 1.46 (t, 3H, J=6.98 Hz, CH₂CH₃), 1.99-2.08 (m, 1H, CH), 3.69 (d, 2H, J=6.73 Hz, OCH₂), 4.25-4.31 (m, 4H, OCH₂ and CH₂), 6.37 (s, 2H, ArH), 7.53 (d, 1H, J=9.37 Hz, ArH), 7.96 (d, 2H, J=9.32 Hz, ArH), 8.37 (s, 1H, ArH), 9.34 (s, 1H, ArH).

MS-ESI m/z (% rel. Int.): 544.2 ([MH]⁺, 100).

HPLC: Method A, XBridge™ column, detection UV 254 nm, RT=6.41 min, peak area 98%.

### 4-(4-Isobutoxy-3,5-dimethoxybenzyl)-7-ethoxyisoquinolin-8-amine dihydrochloride 31

A mixture of (±) BINAP (53.7 mg, 0.086 mmol), Pd₂(dba)₃ (23.7 mg, 0.026 mmol), Cs₂CO₃ (280.9 mg, 0.862 mmol), 7-ethoxy-4-(4-isobutoxy-3,5-dimethoxybenzyl)isoquinolin-8-yl trifluoromethanesulfonate LPO 37048E (234 mg, 0.43 mmol) and benzophenone imine (172 mg, 0.95 mmol) in dry toluene (15 mL) in a 20 mL microwave vial equipped with a magnetic stirrer was stirred for 5 h at 150° C. under microwave irradiation. After cooling to RT, the reaction mixture was diluted with THF (50 mL) and filtered through celite. The filtrate was poured in a 250 mL round-bottomed flask equipped with a magnetic stirrer before addition of a 1 N HCl solution (20 mL) and the mixture was stirred for 1 h at RT. The volatiles were then removed at 40° C. under vacuum and the residue was taken up in CH₂Cl₂ (100 mL), washed with a saturated solution of K₂CO₃ (20 mL), brine (20 mL), dried over MgSO₄, filtered and concentrated at 40° C. under vacuum. Purification by column chromatography (SiO₂, eluent cyclohexane:EtOAc=100:0 to 0:100) gave, after evaporation and drying, 4-(4-isobutoxy-3,5-dimethoxybenzyl)-7-ethoxyisoquinolin-8-amine (26.9 mg). This solid was dissolved in MeOH (2 mL) and a 0.49 N HCl solution in MeOH (281 μL, 0.138 mmol) was slowly added. The reaction mixture was stirred at 4° C. for 15 min. After evaporation of solvent and drying under vacuum pump under P₂O₅, 4-(4-isobutoxy-3,5-dimethoxybenzyl)-7-ethoxyisoquinolin-8-amine dihydrochloride 31 (28.7 mg, 14% yield) was obtained as a red solid.

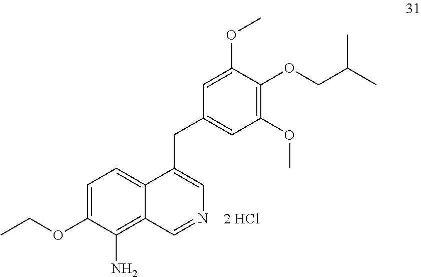 [SMILES: CCOc1ccc2c(Cc3cc(OC)c(OCC(C)C)c(OC)c3)cncc2c1N ; "2 HCl"]

MW: 483.43; Yield: 14%; Red Solid; Mp (° C.): 263.3

R_{f}: 0.25 (EtOAc=100%, free base).

¹H-NMR (CD₃OD, δ): 0.99 (s, 3H, CH₃), 1.02 (s, 3H, CH₃), 1.52 (t, 3H, J=6.90 Hz, CH₂CH₃), 1.93-2.00 (m, 1H, CH(CH₃)₂), 3.66 (d, 2H, J=6.6 Hz, CH₂CH₃), 3.77 (s, 6H, 2×OCH₃), 4.30 (q, 2H, J=7.0 Hz, CH₂CH₃), 4.40 (s, 2H, CH₂), 6.60 (s, 2H, ArH), 7.54 (d, 2H, J=8.8 Hz, ArH), 7.82 (d, 2H, J=8.8 Hz, ArH), 7.90 (s, 1H, ArH), 9.66 (s, 1H, ArH).

¹³C-NMR (CD₃OD, δ): 15.1, 19.6 (2×C), 37.2, 56.7 (2×C), 66.3, 81.2, 107.6 (2×C), 112.3, 116.6, 123.0, 123.9, 127.0, 132.1, 134.8, 137.4, 137.7, 140.2, 142.4, 145.3, 155.1 (2×C).

MS-ESI m/z (% rel. Int.): 411.1 ([MH]⁺, 100).

HPLC: Method A, XBridge™ column, detection UV 254 nm, RT=5.22 min, peak area 98.0%.

### Preparation of 4-(4-(allyloxy)-3,5-dimethoxybenzyl)-7-ethoxyisoquinolin-8-ol hydrochloride 32

### 4-(Allyloxy)-3,5-dimethoxy-benzaldehyde ECO 33176

3,5-Dimethoxy-4-hydroxybenzaldehyde (500 mg, 2.75 mmol) was dissolved in DMF anhydrous (8 mL) and cesium carbonate (900 mg, 2.75 mmol) was added at RT and the reaction mixture was stirred for 5 min under N₂. Allyl bromide (261 μL, 3.03 mmol) was added and the reaction mixture was stirred at 40° C. for 15 h. after cooling to RT, the reaction mixture was diluted in ether (300 mL) and water (50 mL). The organic layer was washed with brine, dried over MgSO₄, and filtered. After evaporation of the solvent and drying under vacuum pump under P₂O₅, 4-(allyloxy)-3,5-dimethoxy-benzaldehyde ECO 33176 was obtained as an off-white solid (607 mg, 98% yield).

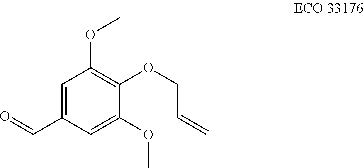 [SMILES: C=CCOc1c(OC)cc(C=O)cc1OC]

MW=222.24; Yield: 98%; Off-white solid.

¹H-NMR (CDCl₃, δ): 3.93 (s, 6H, 2×CH₃O), 4.63 (d, 2H, J=6.2 Hz, OCH₂), 5.17 (m, 2H, CH═CH₂), 6.08 (m, 1H, CH═CH₂), 7.13 (s, 2H, 2×ArH), 9.87 (s, 1H, CHO).

HPLC: Method A, XBridge™ column, detection UV 254 nm, RT=4.92 min, peak area 99.9%.

### 4-(4-(Allyloxy)-3,5-dimethoxybenzyl)-7-ethoxyisoquinolin-8-ol hydrochloride 32

In an ace pressure tube, 2-((2,2-diethoxyethylamino)methyl)-6-ethoxyphenol SAO 33014 (325 mg, 1.15 mmol) and 4-(allyloxy)-3,5-dimethoxy-benzaldehyde ECO 33176 (255 mg, 1.15 mmol) were dissolved in EtOH (1.6 mL) and a 37% HCl solution (1.6 mL) was added. The reaction mixture was stirred at 80° C. for 10 min, cooled at 4° C. and concentrated. The crude product was transformed into its freebase with a 18 N NH₄OH solution (0.5 mL) and CH₂Cl₂ was added (150 mL). The organic layer was washed with brine (30 mL), dried over MgSO₄, filtered and evaporated to give a brown solid. This crude product was purified by column chromatography (SiO₂, eluent CH₂Cl₂:MeOH=98:2 to 95:5) to give, after evaporation and drying, 4-(4-(allyloxy)-3,5-dimethoxybenzyl)-7-ethoxyisoquinolin-8-ol (39.2 mg). This solid was dissolved in MeOH (2 mL) and a 0.49 N HCl solution (212 μL, 0.104 mmol) was slowly added. The reaction mixture was stirred at 4° C. for 15 min. After evaporation and drying under vacuum pump under P₂O₅, 4-(4-(allyloxy)-3,5-dimethoxybenzyl)-7-ethoxyisoquinolin-8-ol hydrochloride 32 (39.8 mg, 8% yield) was obtained as a yellow solid.

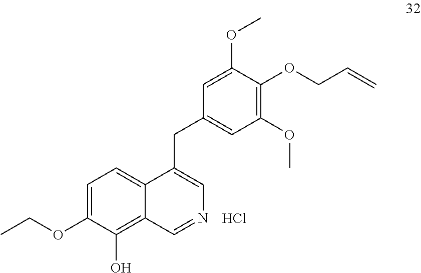 [SMILES: C=CCOc1c(OC)cc(Cc2cncc3c(O)c(OCC)ccc23)cc1OC ; Cl]

MW: 431.91; Yield: 8%; Yellow solid; Mp (° C.): 67.7

R_{f}: 0.25 (CH₂Cl₂:MeOH=95:5, free base).

¹H-NMR (CD₃OD, δ): 1.50 (t, 3H, J=6.7 Hz, CH₂CH₃), 3.78 (s, 6H, 2×OCH₃), 4.31-4.49 (m, 6H, CH₂+2×OCH₂), 5.23 (dd, 2H, J=43.8 Hz, J=10.3 Hz, CH═CH₂), 6.02-6.10 (m, 1H, CH═CH₂), 6.62 (s, 2H, 2×ArH), 7.88 (d, 1H, J=9.1 Hz, ArH), 8.02-8.06 (m, 2H, 2×ArH), 9.68 (s, 1H, ArH).

¹³C-NMR (CD₃OD, δ): 15.0, 37.0, 56.7 (2×C), 66.9, 75.1, 107.4 (2×C), 116.5, 117.9, 120.5, 126.6, 128.4, 132.8, 135.1, 135.8, 136.7, 137.9, 142.5, 146.4, 146.8, 155.1 (2×C).

MS-ESI m/z (% rel. Int.): 396.3 ([MH]⁺, 100).

HPLC: Method A, XBridge™ column, detection UV 254 nm, RT=4.40 min, peak area 97.0%.

### Preparation of 7-ethoxy-4-(4-(isopentyloxy)-3,5-dimethoxybenzyl)isoquinolin-8-ol hydrochloride 33

### 4-(Isopentyloxy)-3,5-dimethoxybenzaldehyde EMC 38030

4-Hydroxy-3,5-dimethoxybenzaldehyde (500 mg, 2.74 mmol) was dissolved in DMF (7.5 mL) and Cs₂CO₃ (894 mg, 2.74 mmol) was added at RT. 1-Bromo-3-methylbutane (456 mg, 3.02 mmol) was added and the reaction was stirred at 40° C. for 10 h under a nitrogen atmosphere. After cooling at RT, the reaction mixture was poured into water (75 mL) and extracted by Et₂O (50 mL). The organic layer was washed with H₂O (2×50 mL), brine (50 mL), dried over MgSO₄, filtered and dried under reduced pressure to give a yellow oil. After overnight drying, 4-(isopentyloxy)-3,5-dimethoxybenzaldehyde EMC 38030 (536 mg, 78% yield) was obtained as a yellow oil.

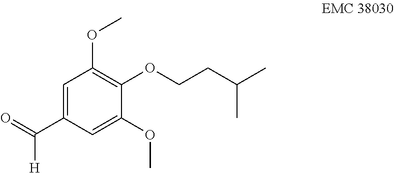 [SMILES: COc1cc(C=O)cc(OC)c1OCCC(C)C]

MW: 252.31; Yield=78%; Yellow oil.

¹H-NMR (CDCl₃, δ): 0.97 (d, 6H, 2×CH₃, J=13.49 Hz), 1.58-1.70 (m, 2H, CHCH₂), 1.81-1.95 (m, 1H, CHCH₂), 3.92 (s, 6H, 2×OCH₃), 4.12 (t, 2H, OCH₂, J=6.84 Hz), 7.13 (m, 2H, ArH), 9.87 (s, 1H, CHO).

¹³C-NMR (CDCl₃, δ): 22.5 (2×C), 24.8, 38.9, 56.2, 72.1, 106.8, 131.6, 143.1, 154.0, 191.1

MS-ESI m/z (% rel. Int.): 253 ([MH]⁺, 50), 183 (100).

HPLC: Method A, XBridge™ column, detection UV 254 nm, RT=6.14 min, peak area 99.9%.

### 7-Ethoxy-4-(4-(isopentyloxy)-3,5-dimethoxybenzyl)isoquinolin-8-ol hydrochloride 33

In an ace pressure tube, 2-((2,2-diethoxyethylamino)methyl)-6-ethoxyphenol SAO 33014 (281 mg, 0.99 mmol, 1.0 eq) and 4-(isopentyloxy)-3,5-dimethoxybenzaldehyde EMC 38030 (250 mg, 0.99 mmol, 1.0 eq) were dissolved in EtOH (1.6 mL) and a 37% HCl solution (1.6 mL) was added. The reaction mixture was stirred at 90° C. for 20 min then cooled at 4° C., and concentrated. The crude product was transformed into its freebase with a 18 N NH₄OH solution (900 μL) and CH₂Cl₂ (150 mL) was added. The organic layer was washed with brine (30 mL), dried over MgSO₄, filtered and evaporated to give brown solid (407 mg). This crude product was purified by column chromatography (SiO₂, eluent CH₂Cl₂:MeOH=100:0 to 98:2) to give, after evaporation and drying, 7-ethoxy-4-(4-(isopentyloxy)-3,5-dimethoxybenzyl)isoquinolin-8-ol (117.4 mg) as a yellow oil. This oil was dissolved in MeOH (2 mL) and a 1.34 N HCl solution in MeOH (216 μL, 0.290 mmol) was slowly added. The reaction mixture was stirred at 4° C. for 15 min. After evaporation and drying under vacuum pump under P₂O₅, 7-ethoxy-4-(4-(isopentyloxy)-3,5-dimethoxybenzyl)isoquinolin-8-ol hydrochloride 33 (119.4 mg, 26% yield) was obtained as a yellow solid.

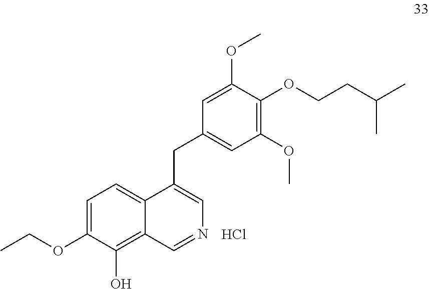 [SMILES: CCOc1ccc2c(Cc3cc(OC)c(OCCC(C)C)c(OC)c3)cncc2c1O ; Cl]

MW: 461.98; Yield: 26%; Yellow solid; Mp (° C.): 238.2

R_{f}: 0.2 (CH₂Cl₂:MeOH=98:2, free base).

¹H-NMR (CD₃OD, δ): 0.82 (d, 6H, J=6.58 Hz, 2×CH₃), 1.36 (t, 3H, J=7.02 Hz, CH₂CH₃), 1.42 (q, 2H, J=6.75 Hz, CH₂CH), 1.70-1.74 (m, 1H, CH), 3.62 (s, 6H, 2×OCH₃), 3.77 (t, 2H, J=6.61 Hz, OCH₂CH₂), 4.20 (q, 2H, J=7.0 Hz, OCH₂CH₃), 4.33 (s, 2H, CH₂), 6.46 (s, 2H, 2×ArH), 7.74 (d, 1H, J=9.08 Hz, ArH), 7.89 (d, 1H, J=9.22 Hz, ArH), 7.92 (s, 1H, ArH), 9.51 (s, 1H, ArH).

¹³C-NMR (CD₃OD, δ): 15.0, 22.9 (2×C), 25.9, 37.0, 40.1, 56.7, 66.9, 72.8, 107.5, 116.6, 120.5, 126.7, 128.3, 132.8, 134.9, 137.4, 138.0, 142.5, 146.4, 146.8, 155.2 (2×C).

MS-ESI m/z (% rel. Int.): 426.3 ([MH]⁺, 100).

HPLC: Method A, XBridge™ column, detection UV 254 nm, RT=5.24 min, peak area 98.0%.

### Preparation of 4-(4-(cyclobutylmethoxy)-3,5-dimethoxybenzyl)-7-ethoxyisoquinolin-8-ol hydrochloride 34

### 4-(Cyclobutylmethoxy)-3,5-dimethoxy-benzaldehyde EMC 38036

4-Hydroxy-3,5-dimethoxybenzaldehyde (500 mg, 2.74 mmol) was dissolved in DMF (7.5 mL) and Cs₂CO₃ (894 mg, 2.74 mmol) was added at 20° C. (Bromomethyl)cyclobutane (450 mg, 3.02 mmol) was added and the reaction mixture was stirred at 40° C. for 10 h under a nitrogen atmosphere. After cooling at RT, the reaction mixture was poured into water (75 mL) and extracted by Et₂O (50 mL). The organic layer was washed with water (2×50 mL), brine (50 mL), dried over MgSO₄, filtered and dried overnight under reduced pressure to give 4-(cyclobutylmethoxy)-3,5-dimethoxy-benzaldehyde EMC 38036 (194 mg, 28% yield) as a yellow oil.

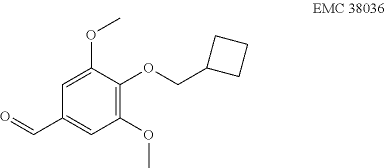 [SMILES: COc1cc(C=O)cc(OC)c1OCC1CCC1]

MW: 250.30; Yield: 28%; Yellow oil.

¹H-NMR (CDCl₃, δ): 1.87-2.13 (m, 6H, 3×CH₂ cyclobutane), 2.73-2.78 (m, 1H, CH), 3.92 (s, 6H, 2×OMe), 4.05-4.07 (d, 2H, OCH₂), 7.12 (s, 2H, 2×ArH), 9.87 (s, 1H, CHO).

¹³C-NMR (CDCl₃, δ): 18.5, 24.9 (2×C), 35.4, 56.3, 77.8, 106.8 (2×C), 131.6 (2×C), 143.2, 154.0 (2×C), 191.1.

MS-ESI m/z (% rel. Int.): 251 ([MH]⁺, 10), 183 (100).

HPLC: Method A, XBridge™ column, detection UV 254 nm, RT=5.90 min, peak area 99.9%.

### 4-(4-(Cyclobutylmethoxy)-3,5-dimethoxybenzyl)-7-ethoxyisoquinolin-8-ol hydrochloride 34

In an ace pressure tube, 2-((2,2-diethoxyethylamino)methyl)-6-ethoxyphenol SAO 33014 (218 mg, 0.77 mmol, 1.0 eq) and 4-(cyclobutylmethoxy)-3,5-dimethoxy-benzaldehyde EMC 38036 (193 mg, 0.77 mmol, 1.0 eq) were dissolved in EtOH (1.4 mL) and a 37% HCl solution (1.4 mL) was added. The reaction mixture was stirred at 90° C. for 20 min, cooled at 4° C. and concentrated. The crude product was transformed into its freebase with a 18 N NH₄OH solution (500 μL) and CH₂Cl₂ (150 mL) was added. The separated organic layer was washed with brine (30 mL), dried over MgSO₄, filtered and evaporated to give brown oil (305 mg). This crude oil was purified by column chromatography (SiO₂, eluent CH₂Cl₂:MeOH=100:0 to 98:2) to give, after evaporation and drying, 4-(4-cyclobutylmethoxy-3,5-dimethoxybenzyl)-7-ethoxyisoquinolin-8-ol (91.3 mg) as an orange oil. This oil was dissolved in MeOH (2 mL) and a 1.34 N HCl solution in MeOH (169 μL, 0.226 mmol, 1.05 eq) was slowly added. The reaction mixture was stirred at 4° C. for 15 min. After evaporation and drying under vacuum pump under P₂O₅, 4-(4-cyclobutylmethoxy-3,5-dimethoxybenzyl)-7-ethoxyisoquinolin-8-ol hydrochloride 34 (95.6 mg, 27% yield) was obtained as a yellow solid.

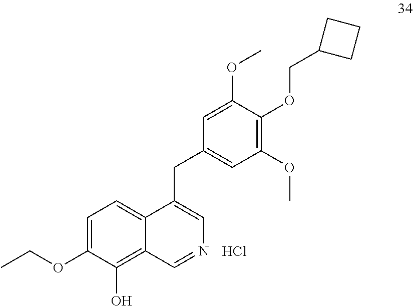 [SMILES: CCOc1ccc2c(Cc3cc(OC)c(OCC4CCC4)c(OC)c3)cncc2c1O ; Cl]

MW: 459.96; Yield: 27%; Yellow Solid; Mp (° C.): 225.1

R_{f}: 0.2 (CH₂Cl₂:MeOH=98:2, free base).

¹H-NMR (CD₃OD, δ): 1.51 (t, 3H, J=6.95 Hz, CH₂CH₃), 1.84-2.08 (m, 6H, 3×CH₂), 2.66-2.71 (m, 1H, CH), 3.78 (s, 6H, 2×OCH₃), 3.88 (d, 2H, J=6.83 Hz, OCH₂CH), 4.35 (q, 2H, J=6.96 Hz, OCH₂CH₃), 4.49 (s, 2H, CCH₂), 6.61 (s, 2H, 2×ArH), 7.89 (d, 1H, J=9.05 Hz, ArH), 8.04 (d, 1H, J=9.26 Hz, ArH), 8.07 (s, 1H, ArH), 9.51 (s, 1H, ArH).

¹³C-NMR (CD₃OD, δ): 15.0, 19.4, 25.8 (2×C), 36.7, 37.0, 56.7 (2×C), 66.9, 78.6, 107.5 (2×C), 116.6, 120.5, 126.7, 128.3, 132.8, 134.9, 137.3, 138.0, 142.5, 146.4, 146.8, 155.2 (2×C).

MS-ESI m/z (% rel. Int.): 424.1 ([MH]⁺, 100).

HPLC: Method A, XBridge™ column, detection UV 254 nm, RT=5.03 min, peak area 98.0%.

### Preparation of 4-(4-(2-cyclohexylethoxy)-3,5-dimethoxybenzyl)-7-ethoxyisoquinolin-8-ol hydrochloride 35

### 4-(2-Cyclohexylethoxy)-3,5-dimethoxybenzaldehyde EMC 38042

4-Hydroxy-3,5-dimethoxybenzaldehyde (500 mg, 2.74 mmol) was dissolved in DMF (7.5 mL) and Cs₂CO₃ (894 mg, 2.74 mmol) was added at 20° C. 2-Cyclohexyl ethyl bromide (577 mg, 3.02 mmol) was added and the reaction mixture was stirred at 40° C. for 10 h under a nitrogen atmosphere. After cooling to RT, the reaction mixture was poured into water (75 mL) and extracted by Et₂O (50 mL). The separated organic layer was washed with water (2×50 mL), brine (50 mL), dried over MgSO₄, filtered and dried overnight under reduced pressure to give 4-(2-cyclohexylethoxy)-3,5-dimethoxybenzaldehyde EMC 38042 (640 mg, 80% yield) as white solid.

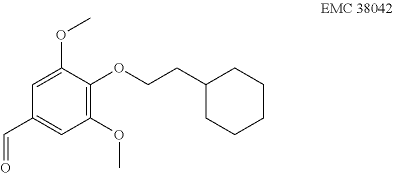 [SMILES: COc1cc(C=O)cc(OC)c1OCCC1CCCCC1]

MW: 292.38; Yield=80%; White solid; Mp (° C.)=43.1

¹H-NMR (CDCl₃, δ): 0.91-0.95 (m, 2H, CH₂CH₂CH), 1.20-1.79 (m, 11H, 5×CH₂ & CH), 3.91 (s, 6H, 2×OCH₃), 4.08-4.13 (t, 2H, J=6.74 Hz, OCH₂), 7.12 (s, 2H, 2×ArH), 9.87 (s, 1H, CHO).

¹³C-NMR (CDCl₃, δ): 26.4 (2×C), 26.6, 33.3 (2×C), 34.2, 37.5, 56.3 (2×C), 71.7, 106.8 (2×C), 131.6, 143.2, 154.0 (2×C), 191.1.

MS-ESI m/z (% rel. Int.): 293 ([MH]⁺, 60), 183 (100).

HPLC: Method A, XBridge™ column, detection UV 254 nm, RT=3.96 min, peak area 99.9%.

### 4-(4-(2-Cyclohexylethoxy)-3,5-dimethoxybenzyl)-7-ethoxyisoquinolin-8-ol hydrochloride 35

In an ace pressure tube, 2-((2,2-diethoxyethylamino)methyl)-6-ethoxyphenol SAO 33014 (242 mg, 0.88 mmol, 1.0 eq) and 4-(2-cyclohexylethoxy)-3,5-dimethoxybenzaldehyde EMC 38042 (250 mg, 0.85 mmol, 1.0 eq) were dissolved in EtOH (1.6 mL) and a 37% HCl solution (1.6 mL) was added. The reaction mixture was stirred at 90° C. for 15 min, cooled to 4° C. and concentrated. The crude product was transformed into its freebase with a 18 N NH₄OH solution (1 mL) and CH₂Cl₂ (150 mL) was added. The separated organic layer was washed with brine (30 mL), dried over MgSO₄, filtered and evaporated to give an orange solid (506.6 mg). This crude material was purified by column chromatography (SiO₂, eluent CH₂Cl₂:MeOH=100:0 to 98:2) to give, after evaporation and drying 4-(4-(2-cyclohexylethoxy)-3,5-dimethoxybenzyl)-7-ethoxyisoquinolin-8-ol (104 mg) as an orange solid. This solid was dissolved in MeOH (2 mL) and a 1.34 N HCl solution in MeOH (175 μL, 0.235 mmol, 1.05 eq) was slowly added. The reaction mixture was stirred at 4° C. for 15 min. After evaporation and drying under vacuum pump under P₂O₅, 4-(4-(2-cyclohexylethoxy)-3,5-dimethoxybenzyl)-7-ethoxyisoquinolin-8-ol hydrochloride 35 (104.2 mg, 24% yield) was obtained as a yellow solid.

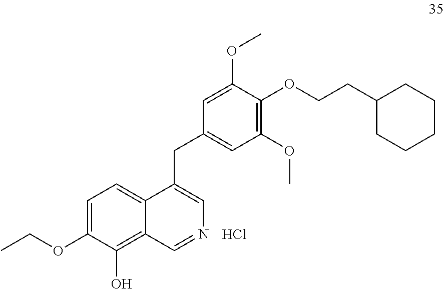 [SMILES: CCOc1ccc2c(Cc3cc(OC)c(OCCC4CCCCC4)c(OC)c3)cncc2c1O ; Cl]

MW: 502.04; Yield: 24%; Yellow Solid; Mp (° C.): 234.5

R_{f}: 0.2 (CH₂Cl₂:MeOH=98:2, free base).

¹H-NMR (CD₃OD, δ): 0.91-0.94 (m, 2H, CH₂), 1.16-1.30 (m, 3H, CH₃CH₂O), 1.48-1.79 (m, 11H, 5×CH₂ & CH), 3.77 (s, 6H, 2×OCH₃), 3.91-3.94 (m, 2H, OCH₂CH₂), 4.33-4.39 (m, 2H, OCH₂CH₃), 4.48 (s, 2H, CH₂), 6.61 (s, 2H, 2×ArH), 7.87 (d, 1H, J=9.01 Hz, ArH), 8.02 (d, 1H, J=9.13 Hz, ArH), 8.08 (s, 1H, ArH), 9.65 (s, 1H, ArH).

¹³C-NMR (CD₃OD, δ): 15.1, 27.5 (2×C), 27.7, 34.4 (2×C), 35.5, 37.0, 38.7, 56.7 (2×C), 66.9, 72.2, 107.5 (2×C), 116.5, 120.5, 126.6, 128.5, 132.8, 134.9, 137.3, 137.9, 142.5, 146.3, 146.8, 155.2 (2×C).

MS-ESI m/z (% rel. Int.): 466.2 ([MH]⁺, 100).

HPLC: Method A, XBridge™ column, detection UV 254 nm, RT=5.86 min, peak area 98.0%.

### Preparation of 4-(4-(cyclohexylmethoxy)-3,5-dimethoxybenzyl)-7-ethoxyisoquinolin-8-ol hydrochloride 36

### 4-(Cyclohexylmethoxy)-3,5-dimethoxybenzaldehyde EMC 38032

4-Hydroxy-3,5-dimethoxybenzaldehyde (500 mg, 2.74 mmol) was dissolved in DMF (7.5 mL) and Cs₂CO₃ (894 mg, 2.74 mmol) and cyclohexanemethyl bromide (535 mg, 3.02 mmol) were added at RT and the reaction mixture was stirred at 40° C. for 10 h under a nitrogen atmosphere. After cooling to RT, the reaction mixture was poured into water (75 mL) and extracted by Et₂O (50 mL). The separated organic layer was washed with water (2×50 mL), brine (50 mL), dried over MgSO₄, filtered and dried under reduced pressure to give 4-(cyclohexylmethoxy)-3,5-dimethoxy-benzaldehyde EMC 38032 (413 mg, 54% yield) as yellow oil.

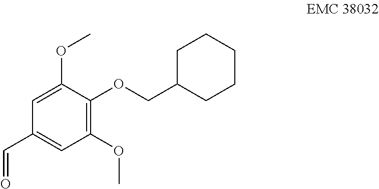 [SMILES: COc1cc(C=O)cc(OC)c1OCC1CCCCC1]

MW: 278.35; Yield: 54%; Yellow oil.

¹H-NMR (CDCl₃, δ): 0.92-1.31 (m, 11H, CH & 5×CH₂), 3.86 (d, 2H, OCH₂, J=6.51 Hz), 3.91 (s, 6H, 2×OCH₃), 7.12 (s, 2H, 2×ArH), 9.86 (s, 1H, CHO).

¹³C-NMR (CDCl₃, δ): 25.8, 26.2, 26.6, 29.8, 31.8, 38.6, 40.1, 40.8, 56.3, 79.2, 107, 131.5, 143.6, 153.9, 191.1

MS-ESI m/z (% rel. Int.): 279 ([MH]⁺, 20), 183 (100).

HPLC: Method A, XBridge™ column, detection UV 254 nm, RT=6.75 min, peak area 99.9%.

### 4-(4-(Cyclohexylmethoxy)-3,5-dimethoxybenzyl)-7-ethoxyisoquinolin-8-ol hydrochloride 36

In an ace pressure tube 2-((2,2-diethoxyethylamino)methyl)-6-ethoxyphenol SAO 33014 (333 mg, 1.17 mmol, 1.0 eq) and 4-(cyclohexylmethoxy)-3,5-dimethoxy-benzaldehyde EMC 38032 (327 mg, 1.17 mmol, 1.0 eq) were dissolved in EtOH (2 mL) and a 37% HCl solution (2 mL) was added. The reaction mixture was stirred at 90° C. for 25 min, cooled to 4° C. and concentrated. The crude product was transformed into its freebase with a 18 N NH₄OH solution (800 μL) and CH₂Cl₂ (200 mL) was added. The separated organic layer was washed with brine (30 mL), dried over MgSO₄, filtered and evaporated to give a crude product (407.5 mg). This crude material was purified by column chromatography (SiO₂, eluent cyclohexane:EtOAc=100:0 to 60:40) to give, after evaporation and drying, 4-(4-(cyclohexylmethoxy)-3,5-dimethoxybenzyl)-7-ethoxyisoquinolin-8-ol (52 mg) as a yellow solid. This solid was dissolved in MeOH (2 mL) and a 1.34 N HCl solution in MeOH (90 μL, 0.121 mmol, 1.05 eq) was slowly added. The reaction mixture was stirred at 4° C. for 15 min. After evaporation and drying under vacuum pump under P₂O₅, 4-(4-(cyclohexylmethoxy)-3,5-dimethoxybenzyl)-7-ethoxyisoquinolin-8-ol hydrochloride 36 (52.3 mg, 9% yield) was obtained as a yellow solid.

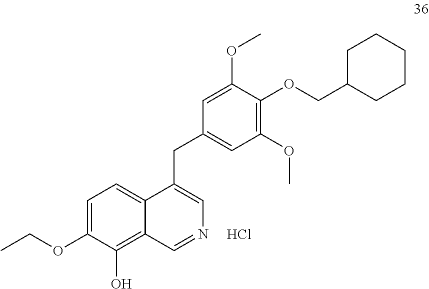 [SMILES: CCOc1ccc2c(Cc3cc(OC)c(OCC4CCCCC4)c(OC)c3)cncc2c1O ; Cl]

MW: 488.02; Yield: 9%; Yellow Solid; Mp (° C.): 241.0

R_{f}: 0.2 (cyclohexane:EtOAc=6:4, free base).

¹H-NMR (CD₃OD, δ): 0.97-1.09 (m, 2H, CH₂), 1.19-1.32 (m, 2H, CH₂), 1.51 (t, 3H, J=6.97 Hz, CH₂CH₃), 1.69-1.77 (m, 5H, 2×CH₂ & CH), 1.87-1.91 (m, 2H, CH₂), 3.69 (d, 2H, J=6.44 Hz, OCH₂CH), 3.77 (s, 6H, 2×OCH₃), 4.36 (q, 2H, J=6.97 Hz, OCH₂CH₃), 4.48 (s, 2H, CH₂), 6.61 (s, 2H, 2×ArH), 7.89 (d, 1H, J=9.08 Hz, ArH), 8.04 (d, 1H, J=9.18 Hz, ArH), 8.07 (s, 1H, ArH), 9.66 (s, 1H, ArH).

¹³C-NMR (CD₃OD, δ): 15.0, 27.0 (2×C), 27.7, 31.0, 37.0, 39.8, 56.7, 66.9, 80.2, 107.6, 116.5, 120.5, 126.7, 128.4, 132.8, 134.7, 137.7, 138.0, 142.5, 146.4, 146.8, 155.1 (2×C).

MS-ESI m/z (% rel. Int.): 452.2 ([MH]⁺, 100).

HPLC: Method A, XBridge™ column, detection UV 254 nm, RT=5.54 min, peak area 98.0%.

### Preparation of 7-ethoxy-4-(4-(2-ethylbutoxy)-3,5-dimethoxybenzyl)isoquinolin-8-ol hydrochloride 37

### 4-(2-Ethylbutoxy)-3,5-dimethoxybenzaldehyde EMC 38034

4-Hydroxy-3,5-dimethoxybenzaldehyde (500 mg, 2.74 mmol) was dissolved in DMF (7.5 mL) and Cs₂CO₃ (894 mg, 2.74 mmol) and 1-bromo-2-ethylbutane (494 mg, 3.02 mmol) were successively added and the reaction mixture was stirred at 40° C. for 10 h under a nitrogen atmosphere. After cooling to RT, the reaction mixture was poured into water (75 mL) and extracted by Et₂O (50 mL). The separated organic layer was washed with water (2×50 mL), brine (50 mL), dried over MgSO₄, filtered and dried overnight under reduced pressure to give 4-(2-ethylbutoxy)-3,5-dimethoxybenzaldehyde EMC 38034 (273 mg, 37% yield) as yellow oil.

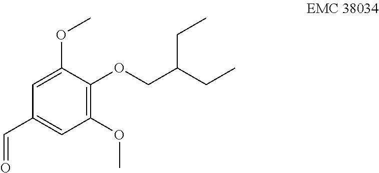 [SMILES: CCC(CC)COc1c(OC)cc(C=O)cc1OC]

MW: 266.34; Yield: 37%; Yellow oil.

¹H-NMR (CDCl₃, δ): 0.91-0.96 (t, 6H, J=7.26 Hz, CH₂CH₃), 1.43-1.64 (m, 5H, CH & 2×CH₃CH₂CH), 3.91 (s, 6H, 2×OMe), 3.94-3.98 (m, 2H, OCH₂), 7.12-7.13 (s, 2H, 2×ArH), 9.87 (s, 1H, CHO).

¹³C-NMR (CDCl₃, δ): 11.0 (2×C), 23.0 (2×C), 41.9, 56.3, 75.6, 106.7 (2×C), 131.5 (2C), 143.6, 154.0 (2×C), 191.2.

MS-ESI m/z (% rel. Int.): 267 ([MH]⁺, 10), 183 (100).

HPLC: Method A, XBridge™ column, detection UV 254 nm, RT=6.66 min, peak area 99.9%.

### 7-Ethoxy-4-(4-(2-ethylbutoxy)-3,5-dimethoxybenzyl)isoquinolin-8-ol hydrochloride 37

In an ace pressure tube, 2-((2,2-diethoxyethylamino)methyl)-6-ethoxyphenol SAO 33014 (248.7 mg, 0.878 mmol, 1.0 eq) and 4-(2-ethylbutoxy)-3,5-dimethoxybenzaldehyde EMC 38034 (233.8 mg, 0.878 mmol, 1.0 eq) were dissolved in EtOH (1.6 mL) and a 37% HCl solution (1.6 mL) was added. The reaction mixture was stirred at 90° C. for 20 min, cooled to 4° C. and concentrated. The crude product was transformed into its freebase with a 18 N NH₄OH solution (0.5 mL) and CH₂Cl₂ (150 mL) was added. The separated organic layer was washed with brine (30 mL), dried over MgSO₄, filtered and evaporated to give orange oil (374 mg). This crude material was purified by column chromatography (SiO₂, eluent CH₂Cl₂:MeOH=100:0 to 98:2) to give, after evaporation and drying, 7-ethoxy-4-(4-(2-ethylbutoxy)-3,5-dimethoxybenzyl)isoquinolin-8-ol (133.6 mg) as a yellow oil. This oil was dissolved in MeOH (2 mL) and a 1.34 N HCl solution in MeOH (238 μL, 0.32 mmol, 1.05 eq) was slowly added. The reaction mixture was stirred at 4° C. for 15 min. After evaporation of solvent and drying under vacuum pump under P₂O₅, 7-ethoxy-4-(4-(2-ethylbutoxy)-3,5-dimethoxybenzyl)isoquinolin-8-ol hydrochloride 37 (135.4 mg, 32% yield) was obtained as a yellow solid.

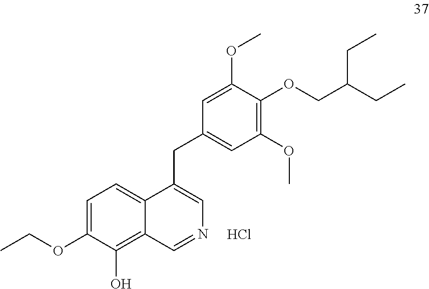 [SMILES: CCOc1ccc2c(Cc3cc(OC)c(OCC(CC)CC)c(OC)c3)cncc2c1O ; Cl]

MW: 476.0; Yield: 32%; Yellow solid; Mp (° C.): 245.6

R_{f}: 0.2 (CH₂Cl₂:MeOH=98:2, free base).

¹H-NMR (CD₃OD, δ): 0.94 (t, 6H, J=6.0 Hz, 2×CH₂CH₃), 1.38-1.53 (m, 8H, CH & 2×CHCH₂CH₃ & OCH₂CH₃), 3.77-3.78 (m, 8H, OCH₂CH & 2×OMe), 4.36 (q, 2H, J=6.0 Hz, OCH₂CH₃), 4.48 (s, 2H, CH₂), 6.61 (s, 2H, 2×ArH), 7.89 (d, 1H, J=9.0 Hz, ArH), 8.04 (d, 1H, J=9.0 Hz, ArH), 8.08 (s, 1H, ArH), 9.65 (s, 1H, ArH).

¹³C-NMR (CD₃OD, δ): 11.5 (2×C), 15.1, 24.3 (2×C), 37.0, 43.3, 56.7 (2×C), 66.9, 76.1, 107.6, 116.6, 120.5, 126.7, 128.3, 132.8, 134.7, 137.7, 138.0, 142.4, 146.4, 146.8, 155.2 (2×C).

MS-ESI m/z (% rel. Int.): 440.2 ([MH]⁺, 100).

HPLC: Method A, XBridge™ column, detection UV 254 nm, RT=5.58 min, peak area 98.0%

### Preparation of 4-(4-butoxy-3,5-dimethoxybenzyl)-7-ethoxyisoquinolin-8-ol hydrochloride 38

### 4-Butoxy-3,5-dimethoxy-benzaldehyde EMC 38046

4-Hydroxy-3,5-dimethoxybenzaldehyde (500 mg, 2.74 mmol) was dissolved in DMF (7.5 mL) and Cs₂CO₃ (894 mg, 2.74 mmol) and bromobutane (414 mg, 3.02 mmol) were added at RT and the reaction mixture was stirred at 40° C. for 10 h under a nitrogen atmosphere. After cooling to RT, the reaction mixture was poured into water (75 mL) and extracted by Et₂O (50 mL). The separated organic layer was washed with water (2×50 mL), brine (50 mL), dried over MgSO₄, filtered and dried overnight under reduced pressure to give 4-butoxy-3,5-dimethoxy-benzaldehyde EMC 38046 (625 mg, 96% yield) as yellow oil.

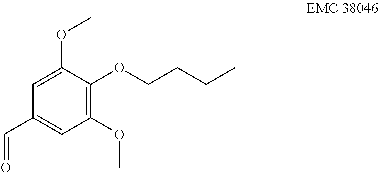 [SMILES: CCCCOc1c(OC)cc(C=O)cc1OC]

MW: 238.29; Yield: 96%; Yellow oil.

¹H-NMR (CDCl₃, δ): 0.96 (t, 3H, CH₃, J=7.34 Hz), 1.46-1.51 (m, 2H, CH₂), 1.70-1.77 (m, 2H, CH₂), 3.92 (s, 6H, 2×OCH₃), 4.08 (t, 2H, OCH₂, J=6.72 Hz), 7.13 (s, 2H, 2×ArH), 9.87 (s, 1H, CHO).

¹³C-NMR (CDCl₃, δ): 13.8, 19.0, 32.2, 56.3, 73.4, 106.8 (2×C), 131.6, 143.2, 154.0 (2×C), 191.1.

MS-ESI m/z (% rel. Int.): 239 ([MH]⁺, 75), 183 (100).

HPLC: Method A, XBridge™ column, detection UV 254 nm, RT=5.77 min, peak area 99.9%.

### 4-(4-Butoxy-3,5-dimethoxybenzyl)-7-ethoxyisoquinolin-8-ol hydrochloride 38

In an ace pressure tube, 2-((2,2-diethoxyethylamino)methyl)-6-ethoxyphenol SAO 33014 (357 mg, 1.26 mmol, 1.0 eq) and 4-butoxy-3,5-dimethoxy-benzaldehyde EMC 38046 (300 mg, 1.26 mmol, 1.0 eq) were dissolved in EtOH (1.6 mL) and a 37% HCl solution (1.6 mL) was added. The reaction mixture was stirred at 90° C. for 20 min, cooled to 4° C. and concentrated. The crude product was transformed into its freebase with a 18 N NH₄OH solution (0.5 mL) and CH₂Cl₂ (150 mL) was added. The separated organic layer was washed with brine (30 mL), dried over MgSO₄, filtered and evaporated to give a yellow oil (320 mg). This crude oil was purified by column chromatography (SiO₂, eluent cyclohexane:EtOAc=100:0 to 50:50) to give, after evaporation and drying, 4-(4-butoxy-3,5-dimethoxybenzyl)-7-ethoxyisoquinolin-8-ol (207.4 mg) as a yellow solid. This solid was dissolved in MeOH (2 mL), and a 1.34 N HCl solution in MeOH (395 μL, 0.53 mmol, 1.05 eq) was slowly added. The reaction mixture was stirred at 4° C. for 15 min. After evaporation and drying under vacuum pump under P₂O₅, 4-(4-butoxy-3,5-dimethoxybenzyl)-7-ethoxyisoquinolin-8-ol hydrochloride 38 was obtained (211 mg, 37% yield) as a yellow solid.

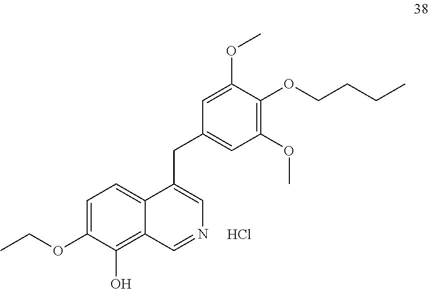 [SMILES: CCCCOc1c(OC)cc(Cc2cncc3c(O)c(OCC)ccc23)cc1OC ; Cl]

MW: 447.95; Yield: 37%; Yellow solid; Mp (° C.): 237.3

R_{f}: 0.25 (cyclohexane:EtOAc=5:5, free base).

¹H-NMR (CD₃OD, δ): 0.92 (m, 3H, OCH₂CH₃), 1.47-1.52 (m, 5H, CH₂CH₃ & OCH₂CH₃), 1.64-1.66 (m, 2H, CH₂), 3.76 (s, 6H, 2×OMe), 3.86-3.91 (m, 2H, OCH₂CH₂), 4.30-4.38 (m, 2H, OCH₂CH₃), 4.47 (s, 2H, CH₂), 6.60 (s, 2H, 2×ArH), 7.87 (d, 1H, J=8.99 Hz, ArH), 8.03 (m, 1H, ArH), 8.05 (s, 1H, ArH), 9.64 (s, 1H, ArH).

¹³C-NMR (CD₃OD, δ): 14.2, 15.0, 20.1, 33.3, 37.0, 56.7 (2×C), 66.9, 74.1, 107.5, 116.5, 120.5, 126.7, 128.4, 132.8, 134.9, 137.3, 138.0, 142.5, 146.4, 146.8, 155.2 (2×C).

MS-ESI m/z (% rel. Int.): 412.1 ([MH]⁺, 100).

HPLC: Method A, XBridge™ column, detection UV 254 nm, RT=4.97 min, peak area 99.9%.

### Preparation of 4-(3,5-dimethoxy-4-(neopentyloxy)benzyl)-7-ethoxyisoquinolin-8-ol hydrochloride 39

### 3,5-Dimethoxy-4-(neopentyloxy)benzaldehyde EMC 38048

4-Hydroxy-3,5-dimethoxybenzaldehyde (500 mg, 2.74 mmol) was dissolved in DMF (7.5 mL) and Cs₂CO₃ (894 mg, 2.74 mmol) and neopentyl bromide (456 mg, 3.02 mmol) were added at RT and the reaction was heated at 150° C. for 10 min under microwave irradiation. After cooling to RT, the reaction mixture was poured into water (75 mL) and extracted by Et₂O (50 mL). The separated organic layer was washed with water (2×50 mL), brine (50 mL), dried over MgSO₄, filtered and dried overnight under reduced pressure to give 3,5-dimethoxy-4-(neopentyloxy)benzaldehyde EMC 38048 (274 mg, 40% yield) as yellow oil.

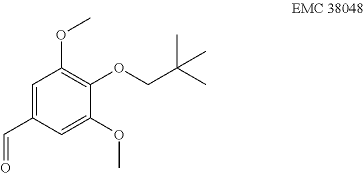 [SMILES: COc1cc(C=O)cc(OC)c1OCC(C)(C)C]

MW: 252.31; Yield: 40%; Yellow oil.

¹H-NMR (CDCl₃, δ): 1.05 (s, 9H, 3×CH₃), 3.72 (s, 2H, OCH₂), 3.90 (s, 6H, 2×OCH₃), 7.12 (s, 2H, 2×ArH), 9.85 (s, 1H, CHO).

¹³C-NMR (CDCl₃, δ): 26.4 (3×C), 32.6, 56.4 (2×C), 83.7, 107.2 (2×C), 131.3, 144.2, 153.8, 191.2.

MS-ESI m/z (% rel. Int.): 253 ([MH⁺], 20), 183 (100).

HPLC: Method A, XBridge™ column, detection UV 254 nm, RT=6.36 min, peak area 99.9%.

### 4-(3,5-Dimethoxy-4-(neopentyloxy)benzyl)-7-ethoxyisoquinolin-8-ol hydrochloride 39

In an ace pressure tube, 2-((2,2-diethoxyethylamino)methyl)-6-ethoxyphenol SAO 33014 (257 mg, 0.91 mmol, 1.0 eq) and 3,5-dimethoxy-4-(neopentyloxy)benzaldehyde EMC 38048 (229 mg, 0.91 mmol, 1.0 eq) were dissolved in EtOH (1.4 mL) and a 37% HCl solution (1.4 mL) was added. The reaction mixture was stirred at 90° C. for 20 min, cooled to 4° C. and concentrated. The crude product was transformed into its freebase with a 18 N NH₄OH solution (0.5 mL) and extracted with CH₂Cl₂ (250 mL). The separated organic layer was washed with brine (50 mL), dried over MgSO₄, filtered and evaporated to give a yellow oil. This crude oil was purified by column chromatography (SiO₂, eluent CH₂Cl₂:MeOH=98:2) to give, after evaporation and drying, 4-(3,5-dimethoxy-4-(neopentyloxy)benzyl)-7-ethoxyisoquinolin-8-ol (89.1 mg) as a yellow solid. This solid was dissolved in MeOH (3 mL) and a 1.34 N HCl solution in MeOH (164 μL, 0.22 mmol, 1.05 eq) was slowly added. The reaction mixture was stirred at 4° C. for 15 min. After evaporation and drying under vacuum pump under P₂O₅, 4-(3,5-dimethoxy-4-(neopentyloxy)benzyl)-7-ethoxyisoquinolin-8-ol hydrochloride 39 (97.2 mg, 23% yield) was obtained as a yellow solid.

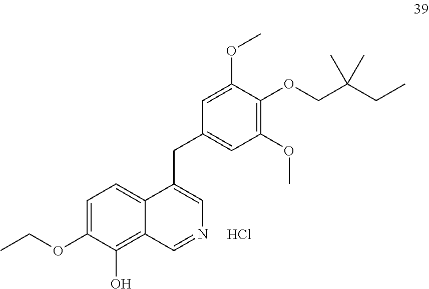 [SMILES: CCOc1ccc2c(Cc3cc(OC)c(OCC(C)(C)CC)c(OC)c3)cncc2c1O ; Cl]

MW: 461.98; Yield: 23%; Yellow Solid; Mp (° C.): 254.9

R_{f}: 0.25 (CH₂Cl₂:MeOH=98:2, free base).

¹H-NMR (CD₃OD, δ): 1.01 (s, 9H, 3×CH₃), 1.50 (t, 3H, J=6.94 Hz, OCH₂CH₃), 3.54 (s, 2H, OCH₂), 3.76 (s, 6H, 2×OMe), 4.35 (q, 2H, J=6.98 Hz, OCH₂CH₃), 4.47 (s, 2H, CH₂), 6.59 (s, 2H, 2×ArH), 7.87 (d, 1H, J=9.07 Hz, ArH), 8.02 (d, 1H, J=9.15 Hz, ArH), 8.06 (s, 1H, ArH), 9.64 (s, 1H, ArH).

¹³C-NMR (CD₃OD, δ): 12.7, 24.6 (3×C), 30.9, 34.6, 54.4 (2×C), 64.5, 82.1, 105.5, 114.2, 118.1, 124.2, 126.3, 130.4, 132.2, 135.5, 136.0, 140.2, 143.9, 144.4, 152.8 (2×C).

MS-ESI m/z (% rel. Int.): 426.3 ([MH]⁺, 100).

HPLC: Method A, XBridge™ column, detection UV 254 nm, RT=5.38 min, peak area 99.9%.

### Preparation of (7-ethoxyisoquinolin-4-yl)(3,4,5-trimethoxyphenyl)methanone hydrochloride 40

### (7-Ethoxyisoquinolin-4-yl)(3,4,5-trimethoxyphenyl)methanone hydrochloride 40

7-Ethoxy-4-(3,4,5-trimethoxybenzyl)isoquinoline (16 free base, 1.0 g, 2.829 mmol, 1.0 eq) was dissolved in anhydride acetic (10 mL) at 4° C. under a nitrogen atmosphere. Concentrated HNO₃ (400 μL, 6.27 μmol, 2.0 eq) was added dropwise. The reaction mixture was stirred at 4° C. to RT overnight and a precipitate appeared. The reaction mixture was filtered to give a crude material (704.7 mg) as a beige solid. The solid was transformed into its freebase a saturated aqueous K₂CO₃ solution (30 mL) and extracted with EtOAc (200 mL). The separated organic layer was washed with brine (30 mL), dried over MgSO₄, filtered and evaporated to give (7-ethoxyisoquinolin-4-yl)(3,4,5-trimethoxyphenyl)methanone (545 mg). This solid was dissolved in MeOH (2 mL) and a 1.34 N HCl solution in MeOH (153 μL, 0.21 mmol, 1.05 eq) was slowly added. The reaction mixture was stirred at 4° C. for 15 min. After evaporation and drying under vacuum pump under P₂O₅, (7-ethoxyisoquinolin-4-yl)(3,4,5-trimethoxyphenyl)methanone hydrochloride 40 (76.4 mg, 50% yield) was obtained as a yellow solid.

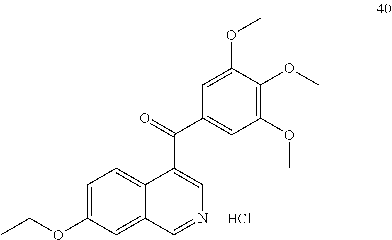 [SMILES: CCOc1ccc2c(C(=O)c3cc(OC)c(OC)c(OC)c3)cncc2c1 ; Cl]

MW: 403.86; Yield: 50%; Yellow Solid; Mp (° C.): 215.9

¹H-NMR (CD₃OD, δ): 1.54 (t, 3H, J=6.95 Hz, OCH₂CH₃), 3.82 (s, 6H, 2×OMe), 3.89 (s, 3H, OMe), 4.35 (q, 2H, J=6.99 Hz, OCH₂CH₃), 7.21 (s, 2H, 2×ArH), 7.85 (d, 1H, J=9.29 Hz, ArH), 7.96 (s, 1H, ArH), 8.14 (d, 1H, J=9.32 Hz, ArH), 8.62 (s, 1H, ArH), 9.75 (s, 1H, ArH).

¹³C-NMR (CD₃OD, δ): 14.7, 56.9 (2×C), 61.3, 66.1, 109.4 (2×C), 109.7, 128.2, 130.8, 131.7, 132.3, 132.9, 133.4, 135.8, 145.6, 148.2, 154.9 (2×C), 162.0, 192.3.

MS-ESI m/z (% rel. Int.): 368.1 ([MH]⁺, 100).

HPLC: Method A, XBridge™ column, detection UV 254 nm, RT=4.69 min, peak area 98.0%.

### Preparation of (7-ethoxyisoquinolin-4-yl)(3,4,5-trimethoxyphenyl)methanone oxime hydrochloride 41

### (7-ethoxyisoquinolin-4-yl)(3,4,5-trimethoxyphenyl)methanone oxime hydrochloride 41

A solution of (7-ethoxyisoquinolin-4-yl)(3,4,5-trimethoxyphenyl)methanone hydrochloride 40 (78.1 mg, 0.21 mmol, 1.0 eq) and hydroxylamine hydrochloride (148 mg, 2.12 mmol, 10.0 eq) in pyridine (2 mL) was stirred at RT for 48 h under a nitrogen atmosphere. Pyridine was evaporated at 30° C. and the crude product was purified by column chromatography (SiO₂, eluent CH₂Cl₂:EtOAc=70:30) to give, after evaporation and drying, ((7-ethoxyisoquinolin-4-yl)(3,4,5-trimethoxyphenyl)methanone oxime (30.1 mg) as a pale yellow solid. This solid was dissolved in MeOH (1 mL) and a 1.45 N HCl solution in MeOH (0.6 mL, 0.087 mmol, 1.1 eq) was slowly added. After evaporation and drying, (7-ethoxyisoquinolin-4-yl)(3,4,5-trimethoxyphenyl)methanone oxime hydrochloride 41 (28.6 mg, 32% yield) was obtained as as an off-white solid.

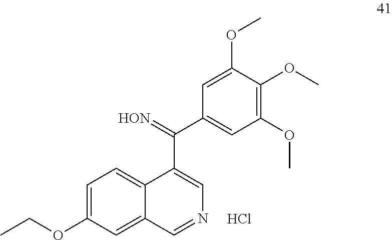

MW: 418.87; Yield: 32%; Off-White Solid; Mp (° C.): 241.6

R_{f}: 0.25 (CH₂Cl₂:EtOAc=70:30, free base).

¹H-NMR (DMSO, δ): 1.44 (t, 3H, J=7 Hz, CH₃), 3.67 (d, 9H, 3×CH₃), 4.25 (q, 2H, J=7 Hz, OCH₂), 6.72 (s, 2H, 2×ArH), 7.58-7.69 (m, 2H, 2×ArH), 8.44 (s, 1H, ArH), 9.62 (s, 1H, ArH), 11.83 (s, 1H, OH).

¹³C-NMR (DMSO, δ): 14.4, 56.0 (2×C), 60.1, 64.1, 104.0 (2×C), 107.8, 126.6, 127.7, 128.2, 129.3, 130.3, 130.7, 133.0, 139.1, 147.7, 150.0, 153.0 (2×C), 158.6.

MS-ESI m/z (% rel. Int.): 383.1 ([MH]⁺, 100).

HPLC: Method A, XBridge™ column, detection UV 254 nm, RT=4.20 min, peak area 99.9%.

### Preparation of (7-ethoxyisoquinolin-4-yl)(3,4,5-trimethoxyphenyl)methanethione hydrochloride 42

### (7-Ethoxyisoquinolin-4-yl)(3,4,5-trimethoxyphenyl)methanethione hydrochloride 42

A solution of (7-ethoxyisoquinolin-4-yl)(3,4,5-trimethoxyphenyl)methanone hydrochloride 40 (50 mg, 0.135 mmol, 1.0 eq) in anhydrous toluene (13 mL) was added Lawesson's reagent (110 mg, 0.27 mmol, 2.0 eq) under a nitrogen atmosphere. The solution was stirred at 130° C. for 2 h. After cooling to RT, the solvent was evaporated and the residue was dried. The residue was extracted with EtOAc (50 mL) and washed with water (15 mL). The separated organic layer was washed with brine (10 mL), dried over MgSO₄, filtered and evaporated to give a green oil (109 mg). This crude oil was purified by column chromatography (SiO₂, eluent cyclohexane:EtOAc=100:0 to 50:50) to give, after evaporation and drying, (7-ethoxyisoquinolin-4-yl)(3,4,5-trimethoxyphenyl)methanethione hydrochloride (17.3 mg) as a green solid. This solid was dissolved in MeOH and a 0.15 N HCl solution in MeOH (331 μL, 0.05 mmol, 1.1 eq) was added. The solution was stirred at 4° C. for 10 min then the solvent was evaporated and the residue was dried to give (7-ethoxyisoquinolin-4-yl)(3,4,5-trimethoxyphenyl)methanethione hydrochloride 42 (10.7 mg, 21% yield) as a green solid.

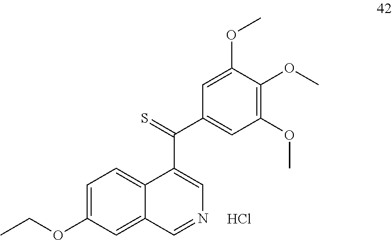 [SMILES: CCOc1ccc2c(C(=S)c3cc(OC)c(OC)c(OC)c3)cncc2c1 ; Cl]

MW: 383.46; Yield: 21%; Green solid; Mp (° C.): 189.0

R_{f}: 0.25 (cyclohexane: EtOAc=5:5, freebase).

¹H-NMR (CD₃OD, δ): 1.51 (t, 3H, CH₃); 3.72 (s, 6H, 2×OMe); 3.91 (s, 3H, OMe); 4.36 (q, 2H, CH₂); 7.24 (s, 2H, 2×ArH); 7.69-7.95 (m, 3H, 3×ArH); 8.51 (s, 1H, ArH), 9.66 (s, 1H, ArH).

¹³C-NMR (CD₃OD, δ): 14.7, 56.9 (2×C), 61.4, 66.0, 108.7 (2×C), 109.2, 128.2, 129.0, 131.3, 131.6, 132.1, 141.6, 145.8, 146.5, 146.7, 154.6 (2×C), 161.8, C═S not observed.

MS-ESI m/z (% rel. Int.): 384.1 ([MH]⁺, 100), 385.1 (24), 386.1 (8).

HPLC: Method A, XBridge™ column, detection UV 391 nm, RT=5.08 min, peak area 97.0%.

### Preparation of (7-ethoxyisoquinolin-4-yl)(3,4,5-trimethoxyphenyl)methanol hydrochloride hydrochloride 43

To a solution of (7-ethoxyisoquinolin-4-yl)(3,4,5-trimethoxyphenyl)methanone (40 free base, 100 mg, 0.27 mmol, 1.0 eq) in MeOH (10 mL) was added slowly at RT under a nitrogen atmosphere sodium borohydride (11.3 mg, 0.30 mmol, 1.1 eq). The reaction mixture was stirred at RT for 1 h then evaporated, H₂O (10 mL) was added and the resulting solution was extracted by EtOAc (80 mL). The separated organic layer was washed with brine (10 mL), dried over MgSO₄, filtered and evaporated to give (7-ethoxyisoquinolin-4-yl)(3,4,5-trimethoxyphenyl)methanol as a white solid (111 mg, 0.30 mmol). This solid was dissolved in MeOH and a 1.34 HCl solution in MeOH (236 μL, 0.32 mmol, 1.05 eq) was added at 4° C. The reaction mixture was stirred at 4° C. for 10 min. After evaporated and drying, (7-ethoxy-isoquinolin-4-yl)-(3,4,5-trimethoxy-phenyl)-methanol hydrochloride 43 (110.4 mg, 100% yield) was obtained as a yellow solid.

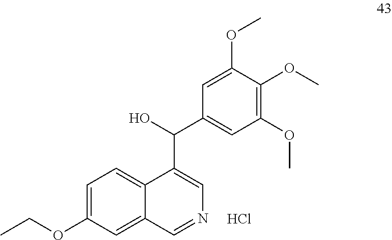 [SMILES: CCOc1ccc2c(C(O)c3cc(OC)c(OC)c(OC)c3)cncc2c1 ; Cl]

MW: 405.87; Yield: 100%; Yellow solid; Mp (° C.): 212.5

¹H-NMR (CD₃OD, δ): 1.50 (t, 3H, J=6.95 Hz, OCH₂CH₃), 3.73 (s, 3H, OMe), 3.78 (s, 6H, 2×OMe), 4.29 (q, 2H, J=6.99 Hz, OCH₂CH₃), 6.49 (s, 1H, HOCH), 6.76 (s, 2H, 2×ArH), 7.78 (dd, 1H, J=2.54 Hz, J=9.33 Hz, ArH), 7.84 (s, 1H, ArH), 8.38 (d, 1H, J=9.34 Hz, ArH), 8.55 (s, 1H, ArH), 9.54 (s, 1H, ArH).

¹³C-NMR (CD₃OD, δ): 14.7, 56.7 (2×C), 61.1, 65.9, 72.1, 105.9 (2×C), 109.6, 127.5, 128.4, 131.2 (2×C), 133.5, 139.1, 139.2, 141.3, 145.7, 154.9 (2×C), 161.4.

MS-ESI m/z (% rel. Int.): 370.3 ([MH]⁺, 100).

HPLC: Method A, XBridge™ column, detection UV 254 nm, RT=3.97 min, peak area 99.9%.

### Preparation of 4-(3,5-dimethoxy-4-(3-phenylpropoxy)benzyl)-7-ethoxyisoquinolin-8-ol hydrochloride 44

### 3,5-Dimethoxy-4-(3-phenylpropoxy)benzaldehyde EMC 38052

4-Hydroxy-3,5-dimethoxybenzaldehyde (500 mg, 2.74 mmol) was dissolved in DMF (7.5 mL) and Cs₂CO₃ (894 mg, 2.74 mmol) was added at RT. 1-Bromo-3-phenylpropane (601 mg, 3.02 mmol) was added and reaction was stirred at 40° C. overnight under nitrogen atmosphere. After cooling to RT, the reaction mixture was poured into H₂O (75 mL) and extracted by Et₂O (70 mL). The organic layer was washed with H₂O (2×50 mL), brine (50 mL), dried over MgSO₄, filtered and dried overnight under reduced pressure to give 3,5-dimethoxy-4-(3-phenylpropoxy)-benzaldehyde EMC 38052 (745 mg, 90% yield) as yellow oil.

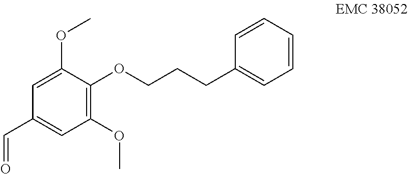 [SMILES: COc1cc(C=O)cc(OC)c1OCCCc1ccccc1]

MW: 300.36; Yield: 90%; Yellow oil.

¹H-NMR (CDCl₃, δ): 2.05-2.10 (m, 2H, CH₂CH₂CH₂), 2.82-2.87 (t, 2H, J=7.88 Hz, PhCH₂), 3.91 (s, 6H, 2×OMe), 4.09-4.13 (t, 2H, J=5.92 Hz, OCH₂), 7.13 (s, 1H, ArH), 7.18-7.29 (m, 2H, 2×ArH), 9.87 (s, 1H, CHO).

¹³C-NMR (CDCl₃, δ): 31.8, 32.1, 56.2 (2×C), 72.8, 106.8 (2×C), 125.8, 128.3 (2×C), 128.5 (2×C), 131.6, 141.9, 143, 153.9 (2×C), 191.1.

MS-ESI m/z (% rel. Int.): 301 ([MH]⁺, 60), 183 (100).

HPLC: Method A, XBridge™ column, detection UV 254 nm, RT=6.23 min, peak area 99.9%.

### 4-(3,5-Dimethoxy-4-(3-phenylpropoxy)benzyl)-7-ethoxyisoquinolin-8-ol hydrochloride 44

In an ace pressure tube, 2-((2,2-diethoxyethylamino)methyl)-6-ethoxyphenol SAO 33014 (617 mg, 2.18 mmol, 1.0 eq) and 3,5-dimethoxy-4-(3-phenylpropoxy)-benzaldehyde EMC 38052 (654 mg, 2.18 mmol, 1.0 eq) were dissolved in EtOH (2.8 mL) and a 37% HCl solution (2.8 mL) was added. The reaction mixture was stirred at 90° C. for 20 min then cooled to 4° C. and concentrated. The crude product was treated by a 18 N NH₄OH solution (0.5 mL) and CH₂Cl₂ (250 mL) was added. The separated organic layer was washed with brine (50 mL), dried over MgSO₄, filtered and evaporated to give a yellow oil. This oil was purified by column chromatography (SiO₂, eluent CH₂Cl₂:MeOH=100:0 to 98:2) to give, after evaporation and drying, 4-(3,5-dimethoxy-4-(3-phenylpropoxy)benzyl)-7-ethoxyisoquinolin-8-ol (215.9 mg) as a yellow solid. This solid was dissolved in MeOH (3 mL) and a 1.34 N HCl solution in MeOH (357 μL, 0.48 mmol, 1.05 eq) was slowly added. The reaction mixture was stirred at 4° C. for 15 min. After evaporation and drying under vacuum pump under P₂O₅, 4-(3,5-dimethoxy-4-(3-phenylpropoxy)benzyl)-7-ethoxyisoquinolin-8-ol hydrochloride 44 (198 mg, 18% yield) was obtained as a yellow solid.

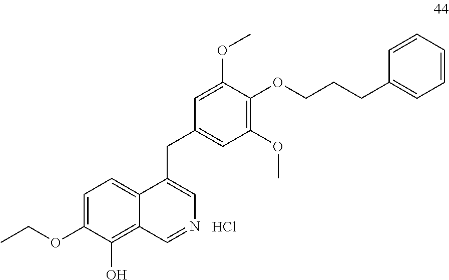 [SMILES: CCOc1ccc2c(Cc3cc(OC)c(OCCCc4ccccc4)c(OC)c3)cncc2c1O ; Cl]

MW: 510.02; Yield: 18%; Yellow Solid; Mp (° C.): 228.3

R_{f}: 0.25 (CH₂Cl₂:MeOH=98:2, free base).

¹H-NMR (CD₃OD, δ): 1.49 (t, 3H, J=6.95 Hz, OCH₂CH₃), 1.95 (m, 2H, J=8.1 Hz, OCH₂CH₂CH₂), 2.78 (t, 2H, J=7.4 Hz, OCH₂CH₂CH₂), 3.76 (s, 6H, 2×OMe), 3.91 (t, 2H, J=6.22 Hz, OCH₂CH₂CH₂), 4.34 (q, 2H, J=6.96 Hz, OCH₂CH₃), 4.48 (s, 2H, CH₂), 6.61 (s, 2H, 2×ArH), 7.13-7.23 (m, 5H, 5×ArH), 7.88 (d, 1H, J=9.08 Hz, ArH), 8.02 (d, 1H, J=9.10 Hz, ArH), 8.06 (s, 1H, ArH), 9.65 (s, 1H, ArH).

¹³C-NMR (CD₃OD, δ): 15.0, 33.1 (2×C), 37.0, 56.7 (2×C), 66.9, 73.5, 107.6 (2×C), 116.6, 120.4, 126.8 (2×C), 128.3, 129.3 (2×C), 129.5 (2×C), 132.8, 134.9, 137.2, 138.0, 142.5, 143.3, 146.4, 146.8, 155.2 (2×C).

MS-ESI m/z (% rel. Int.): 474.2 ([MH]⁺, 100).

HPLC: Method A, XBridge™ column, detection UV 254 nm, RT=5.37 min, peak area 99.9%.

### Preparation of 4-(3,5-dimethoxy-4-phenethoxybenzyl)-7-ethoxyisoquinolin-8-ol hydrochloride 45

### 3,5-Dimethoxy-4-phenethyloxybenzaldehyde EMC 38066

4-Hydroxy-3,5-dimethoxybenzaldehyde (500 mg, 2.74 mmol, 1.0 eq) was dissolved in DMF (7.5 mL) and Cs₂CO₃ (894 mg, 2.74 mmol, 1.0 eq) was added at RT. (2-Bromoethyl)benzene (561 mg, 3.02 mmol, 1.1 eq) was added and the reaction mixture was stirred at 40° C. for 10 h under a nitrogen atmosphere. After cooling to RT, the reaction mixture was poured into H₂O (75 mL) and extracted by Et₂O (50 mL). The separated organic layer was washed with H₂O (2×50 mL), brine (50 mL), dried over MgSO₄, filtered and dried overnight under reduced pressure to give 3,5-dimethoxy-4-phenethyloxybenzaldehyde EMC 38066 (475 mg, 61% yield) as a yellow oil.

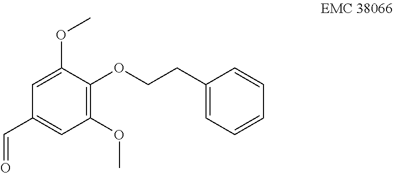 [SMILES: COc1cc(C=O)cc(OC)c1OCCc1ccccc1]

MW: 286.33; Yield: 61%; Yellow oil.

¹H-NMR (CDCl₃, δ): 3.10 (t, 2H, J=7.51 Hz, CH₂), 3.88 (s, 6H, 2×OMe), 4.31 (t, 2H, J=7.93 Hz, OCH₂), 7.11 (s, 2H, 2×ArH), 7.19-7.32 (m, 5H, 5×ArH), 9.86 (s, 1H, CHO).

¹³C-NMR (CDCl₃, δ): 36.6, 56.3 (2×C), 74.0, 106.8 (2×C), 126.3, 128.3 (2×C), 129.0 (2×C), 131.7, 138.1, 142.8, 147.4, 153.8, 191.1.

MS-ESI m/z (% rel. Int.): 287 ([MH]⁺, 90), 183 (100).

HPLC: Method A, XBridge™ column, detection UV 254 nm, RT=5.99 min, peak area 99.9%.

### 4-(3,5-Dimethoxy-4-phenethoxybenzyl)-7-ethoxyisoquinolin-8-ol hydrochloride 45

In an ace pressure tube, 2-((2,2-diethoxyethylamino)methyl)-6-ethoxyphenol SAO 33014 (412 mg, 1.45 mmol, 1.0 eq) and 3,5-dimethoxy-4-phenethoxybenzaldehyde EMC 38066 (417 mg, 1.45 mmol, 1.0 eq) were dissolved in EtOH (2.8 mL) and a 37% HCl solution (2.8 mL) was added. The reaction mixture was stirred at 90° C. for 20 min, cooled to 4° C. and concentrated. The crude product was treated with a 18 N NH₄OH solution (0.5 mL) and CH₂Cl₂ (300 mL) was added. The separated organic layer was washed with brine (50 mL), dried over MgSO₄, filtered and evaporated to give a yellow solid. This crude material was purified by column chromatography, (SiO₂, eluent cyclohexane:EtOAc=100:0 to 50:50) to give, after evaporation and drying, 4-(3,5-dimethoxy-4-phenethoxybenzyl)-7-ethoxyisoquinolin-8-ol (117 mg). This crude product was dissolved in MeOH (2 mL) and a 1.34 N HCl solution in MeOH (200 μL, 0.268 mmol, 1.05 eq) was slowly added. The reaction mixture was stirred at 4° C. for 15 min. After evaporation and drying under vacuum pump under P₂O₅, 4-(3,5-dimethoxy-4-phenethoxybenzyl)-7-ethoxyisoquinolin-8-ol hydrochloride 45 (123 mg, 17% yield) was obtained as a yellow solid.

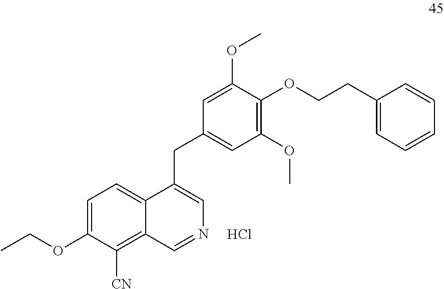 [SMILES: CCOc1ccc2c(Cc3cc(OC)c(OCCc4ccccc4)c(OC)c3)cncc2c1C#N ; Cl]

MW: 495.99; Yield: 17%; Yellow solid; Mp (° C.): 250.1

R_{f}: 0.25 (cyclohexane:EtOAc=5:5, free base).

¹H-NMR (CD₃OD, δ): 1.54 (t, 3H, J=6.96 Hz, OCH₂CH₃), 3.03 (t, 2H, J=7.17 Hz, OCH₂CH₂), 3.77 (s, 6H, 2×OMe), 4.14 (t, 2H, J=7.19 Hz, OCH₂CH₂), 4.38 (q, 2H, J=6.93 Hz, OCH₂CH₃), 4.51 (s, 2H, CH₂), 6.64 (s, 2H, 2×ArH), 7.18-7.23 (m, 1H, ArH), 7.28-7.30 (m, 4H, 4×ArH), 7.92 (d, 1H, J=9.10 Hz, ArH), 8.07 (d, 1H, J=9.23 Hz, ArH), 8.09 (s, 1H, ArH), 9.69 (s, 1H, ArH).

¹³C-NMR (CD₃OD, δ): 15.0, 37.0, 37.5, 56.7 (2×C), 66.9, 75.0, 107.6 (2×C), 116.5, 120.5, 126.7, 127.2, 128.3, 129.3 (2×C), 130.0 (2×C), 132.8, 135.0, 137.3, 137.9, 139.9, 142.5, 146.4, 146.8, 155.1 (2×C).

MS-ESI m/z (% rel. Int.): 460.2 ([MH]⁺, 100).

HPLC: Method A, XBridge™ column, detection UV 254 nm, RT=5.17 min, peak area 99.9%.

### Preparation of 7-ethoxy-4-(4-ethoxy-3,5-dimethoxybenzyl)-8-methoxyisoquinoline hydrochloride 46

### 3-Ethoxy-2-methoxybenzaldehyde ECO 39016

3-Ethoxysalicylaldehyde (9.22 g, 5.5 mmol) was dissolved in DMF (120 mL) and Cs₂CO₃ (18.0 g, 55.5 mmol, 1.0 eq) was added then the reaction mixture was stirred at RT for 5 min. Iodomethane (3.80 mL, 61.5 mmol) was added and the reaction mixture was stirred overnight at 40° C. under a nitrogen atmosphere. After cooling to RT, the reaction mixture was diluted with H₂O (300 mL) and extracted with Et₂O (200 mL). The separated organic layer was washed with brine (30 mL), dried over MgSO₄ and filtered. After evaporation and drying under P₂O₅, 3-ethoxy-2-methoxybenzaldehyde ECO 39016 (13.5 g, 100% yield) was obtained as a brown oil.

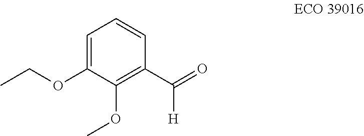

MW: 180.21; Yield: 100%; Brown oil.

¹H-NMR (CDCl₃, δ): 1.48 (t, J=7.0 Hz, 3H, CH₃CH₂), 4.00 (s, 1H, CH₃O), 4.12 (q, 2H, CH₂CH₃), 7.12 (m, 2H, 2×ArH), 7.36 (m, 1H, ArH), 10.41 (s, 1H, CHO).

MS-ESI m/z (% rel. Int.): 181.1 ([MH]⁺, 100).

HPLC: Method A, XBridge™ column, detection UV 254 nm, RT=3.00 min, peak area 99.9%.

### (E)-1,1-diethoxy-N-(3-ethoxy-2-methoxybenzylidene)methanamine ECO 39026

In a Dean Stark apparatus, 3-ethoxy-2-methoxybenzaldehyde ECO 39016 (13.6 g, 75.4 mmol) was dissolved in toluene (220 mL) and aminoacetaldehyde diethylacetal (16.5 mL, 113.2 mmol) was added via syringe at RT under a nitrogen atmosphere. The reaction was stirred for 4 h at 150° C. After evaporation of the solvent (bath: 60° C.) and drying under vacuum pump under P₂O₅, (E)-1,1-diethoxy-N-(3-ethoxy-2-methoxybenzylidene)methanamine ECO 39026 was obtained (18.97 g, 90% yield) as a yellow oil.

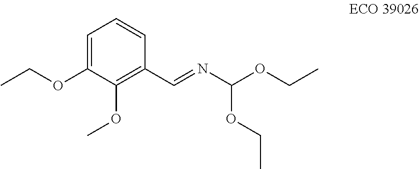 [SMILES: CCOc1cccc(/C=N/C(OCC)OCC)c1OC]

MW: 281.35; Yield: 90%; Yellow oil.

¹H-NMR (CDCl₃, δ): 1.23 (m, 6H, 2×CH₂CH₃), 1.47 (t, J=5.5 Hz, 3H, CH₂CH₃), 2.74 (d, J=6.1 Hz, 2H, CH₂CH), 3.48-3.76 (m, 6H, 3×OCH₂), 3.86 (s, 3H, OMe), 4.10 (q, J=7.5 Hz, CHCH₂), 4.81 (t, 1H, J=5.4 Hz, CH₂CH(OEt)₂), 6.93 (d, 1H, ArH), 7.03 (t, J=6.0 Hz, ArH), 7.54-7.51 (d, J=6.5 Hz, 1H, ArH), 8.66 (s, 1H, CH═N).

### 1,1-Diethoxy-N-(3-ethoxy-2-methoxybenzyl)methanamine ECO 39028

(E)-1,1-Diethoxy-N-(3-ethoxy-2-methoxybenzylidene)methanamine ECO 39026 (18.97 g, 64.2 mmol) was dissolved in EtOH (160 mL) and NaBH₄ (4.61 g, 122 mmol) was slowly added at 4° C. under a nitrogen atmosphere. The reaction mixture was stirred at 100° C. for 1 h, cooled to RT and H₂O (5 mL) was slowly added. The reaction mixture was evaporated to give an off-white solid. The residue was partitioned with CH₂Cl₂ (300 mL) and H₂O (200 mL). The organic layer was washed with brine (30 mL), dried over MgSO₄ and filtered. After evaporation and drying under P₂O₅, 1,1-diethoxy-N-(3-ethoxy-2-methoxybenzyl)methanamine ECO 39028 (15.04 g, 82% yield) was obtained as a yellow oil.

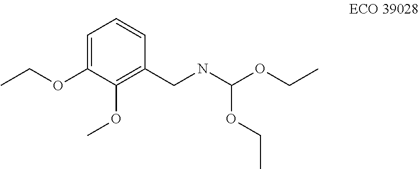 [SMILES: CCOc1cccc(CNC(OCC)OCC)c1OC]

MW: 283.37; Yield: 82%; Yellow oil.

¹H-NMR (CDCl₃, δ): 1.23 (m, 6H, 2×CH₂CH₃), 1.47 (m, 3H, CH₂CH₃), 3.48-3.67 (m, 4H, 2×OCH₂), 3.71 (m, 2H, NCH₂), 3.86 (s, 3H, OMe), 4.09 (m, 2H, OCH₂), 4.60 (m, 1H, CH₂—CH(OEt)₂), 6.79 (m, 3H, 3×ArH).

### 7-Ethoxy-4-(4-ethoxy-3,5-dimethoxybenzyl)-8-methoxyisoquinoline hydrochloride 46

In an ace pressure tube, to a solution of 1,1-diethoxy-N-(3-ethoxy-2-methoxybenzyl)methanamine ECO 39028 (707 mg, 2.38 mmol) in EtOH (3 mL) was added at RT 4-ethoxy-3,5-dimethoxybenzaldehyde TTA 24126 (500 mg, 2.38 mmol) and a 37% HCl solution (3 mL). The reaction mixture was stirred at 100° C. for 20 min and the reaction mixture was cooled to RT. The solvent was evaporated and a 1 M K₂CO₃ aqueous solution (40 mL) was added and the resulting solution was extracted with EtOAc (200 mL). The separated organic layer was washed with brine (30 mL), dried over MgSO₄, filtered and evaporated to give a solid (869.4 mg). This crude solid was purified by column chromatography (SiO₂, eluent CH₂Cl₂:MeOH=100:0 to 98:2) to give, after evaporation and drying, 7-ethoxy-4-(4-ethoxy-3,5-dimethoxy-benzyl)-8-methoxy-isoquinoline (253.3 mg) as a yellow solid. This solid (50.3 mg) was dissolved in MeOH and a 1.35 N HCl solution in MeOH (100 μL, 0.13 mmol, 1.05 eq) was added at 4° C. The solution was stirred at 4° C. for 10 min then the solvent was evaporated and the residue was dried to give 7-ethoxy-4-(4-ethoxy-3,5-dimethoxybenzyl)-8-methoxyisoquinoline hydrochloride 46 (55.2 mg, 27% yield) as a yellow solid.

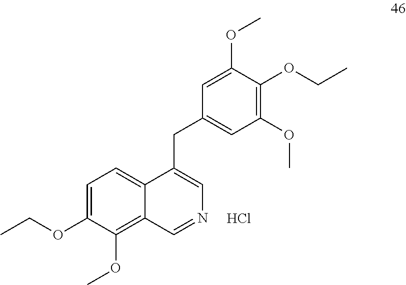 [SMILES: CCOc1ccc2c(Cc3cc(OC)c(OCC)c(OC)c3)cncc2c1OC ; Cl]

MW: 433.93; Yield: 27%; Yellow solid; Mp (° C.)=173.3

R_{f}: 0.25 (CH₂Cl₂:MeOH=98:2, free base).

¹H-NMR (CD₃OD, δ): 1.30 (t, 3H, J=7.0 Hz, OCH₂CH₃), 1.54 (t, 3H, J=6.96 Hz, OCH₂CH₃), 3.78 (s, 6H, 2×OMe), 3.96 (q, 2H, J=7.13 Hz, OCH₂CH₃), 4.23 (s, 3H, OMe); 4.38 (q, 2H, J=6.93 Hz, OCH₂CH₃), 4.54 (s, 2H, CH₂), 6.63 (s, 2H, 2×ArH), 8.16-8.20 (m, 3H, 3×ArH), 9.66 (s, 1H, ArH).

¹³C-NMR (CD₃OD, δ): 15.2, 15.7, 37.0, 56.7 (2×C), 62.6, 67.0, 69.9, 107.5 (2×C), 121.5, 124.9, 128.3, 129.3, 133.6, 134.9, 137.0, 138.4, 142.4, 146.9, 151.5, 155.2 (2×C).

MS-ESI m/z (% rel. Int.): 398.3 ([MH]⁺, 100).

HPLC: Method A, XBridge™ column, detection UV 254 nm, RT=4.46 min, peak area 99.9%.

### Preparation (4-ethoxy-3,5-dimethoxyphenyl)-(7-ethoxy-8-methoxyisoquinolin-4-yl)-methanone hydrochloride 47

### (4-Ethoxy-3,5-dimethoxyphenyl)-(7-ethoxy-8-methoxyisoquinolin-4-yl)-methanone hydrochloride 47

7-Ethoxy-4-(4-ethoxy-3,5-dimethoxybenzyl)-8-methoxyisoquinoline hydrochloride (46 free base, 203 mg, 0.51 mmol, 1.0 eq) was dissolved in anhydride acetic (2 mL) at 4° C. under a nitrogen atmosphere. HNO₃ (132 μL, 2.04 μmol) was added by dropwise. The reaction mixture was stirred from 4° C. to RT for 5 h. A cold solution of 1 M K₂CO₃ (20 mL) and EtOAc (300 mL) were added. The separated organic layer was washed with brine (20 mL), dried over MgSO₄, filtered and evaporated. The crude product was purified by column chromatography (SiO₂, eluent cyclohexane:EtOAc=100:0 to 60:40) to give, after evaporation and drying, (4-ethoxy-3,5-dimethoxyphenyl)-(7-ethoxy-8-methoxyisoquinolin-4-yl)-methanone (50.2 mg, 0.122 mmol). This solid (15 mg, 0.036 mmol) was dissolved in MeOH (1 mL) and a 1.34 N HCl solution in MeOH (29 μL, 0.04 mmol) was slowly added. The reaction mixture was stirred at 4° C. for 15 min. After evaporation and drying under vacuum pump under P₂O₅, (4-ethoxy-3,5-dimethoxyphenyl)-(7-ethoxy-8-methoxyisoquinolin-4-yl)-methanone hydrochloride 47 (15.3 mg, 22% yield) was obtained as a yellow solid.

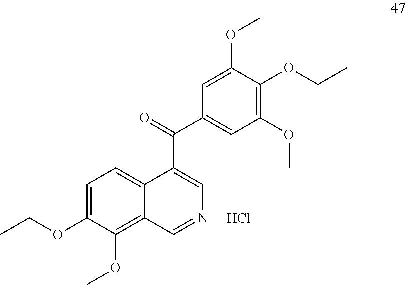 [SMILES: CCOc1ccc2c(C(=O)c3cc(OC)c(OCC)c(OC)c3)cncc2c1OC ; Cl]

MW: 447.91; Yield: 22%; Yellow Solid; Mp (° C.): 162.0

R_{f}: 0.3 (cyclohexane:EtOAc=60:40, free base).

¹H-NMR (CD₃OD, δ): 1.35 (t, 3H, J=7.05 Hz, OCH₂CH₃), 1.55 (t, 3H, J=6.96 Hz, OCH₂CH₃), 3.82 (s, 6H, 2×OMe), 4.14 (q, 2H, J=7.06 Hz, OCH₂CH₃), 4.29 (s, 3H, OMe), 4.40 (q, 2H, J=6.99 Hz, OCH₂CH₃), 7.21 (s, 2H, 2×ArH), 7.91 (d, 1H, J=9.25 Hz, ArH), 8.15 (d, 1H, J=9.31 Hz, ArH), 8.56 (s, 1H, ArH), 9.89 (s, 1H, ArH).

¹³C-NMR (CD₃OD, δ): 15.2, 15.9, 57.0 (2×C), 62.7, 67.1, 70.3, 109.3 (2×C), 122.3, 125.4, 129.0, 130.5, 131.7, 132.9, 135.5, 144.6, 145.8, 146.9, 151.8, 155.2 (2×C), 192.4.

MS-ESI m/z (% rel. Int.): 412.2 ([MH]⁺, 100).

HPLC: Method A, XBridge™ column, detection UV 254 nm, RT=4.92 min, peak area 98%.

### Preparation of methyl 2-(4-((7-ethoxy-8-hydroxyisoquinolin-4-yl)methyl)-2,6-dimethoxyphenoxy)acetate hydrochloride 48

### 2-(4-Formyl-2,6-dimethoxyphenoxy)acetamide RBO 40048

4-Hydroxy-3,5-dimethoxybenzaldehyde (1.0 g, 5.49 mmol) was dissolved in DMF (50 mL) and Cs₂CO₃ (3.58 g, 10.98 mmol) was added at RT. 2-Bromoacetamide (909 mg, 6.59 mmol) was added and reaction was stirred at 40° C. overnight under a nitrogen atmosphere. DMF was removed and EtOAc (50 mL) was added. The organic layer was washed with H₂O (2×100 mL), brine (50 mL), dried over Na₂SO₄, filtered and concentrated to dryness under reduced pressure to give an off-white solid. This solid was purified by column chromatography (SiO₂, eluent CH₂Cl₂:EtOAc=1:1) to give, after evaporation and drying, 2-(4-formyl-2,6-dimethoxyphenoxy)acetamide RBO 40048 (550 mg, 42% yield) as an off-white solid.

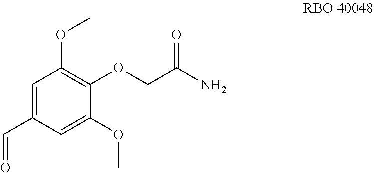 [SMILES: COc1cc(C=O)cc(OC)c1OCC(N)=O]

MW: 239.23; Yield: 42%; Off-white solid; Mp (° C.): 208.8

R_{f}=0.2 (CH₂Cl₂:EtOAc=1:1).

¹H-NMR (CDCl₃, δ): 3.95 (s, 6H, 2×OMe), 4.60 (s, 2H, CH₂), 5.90 (broad s, 1H, NH), 7.55 (broad s, 1H, NH), 7.16 (s, 2H, 2×ArH), 9.89 (s, 1H, CHO).

MS-ESI m/z (% rel. Int.): 240.1 ([MH]⁺, 62), 183.0 (100).

HPLC: Method A, XBridge™ column, detection UV 254 nm, RT=3.61 min, peak area 99.0%.

### Methyl 2-(4-((7-ethoxy-8-hydroxyisoquinolin-4-yl)methyl)-2,6-dimethoxyphenoxy)acetate hydrochloride 48

A mixture of 2-((2,2-diethoxyethylamino)methyl)-6-ethoxyphenol SAO 33014 (238 mg, 0.84 mmol) and 2-(4-formyl-2,6-dimethoxyphenoxy)acetamide RBO 40048 (200 mg, 0.84 mmol) in EtOH (5 mL) and a 37% HCl solution (5 mL) were heated at 100° C. for 20 min under microwave irradiation. An orange precipitate appeared. The orange precipitate was filtered and dissolved in MeOH (20 mL). The reaction mixture was refluxing for another 20 min under microwave irradiation then EtOH was removed under pressure vacuum at 45° C. and the residue was partitioned between EtOAc (50 mL) and a saturated NaHCO₃ solution (10 mL). The organics layers were collected, washed with H₂O (3×20 mL), brine (20 mL), dried over Na₂SO₄, filtered and evaporated to give a yellow solid (93.5 mg). This crude product was purified by column chromatography (SiO₂, eluent CH₂Cl₂:EtOAc=1:1) to give, after evaporation and drying, methyl 2-(4-((7-ethoxy-8-hydroxyisoquinolin-4-yl)methyl)-2,6-dimethoxyphenoxy)acetate (100 mg). This solid (100 mg, 0.233 mmol) was dissolved in a 0.49 N HCl solution (0.5 mL, 0.235 mmol) in MeOH (5 mL) and the reaction mixture was stirred at 4° C. for 2 min. After evaporation and drying under P₂O₅, 2-(4-((7-ethoxy-8-hydroxyisoquinolin-4-yl)methyl)-2,6-dimethoxyphenoxy)acetate hydrochloride 48 (102 mg, 26% yield) was obtained as a yellow solid.

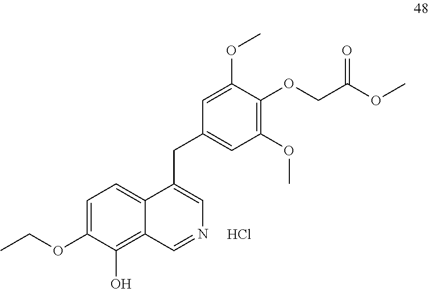 [SMILES: CCOc1ccc2c(Cc3cc(OC)c(OCC(=O)OC)c(OC)c3)cncc2c1O ; Cl]

MW: 463.92; Yield: 26%; Yellow solid; Mp (° C.): 208.8

¹H NMR (CD₃OD, δ): 1.49 (t, 3H J=7.5 Hz, CH₃), 3.76 (s, 9H, 3×OMe), 4.34 (q, 2H J=7.0 Hz, CH₂), 4.48 (s, 2H, CH₂), 4.51 (s, 2H, CH₂), 6.61 (s, 2H, 2×ArH), 7.86 (d, 1H, J=9.0 Hz, ArH), 8.03 (m, 2H, 2×ArH), 9.65 (s, 1H, ArH).

¹H NMR (CD₃OD, δ): 15.0, 37.0, 52.4, 56.7 (2×C), 66.9, 70.3, 107.6 (2×C), 116.5 (2×C), 120.5, 126.7, 128.4, 132.8, 135.5, 137.8, 142.6, 146.4, 146.9, 154.5, 171.7.

MS-ESI m/z (rel. int.): 428 [M+H]⁺ (100).

HPLC: Method A, XBridge™ column, detection UV 254 nm, RT=4.16 min, peak area 99.9%.

### Preparation of 2-(4-((7-ethoxy-8-hydroxyisoquinolin-4-yl)methyl)-2,6-dimethoxyphenoxy)acetamide hydrochloride 49

### 2-(4-((7-Ethoxy-8-hydroxyisoquinolin-4-yl)methyl)-2,6-dimethoxyphenoxy)acetamide hydrochloride 49

In a 20 mL microwave vial, 2-((2,2-diethoxyethylamino)methyl)-6-ethoxyphenol SAO 33014 (237 mg, 0.84 mmol) and 2-(4-formyl-2,6-dimethoxyphenoxy)acetamide RBO 40048 (200 mg, 0.84 mmol) were dissolved in acetic acid (16 mL) and 98% H₂SO₄ (5 drops) was added. The reaction mixture was heated at 100° C. for 15 min under microwave irradiation and cooled to 4° C. The mixture was neutralized with an aqueous solution of Na₂CO₃ (20 mL) and extracted with EtOAC (50 mL). The separated organic layer was washed with H₂O (3×30 mL), brine (30 mL), dried over Na₂SO₄, filtered and evaporated to give a yellow solid. This solid was purified by column chromatography (SiO₂, eluent CH₂Cl₂:MeOH with NH₃ 7N=100:0 to 50:50) to give, after evaporation and drying, 2-(4-((7-ethoxy-8-hydroxyisoquinolin-4-yl)methyl)-2,6-dimethoxyphenoxy)acetamide (58 mg). This solid was dissolved in MeOH (2 mL) and a 0.49 N HCl solution in MeOH (287 μL, 0.14 mmol) was slowly added. The reaction mixture was stirred at 4° C. for 15 min. After evaporation and drying under vacuum under P₂O₅, 2-(4-((7-ethoxy-8-hydroxyisoquinolin-4-yl)methyl)-2,6-dimethoxyphenoxy)acetamide hydrochloride 49 (51.2 mg, 14% yield) was obtained as a yellow solid.

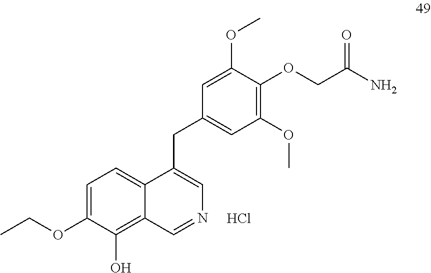 [SMILES: CCOc1ccc2c(Cc3cc(OC)c(OCC(N)=O)c(OC)c3)cncc2c1O ; Cl]

MW: 448.91; Yield: 14%; Yellow solid; Mp (° C.): 148.1

R_{f}: 0.3 (CH₂Cl₂:MeOH (with NH₃ 7N)=70:30, free base).

¹H-NMR (CD₃OD, δ): 1.47 (t, 3H, J=6.0 Hz, CH₃), 3.78 (s, 6H, 2×OMe), 4.36 (t, 2H, J=6.0 Hz, OCH₂), 4.36 (s, 2H, CH₂), 4.41 (s, 2H, CH₂) 6.61 (s, 2H, 2×ArH), 7.67 (d, 1H, J=9.0 Hz, ArH), 7.79 (d, 1H, J=9.0 Hz, ArH), 8.09 (s, 1H, ArH), 9.52 (s, 1H, ArH).

¹³C-NMR (CD₃OD, δ): 15.1, 37.2, 56.6 (2×C), 66.9, 73.1, 107.1 (2×C), 116.1, 121.1, 124.2, 132.3, 134.5, 134.9, 136.7, 137.0, 145.0, 145.5, 154.0 (2×C), 175.5.

MS-ESI m/z (% rel. Int.): 412.0 ([MH]⁺, 100).

HPLC: Method A, XBridge™ column, detection UV 254 nm, RT=3.63 min, peak area 99.0%.

### Preparation of 2-(4-((7-ethoxy-8-hydroxyisoquinolin-4-yl)methyl)-2,6-dimethoxyphenoxy)acetonitrile hydrochloride 50

### 2-(4-((7-Ethoxy-8-hydroxyisoquinolin-4-yl)methyl)-2,6-dimethoxyphenoxy)acetonitrile hydrochloride 50

To a solution of 2-(4-((7-ethoxy-8-hydroxyisoquinolin-4-yl)methyl)-2,6-dimethoxyphenoxy)acetamide 49 free base (809 mg, 1.96 mmol, 1.0 eq) in anhydrous CH₂Cl₂ (40 mL) was slowly added pyridine (792 μL, 9.80 mmol, 5.0 eq) and trifluoroacetic anhydride (832 μL, 5.98 mmol, 3.1 eq) at RT under a nitrogen atmosphere. The reaction mixture was stirred at RT for 1 h. The solvent was evaporated and the residue was dissolved in EtOAc (200 mL) and a 1 M K₂CO₃ solution was added (20 mL). The separated organic layer was washed with brine (20 mL), dried over MgSO₄, filtered and evaporated. The obtained crude product was purified by column chromatography (SiO₂, eluent cyclohexane:EtOAc=100:0 to 60:40) to give after evaporation and drying 2-(4-((7-ethoxy-8-hydroxyisoquinolin-4-yl)methyl)-2,6-dimethoxyphenoxy)acetonitrile (122 mg). This solid was dissolved in MeOH (5 mL) and a 0.15 N HCl in MeOH (2.3 mL, 0.34 mmol, 1.1 eq) was slowly added and the reaction mixture was stirred at 4° C. for 15 min. After evaporation and drying, 2-(4-((7-ethoxy-8-hydroxyisoquinolin-4-yl)methyl)-2,6-dimethoxyphenoxy)acetonitrile hydrochloride 50 (127 mg, 15% yield) was obtained as a brown solid.

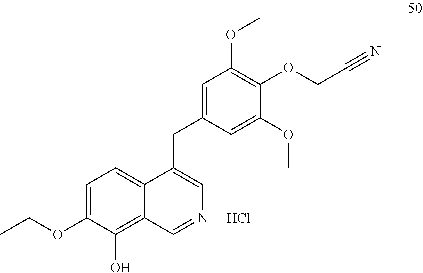 [SMILES: CCOc1ccc2c(Cc3cc(OC)c(OCC#N)c(OC)c3)cncc2c1O ; Cl]

MW: 430.88; Yield: 15%; Brown Solid, Mp (° C.): 227.3

¹H-NMR (CD₃OD, δ): 1.50 (t, 3H, J=7 Hz, CH₃), 3.80 (s, 6H, 2×CH₃), 4.33 (q, 2H, J=7 Hz, OCH₂), 4.50 (s, 2H, CH₂), 4.78 (s, 2H, CH₂C≡N), 6.65 (s, 2H, 2×ArH), 7.86 (d, 1H, J=9 Hz, ArH), 8.02 (d, 1H, J=9 Hz, ArH), 8.08 (s, 1H, ArH), 9.66 (s, 1H, ArH), 8.42 (s, 1H, ArH).

¹³C-NMR (CD₃OD, δ): 15.0, 37.0, 56.7, 58.2, 66.9, 107.3, 116.5, 117.3, 118.8, 120.5, 126.7, 128.4, 132.8, 135.1, 136.8, 137.7, 142.5, 146.4, 146.9, 154.9.

MS-ESI m/z (% rel. Int.): 395.1 ([MH]⁺, 100).

HPLC: Method A, XBridge™ column, detection UV 254 nm, RT=4.18 min, peak area 97%.

### Preparation of 4-(4-(benzylamino)-3,5-dimethoxybenzyl)-7-ethoxyisoquinolin-8-ol hydrochloride 51

### 4-Formyl-2,6-dimethoxyphenyl trifluoromethanesulfonate RBO 40040

To a solution of 4-hydroxy-3,5-dimethoxybenzaldehyde (1.0 g, 5.5 mmol) in DMF (20 mL) were added Et₃N (1.55 mL, 11.0 mmol) and N-phenyl-bis(trifluoromethanesulfonimide) (2.95 g, 8.25 mmol) and the mixture was stirred for 1 h at RT. The volatiles were evaporated and the crude product was diluted in Et₂O (50 mL). The organic layer was washed with water (3×20 mL), brine (10 mL), dried over Na₂SO₄, filtered and evaporated at 45° C. The obtained crude product was purified by column chromatography (SiO₂, eluent cyclohexane:CH₂Cl₂=1:1) to give, after evaporation and drying, 4-formyl-2,6-dimethoxyphenyl trifluoromethanesulfonate RBO 40040 (1.5 g, 87% yield) as an off-white solid.

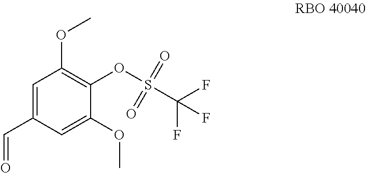 [SMILES: COc1cc(C=O)cc(OC)c1OS(=O)(=O)C(F)(F)F]

MW: 314.24; Yield: 87%; Off-white solid.

R_{f}: 0.25 (cyclohexane:CH₂Cl₂=1:1).

¹H-NMR (CDCl₃, δ): 3.98 (s, 6H, 2×OCH₃), 7.17 (s, 2H, 2×ArH), 9.94 (s, 1H, CHO).

MS-ESI m/z (% rel. Int.): 315.2 ([MH]⁺, 10).

HPLC: Method (5 min), XBridge™ column, detection UV 254 nm, RT=3.31 min, peak area 99.0%.

### 4-(Benzylamino)-3,5-dimethoxybenzaldehyde RBO 40056

To a solution of 4-formyl-2,6-dimethoxyphenyl trifluoromethanesulfonate RBO 40040 (200 mg, 0.64 mmol) in dioxane (15 mL) in a 20 mL microwave vial was added benzylamine (140 μL, 1.28 mmol), cesium carbonate (293 mg, 0.90 mmol), BINAP (18.7 mg, 0.03 mmol, 4.5% mol) and palladium acetate (4.49 mg, 0.02 mmol, 3% mol). The vial was sealed and the mixture was heated for 20 min at 140° C. under microwave irradiation. After filtration through celite, a 1 N HCl solution (20 mL) and EtOAc (20 mL) were added and the solution was stirred for 1 h at RT (the imine disappearance was monitoring by HPLC). The mixture was neutralized with a 10% NaHCO₃ aqueous solution until pH=7 (10 mL) and the title compound was extracted with EtOAc (3×40 mL). The organic layer was washed with water (3×30 mL), brine (30 mL), dried over Na₂SO₄, filtered and evaporated. The obtained crude product was purified by column chromatography (SiO₂, eluent CH₂Cl₂) to give, after evaporation and drying, 4-benzylamino-3,5-dimethoxy-benzaldhehyde RBO 40056 (118 mg, 68% yield) as a yellow oil.

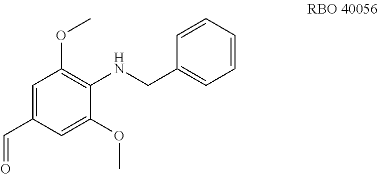 [SMILES: COc1cc(C=O)cc(OC)c1NCc1ccccc1]

MW: 271.32; Yield: 68%; Yellow oil.

R_{f}: 0.2 (CH₂Cl₂).

¹H-NMR (CDCl₃, δ): 3.86 (s, 6H, 2×OMe), 4.70 (5, 2H, CH₂), 7.06 (s, 2H, 2×ArH), 7.24-7.32 (m, 5H, 5×ArH), 9.72 (5, 1H, CHO).

MS-ESI m/z (% rel. Int.): 272.1 ([MH]⁺, 30) 244 (100).

HPLC: Method (5 min), XBridge™ column, detection UV 254 nm, RT=2.92 min, peak area 96.0%.

### 4-(4-(Benzylamino)-3,5-dimethoxybenzyl)-7-ethoxyisoquinolin-8-ol hydrochloride 51

In a 20 mL microwave vial, 4-benzylamino-3,5-dimethoxybenzaldhehyde RBO 40056 (118 mg, 0.44 mmol) and 2-((2,2-diethoxyethylamino)methyl)-6-ethoxyphenol SAO 33014 (123 mg, 0.44 mmol) were dissolved in EtOH (5 mL) and a 37% HCl solution (5 mL) was added. The reaction mixture was heated at 100° C. for 20 min under microwave irradiation, cooled to RT and concentrated. The crude product was neutralized with a 10% NaHCO₃ aqueous solution and extracted with CH₂Cl₂ (3×20 mL). The combined organic layers were washed with water (3×20 mL), brine (20 mL), dried over Na₂SO₄, filtered and evaporated to give a yellow solid. This crude product was purified by column chromatography (SiO₂, eluent CH₂Cl₂:EtOAc=1:1) to give, after evaporation and drying, 4-(4-benzylamino-3,5-dimethoxy-benzyl)-7-ethoxyisoquinolin-8-ol (266 mg). The above compound was dissolved in MeOH (5 mL) and a 0.49 N HCl solution in MeOH (2.5 mL, 1.2 mmol) was slowly added. The reaction mixture was stirred at 4° C. for 15 min. After evaporation and drying under P₂O₅, 4-(4-benzylamino-3,5-dimethoxybenzyl)-7-ethoxy-isoquinolin-8-ol hydrochloride 51 (98.5 mg, 44% yield) was obtained as a yellow solid.

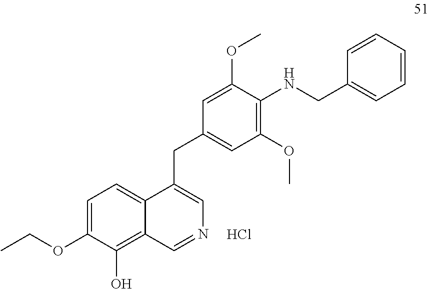 [SMILES: CCOc1ccc2c(Cc3cc(OC)c(NCc4ccccc4)c(OC)c3)cncc2c1O ; Cl]

MW: 480.98; Yield: 44%; Yellow solid; Mp (° C.): 213.0

R_{f}: 0.35 (CH₂Cl₂:EtOAc=1:1, free base).

¹H-NMR (CD₃OD, δ): 1.52 (t, 3H, J=6.0 Hz, CH₃), 3.78 (s, 6H, 2×OMe), 4.37 (q, 2H, J=6.0 Hz, CH₂CH₃), 4.44 (s, 2H, CH₂), 4.58 (s, 2H, CH₂), 6.75 (s, 2H, 2×ArH), 7.35 (m, 5H, 5×ArH), 7.83 (d, 2H, J=9.0 Hz, 2×ArH), 8.07 (d, 2H, J=9.0 Hz, 2×ArH), 9.70 (s, 1H, ArH).

¹³C-NMR (CD₃OD, δ): 15.1, 37.2, 54.4, 57.2 (2×C), 67.0, 106.6 (2×C), 110.8, 116.5, 120.5, 126.8, 128.6, 129.6 (2×C), 130.8, 131.7, 131.9 (2×C), 132.6, 136.8, 142.9, 143.7, 146.5, 147.0, 155.2 (2×C).

MS-ESI m/z (% rel. Int.): 445.1 ([MH]⁺, 100).

HPLC: Method A, XBridge™ column, detection UV 254 nm, RT=3.64 min, peak area 99.0%.

### Preparation of 4-(3,5-dimethoxy-4-(phenylamino)benzyl)-7-ethoxyisoquinolin-8-ol hydrochloride 52

### 3,5-Dimethoxy-4-(phenylamino)benzaldehyde RBO 40078

To a solution of 4-formyl-2,6-dimethoxyphenyl trifluoromethanesulfonate RBO 40040 (500 mg, 1.60 mmol) in dioxane (16 mL) in a 20 mL microwave vial was added aniline (292 μL, 3.20 mmol), Cs₂CO₃ (730 mg, 2.24 mmol), BINAP (44 mg, 0.07 mmol 4.5% mol) and palladium acetate (11 mg, 0.05 mmol, 3% mol). The vial was sealed and the mixture was heated for 20 min at 140° C. under microwave irradiation. After filtration through celite, the solvent was removed and the residue was taken back in CH₂Cl₂ (30 mL), washed with a 1 N HCl aq. solution (3×30 mL), brine (30 mL), dried over Na₂SO₄, filtered and evaporated. The obtained crude product was purified by column chromatography (SiO₂, eluent CH₂Cl₂) to give, after evaporation and drying, 3,5-dimethoxy-4-(phenylamino)benzaldehyde RBO 40078 (344 mg, 83% yield) as orange solid.

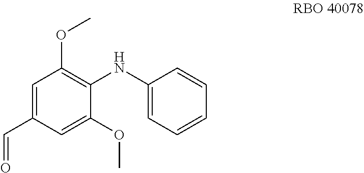 [SMILES: COc1cc(C=O)cc(OC)c1Nc1ccccc1]

MW: 257.29; Yield: 83%; Orange solid.

R_{f}: 0.4 (CH₂Cl₂).

¹H-NMR (CDCl₃, δ): 3.88 (s, 6H, 2×OMe), 6.19 (broad s, 1H, NH), 6.77 (s, 1H, ArH), 6.80 (s, 1H, ArH), 6.93 (m, 1H, ArH), 7.17 (s, 2H, 2×ArH), 7.20-7.25 (m, 2H, ArH), 9.88 (s, 1H, CHO).

MS-ESI m/z (% rel. Int.): 258.1 ([MH]⁺, 100).

HPLC: Method (5 min), XBridge™ column, detection UV 254 nm, RT=3.12 min, peak area 99.0%.

### 4-(3,5-Dimethoxy-4-(phenylamino)benzyl)-7-ethoxyisoquinolin-8-ol hydrochloride 52

In a 20 mL microwave vial, 3,5-dimethoxy-4-(phenylamino)benzaldehyde RBO 40078 (220 mg, 0.77 mmol) and 2-((2,2-diethoxyethylamino)methyl)-6-ethoxyphenol SAO 33014 (200 mg, 0.77 mmol) were dissolved in EtOH (5 mL) and a 37% HCl solution (5 mL) was added. The reaction mixture was heated at 100° C. for 20 min under microwave irradiation, then cooled at RT and concentrated. The residue was neutralized with a 10% Na₂CO₃ solution (10 mL) and extracted with EtOAc (3×20 mL). The combined organic layers were washed with water (3×20 mL), brine (20 mL), dried over Na₂SO₄, filtered and evaporated to give a yellow solid. This solid was purified by column chromatography (SiO₂, eluent CH₂Cl₂:EtOAc=1:1) to give, after evaporation and drying, 4-(3,5-dimethoxy-4-(phenylamino)benzyl)-7-ethoxyisoquinolin-8-ol (266 mg). The above compound was dissolved in MeOH (5 mL) and a 0.49 N HCl solution in MeOH (2.5 mL, 1.2 mmol) was slowly added. The reaction mixture was stirred at 4° C. for 15 min. After evaporation and drying under P₂O₅, 4-(3,5-dimethoxy-4-(phenylamino)benzyl)-7-ethoxyisoquinolin-8-ol hydrochloride 52 (10 mg, 2.6% yield) was obtained as a yellow solid.

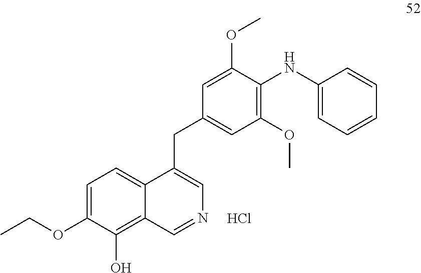 [SMILES: CCOc1ccc2c(Cc3cc(OC)c(Nc4ccccc4)c(OC)c3)cncc2c1O ; Cl]

MW: 466.97; Yield: 2.6%; Yellow Solid.

R_{f}: 0.45 (CH₂Cl₂:EtOAc=1:1, free base).

¹H-NMR (CD₃OD, δ): 1.50 (t, 3H, J=6.0 Hz, CH₃), 3.84 (s, 6H, 2×OMe), 4.35 (q, 2H, J=6.0 Hz, CH₂CH₃), 4.59 (s, 2H, CH₂), 6.79 (s, 2H, 2×ArH), 7.14 (m, 3H, 3×ArH), 7.33 (m, 2H, 2×ArH), 7.89 (d, 1H J=6.0 Hz, ArH), 8.04 (d, 1H J=6.0 Hz, ArH). 8.15 (s, 1H, ArH), 9.68 (s, 1H, ArH).

¹³C-NMR (CD₃OD, δ): 15.0, 37.2, 54.8, 57.0 (2×C), 66.9, 106.9 (2×C), 116.5, 120.5 (3×C), 126.2, 126.8, 128.6, 130.4, 130.5 (2×C), 132.8, 137.2, 140.7, 142.2, 142.7, 146.5, 147.0, 155.6.

MS-ESI m/z (% rel. Int.): 431 ([MH]⁺, 100).

HPLC: Method A, XBridge™ column, detection UV 254 nm, RT=4.85 min, peak area 96.0%.

### Preparation of 4-((2,6-dimethoxy-[1,1′-biphenyl]-4-yl)methyl)-7-ethoxyisoquinolin-8-ol hydrochloride 53

### 2,6-Dimethoxy-[1,1′-biphenyl]-4-carbaldehyde RBO 40134

To a solution of 4-formyl-2,6-dimethoxyphenyl trifluoromethanesulfonate RBO 40040 (1.0 g, 3.18 mmol) in DMF (12.5 mL) and water (2.5 mL) in a 30 mL round bottom flask, was successively added phenyl boronic acid (441 mg, 3.62 mmol), palladium tetrakis (190 mg, 0.16 mmol), and Na₂CO₃ (695 mg, 6.56 mmol) under a nitrogen atmosphere. The resulting mixture was stirred for 2.5 h at 80° C. After cooling to RT, the mixture was filtered through celite, H₂O (50 mL) and the resulting solution was extracted with EtOAc (3×50 mL). The combined organic layers were washed with H₂O (3×30 mL), brine (30 mL), dried over Na₂SO₄, filtered and concentrated to dryness to give a brown oil. This oil was purified by column chromatography (SiO₂, eluent cyclohexane:EtOAc=90:10) to give, after evaporation and drying, 2,6-dimethoxy-[1,1′-biphenyl]-4-carbaldehyde RBO 40134 (405 mg, 53% yield) as a white solid.

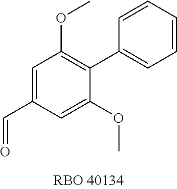 [SMILES: COc1cc(C=O)cc(OC)c1-c1ccccc1]

MW: 242.28; Yield: 53%; White solid.

R_{f}: 0.20 (cyclohexane:EtOAc=9:1).

¹H-NMR (CDCl₃, δ): 3.83 (s, 6H, 2×OMe), 7.15-7.25 (m, 2H, 2×ArH), 7.30-7.50 (m, 5H, 5×ArH), 9.98 (s, 1H, CHO).

MS-ESI m/z (% rel. Int.): 243.0 ([MH]⁺, 25), 215.0 (100).

HPLC: Method A, XBridge™ column, detection UV 254 nm, RT=5.70 min, peak area 99.0%.

### 4-((2,6-Dimethoxy-[1,1′-biphenyl]-4-yl)methyl)-7-ethoxyisoquinolin-8-ol hydrochloride 53

In an ace pressure tube (20 mL, Aldrich), 2-((2,2-diethoxyethylamino)methyl)-6-ethoxyphenol SAO 33014 (293 mg, 1.03 mmol) and 2,6-dimethoxy-[1,1′-biphenyl]-4-carbaldehyde RBO 40134 (250 mg, 1.03 mmol) were dissolved in EtOH:37% HCl solution=1:1 (4 mL) and the reaction mixture was stirred at 110° C. for 10 min then cooled to RT. The volatiles were removed under vacuum and EtOAc (120 mL) was added and the resulting solution was neutralized with a 10% NaHCO₃ aq. solution (10 mL). The aqueous layer was further extracted with EtOAc (3×30 mL). The combined organic layers were washed with H₂O (3×30 mL), brine (30 mL), dried over Na₂SO₄, filtered and evaporated to give yellow solid. This solid was purified by column chromatography (SiO₂, eluent CH₂Cl₂:EtOAc=100:0 to 50:50) to give, after evaporation and drying, 4-((2,6-dimethoxy-[1,1′-biphenyl]-4-yl)methyl)-7-ethoxyisoquinolin-8-ol (70 mg). This solid was dissolved in MeOH (2 mL) and a 0.49 N HCl solution in MeOH (344 μL, 0.17 mmol) was slowly added and the reaction mixture was stirred at 4° C. for 15 min. After evaporation and drying under P₂O₅, 4-((2,6-dimethoxy-[1,1′-biphenyl]-4-yl)methyl)-7-ethoxyisoquinolin-8-ol hydrochloride 53 (74 mg, 16% yield) was obtained as a yellow solid.

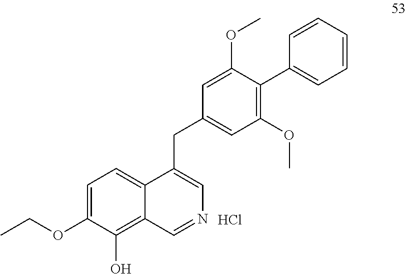 [SMILES: CCOc1ccc2c(Cc3cc(OC)c(-c4ccccc4)c(OC)c3)cncc2c1O ; Cl]

MW: 415.49; Yield: 16%; Yellow Solid; Mp (° C.): 230.4

R_{f}: 0.50 (CH₂Cl₂:EtOAc=50:50, free base).

¹H-NMR (CD₃OD, δ): 1.50 (t, 3H, J=6 Hz, CH₃), 3.62 (s, 6H, 2×OMe), 4.35 (q, 2H, J=6 Hz OCH₂), 4.57 (s, 2H, CH₂), 6.66 (s, 2H, 2×ArH), 7.14-7.35 (m, 5H, 5×ArH), 7.92 (d, 1H, J=9 Hz, ArH), 8.05 (d, 1H, J=9 Hz, ArH), 8.14 (s, 1H, ArH), 9.67 (s, 1H, ArH).

¹³C-NMR (CD₃OD, δ): 15.0, 37.3, 56.3 (2×C), 66.9, 106.4 (2×C), 116.6, 119.9, 120.5, 126.8, 127.5, 128.4 (2×C), 132.0 (2×C), 132.9, 135.5, 137.9, 140.0, 140.1, 142.5, 146.4, 146.9, 159.4 (2×C).

MS-ESI m/z (% rel. Int.): 416.0 ([MH]⁺, 100).

HPLC: Method A, XBridge™ column, detection UV 254 nm, RT=4.93 min, peak area 95.0%.

### Preparation of (7-methoxyisoquinolin-4-yl)(3,4,5-trimethoxy-2-nitrophenyl)methanone hydrochloride 54

### (7-Methoxyisoquinolin-4-yl)(3,4,5-trimethoxy-2-nitrophenyl)methanone hydrochloride 54

(7-Methoxyisoquinolin-4-yl)(3,4,5-trimethoxyphenyl)methanone (28 free base, 90.6 mg, 0.26 mmol, 1.0 eq) was dissolved in CH₃CN (1.5 mL) and CH₂Cl₂ (2 mL). Nitronium tetrafluoroborate (56.5 mg, 0.41 mmol, 1.6 eq) was added. The reaction mixture was stirred at RT under nitrogen atmosphere for 36 h, cooled to 4° C. and concentrated. The crude product was transformed into its freebase with a 1 M K₂CO₃ aqueous solution (3 mL) and the resulting solution was extracted with CH₂Cl₂ (2×200 mL). The combined organic layers were washed with brine (50 mL), dried over MgSO₄, filtered and evaporated to give a yellow oil. This crude product was purified by column chromatography (SiO₂, eluent cyclohexane:EtOAc=50:50 to 0:100) to give, after evaporation and drying, (7-methoxyisoquinolin-4-yl)(3,4,5-trimethoxy-2-nitrophenyl)methanone (30.7 mg). This solid was dissolved in MeOH (2 mL) and a 1.34 N HCl solution in MeOH (60 μL, 0.081 mmol, 1.05 eq) was slowly added. The reaction mixture was stirred at 4° C. for 15 min. After evaporation and drying under P₂O₅, (7-methoxyisoquinolin-4-yl)(3,4,5-trimethoxy-2-nitrophenyl)methanone hydrochloride 54 (31.2 mg, 31% yield) was obtained as a yellow solid.

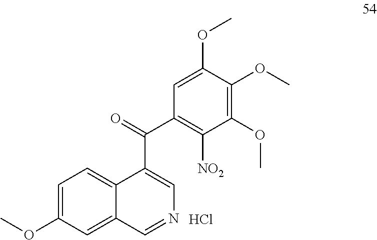

MW: 434.83; Yield: 31%; Yellow Solid; Mp (° C.): 176.9

R_{f}: 0.15 (cyclohexane:EtOAc=5:5) free base.

¹H-NMR (CD₃OD, δ): 3.90 (s, 3H, OMe), 4.02 (s, 6H, 2×OMe), 4.10 (s, 3H, OMe), 7.13 (s, 1H, ArH), 7.92 (dd, 1H, J=9.1 Hz, J=1.9 Hz, ArH), 7.98 (s, 1H, ArH), 8.56 (d, 1H, J=9.3 Hz, ArH), 8.64 (s, 1H, ArH), 9.80 (s, 1H, ArH).

¹³C-NMR (CD₃OD, δ): 57.0, 57.5, 61.9, 63.3 (4×C), 109.4, 110.7, 128.2, 128.7, 131.8, 132.6, 132.7, 132.9, 133.7, 140.0, 148.0, 148.4, 150.2, 157.1, 162.6, 190.6.

MS-ESI m/z (% rel. Int.): 399.2 ([MH]⁺, 100).

HPLC: Method A, XBridge™ column, detection UV 254 nm, RT=5.18 min, peak area 99.9%.

### Preparation of (8-(benzyloxy)-7-ethoxyisoquinolin-4-yl)(4-ethoxy-3,5-dimethoxyphenyl)methanone 55

### 8-(Benzyloxy)-7-ethoxy-4-(4-ethoxy-3,5-dimethoxybenzyl)isoquinoline LPO 37138C

7-Ethoxy-4-(4-ethoxy-3,5-dimethoxybenzyl)isoquinolin-8-ol hydrochloride 14 (1.0 g, 2.61 mmol) and anhydrous Et₃N (401 μL, 2.87 mmol) were dissolved in dry DMF (9 mL). Cesium carbonate (1.3 g, 3.91 mmol) was added and the reaction mixture was stirred at RT under a nitrogen atmosphere for 5 min before benzylbromide (343 μL, 2.87 mmol) was slowly added. The reaction mixture was stirred at 90° C. for 3 h and evaporated at 50° C. The crude product was diluted with EtOAc (500 mL) and the resulting solution was washed with water (150 mL), brine (50 mL), dried over MgSO₄, filtered and concentrated at 40° C. The crude product was purified by column chromatography (SiO₂, eluent from cyclohexane:EtOAc=100:0 to 70:30), to give, after evaporation and drying, 8-(benzyloxy)-7-ethoxy-4-(4-ethoxy-3,5-dimethoxybenzyl)isoquinoline LPO 37138C (392 mg, 32% yield) as yellow oil.

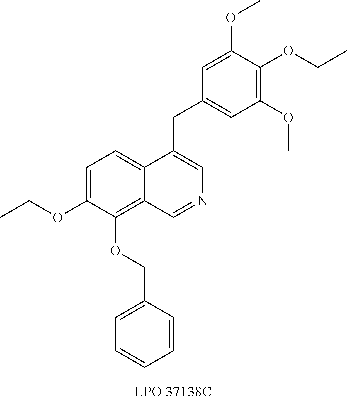 [SMILES: CCOc1ccc2c(Cc3cc(OC)c(OCC)c(OC)c3)cncc2c1OCc1ccccc1]

MW: 473.56; Yield: 32%; Yellow Oil.

R_{f}: 0.25 (cyclohexane:EtOAc=70:30, free base).

¹H-NMR (CDCl₃, δ): 1.33 (t, 3H, J=7.0 Hz, CH₃), 1.50 (t, 3H, J=7.0 Hz, CH₃), 3.73 (s, 6H, 2×OMe), 4.01 (q, 2H, J=7.1 Hz, CH₂), 4.20-4.26 (m, 4H, CH₂ & OCH₂), 5.27 (s, 2H, CH₂), 6.38 (s, 2H, 2×ArH), 7.33-7.54 (m, 6H, 6×ArH), 7.64 (d, 1H, J=9.2 Hz, ArH), 8.25 (s, 1H, ArH), 9.48 (s, 1H, ArH).

### (8-(Benzyloxy)-7-ethoxyisoquinolin-4-yl)(4-ethoxy-3,5-dimethoxyphenyl)methanone 55

8-(Benzyloxy)-7-ethoxy-4-(4-ethoxy-3,5-dimethoxybenzyl)isoquinoline LPO 37138C (217 mg, 0.46 mmol) was dissolved in a CH₃CN:H₂O mixture (14 mL:7 mL) under a nitrogen atmosphere and N-hydroxyphtalimide (15 mg, 0.092 mmol) and NaO₂Cl (58 mg, 0.643 mmol) were added and the reaction mixture was stirred at 50° C. for 3.5 h. After cooling to RT, the solution was diluted with Et₂O (200 mL) and a 10% sodium sulfite aqueous solution (3 mL). The separated organic layer was washed with brine (20 mL), dried over MgSO₄, filtered and evaporated to dryness to give a brown oil (190 mg). This crude oil was purified by column chromatography (SiO₂, eluent cyclohexane:EtOAc=75:25). After evaporation and drying under P₂O₅, (8-(benzyloxy)-7-ethoxyisoquinolin-4-yl)(4-ethoxy-3,5-dimethoxyphenyl)methanone 55 (63 mg, 28% yield) was obtained as a yellow solid.

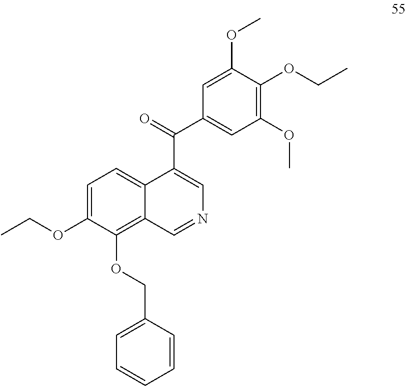 [SMILES: CCOc1ccc2c(C(=O)c3cc(OC)c(OCC)c(OC)c3)cncc2c1OCc1ccccc1]

MW: 487.54; Yield: 28%; Yellow solid, Mp (° C.): 186.1

¹H-NMR (CDCl₃, δ): 1.26-1.43 (m, 3H, CH₂CH₃), 1.51-1.59 (m, 3H, CH₂CH₃), 3.83 (s, 6H, 2×OMe), 4.14-4.32 (m, 4H, 2×CH₂CH₃), 5.32 (s, 2H, CH₂), 7.14 (s, 2H, 2×ArH), 7.35-7.43 (m, 3H, 3×ArH), 7.52-7.58 (m, 3H, 3×ArH), 7.87 (d, 1H J=4.02 Hz, ArH), 8.51 (s, 1H, ArH), 9.65 (s, 1H, ArH).

MS-ESI m/z (% rel. Int.): 488.3 ([MH]⁺, 100).

HPLC: Method A, XBridge™ column, detection UV 254 nm, RT=5.68 min, peak area 99.9%.

### Preparation of (4-ethoxy-3,5-dimethoxyphenyl)(7-ethoxy-8-hydroxyisoquinolin-4-yl)methanone hydrochloride 56

### (4-Ethoxy-3,5-dimethoxyphenyl)(7-ethoxy-8-hydroxyisoquinolin-4-yl)methanone hydrochloride 56

(8-(Benzyloxy)-7-ethoxyisoquinolin-4-yl)(4-ethoxy-3,5-dimethoxyphenyl)methanone 55 (117.5 mg, 0.24 mmol, 1.0 eq) was dissolved in EtOH (5 mL), a 37% HCl solution (5 mL) was added and the reaction mixture was stirred at 50° C. for 6 h, then cooled to 4° C. and concentrated. The obtained crude product was transformed into its freebase with a 1 N K₂CO₃ aq. solution (20 mL) and extracted with CH₂Cl₂ (200 mL). The separated organic layer was washed with brine (30 mL), dried over MgSO₄, filtered and evaporated to give a yellow oil. This crude oil was purified by column chromatography (SiO₂, eluent cyclohexane:EtOAc=9:1 to 6:4) to give, after evaporation and drying, (4-ethoxy-3,5-dimethoxyphenyl)(7-ethoxy-8-hydroxyisoquinolin-4-yl)methanone (67.3 mg) as a yellow oil. This crude oil was dissolved in MeOH (2 mL) and a 1.34 N HCl solution in MeOH (133 μL, 0.18 mmol, 1.05 eq) was slowly added. The reaction mixture was stirred at 4° C. for 15 min. After evaporation and drying under P₂O₅, (4-ethoxy-3,5-dimethoxyphenyl)(7-ethoxy-8-hydroxyisoquinolin-4-yl)methanone hydrochloride 56 (71.9 mg, 69% yield) was obtained as a orange solid.

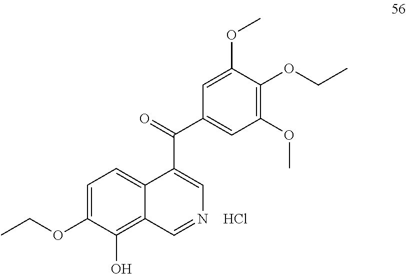 [SMILES: CCOc1ccc2c(C(=O)c3cc(OC)c(OCC)c(OC)c3)cncc2c1O ; Cl]

MW: 433.88; Yield: 69%; Orange Solid; Mp (° C.): 236.2

R_{f}: 0.25 (cyclohexane:EtOAc=6:4, free base).

¹H-NMR (CD₃OD, δ): 1.32 (t, 3H, J=7.0 Hz, OCH₂CH₃), 1.51 (t, 3H, J=7.0 Hz, OCH₂CH₃), 3.80 (s, 6H, 2×OMe), 4.14 (q, 2H, J=7.1 Hz, OCH₂CH₃), 4.36 (q, 2H, J=7.0 Hz, OCH₂CH₃), 7.19 (s, 2H, 2×ArH), 7.60 (d, 1H, J=9.0 Hz, ArH), 8.03 (d, 1H, J=9.1 Hz, ArH), 8.44 (s, 1H, ArH), 9.88 (s, 1H, ArH).

¹³C-NMR (CD₃OD, δ): 15.0, 15.8, 56.9 (2×C), 67.0, 70.2, 109.2 (2×C), 117.5, 120.9, 127.5, 129.3, 130.8, 132.9, 135.3, 144.5, 145.7, 146.9, 147.1, 155.1 (2×C), 192.6.

MS-ESI m/z (% rel. Int.): 398.2 ([MH]⁺, 100).

HPLC: Method A, XBridge™ column, detection UV 254 nm, RT=4.43 min, peak area 99.9%.

### Preparation of 7-ethoxy-4-(4-ethoxy-3,5-dimethoxybenzoyl)isoquinolin-8-yl sulfamate 57

### Sulfamoyl chloride LPO 43022

In anhydrous conditions, acid formic (1.0 mL, 35.3 mmol) was added dropwise to chlorosulfonyl isocyanate (5.0 g, 37.1 mmol) at 4° C. in an ace pressure tube. The reaction mixture was stirred from 4° C. to RT for 1 h to give a white solid. Anhydrous toluene (12 mL) was added and the reaction was stirred overnight at RT under a nitrogen atmosphere. The reaction mixture was evaporated to give sulfamoyl chloride LPO 43022 (1.88 g, 44% yield) as a green solid.

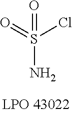

MW: 115.54; Yield: 44%; Hygroscopic green solid.

### 7-Ethoxy-4-(4-ethoxy-3,5-dimethoxybenzoyl)isoquinolin-8-yl sulfamate 57

(4-Ethoxy-3,5-dimethoxyphenyl)(7-ethoxy-8-hydroxyisoquinolin-4-yl)methanone hydrochloride 56 (430 mg, 1.08 mmol) was dissolved in anhydrous CH₂Cl₂ (12 mL). NaH (60% in mineral oil dispersion, 52 mg, 1.30 mmol), anhydrous Et₃N (272 μL, 1.95 mmol) were slowly added at 4° C. under a nitrogen atmosphere. Sulfamoyl chloride LPO 43022 (375 mg, 3.25 mmol) was dissolved in anhydrous CH₂Cl₂ (5 mL) and was slowly added dropwise at 4° C. The reaction mixture was stirred at 4° C. to RT for 3 h. Sulfamoyl chloride LPO 43022 (187.5 mg, 1.62 mmol) and anhydrous Et₃N (136 μL, 0.97 mmol) were slowly added. The reaction mixture was stirred at RT overnight. Sulfamoyl chloride LPO 43022 (125.0 mg, 1.08 mmol) and anhydrous Et₃N (90.8 μL, 0.65 mmol) were slowly added. The reaction mixture was stirred at RT for 5 h. CH₂Cl₂ (500 mL) and K₂CO₃ (40 mL) were added. The separated organic layer was washed with brine (20 mL), dried over MgSO₄, filtered and evaporated to give a brown oil. The crude oil was purified by column chromatography (SiO₂, eluent CH₂Cl₂:MeOH=100:0 to 98:2) to give, after evaporation and drying, 7-ethoxy-4-(4-ethoxy-3,5-dimethoxybenzoyl)isoquinolin-8-yl sulfamate 57 (68.2 mg, 13% yield) as a white solid.

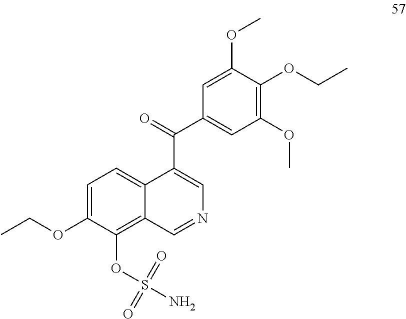 [SMILES: CCOc1ccc2c(C(=O)c3cc(OC)c(OCC)c(OC)c3)cncc2c1OS(N)(=O)=O]

MW: 476.5; Yield: 13%; White solid; Mp (° C.): 207.1

R_{f}: 0.3 (CH₂Cl₂:MeOH=98:2).

¹H-NMR (CDCl₃, δ): 1.40 (t, 3H, J=7.1 Hz, CH₃), 1.53 (t, 3H, J=7.0 Hz, CH₃), 3.85 (s, 6H, 2×OMe), 4.19 (q, 2H, J=7.1 Hz, OCH₂), 4.35 (q, 2H, J=7.0 Hz, OCH₂), 7.14 (s, 2H, 2×ArH), 7.67 (d, 1H, J=9.4 Hz, ArH), 8.08 (d, 1H, J=9.4 Hz, ArH), 8.51 (s, 1H, ArH), 9.67 (s, 1H, ArH).

¹³C-NMR (CDCl₃, δ): 14.8, 15.6, 56.5 (2×C), 66.2, 69.6, 108.2 (2×C), 121.9, 124.8, 125.0, 129.2, 129.3, 132.7, 134.3, 141.7, 142.7, 149.9 (2×C), 153.7 (2×C), 194.9.

MS-ESI m/z (% rel. Int.): 477.1 ([MH]⁺, 100).

HPLC: Method A, XBridge™ column, detection UV 254 nm, RT=4.94 min, peak area 99.0%.

### Preparation of (8-amino-7-ethoxyisoquinolin-4-yl)(4-ethoxy-3,5-dimethoxyphenyl)methanone hydrochloride 58

### N-(Diphenylmethylene)-7-ethoxy-4-(4-ethoxy-3,5-dimethoxybenzyl)isoquinolin-8-amine LPO 37146C

A mixture of (±) BINAP (266 mg, 0.43 mmol), Pd₂(dba)₃ (117 mg, 0.13 mmol), dry Cs₂CO₃ (1.39 g, 4.27 mmol), 4-(4-ethoxy-3,5-dimethoxybenzyl)-7-ethoxyisoquinolin-8-yl trifluoromethanesulfonate LPO 37002C (1.10 g, 2.13 mmol) and benzophenone imine (789 μL, 4.69 mmol) in dry toluene (45 mL) in an ace pressure tube equipped with a magnetic stirrer was stirred for 4.5 h at 160° C. After cooling to RT, the reaction mixture was diluted with EtOAc (250 mL) and filtered through celite. The filtrate was concentrated at 40° C. under vacuum. Purification by column chromatography (SiO₂, eluent cyclohexane:EtOAc=70:30) gave, after evaporation and drying N-(diphenylmethylene)-7-ethoxy-4-(4-ethoxy-3,5-dimethoxybenzyl)isoquinolin-8-amine LPO 37146C (1.0 g, 86% yield) as a yellow solid.

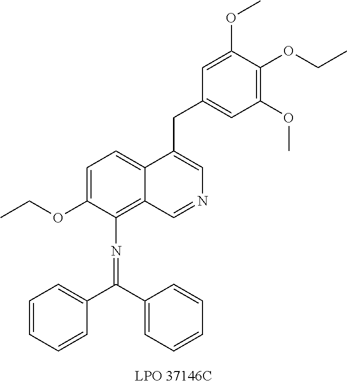 [SMILES: CCOc1ccc2c(Cc3cc(OC)c(OCC)c(OC)c3)cncc2c1N=C(c1ccccc1)c1ccccc1]

MW: 546.66; Yield: 86%; Yellow solid.

R_{f}: 0.15 (cyclohexane:EtOAc=70:30).

¹H-NMR (CDCl₃, δ): 1.32 (q, 6H, J=7.1 Hz, 2×CH₂CH₃), 3.69 (s, 6H, 2×OMe), 4.01 (q, 2H, J=7.1 Hz, 2×OCH₂), 4.25 (s, 2H, CH₂), 6.31 (s, 2H, 2×ArH), 7.09-7.22 (m, 6H, 6×ArH), 7.45-7.54 (m, 4H, 4×ArH), 7.89 (d, 2H, J=7.2 Hz, 2×ArH), 8.23 (s, 1H, ArH), 9.26 (s, 1H, ArH).

¹³C-NMR (CDCl₃, δ): 15.1, 15.5, 36.6, 56.0 (2×C), 64.7, 68.9, 105.5 (2×C), 119.4, 119.6, 122.4, 127.4 (2×C), 128.0 (2×C), 128.3 (2×C), 128.7, 128.8, 129.6 (2×C), 130.1, 131.1, 135.3, 135.4, 136.3, 136.9, 138.9, 141.9, 142.7, 148.2, 153.5 (2×C), 171.8.

MS-ESI m/z (rel. int.): 547.3 ([MH]⁺, 100).

### (18-Amino-7-ethoxyisoquinolin-4-yl)(4-ethoxy-3,5-dimethoxyphenyl)methanone hydrochloride 58

A mixture of N-(diphenylmethylene)-7-ethoxy-4-(4-ethoxy-3,5-dimethoxybenzyl)isoquinolin-8-amine LPO 37146C (1.00 g, 1.83 mmol), N-hydroxyphthalimide (298 mg, 1.83 mmol) and NaClO₂ (80% pure, 289 mg, 2.56 mmol) in a mixture CH₃CN:H₂O=2:1 (105 mL) in a round-bottomed flask with a magnetic stirrer was stirred for 3.5 h at 50° C. NaClO₂ (80% pure, 124 mg, 1.10 mmol) was added and the reaction mixture was heated at 70° C. for another 30 min. After cooling to RT, the mixture was diluted with Et₂O (200 mL), washed with a 10% sodium sulfite aq. solution (15 mL), brine (15 mL), dried over MgSO₄, filtered and concentrated at 40° C. The residue was then taken up in a mixture of 1 N aq. HCl:THF=1:1 (75 mL) and the resulting solution was stirred at RT for 30 min after which THF was removed at 40° C. The residue was neutralized with a saturated aqueous NaHCO₃ (20 mL) before to be extracted with CH₂Cl₂ (2×300 mL) and the combined organic layers were washed with brine (20 mL), dried over MgSO₄, filtered and concentrated. Purification by column chromatography (SiO₂, eluent from cyclohexane:EtOAc=100:0 to 0:100) gave, after evaporation and drying, crude (8-amino-7-ethoxyisoquinolin-4-yl)(4-ethoxy-3,5-dimethoxyphenyl)methanone as an orange solid (344 mg). This solid was dissolved in MeOH (5 mL) in a 50 mL round-bottomed flask equipped with a magnetic stirrer and the solution was cooled to 0° C. in an ice bath before adding a 1.34 N HCl solution in MeOH (1.33 mL). The solution was stirred for 15 min at 4° C. before to be evaporated to dryness to give (8-amino-7-ethoxyisoquinolin-4-yl)(4-ethoxy-3,5-dimethoxyphenyl)methanone hydrochloride 58 (345 mg, 47% yield) as a red solid.

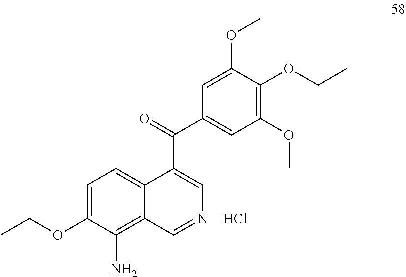 [SMILES: CCOc1ccc2c(C(=O)c3cc(OC)c(OCC)c(OC)c3)cncc2c1N ; Cl]

MW: 432.90; Yield: 47%; Red solid; Mp (° C.): 258.5

R_{f}: 0.15 (cyclohexane:EtOAc=5:5, free base).

¹H-NMR (CD₃OD, δ): 1.33 (t, 3H, J=6.8 Hz, CH₂CH₃), 1.51 (t, 3H, J=6.7 Hz, CH₂CH₃), 3.79 (s, 6H, 2×OCH₃), 4.12 (q, 2H, J=6.5 Hz, CH₂CH₃), 4.30 (q, 2H, J=6.7 Hz, CH₂CH₃), 7.18 (s, 2H, 2×ArH), 7.24 (d, 1H, J=9.0 Hz), 7.80 (d, 1H, J=8.5 Hz), 8.24 (s, 1H, ArH), 9.84 (s, 1H, ArH).

¹³C-NMR (CD₃OD, δ): 15.1, 15.8, 56.9 (2×C), 66.4, 70.2, 109.1 (2×C), 113.0, 116.8, 124.4, 127.7, 130.0, 132.9, 135.2, 141.0, 144.4, 145.4, 145.6, 155.1 (2×C), 193.0.

MS-ESI m/z (rel. int.): 397.3 ([MH]⁺, 100).

HPLC: Method A, XBridge™ column, detection UV 254 nm, RT=4.63 min, peak area 99.0%.

### Preparation of 7-ethoxy-4-(4-ethoxy-3,5-dimethoxybenzoyl)isoquinolin-8-yl sulfamide 59

### 7-Ethoxy-4-(4-ethoxy-3,5-dimethoxybenzoyl)isoquinolin-8-yl sulfamide 59

(8-Amino-7-ethoxyisoquinolin-4-yl)(4-ethoxy-3,5-dimethoxyphenyl)methanone 58 free base (240 mg, 0.61 mmol) was dissolved in anhydrous CH₂Cl₂ (8 mL) and NaH (60% in oil dispersion, 29.0 mg, 0.73 mmol), anhydrous Et₃N (152 μL, 1.09 mmol) were slowly added at 4° C. under a nitrogen atmosphere. Sulfamoyl chloride LPO 43022 (210 mg, 1.81 mmol) was dissolved in anhydrous CH₂Cl₂ (2 mL) and was slowly added dropwise at 4° C. The resulting reaction mixture was stirred from 4° C. to RT for 1 h. CH₂Cl₂ (250 mL) and a 1 M K₂CO₃ aq. solution (20 mL) were added. The separated organic layer was washed with brine (10 mL), dried over MgSO₄, filtered and evaporated to give a yellow oil. This crude oil was purified by column chromatography (SiO₂, eluent CH₂Cl₂:MeOH=98:2) to give, after evaporation and drying, 7-ethoxy-4-(4-ethoxy-3,5-dimethoxybenzoyl)isoquinolin-8-yl sulfamide 59 (119 mg, 41% yield) as a white solid.

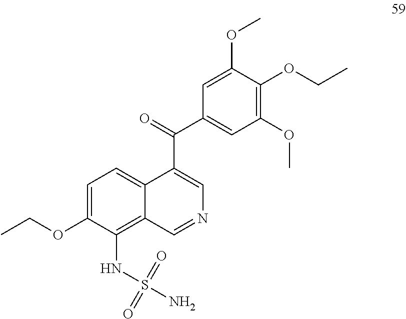 [SMILES: CCOc1ccc2c(C(=O)c3cc(OC)c(OCC)c(OC)c3)cncc2c1NS(N)(=O)=O]

MW: 475.51; Yield: 41%; White solid; Mp (° C.): 147.5

R_{f}: 0.15 (CH₂Cl₂:MeOH=98:2).

¹H-NMR (CDCl₃, δ): 1.40 (t, 3H, J=6.8 Hz, CH₃), 1.54 (t, 3H, J=6.9 Hz, CH₃), 3.84 (s, 6H, 2×MeO), 4.19 (q, 2H, J=7.0 Hz, OCH₂), 4.33 (q, 2H, J=7.1 Hz, OCH₂), 4.88 (s, 2H, NH₂), 6.80 (s, 1H, NH), 7.15 (s, 2H, 2×ArH), 7.57 (d, 1H, J=9.2 Hz, ArH), 8.21 (d, 1H, J=9.1 Hz, ArH), 8.57 (s, 1H, ArH), 9.85 (s, 1H, ArH).

¹³C-NMR (CDCl₃, δ): 14.9, 15.6, 56.4 (2×C), 65.5, 69.4, 108.1 (2×C), 119.4, 119.7, 126.4, 127.5, 128.7, 129.1, 132.6, 141.9, 142.5, 152.2, 152.6, 153.5 (2×0), 195.0.

MS-ESI m/z (% rel. Int.): 476.1 ([MH]⁺, 100).

HPLC: Method A, XBridge™ column, detection UV 254 nm, RT=4.26 min, peak area 99.0%.

### Preparation of 8-(2-(diphenylmethylene)hydrazinyl)-7-ethoxy-4-(4-ethoxy-3,5-dimethoxybenzyl)isoquinoline hydrochloride 60

### 8-(2-(Diphenylmethylene)hydrazinyl)-7-ethoxy-4-(4-ethoxy-3,5-dimethoxybenzyl)isoquinoline hydrochloride 60

A mixture of (±) BINAP (74.0 mg, 0.116 mmol), Pd₂(dba)₃ (33.0 mg, 0.035 mmol), Cs₂CO₃ (381 mg, 1.16 mmol), 4-(4-ethoxy-3,5-dimethoxybenzyl)-7-ethoxyisoquinolin-8-yl trifluoromethanesulfonate LPO 37002C (300 mg, 0.58 mmol) and benzophenone hydrazone (251 mg, 1.28 mmol) in dry toluene (15 mL) was stirred for 5 h at 160° C. in an ace pressure tube equipped with a magnetic stirrer. After cooling to RT, the reaction mixture was diluted with EtOAc (100 mL) and filtered through celite. The filtrate was concentrated at 40° C. under vacuum.

Purification by column chromatography (SiO₂, eluent cyclohexane:EtOAc=100:0 to 50:50), gave 8-(2-(diphenylmethylene)hydrazinyl)-7-ethoxy-4-(4-ethoxy-3,5-dimethoxybenzyl)isoquinoline as a yellow solid (237 mg, 72% yield). This solid (10 mg, 0.018 mmol) was dissolved in MeOH (1 mL) in a 10 mL round-bottomed flask equipped with a magnetic stirrer and the solution was cooled to 0° C. before adding a 1.34 N HCl solution in MeOH (14 μL). The solution was stirred for 15 min at 0° C. After evaporation and drying under under P₂O₅, 8-(2-(diphenylmethylene)hydrazinyl)-7-ethoxy-4-(4-ethoxy-3,5-dimethoxybenzyl)isoquinoline hydrochloride 60 (11.5 mg, 71% yield) was obtained as a red solid.

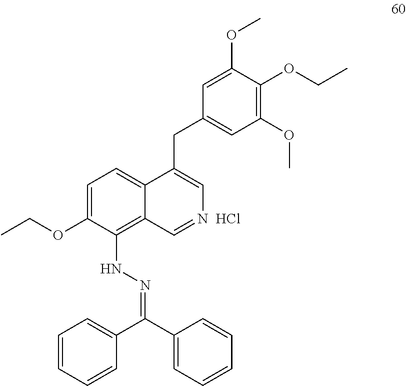 [SMILES: CCOc1ccc2c(Cc3cc(OC)c(OCC)c(OC)c3)cncc2c1NN=C(c1ccccc1)c1ccccc1 ; Cl]

MW: 598.13; Yield: 71%; Red solid; Mp (° C.): 216.9

R_{f}: 0.25 (cyclohexane:EtOAc=70:30, free base).

¹H-NMR (CD₃OD, δ): 1.18 (t, 3H, J=7.3 Hz, CH₂CH₃), 1.29 (t, 3H, J=6.9 Hz, CH₂CH₃), 3.77 (s, 6H, 2×OMe), 3.95 (q, 2H, J=6.9 Hz, OCH₂), 4.13 (q, 2H, J=6.0 Hz, OCH₂), 4.44 (s, 2H, CH₂), 6.62 (s, 2H, 2×ArH), 7.42-7.46 (m, 5H, 5×ArH), 7.65-7.80 (m, 7H, 7×ArH), 8.04 (s, 1H, ArH), 11.06 (s, 1H, ArH).

¹³C-NMR (CD₃OD, δ): 15.0, 15.7, 37.4, 56.8 (2×C), 66.8, 69.9, 107.5 (2×C), 116.4, 117.2, 123.1, 127.7, 128.3 (2×C), 129.8 (3×C), 129.9 (3×C), 130.7, 131.3 (2×C), 132.7, 133.0, 135.0, 137.0, 137.6, 138.3, 145.4, 146.5, 153.6, 155.2 (2×C).

MS-ESI m/z (% rel. Int.): 562.2 ([MH]⁺, 100).

HPLC: Method A, XBridge™ column, detection UV 254 nm, RT=6.16 min, peak area 99.9%.

### Preparation of 7-ethoxy-4-((8-methoxy-2,3-dihydrobenzo[b][1,4]dioxin-6-yl)methyl)isoquinolin-8-ol hydrochloride 61

### 8-Methoxy-2,3-dihydrobenzo[b][1,4]dioxine-6-carbaldehyde TTA 24152C

In a 100 mL round bottom flask 3,4-dihydroxybenzaldehyde (1.5 g, 8.9 mmol) was dissolved in DMF (14 mL) then K₂CO₃ (2.46 g, 17.8 mmol) and 1,2-dibromoethane (0.83 mL, 9.8 mmol) were added. The reaction mixture was stirred at 100° C. for 2 h under a nitrogen atmosphere. After cooling to RT, the reaction mixture was poured into H₂O (120 mL) and extracted with Et₂O (150 mL). The organic layer was washed with H₂O (2×50 mL), brine (50 mL), dried over MgSO₄, filtered and concentrated to dryness under reduced pressure to give 8-methoxy-2,3-dihydrobenzo[b][1,4]dioxine-6-carbaldehyde TTA 24152C (1.30 g, 75% yield) as an white solid.

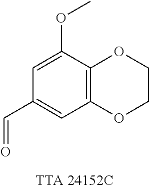

MW: 194.18; Yield: 75%; White solid; Mp (° C.): 82.3

¹H-NMR (CDCl₃, δ): 3.95 (s, 1H, CH₃O), 4.30-4.33 (m, 2H, OCH₂), 4.39-4.42 (m, 2H, OCH₂), 7.08 (d, 2H, 2×ArH), 9.79 (s, 1H, ArH).

¹³C-NMR (CDCl₃, δ): 56.2, 63.9, 64.8, 103.1, 114.4, 129.1, 138.8, 144.0, 149.5, 190.7.

MS-ESI m/z (% rel. Int.): 195.1 ([MH]⁺, 100).

HPLC: Method A, XBridge™ column, detection UV 254 nm, RT=4.15 min, peak area 98.0%.

### 7-Ethoxy-4-((8-methoxy-2,3-dihydrobenzo[b][1,4]dioxin-6-yl)methyl)isoquinolin-8-ol hydrochloride 61

In an ace pressure tube, 2-((2,2-diethoxyethylamino)methyl)-6-ethoxyphenol SAO 33014 (438 mg, 1.54 mmol, 1.0 eq) and 8-methoxy-2,3-dihydrobenzo[b][1,4]dioxine-6-carbaldehyde TTA 24152C (300 mg, 1.54 mmol, 1.0 eq) were dissolved in EtOH (1.4 mL) and a 37% HCl solution (1.4 mL) was added. The reaction mixture was stirred at 90° C. for 10 min, cooled to 4° C. and concentrated. The obtained crude product was transformed into its freebase with a 1 N K₂CO₃ aq. solution (20 mL) and dichloromethane (200 mL) was added. The separated organic layer was washed with brine (30 mL), dried over MgSO₄, filtered and evaporated to give a yellow oil. This oil was purified by column chromatography (SiO₂, eluent CH₂Cl₂:MeOH=98:2) to give, after evaporation and drying, 7-ethoxy-4-((8-methoxy-2,3-dihydrobenzo[b][1,4]dioxin-6-yl)methyl)isoquinolin-8-ol (86.1 mg) as a yellow solid. This solid was dissolved in MeOH (2 mL) and a 1.34 N HCl solution in MeOH (184 μL, 0.25 mmol, 1.05 eq) was slowly added. The reaction mixture was stirred at 4° C. for 15 min. After evaporation and drying under P₂O₅, 7-ethoxy-4-((8-methoxy-2,3-dihydrobenzo[b][1,4]dioxin-6-yl)methyl)isoquinolin-8-ol hydrochloride 61 (87.3 mg, 14% yield) was obtained as a yellow solid.

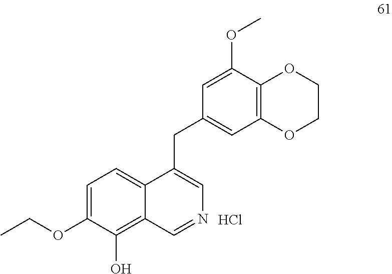 [SMILES: CCOc1ccc2c(Cc3cc(OC)c4c(c3)OCCO4)cncc2c1O ; Cl]

MW: 403.86; Yield: 14%; Yellow Solid; Mp (° C.): 255.9

R_{f}: 0.20 (CH₂Cl₂:MeOH=98:2, free base).

¹H-NMR (CD₃OD, δ): 1.51 (t, 3H, J=6.95 Hz, OCH₂CH₃), 3.78 (s, 3H, OMe), 4.22 (s, 4H, 2×OCH₂), 4.35 (q, 2H, J=7.01 Hz, OCH₂CH₃), 4.40 (s, 2H, CH₂), 6.38 (s, 1H, ArH), 6.54 (s, 1H, ArH), 7.85 (d, 1H, J=9.08 Hz, ArH), 8.02 (d, 1H, J=9.11 Hz, ArH), 8.07 (s, 1H, ArH), 9.65 (s, 1H, ArH).

¹³C-NMR (CD₃OD, δ): 15.0, 36.5, 56.8, 65.5, 65.6, 66.9, 106.6, 111.5, 116.6, 120.5, 126.7, 128.3, 131.2, 132.8, 133.7, 138.1, 142.4, 145.7, 146.4, 146.8, 150.7.

MS-ESI m/z (% rel. Int.): 368.2 ([MH]⁺, 100).

HPLC: Method A, XBridge™ column, detection UV 254 nm, RT=4.07 min, peak area 99.9%.

### Preparation of 7-Ethoxy-4-((7-methoxybenzo[d][1,3]dioxol-5-yl)methyl)isoquinolin-8-ol hydrochloride 62

### 7-Methoxybenzo[d][1,3]dioxole-5-carbaldehyde TTA 24152A

In a 50 mL round bottom flask 3,4-dihydroxy-5-methoxybenzaldehyde (1.00 g, 6.0 mmol) was dissolved in DMF (10 mL) then K₂CO₃ (1.70 g, 12.3 mmol) and CH₂Br₂ (470 μL, 6.6 mmol) were added. The reaction mixture was stirred at 100° C. for 2 h under a nitrogen atmosphere. The reaction mixture was poured into H₂O (120 mL) and extracted with Et₂O (150 mL). The organic layer was washed with H₂O (2×30 mL), brine (30 mL), dried over MgSO₄, filtered and concentrated to dryness to give 7-methoxybenzo[d][1,3]dioxole-5-carbaldehyde TTA 24152A (1.00 g, 93% crude yield) as an off white solid.

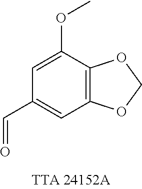

MW: 180.16; Yield: 93%; Off-white solid, Mp (° C.): 129.5

¹H-NMR (CDCl₃, δ): 3.97 (s, 3H, MeO), 6.10 (s, 2H, CH₂O), 7.05 (d, 1H, J=1.3 Hz, ArH), 7.14 (d, 1H, J=1.3 Hz, ArH).

¹³C-NMR (CDCl₃, δ): 56.6, 102.6, 103.6, 110.4, 131.8, 141.0, 144.1, 149.4, 190.1.

MS-ESI m/z (% rel. Int.): 181.1 ([MH]⁺, 75), 153.1 (100).

HPLC: Method A, XBridge™ column, detection UV 254 nm, RT=2.64 min, peak area 90%.

### 7-Ethoxy-4-((7-methoxybenzo[d][1,3]dioxol-5-yl)methyl)isoquinolin-8-ol hydrochloride 62

In an ace pressure tube, 2-((2,2-diethoxyethylamino)methyl)-6-ethoxyphenol SAO 33014 (472.0 mg, 1.66 mmol, 1.0 eq) and 7-methoxybenzo[d][1,3]dioxole-5-carbaldehyde TTA 24152A (300 mg, 1.66 mmol, 1.0 eq) were dissolved in EtOH (1.4 mL) and a 37% HCl solution (1.4 mL) was added. The reaction mixture was stirred at 90° C. for 10 min, cooled to 4° C. and concentrated. The obtained crude product was transformed into its freebase with a 1 N K₂CO₃ solution (20 mL) and extracted with CH₂Cl₂ (200 mL). The separated organic layer was washed with brine (30 mL), dried over MgSO₄, filtered and evaporated to give a yellow oil. This oil was purified by column chromatography (SiO₂, eluent CH₂Cl₂:MeOH=100:0 to 98:2) to give, after evaporation and drying, 7-ethoxy-4-((7-methoxybenzo[d][1,3]dioxol-5-yl)methyl)isoquinolin-8-ol (68.1 mg) as a yellow solid. This solid was dissolved in MeOH (2 mL) and a 1.34 N HCl solution in MeOH (151 μL, 0.20 mmol, 1.05 eq) was slowly added. The reaction mixture was stirred at 4° C. for 15 min. After evaporation of solvent and drying under P₂O₅, 7-ethoxy-4-((7-methoxybenzo[d][1,3]dioxol-5-yl)methyl)isoquinolin-8-ol hydrochloride 62 (65.3 mg, 10% yield) was obtained as a yellow solid.

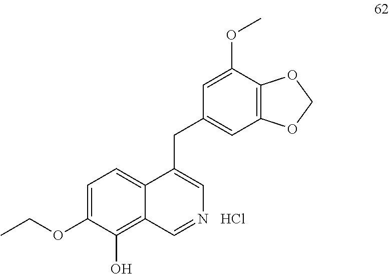 [SMILES: CCOc1ccc2c(Cc3cc(OC)c4c(c3)OCO4)cncc2c1O ; Cl]

MW: 389.83; Yield: 10%; Yellow Solid; Mp (° C.): 260.7

R_{f}: 0.20 (CH₂Cl₂:MeOH=98:2, free base).

¹H-NMR (CD₃OD, δ): 1.49 (t, 3H, J=7.0 Hz, OCH₂CH₃), 3.83 (s, 3H, OMe), 4.33 (q, 2H, J=7.00 Hz, OCH₂CH₃), 4.41 (s, 2H, CH₂), 5.88 (s, 2H, CH₂OCH₂), 6.42 (s, 1H, ArH), 6.56 (s, 1H, ArH), 7.82 (d, 1H, J=9.1 Hz, ArH), 7.99 (d, 1H, J=9.1 Hz, ArH), 8.06 (s, 1H, ArH), 9.63 (s, 1H, ArH).

¹³C-NMR (CD₃OD, δ): 15.0, 36.7, 57.4, 66.9, 102.7, 103.9, 110.4, 116.6, 120.5, 126.7, 128.4, 132.7, 133.5, 135.6, 138.0, 142.5, 145.3, 146.4, 146.8, 150.9.

MS-ESI m/z (% rel. Int.): 354.2 ([MH]⁺, 100).

HPLC: Method A, detection UV 254 nm, RT=4.28 min, peak area 99.9%

### Preparation of 4-(3,5-dimethoxy-4-(pentyloxy)benzyl)-7-ethoxyisoquinolin-8-ol hydrochloride 63

### 3,5-Dimethoxy-4-(pentyloxy)benzaldehyde RBO 40092

4-Hydroxy-3,5-dimethoxybenzaldehyde (500 mg, 2.74 mmol) was dissolved in DMF (20 mL) and Cs₂CO₃ (1.79 g, 5.48 mmol) was added at RT. Bromopentane (497 mg, 3.29 mmol) was added and the reaction mixture was stirred overnight at 40° C. DMF was removed and Et₂O (50 mL) was added. The separated organic layer was washed with water (2×30 mL), brine (30 mL), dried over Na₂SO₄, filtered and concentrated to dryness to give 3,5-dimethoxy-4-(pentyloxy)benzaldehyde RBO 40092 (642 mg, 93% yield) as a colorless oil.

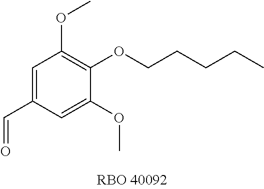 [SMILES: CCCCCOc1c(OC)cc(C=O)cc1OC]

MW: 252.31; Yield: 93%; Colorless oil.

¹H-NMR (CDCl₃, δ): 0.92 (t, 3H J=7.5 Hz, CH₃), 1.42 (m, 4H, 2×CH₂), 1.77 (m, 2H, CH₂), 3.92 (s, 6H, 2×OMe), 4.07 (t, 2H, OCH₂), 7.13 (s, 2H, 2×ArH), 9.87 (s, 1H, CHO).

MS-ESI m/z (% rel. Int.): 253.1 ([MH]⁺, 45), 183.0 (100).

HPLC: Method A, XBridge™ column, detection UV 254 nm, RT=6.23 min, peak area 99.0%.

### 4-(3,5-Dimethoxy-4-(pentyloxy)benzyl)-7-ethoxyisoquinolin-8-ol hydrochloride 63

In an ace pressure tube, 2-((2,2-diethoxyethylamino)methyl)-6-ethoxyphenol SAO 33014 (200 mg, 0.71 mmol) and 3,5-dimethoxy-4-(pentyloxy)benzaldehyde RBO 40092 (178 mg, 0.71 mmol) were dissolved in a mixture of EtOH:37% HCl=1:1 (10 mL). The reaction mixture was stirred at 90° C. for 20 min and cooled to RT. The volatiles were removed under vacuum and CH₂Cl₂ (100 mL) was added and the resulting solution was neutralized with a 28% NH₃ aqueous solution (1 mL). The separated organic layer was washed with water (3×30 mL), brine (30 mL), dried over Na₂SO₄, filtered and evaporated to give a yellow solid. This solid was purified by column chromatography (SiO₂, eluent CH₂Cl₂:EtOAc=100:0 to 50:50) to give, after evaporation and drying, 4-(3,5-dimethoxy-4-(pentyloxy)benzyl)-7-ethoxyisoquinolin-8-ol (42 mg). This solid was dissolved in MeOH (2 mL) and a 0.49 HCl solution in MeOH (204 μL, 0.10 mmol) was slowly added. The reaction mixture was stirred at 4° C. for 15 min. After evaporation and drying under P₂O₅, 4-(3,5-dimethoxy-4-(pentyloxy)benzyl)-7-ethoxyisoquinolin-8-ol hydrochloride 63 (45.2 mg, 14% yield) was obtained as a yellow solid.

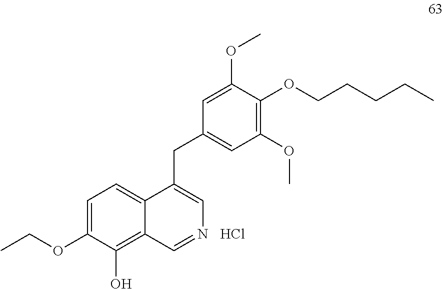 [SMILES: CCCCCOc1c(OC)cc(Cc2cncc3c(O)c(OCC)ccc23)cc1OC ; Cl]

MW: 461.99; Yield: 14%; Yellow solid; Mp (° C.): 226.7

R_{f}: 0.35 (CH₂Cl₂:EtOAc=50:50, free base).

¹H-NMR (DMSO-d₆, δ): 0.87 (t, 3H, J=9.0 Hz, CH₃), 1.28-1.42 (m, 7H, CH₂CH₂CH₃), 1.58 (q, 2H, J=9.0 Hz, CH₂), 3.69 (s, 6H, 2 OMe), 3.79 (t, 2H, J=9.0 Hz, CH₂), 4.29 (q, 2H, OCH₂), 4.42 (s, 2H, CH₂), 6.64 (d, 2H, 2×ArH), 7.84 (d, 1H, J=9.0 Hz, ArH), 8.03 (d, 1H, J=9.0 Hz, ArH), 8.31 (s, 1H, ArH), 9.54 (s, 1H, ArH), 10.98 (broad s, 1H, OH).

¹³C-NMR (DMSO-d₆, δ): 13.9, 14.6, 21.8, 27.5, 29.2, 37.2, 55.9 (2×C), 65.3, 72.2, 106.3 (2×C), 115.2, 119.2, 125.5, 128.5, 131.0, 133.8, 135.1, 135.3, 141.2), 144.4, 144.6, 153.1.

MS-ESI m/z (% rel. Int.): 426.0 ([MH]⁺, 100).

HPLC: Method A, XBridge™ column, detection UV 254 nm, RT=5.26 min, peak area 99.0%.

### Preparation of 4-(3,5-dimethoxy-4-propoxybenzyl)-7-ethoxyisoquinolin-8-amine dihydrochloride 64

### 4-(3,5-Dimethoxy-4-propoxybenzyl)-7-ethoxyisoquinolin-8-yl trifluoromethanesulfonate LPO 37164C

To a suspension of 4-(3,5-dimethoxy-4-propoxybenzyl)-7-ethoxyisoquinolin-8-ol 19 freebase (2.00 g, 5.03 mmol) in anhydrous CH₂Cl₂ (10 mL) in a 100 mL round-bottomed flask equipped with a magnetic stirrer was added anhydrous Et₃N (1.4 mL, 10.06 mmol) and N-phenyl-bis(trifluoromethanesulfonimide) (2.7 g, 7.55 mmol). The reaction mixture was stirred overnight at RT then diluted with CH₂Cl₂ (500 mL) and the organic layer was washed with water (70 mL), brine (50 mL), dried over MgSO₄, filtered and concentrated. Purification by column chromatography (SiO₂, eluent cyclohexane:EtOAc=100:0 to 70:30) gave, after evaporation and drying, 4-(3,5-dimethoxy-4-propoxybenzyl)-7-ethoxyisoquinolin-8-yl trifluoromethanesulfonate LPO 37164C (1.60 g, 60% yield) as a yellow solid.

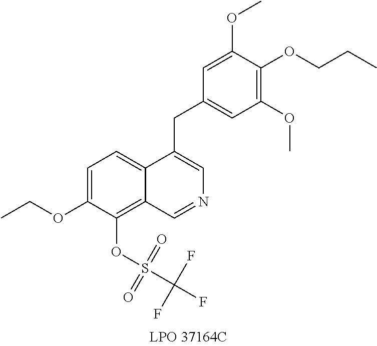 [SMILES: CCCOc1c(OC)cc(Cc2cncc3c(OS(=O)(=O)C(F)(F)F)c(OCC)ccc23)cc1OC]

MW: 529.53; Yield: 60%; Yellow Solid.

R_{f}: 0.25 (cyclohexane:EtOAc=70:30).

¹H-NMR (CD₃OD, δ): 0.99 (t, 3H, J=9.0 Hz, CH₂CH₃), 1.51 (t, 3H, J=6.0 Hz, CH₂CH₃), 1.75 (q, 2H, J=6.0 Hz, CH₂CH₃), 3.74 (s, 6H, 2×OMe), 3.90 (t, 2H, J=6.0 Hz, CH₂O), 4.24-4.31 (m, 4H, CH₂O & CH₂), 6.38 (s, 2H, 2×ArH), 7.51 (d, 1H, J=9.0 Hz, ArH), 7.95 (d, 1H, J=9.0 Hz, ArH), 8.38 (s, 1H, ArH), 9.35 (s, 1H, ArH).

MS-ESI m/z (% rel. Int.): 530.3 ([MH]⁺, 100).

HPLC: Method A, XBridge™ column, detection UV 254 nm, RT=6.03 min, peak area 98.0%.

### 4-(3,5-Dimethoxy-4-propoxybenzyl)-N-(diphenylmethylene)-7-ethoxyisoquinolin-8-amine LPO 37168C

A mixture of (±) BINAP (376 mg, 0.60 mmol), Pd₂(dba)₃ (166 mg, 0.18 mmol), Cs₂CO₃ (1.97 g, 6.04 mmol), 4-(3,5-dimethoxy-4-propoxybenzyl)-7-ethoxyisoquinolin-8-yl trifluoromethanesulfonate LPO 37164C (1.6 g, 3.02 mmol) and benzophenone imine (1.1 mL, 6.65 mmol) in dry toluene (65 mL) was stirred in an ace pressure tube equipped with a magnetic stirrer for 5 h at 160° C. After cooling to RT, the reaction mixture was diluted with EtOAc (200 mL) and filtered through celite. The filtrate was concentrated. Purification by column chromatography (SiO₂, eluent cyclohexane:EtOAc=100:0 to 70:30) gave, after evaporation and drying, 4-(3,5-dimethoxy-4-propoxybenzyl)-N-(diphenylmethylene)-7-ethoxyisoquinolin-8-amine LPO 37168C (1.38 g, 81% yield) as a yellow solid.

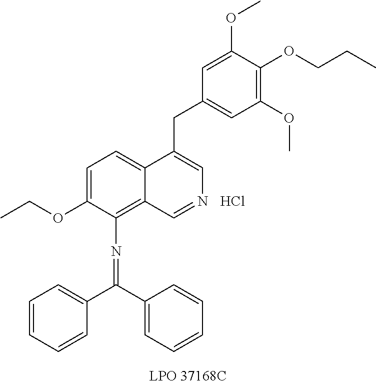 [SMILES: CCCOc1c(OC)cc(Cc2cncc3c(N=C(c4ccccc4)c4ccccc4)c(OCC)ccc23)cc1OC ; Cl]

MW: 560.68; Yield: 81%; Yellow solid; Mp (° C.): 122.2

R_{f}: 0.25 (cyclohexane:EtOAc=70:30).

¹H-NMR (CDCl₃, δ): 0.98 (t, 3H, J=7.3 Hz, CH₂CH₃), 1.30 (t, 3H, J=7.0 Hz, CH₂CH₃), 1.75 (sextuplet, 2H, J=7.3 Hz, CH₂CH₂CH₃), 3.69 (s, 6H, 2×OMe), 3.86-4.02 (m, 4H, 2×OCH₂), 4.25 (s, 2H, CH₂), 6.31 (s, 2H, 2×ArH), 7.06-7.21 (m, 6H, 6×ArH), 7.44-7.56 (m, 4H, 4×ArH), 7.88 (d, 2H, J=7.3 Hz, ArH), 8.23 (s, 1H, ArH), 9.26 (s, 1H, ArH).

¹³C-NMR (CDCl₃, δ): 10.3, 15.1, 23.3, 36.6, 56.1 (2×C), 64.7, 75.1, 105.7 (2×CH), 119.4, 119.7, 122.5, 127.4 (2×C), 128.0 (2×C), 128.3 (2×C), 128.7, 128.8, 129.6 (2×C), 130.1, 131.1, 135.3, 135.7, 136.3, 136.9, 138.9, 141.9, 142.7, 148.2, 153.5 (2×C), 171.5.

MS-ESI m/z (rel. int.): 561.2 ([MH]⁺, 100).

### 4-(3,5-Dimethoxy-4-propoxybenzyl)-7-ethoxyisoquinolin-8-amine dihydrochloride 64

A mixture of 4-(3,5-dimethoxy-4-propoxybenzyl)-N-(diphenylmethylene)-7-ethoxyisoquinolin-8-amine LPO 37168C (200 mg, 0.36 mmol), was dissolved in THF (8 mL) and a 1 N HCl aq. solution (8 mL). The reaction mixture was stirred for 1 h at RT. The volatiles were then removed at 40° C. and CH₂Cl₂ (250 mL) was added and the resulting organic layer was washed with NaHCO₃ (20 mL), brine (20 mL), dried over MgSO₄, filtered and concentrated. Purification by column chromatography (SiO₂, eluent cyclohexane:EtOAc=50:50 to 0:100) gave, after evaporation and drying, 4-(3,5-dimethoxy-4-propoxybenzyl)-7-ethoxyisoquinolin-8-amine (125 mg). This solid was dissolved in MeOH (2 mL) and a 1.34 N HCl solution in MeOH (481 μL, 0.64 mmol) was slowly added. The reaction mixture was stirred at 4° C. for 15 min. After evaporation and drying under P₂O₅, 4-(3,5-dimethoxy-4-propoxybenzyl)-7-ethoxyisoquinolin-8-amine dihydrochloride 64 (115.5 mg, 69% yield) was obtained as a red solid.

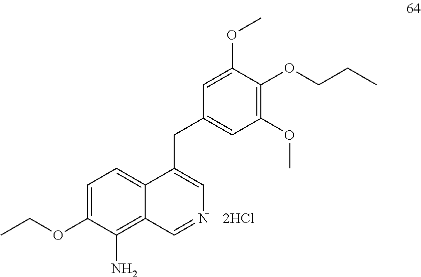 [SMILES: CCCOc1c(OC)cc(Cc2cncc3c(N)c(OCC)ccc23)cc1OC ; "2HCl"]

MW: 469.4; Yield: 69%; Red solid; Mp (° C.): 263.9

R_{f}: 0.25 (cyclohexane:EtOAc=50:50, free base).

¹H-NMR (CD₃OD, δ): 1.02 (t, 3H, J=7.4 Hz, CH₂CH₃), 1.52 (t, 3H, J=7.0 Hz, CH₂CH₃), 1.71 (m, 2H, CH₂CH₂), 3.77 (s, 6H, 2×OMe), 3.86 (t, 2H, J=6.6 Hz, OCH₂CH₂), 4.31 (q, 2H, J=7.0 Hz, CH₂CH₃), 4.40 (s, 2H, CH₂), 6.60 (s, 2H, 2×ArH), 7.54 (d, 1H, J=8.8 Hz, ArH), 7.82 (d, 1H, J=8.9 Hz, ArH), 7.91 (s, 1H, ArH), 9.66 (s, 1H, ArH).

¹³C-NMR (CD₃OD, δ): 8.5, 12.8, 22.0, 34.9, 64.3 (2×C), 64.0, 73.8, 105.2 (2×C), 110.0, 114.3, 121.6, 124.7, 129.8, 132.7, 134.9, 135.1, 137.8, 140.1, 143.0, 152.8 (2×C).

MS-ESI m/z (% rel. Int.): 397.1 ([MH]⁺, 100).

HPLC: Method A, XBridge™ column, detection UV 268 nm, RT=4.93 min, peak area 98.0%.

### Preparation of 4-(3,5-Dimethoxy-4-propoxybenzoyl)-7-ethoxyisoquinolin-8-yl acetate 65

### 4-(3,5-Dimethoxy-4-propoxybenzyl)-7-ethoxyisoquinolin-8-yl acetate LPO 43034C

4-(3,5-Dimethoxy-4-propoxybenzyl)-7-ethoxyisoquinolin-8-ol 19 freebase (2.00 g, 5.03 mmol), DIEA (3.3 mL, 19.93 mmol), DMAP (141 mg, 1.16 mmol), acetic anhydride (1.9 mL, 20.13 mmol) and CH₂Cl₂ (100 mL) were stirred overnight at RT in a round bottom flask under a nitrogen atmosphere. CH₂Cl₂ (500 mL) and H₂O (50 mL) were added. The separated organic layer was washed with brine (30 mL), dried over MgSO₄, filtered and evaporated to give a brown solid. This crude solid was purified by column chromatography (SiO₂, eluent cyclohexane:EtOAc=100:0 to 50:50) to give, after evaporation and drying, 4-(3,5-dimethoxy-4-propoxybenzyl)-7-ethoxyisoquinolin-8-yl acetate LPO 43034C (2.1 g, 96% yield) as a white solid.

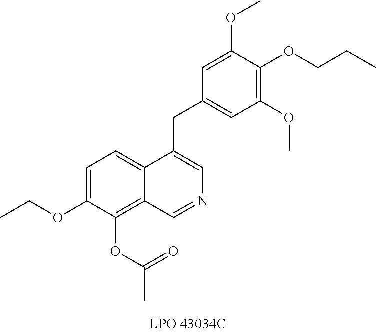 [SMILES: CCCOc1c(OC)cc(Cc2cncc3c(OC(C)=O)c(OCC)ccc23)cc1OC]

MW: 439.5; Yield: 96%; White Solid; Mp (° C.): 107.4

R_{f}: 0.30 (cyclohexane:EtOAc=50:50).

¹H-NMR (CD₃OD, δ): 0.98 (t, 3H, J=7.4 Hz, CH₃), 1.43 (t, 3H, J=7.0 Hz, CH₃), 1.75 (q, 2H, J=7.2 Hz, CH₂), 2.48 (s, 3H, COCH₃), 3.74 (s, 6H, 2×OMe), 3.89 (t, 3H, J=6.9 Hz, OCH₂), 4.19 (q, 2H, J=7.0 Hz, OCH₂), 4.28 (s, 2H, CH₂), 6.39 (s, 2H, 2×ArH), 7.48 (d, 1H, J=9.3 Hz, ArH), 7.82 (d, 1H, J=9.2 Hz, ArH), 8.30 (s, 1H, ArH), 9.21 (s, 1H, ArH).

¹³C-NMR (CDCl₃, δ): 10.3, 15.0, 20.5, 23.3, 36.7, 56.2 (2×C), 65.4, 75.2, 106.1 (2×C), 119.7, 122.6, 123.4, 129.2, 130.2, 134.5, 134.8, 136.1, 142.1, 145.5, 147.5, 153.7 (2×C), 168.6.

MS-ESI m/z (% rel. Int.): 440.1 ([MH]⁺, 100).

HPLC: Method A, XBridge™ column, detection UV 254 nm, RT=4.64 min, peak area 99.0%.

### 4-(3,5-Dimethoxy-4-propoxybenzoyl)-7-ethoxyisoquinolin-8-yl acetate 65

4-(3,5-Dimethoxy-4-propoxybenzyl)-7-ethoxyisoquinolin-8-yl acetate LPO 43034C (2.1 g, 4.78 mmol), N-hydroxyphthalimide (156 mg, 0.96 mmol) and NaClO₂ (80% pure, 756 mg, 6.69 mmol) in CH₃CN:H₂O=1:1 (50 mL:50 mL) in a ace pressure tube for 30 min at 100° C. After cooling to RT, the mixture was diluted with Et₂O (450 mL), washed with a 10% aq. sodium sulfite solution (50 mL), brine (30 mL), dried over MgSO₄, filtered and concentrated. The crude product was purified by column chromatography (SiO₂, eluent cyclohexane=100:0 to 70:30). After evaporation and drying under under P₂O₅, 4-(3,5-dimethoxy-4-propoxybenzoyl)-7-ethoxyisoquinolin-8-yl acetate 65 (753 mg, 35% yield) was obtained as a white solid.

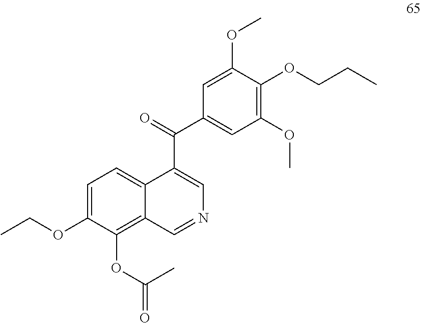 [SMILES: CCCOc1c(OC)cc(C(=O)c2cncc3c(OC(C)=O)c(OCC)ccc23)cc1OC]

MW: 453.48; Yield: 35%; White Solid; Mp (° C.): 125.0

R_{f}: 0.20 (cyclohexane:EtOAc=70:30).

¹H-NMR (CDCl₃, δ): 1.03 (t, 3H, J=7.4 Hz, CH₃), 1.45 (t, 3H, J=7.0 Hz, CH₃), 1.79 (q, 2H, J=7.1 Hz, CH₂), 2.52 (s, 3H COCH₃), 3.84 (s, 6H, 2×OMe), 4.07 (t, 2H, J=6.8 Hz, OCH₂), 4.24 (q, 2H, J=7.0 Hz, OCH₂), 7.15 (s, 2H, 2×ArH), 7.58 (d, 1H, J=9.4 Hz, ArH), 8.08 (d, 1H, J=9.3 Hz, ArH), 8.57 (s, 1H, ArH), 9.41 (s, 1H, ArH).

¹³C-NMR (CDCl₃, δ): 10.3, 14.9, 20.4, 23.4, 56.4 (2×C), 65.5, 75.3, 108.1 (2×C), 120.9, 123.6, 124.0, 128.7, 128.8, 132.6, 134.2, 142.2, 142.8, 148.2, 148.6, 153.4 (2×C), 168.5, 194.7.

MS-ESI m/z (% rel. Int.): 454.1 ([MH]⁺, 100).

HPLC: Method A, XBridge™ column, detection UV 254 nm, RT=5.63 min, peak area 99.0%.

### Preparation of (8-amino-7-ethoxyisoquinolin-4-yl)(3,5-dimethoxy-4-propoxyphenyl)methanone hydrochloride 66

### (8-Amino-7-ethoxyisoquinolin-4-yl)(3,5-dimethoxy-4-propoxyphenyl)methanone hydrochloride 66

A mixture of 4-(3,5-dimethoxy-4-propoxybenzyl)-N-(diphenylmethylene)-7-ethoxyisoquinolin-8-amine LPO 37168C (1.18 g, 2.11 mmol), N-hydroxyphthalimide (343 mg, 2.11 mmol) and NaClO₂ (80% pure, 266 mg, 2.95 mmol) in CH₃CN:H₂O=2:1 (105 mL) was stirred for 4 h at 65° C. After cooling to RT, NaClO₂ (80% pure, 57 mg, 0.63 mmol) was added and mixture was heated at 70° C. for another 5 h. After cooling to RT, the mixture was diluted with Et₂O (200 mL), washed with a 10% aq. sodium sulfite solution (15 mL), washed with brine (15 mL), dried over MgSO₄, filtered and concentrated. A mixture of 1N aq. HCl:THF=1:1 (46 mL) was added to the residue and the reaction mixture was stirred at RT for 40 min after which THF was removed at 40° C. under vacuum. The residue was neutralized with a saturated aqueous NaHCO₃ solution (30 mL) before to be extracted with CH₂Cl₂ (2×150 mL) and the organic solution was washed with brine (20 mL), dried over MgSO₄, filtered and concentrated. Purification by column chromatography (SiO₂, eluent from cyclohexane:EtOAc=100:0 to 0:100) gave, after evaporation and drying, (8-amino-7-ethoxyisoquinolin-4-yl)(3,5-dimethoxy-4-propoxyphenyl)methanone (321 mg) as an orange solid. This solid was dissolved in MeOH (5 mL) at 0° C. before adding a 1.34 N HCl solution in MeOH (0.61 mL). The solution was stirred for 15 min at 0° C. After evaporation and drying under P₂O₅, (8-amino-7-ethoxyisoquinolin-4-yl)(3,5-dimethoxy-4-propoxyphenyl)methanone hydrochloride 66 (115.5 mg, 69% yield) was obtained as a red solid.

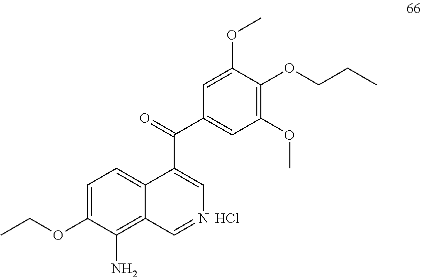 [SMILES: CCCOc1c(OC)cc(C(=O)c2cncc3c(N)c(OCC)ccc23)cc1OC ; Cl]

MW: 446.92; Yield: 29%; Red solid; Mp (° C.): 222.5

R_{f}: 0.30 (cyclohexane:EtOAc=50:50, free base).

¹H-NMR (CD₃OD, δ): 1.02 (t, 3H, J=7.0 Hz, CH₂CH₃), 1.51 (t, 3H, J=6.3 Hz, CH₂CH₃), 1.71 (t, 3H, J=6.6 Hz, CH₂CH₂), 3.79 (s, 6H, 2×OMe), 4.01 (m, 2H, CH₂CH₃), 4.30 (q, 2H, J=6.8 Hz, CH₂CH₃), 7.18 (s, 2H, 2×ArH), 7.23 (d, 1H, J=9.1 Hz), 7.78 (d, 1H, J=8.6 Hz), 8.23 (s, 1H, ArH), 9.84 (s, 1H, ArH).

¹³C-NMR (CD₃OD, δ): 10.8, 15.1, 24.5, 56.9 (2×C), 66.3, 76.4, 109.1 (2×C), 113.0, 116.7, 124.4, 127.6, 130.0, 132.8, 135.2, 141.0, 144.8, 145.4, 145.6, 155.0 (2×C), 193.0.

MS-ESI m/z (% rel. int.): 411.2 ([MH]⁺, 100).

HPLC: Method A, XBridge™ column, detection UV 254 nm, RT=4.99 min, peak area 99.0%.

### Preparation of (3,5-dimethoxy-4-propoxyphenyl)(7-ethoxy-8-hydroxyisoquinolin-4-yl)methanone hydrochloride 67

### 8-(Benzyloxy)-4-(3,5-dimethoxy-4-propoxybenzyl)-7-ethoxyisoquinoline LPO 37184C

A mixture of 4-(3,5-dimethoxy-4-propoxybenzyl)-7-ethoxyisoquinolin-8-ol 19 freebase (500 mg, 1.26 mmol) and anhydrous Et₃N (193 μL, 1.38 mmol) were dissolved in dry DMF (10 mL) and cesium carbonate (615 mg, 1.89 mmol) was added. The reaction mixture was stirred at RT for 5 min under a nitrogen atmosphere. Benzylbromide (165 μL, 1.38 mmol) was slowly added and the reaction mixture was abandoned overnight at RT. After evaporation at 50° C., the residue was diluted with EtOAc (250 mL), washed with water (70 mL), brine (30 mL), dried over MgSO₄, filtered and concentrated. The crude product was purified by column chromatography (SiO₂, eluent cyclohexane:EtOAc=100:0 to 70:30), to give after evaporation and drying, 8-(benzyloxy)-4-(3,5-dimethoxy-4-propoxybenzyl)-7-ethoxyisoquinoline LPO 37184C (292 mg, 48% yield) as a yellow oil.

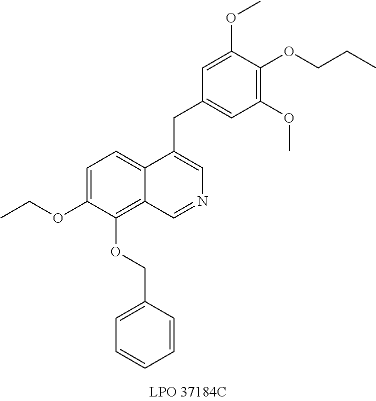 [SMILES: CCCOc1c(OC)cc(Cc2cncc3c(OCc4ccccc4)c(OCC)ccc23)cc1OC]

MW: 487.59; Yield: 48%; Yellow Oil.

R_{f}: 0.25 (cyclohexane:EtOAc=70:30).

¹H-NMR (CDCl₃, δ): 0.98 (t, 3H, J=7.4 Hz, CH₃), 1.49 (t, 3H, J=7.0 Hz, CH₃), 1.75 (q, 2H, J=7.2 Hz, OCH₂CH₂), 3.72 (s, 6H, 2×OMe), 3.89 (t, 2H, J=6.9 Hz, OCH₂CH₂), 4.10-4.25 (m, 4H, CH₂ & OCH₂), 5.27 (s, 2H, CH₂), 6.38 (s, 2H, 2×ArH), 7.31-7.45 (m, 4H, 4×ArH), 7.52 (d, 1H, J=8.0 Hz, ArH), 7.63 (d, 1H, J=9.2 Hz, ArH), 8.25 (s, 1H, ArH), 9.49 (s, 1H, ArH).

MS-ESI m/z (% rel. Int.): 488.2 ([MH]⁺, 100).

HPLC: Method A, XBridge™ column, detection UV 254 nm, RT=5.49 min, peak area 99.0%.

### (8-(Benzyloxy)-7-ethoxyisoquinolin-4-yl)(3,5-dimethoxy-4-propoxyphenyl)methanone LPO 37196C

A mixture of 4-(3,5-dimethoxy-4-propoxybenzyl)-8-(benzyloxy)-7-ethoxyisoquinoline LPO 37184C (292 mg, 599 μmol), N-hydroxyphthalimide (98 mg, 599 μmol) and NaClO₂ (80% pure, 115 mg, 1.02 μmol) in CH₃CN:H₂O=2:1 (18 mL:9 mL) was stirred in an ace pressure tube for 6 h at 70° C. After cooling to RT, the mixture was diluted with Et₂O (60 mL), washed with a 10% aq. sodium sulfite solution (10 mL), brine (10 mL), dried over MgSO₄, filtered and concentrated. The crude material was purified by column chromatography (SiO₂, eluent cyclohexane:EtOAc=100:0 to 8:2) to give, after evaporation and drying, (8-(benzyloxy)-7-ethoxyisoquinolin-4-yl)(3,5-dimethoxy-4-propoxyphenyl)methanone LPO 37196C (127 mg, 42% yield) as a yellow solid.

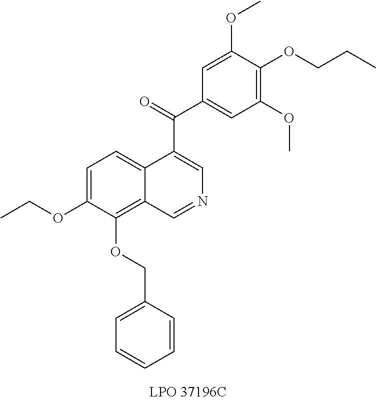 [SMILES: CCCOc1c(OC)cc(C(=O)c2cncc3c(OCc4ccccc4)c(OCC)ccc23)cc1OC]

MW: 501.57; Yield: 42%; Yellow Solid; Mp (° C.): 232.9

R_{f}: 0.2 (cyclohexane:EtOAc=70:30).

MS-ESI m/z (% rel. Int.): 502.2 ([MH]⁺, 100).

HPLC: Method A, XBridge™ column, detection UV 254 nm, RT=6.04 min, peak area 80%.

(3,5-Dimethoxy-4-propoxyphenyl)(7-ethoxy-8-hydroxyisoquinolin-4-yl)methanone hydrochloride 67

(8-(Benzyloxy)-7-ethoxyisoquinolin-4-yl)(3,5-dimethoxy-4-propoxyphenyl)methanone LPO 37196C (127 mg, 0.25 mmol, 1.0 eq) was dissolved in EtOH (5.4 mL), a 37% HCl solution (5.4 mL) was added, and the reaction mixture was stirred at 70° C. for 6 h, cooled to 4° C. and concentrated. A 1 N K₂CO₃ aq. solution (15 mL) was slowly added and the resulting solution was extracted with EtOAc (150 mL). The organic layer was washed with brine (20 mL), dried over MgSO₄, filtered and evaporated to give yellow oil. This oil was purified by column chromatography (SiO₂, eluent cyclohexane:EtOAc=90:10 to 60:40) to give after evaporation and drying (3,5-dimethoxy-4-propoxyphenyl)(7-ethoxy-8-hydroxyisoquinolin-4-yl)methanone (53.5 mg). This solid was dissolved in MeOH (2 mL), a 1.34 N HCl solution in MeOH (102 μL, 0.14 mmol, 1.05 eq) was slowly added, and the reaction mixture was stirred at 4° C. for 15 min. After evaporation and drying under under P₂O₅, (3,5-dimethoxy-4-propoxyphenyl)(7-ethoxy-8-hydroxyisoquinolin-4-yl)methanone hydrochloride 67 (49.6 mg, 44% yield) was obtained as a orange solid.

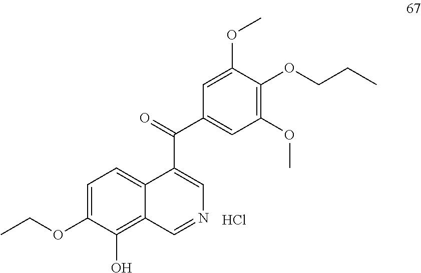 [SMILES: CCCOc1c(OC)cc(C(=O)c2cncc3c(O)c(OCC)ccc23)cc1OC ; Cl]

MW: 447.91; Yield: 44%; Orange Solid; Mp (° C.): 226.7

R_{f}: 0.30 (cyclohexane:EtOAc=6:4, free base).

¹H-NMR (CD₃OD, δ): 0.99-1.04 (m, 3H, CH₃), 1.47-1.51 (m, 3H, OCH₂CH₃), 1.69-1.75 (m, 2H, CH₂), 3.78 (s, 6H, 2×OMe), 3.98-4.03 (m, 2H, OCH₂CH₃), 4.34 (q, 2H, J=7.0 Hz, OCH₂CH₃), 7.18 (s, 2H, 2×ArH), 7.60 (d, 1H, J=9.0 Hz, ArH), 8.01 (d, 1H, J=9.1 Hz, ArH), 8.42 (s, 1H, ArH), 9.86 (s, 1H, ArH).

¹³C-NMR (CD₃OD, δ): 10.8, 15.0, 24.5, 56.9 (2×C), 66.9, 76.4, 109.3 (2×C), 117.5, 120.9, 127.4, 129.4, 130.8, 132.8, 135.3, 144.9, 145.8, 146.8, 147.1, 155.1 (2×C), 192.6.

MS-ESI m/z (% rel. Int.): 412.1 ([MH]⁺, 100).

HPLC: Method A, XBridge™ column, detection UV 254 nm, RT=4.77 min, peak area 99.0%.

### Preparation of O-(4-(3,5-dimethoxy-4-propoxybenzyl)-7-ethoxyisoquinolin-8-yl) dimethylcarbamothioate hydrochloride 68

### O-(4-(3,5-Dimethoxy-4-propoxybenzyl)-7-ethoxyisoquinolin-8-yl) dimethylcarbamothioate hydrochloride 68

4-(3,5-Dimethoxy-4-propoxybenzyl)-7-ethoxyisoquinolin-8-ol 19 freebase (205 mg, 0.52 mmol) was dissolved at 4° C. in a 1.5 N aq. KOH solution (0.38 mL, 0.70 mmol) and THF (1 mL) and a dimethylthiocarbamoyl chloride (90 mg, 0.72 mmol) solution in THF (1 mL) was added dropwise for 5 min. The reaction mixture was stirred for 1 h at 4° C. then for 0.5 h at RT. A 0.5 N aq. KOH solution (1 mL) was added (until pH basic) and the reaction mixture was stirred for another 10 min and partitioned between ice-water (20 mL) and CH₂Cl₂ (60 mL). The separated organic layer was washed with water (20 mL), brine (20 mL), dried over MgSO₄, filtered and evaporated to give a pale yellow solid (109 mg). This material was purified by column chromatography (SiO₂, eluent cyclohexane:EtOAc=100:0 to 75:25) to give, after evaporation and drying, O-(4-(3,5-dimethoxy-4-propoxybenzyl)-7-ethoxyisoquinolin-8-yl) dimethylcarbamothioate (53 mg) as a pale yellow solid. This solid was dissolved in MeOH (2 mL), a 0.34 N HCl in MeOH (326 μL, 0.11 mmol) was slowly added and the reaction mixture was stirred at 4° C. for 5 min. After evaporation and drying under P₂O₅, O-(4-(3,5-dimethoxy-4-propoxybenzyl)-7-ethoxyisoquinolin-8-yl) dimethylcarbamothioate hydrochloride 68 (54 mg, 21% yield) was obtained as a yellow solid.

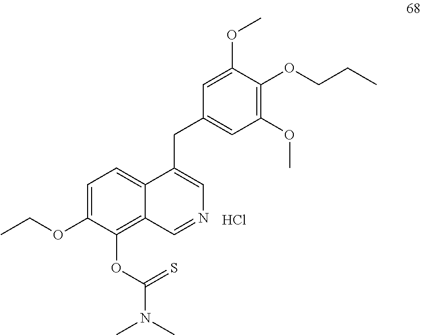 [SMILES: CCCOc1c(OC)cc(Cc2cncc3c(OC(=S)N(C)C)c(OCC)ccc23)cc1OC ; Cl]

MW: 521.07; Yield: 21%; Yellow solid; Mp (° C.): 187.7

R_{f}: 0.17 (cyclohexane:acetone=5:5, free base).

¹H-NMR (CD₃OD, δ): 1.05 (t, 3H, J=7.4 Hz, CH₃), 1.46 (t, 3H, J=7.0 Hz, CH₃), 1.75 (six, 2H, J=7.0 Hz, CH₂), 3.53 (s, 3H, NCH₃), 3.58 (s, 3H, NCH₃), 3.82 (s, 6H, 2×OMe), 4.05 (t, 2H, J=6.5 Hz, OCH₂), 4.30-4.40 (m, 2H, OCH₂), 7.22 (s, 2H, 2×ArH), 8.11 (s, 2H, 2×ArH), 8.59 (s, 1H, ArH), 9.68 (s, 1H, ArH).

¹³C-NMR (CD₃OD, δ): 10.76, 15.16, 24.50, 39.55, 44.01, 56.92 (2×C), 67.07, 76.40, 109.43 (2×C), 125.64, 126.26, 127.17, 131.15, 132.69, 132.90, 135.06, 140.00, 144.90, 146.48, 152.52, 155.05 (2×C), 188.00, 192.78.

MS-ESI m/z (% rel. Int.): 499.1 ([MH]⁺, 100).

HPLC: Method A, XBridge™ column, detection UV 254 nm, RT=6.50 min, peak area 96.0%.

### Preparation of 4-(3,5-dichloro-4-methoxybenzyl)-7-ethoxyisoquinolin-8-ol hydrochloride 69

### 3,5-Dichloro-4-methoxybenzaldehyde RBO 40104

In a 100 mL round bottom flask, 3,5-dichloro-4-hydroxybenzaldehyde (1.0 g, 5.23 mmol) was dissolved in acetone (50 mL) and K₂CO₃ (1.01 g, 7.32 mmol) was added at RT. The mixture was stirred at RT for 30 min, then MeI (1.34 g, 9.41 mmol) was added and reaction was stirred at reflux for another 5 h and finally at RT overnight. Acetone was evaporated and the residue was taken back in EtOAc (200 mL) and water (200 mL). The aqueous layer was further extracted with EtOAc (3×100 mL). The combined organic layers were washed with brine (100 mL), dried over Na₂SO₄, filtered and concentrated to dryness to give 3,5-dichloro-4-methoxybenzaldehyde RBO 40104 (257 mg, 24% yield) as an off-white solid.

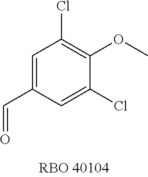

MW: 205.04; Yield: 24%; Off-white solid.

¹H-NMR (CDCl₃, δ): 3.99 (s, 3H, OMe), 7.83 (s, 2H, 2×ArH), 9.87 (s, 1H, CHO).

MS-ESI m/z (% rel. Int.): 205.0/207.0 ([MH]⁺, 100/65).

HPLC: Method A, XBridge™ column, detection UV 254 nm, RT=5.68 min, peak area 99.0%.

### 4-(3,5-Dichloro-4-methoxybenzyl)-7-ethoxyisoquinolin-8-ol hydrochloride 69

In an ace pressure tube, 2-((2,2-diethoxyethylamino)methyl)-6-ethoxyphenol SAO 33014 (355 mg, 1.25 mmol) and 3,5-dichloro-4-methoxybenzaldehyde RBO 40104 (257 mg, 1.25 mmol) were dissolved in EtOH:37% HCl solution=1:1 (10 mL). The reaction mixture was stirred at 90° C. for 10 min, and then cooled to RT. The volatiles were removed under vacuum and the residue was taken back in EtOAc (200 mL) and neutralized with a 10% NaHCO₃ aqueous solution (20 mL). The separated organic layer was washed with water (3×30 mL), brine (30 mL), dried over Na₂SO₄, filtered and evaporated to give a yellow solid. This solid was purified by column chromatography (SiO₂, eluent dichloromethane:EtOAc=100:0 to 50:50) to give, after evaporation and drying, 4-(3,5-dichloro-4-methoxybenzyl)-7-ethoxyisoquinolin-8-ol (89 mg). This solid was dissolved in MeOH (2 mL) and a 0.49 HCl solution in MeOH (480 μL, 0.24 mmol) was slowly added. The reaction mixture was stirred at 4° C. for 15 min. After evaporation and drying under P₂O₅, 4-(3,5-dichloro-4-methoxybenzyl)-7-ethoxyisoquinolin-8-ol hydrochloride (58 mg, 11% yield) 69 was obtained as a yellow solid.

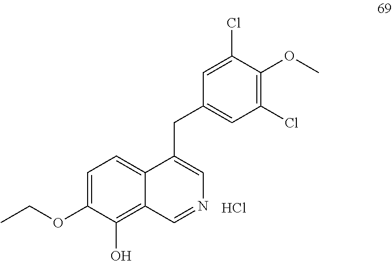 [SMILES: CCOc1ccc2c(Cc3cc(Cl)c(OC)c(Cl)c3)cncc2c1O ; Cl]

MW: 414.72; Yield: 11%; Yellow Solid; Mp (° C.): 216.4

R_{f}: 0.35 (CH₂Cl₂:EtOAc=50:50, free base).

¹H-NMR (CD₃OD, δ): 1.49 (t, 3H, J=6 Hz, CH₃), 3.83 (s, 3H, OMe), 4.32 (q, 2H, J=6 Hz OCH₂), 4.49 (s, 2H, CH₂), 7.33 (s, 2H, 2×ArH), 7.76 (d, 1H, J=12 Hz, ArH), 8.01 (d, 1H, J=12 Hz, ArH), 8.18 (s, 1H, ArH), 9.68 (s, 1H, ArH).

¹³C-NMR (CD₃OD, δ): 13.7, 34.2, 59.9, 65.6, 115.1, 119.2, 125.5, 127.4, 129.1, 129.4, 131.1, 135.1, 136.2, 141.8, 145.3, 145.8, 151.2.

MS-ESI m/z (% rel. Int.): 378.0/380.0 ([MH]⁺, 100/65).

HPLC: Method A, XBridge™ column, detection UV 254 nm, RT=4.76 min, peak area 99.0%.

### Preparation of 4-(3,5-diiodo-4-methoxybenzyl)-7-ethoxyisoquinolin-8-ol hydrochloride 70

### 4-Hydroxy-3,5-diiodobenzaldehyde RBO 40102

In a 100 mL round bottom flask 4-hydroxybenzaldehyde (1.0 g, 8.19 mmol), sodium periodate (1.75 g, 8.19 mmol), and NaCl (957 mg, 16.38 mmol) were dissolved in acetic acid (30 mL) and H₂O (3 mL) at RT. The reaction mixture was stirred at RT for 10 min, then potassium iodide (2.72 g, 16.4 mmol) was added and the reaction mixture was stirred at RT for 96 h. The reaction mixture was diluted with EtOAc (25 mL) and a 1 M sodium thiosulfate aq. solution (25 mL) was added the resulting mixture was stirred for 15 min. This solution was poured into a 250 mL Erlenmeyer with EtOAc (50 mL) and a 1 M sodium thiosulfate aq. solution (50 mL) and the resulting solution stirred for another 15 min. The organic layer was separated and the aqueous phase was further extracted with EtOAc (3×50 mL). The combined organic layers were washed with brine (100 mL), dried over Na₂SO₄, filtered and concentrated to dryness to give 4-hydroxy-3,5-diiodobenzaldehyde RBO 40102 (2.91 g, 95% yield) as a pale yellow solid.

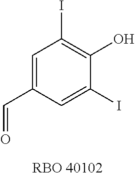 [SMILES: O=Cc1cc(I)c(O)c(I)c1]

MW: 373.92; Yield: 95%; Pale yellow solid.

¹H-NMR (CDCl₃, δ): 8.20 (s, 2H, ArH), 9.74 (s, 1H, CHO).

MS-ESI m/z (% rel. Int.): 374 ([MH]⁺, 10), 361 (100).

HPLC: Method A, XBridge™ column, detection UV 254 nm, RT=5.20 min, peak area 99.0%.

### 3,5-Diiodo-4-methoxybenzaldehyde RBO 40112

In a 100 mL round bottom flask 4-hydroxy-3,5-diiodobenzaldehyde RBO 40102 (1.0 g, 2.67 mmol) was dissolved in acetone (15 mL) and K₂CO₃ (553 mg, 4.0 mmol) was added at RT. The mixture was stirred at RT for 30 min, then iodomethane (758 mg, 5.34 mmol) was added and reaction was stirred at reflux for 3.5 h. After this time iodomethane (2 eq, 758 mg, 5.34 mmol) was added and mixture was stirred at reflux for another 1.5 h. Acetone was evaporated and the residue was taken back in EtOAc (40 mL) and water (60 mL). The aqueous layer was further extracted with EtOAc (3×40 mL). The combined organic layers were washed with brine (60 mL), dried over Na₂SO₄, filtered and concentrated to dryness to give a yellow oil. This oil was purified by column chromatography (SiO₂, eluent cyclohexane:EtOAc=100:0 to 90:10) to give, after evaporation and drying, 3,5-diiodo-4-methoxybenzaldehyde RBO 40112 (493 mg, 48% yield) as a white solid.

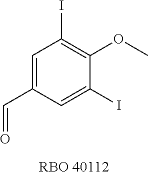

MW: 387.94; Yield: 48%; White solid.

¹H-NMR (CDCl₃, δ): 3.93 (s, 3H, OMe), 8.27 (s, 2H, 2×ArH), 9.81 (s, 1H, CHO).

MS-ESI m/z (% rel. Int.): 388 ([MH]⁺, 10), 153 (100).

### 4-(3,5-Diiodo-4-methoxybenzyl)-7-ethoxyisoquinolin-8-ol hydrochloride 70

In an 20 mL ace pressure tube, 2-((2,2-diethoxyethylamino)methyl)-6-ethoxyphenol SAO 33014 (183 mg, 0.64 mmol) and 3,5-diiodo-4-methoxybenzaldehyde RBO 40112 (250 mg, 0.64 mmol) were dissolved in EtOH: 37% HCl solution=1:1 (10 mL). The reaction mixture was stirred at 100° C. for 10 min, and cooled to RT. The volatiles were removed under vacuum and the residue was taken back in EtOAc (150 mL) and neutralized with a 10% NaHCO₃ aq. solution (20 mL). The organic layer was washed with water (3×30 mL), brine (30 mL), dried over Na₂SO₄, filtered and evaporated to give a yellow solid. This solid was purified by column chromatography (SiO₂, eluent CH₂Cl₂:EtOAc=100:0 to 50:50) to give, after evaporation and drying, 4-(3,5-diiodo-4-methoxybenzyl)-7-ethoxyisoquinolin-8-ol (188 mg). This solid was dissolved in MeOH (2 mL) and a 0.49 N HCl solution in MeOH (674 μL, 0.33 mmol) was slowly added. The reaction mixture was stirred at 4° C. for 15 min. After evaporation and drying under P₂O₅, 4-(3,5-diiodo-4-methoxybenzyl)-7-ethoxyisoquinolin-8-ol hydrochloride EHT 6282 (200 mg, 52% yield) was obtained as a yellow solid.

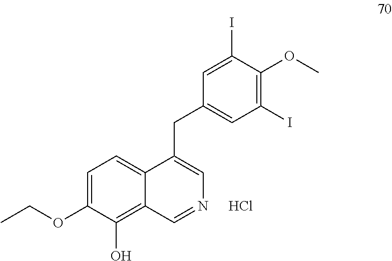 [SMILES: CCOc1ccc2c(Cc3cc(I)c(OC)c(I)c3)cncc2c1O ; Cl]

MW: 597.62; Yield: 52%; Yellow Solid; Mp (° C.): 203.3

R_{f}: 0.57 (CH₂Cl₂:EtOAc=50:50, free base).

¹H-NMR (CD₃OD, δ): 1.50 (t, 3H, J=9 Hz, CH₃), 3.79 (s, 3H, OMe), 4.35 (q, 2H, J=9 Hz OCH₂), 4.45 (s, 2H, CH₂), 7.73-7.80 (m, 3H, 3×ArH), 8.03 d, 1H, J=9 Hz, ArH), 8.13 (s, 1H, ArH), 9.68 (s, 1H, ArH).

¹³C-NMR (CD₃OD, δ): 15.0, 34.7, 61.1, 66.9, 91.5 (2×C), 116.4, 120.5, 126.8, 128.6, 132.4, 136.6, 139.4, 141.3 (2×C), 143.1, 146.6, 147.1, 159.6.

MS-ESI m/z (% rel. Int.): 561.0 ([MH]⁺, 100).

HPLC: Method A, XBridge™ column, detection UV 254 nm, RT=5.01 min, peak area 98.0%.

### Preparation of 4-(3-bromo-4,5-dimethoxybenzyl)-7-ethoxyisoquinolin-8-ol hydrochloride 71

### 3-Bromo-4,5-dimethoxybenzaldehyde RBO 40106

In a 100 mL round bottom flask 3-bromo-4-hydroxy-5-methoxybenzaldehyde (1.0 g, 4.33 mmol) was dissolved in acetone (10 mL) and K₂CO₃ (898 mg, 6.50 mmol) was added at RT. The mixture was stirred at RT for 30 min, then iodomethane (923 mg, 6.50 mmol) was added and reaction was stirred at reflux for 4 h. Acetone was removed and the residue was taken back in EtOAc (10 mL) and H₂O (10 mL). The aqueous layer was further extracted with EtOAc (3×20 mL). The combined organic layers were washed with brine (10 mL), dried over Na₂SO₄, filtered and concentrated to dryness to give 3-bromo-4,5-dimethoxybenzaldehyde RBO 40106 (483 mg, 46% yield) as a yellow solid.

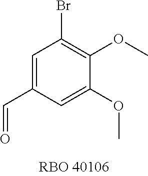

MW: 245.07; Yield: 46%; Yellow solid.

¹H-NMR (CDCl₃, δ): 3.95 (s, 6H, 2×OMe), 7.41 (s, 1H, ArH), 7.65 (s, 1H, ArH), 9.85 (s, 1H, CHO).

MS-ESI m/z (% rel. Int.): 245.0/247.0 ([MH]⁺, 50/50).

HPLC: Method A, XBridge™ column, detection UV 254 nm, RT=5.36 min, peak area 97.0%.

### 4-(3-Bromo-4,5-dimethoxybenzyl)-7-ethoxyisoquinolin-8-ol hydrochloride 71

In an 20 mL ace pressure tube, 2-((2,2-diethoxyethylamino)methyl)-6-ethoxyphenol SAO 33014 (289 mg, 1.02 mmol) and 3-bromo-4,5-dimethoxybenzaldehyde RBO 40106 (250 mg, 1.02 mmol) were dissolved in a mixture EtOH:37% HCl solution=1:1 (10 mL). The reaction mixture was stirred at 90° C. for 10 min, and cooled to RT. The volatiles were removed under vacuum and the residue was taken back in EtOAc (150 mL) and neutralized with a 10% NaHCO₃ aq. solution (15 mL). The separated organic layer was washed with water (3×30 mL) and brine (30 mL), dried over MgSO₄, filtered and evaporated to give a yellow solid. This solid was purified by column chromatography (SiO₂, eluent CH₂Cl₂:EtOAc=100:0 to 50:50) to give, after evaporation and drying, 4-(3-bromo-4,5-dimethoxybenzyl)-7-ethoxyisoquinolin-8-ol (93 mg). This solid was dissolved in MeOH (2 mL) and a 0.49 N HCl solution in MeOH (450 μL, 0.22 mmol) was slowly added. The reaction mixture was stirred at 4° C. for 15 min. After evaporation and drying under P₂O₅, 4-(3-bromo-4,5-dimethoxybenzyl)-7-ethoxyisoquinolin-8-ol hydrochloride 71 (100 mg, 22% yield) was obtained as a yellow solid.

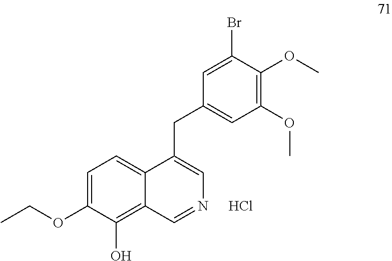 [SMILES: CCOc1ccc2c(Cc3cc(Br)c(OC)c(OC)c3)cncc2c1O ; Cl]

MW: 454.75; Yield: 22%; Yellow Solid; Mp (° C.): 222.4

R_{f}: 0.35 (CH₂Cl₂:EtOAc=50:50, free base).

¹H-NMR (CD₃OD, δ): 1.50 (t, 3H, J=9 Hz, CH₃), 3.76 (s, 3H, OMe), 3.82 (s, 3H, OMe), 4.34 (q, 2H, J=6 Hz OCH₂), 4.48 (s, 2H, CH₂), 7.00 (s, 2H, 2×ArH), 7.81 (d, 1H, J=9 Hz, ArH), 8.02 (d, 1H, J=9 Hz ArH), 8.11 (s, 1H, ArH), 9.66 (s, 1H, ArH)

¹³C-NMR (CD₃OD, δ): 15.0, 36.1, 56.7, 60.8, 66.9, 114.0, 116.5, 118.6, 120.5, 125.7, 126.8, 128.5, 132.6, 136.7, 137.2, 142.8, 146.5, 146.7, 147.0, 155.4.

MS-ESI m/z (% rel. Int.): 420.0/418.0 ([MH]⁺, 50/50).

HPLC: Method A, XBridge™ column, detection UV 254 nm, RT=4.52 min, peak area 99.0%.

### Preparation of 7-ethoxy-4-(3-iodo-4,5-dimethoxybenzyl)isoquinolin-8-ol hydrochloride 72

### 3-Iodo-4,5-dimethoxybenzaldehyde RBO 40110

In a 100 mL round bottom flask 3-iodo-4-hydroxy-5-methoxybenzaldehyde (1.0 g, 3.60 mmol) was dissolved in acetone (50 mL) and K₂CO₃ (746 mg, 5.40 mmol) was added at RT. The reaction mixture was stirred at RT for 30 min, then iodomethane (766 mg, 5.40 mmol) was added and reaction was stirred at reflux for another 4 h. Acetone was removed and the residue was taken back in EtOAc (50 mL) and H₂O (50 mL). The aqueous layer was further extracted with EtOAc (3×50 mL). The combined rganics layers were washed with brine (50 mL), dried over Na₂SO₄, filtered and concentrated to dryness to give 3-iodo-4,5-dimethoxybenzaldehyde RBO 40110 (603 mg, 57% yield) as a brown oil.

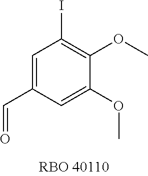

MW: 292.07; Yield: 57%; Brown oil.

¹H-NMR (CDCl₃, δ): 3.93 (s, 6H, 2×OMe), 7.41 (s, 1H, ArH), 7.84 (s, 1H, ArH), 9.82 (s, 1H, CHO).

MS-ESI m/z (% rel. Int.): 293 ([MH]⁺, 100).

HPLC: Method A, XBridge™ column, detection UV 254 nm, RT=5.53 min, peak area 97.0%.

### 7-Ethoxy-4-(3-iodo-4,5-dimethoxybenzyl)isoquinolin-8-ol hydrochloride 72

In an ace pressure tube, 2-((2,2-diethoxyethylamino)methyl)-6-ethoxyphenol SAO 33014 (244 mg, 0.86 mmol) and 3-iodo-4,5-dimethoxybenzaldehyde RBO 40110 (250 mg, 0.86 mmol) were dissolved in a mixture EtOH:37% HCl solution=1:1 (10 mL). The reaction mixture was stirred at 100° C. for 10 min then cooled to RT. The volatiles were removed under vacuum and the residue was taken back in EtOAc (150 mL) and neutralized with a 10% NaHCO₃ aq. solution (15 mL). The separated organic layer was washed with water (3×30 mL), brine (30 mL), dried over Na₂SO₄, filtered and evaporated to give a yellow solid. This solid was purified by column chromatography (SiO₂, eluent CH₂Cl₂:EtOAc=100:0 to 50:50) to give, after evaporation and drying, 7-ethoxy-4-(3-iodo-4,5-dimethoxybenzyl)isoquinolin-8-ol (37 mg). This solid was dissolved in MeOH (2 mL) and a 0.49 N HCl solution in MeOH (160 μL, 0.08 mmol) was slowly added. The reaction mixture was stirred at 4° C. for 15 min. After evaporation and drying under P₂O₅, 7-ethoxy-4-(3-iodo-4,5-dimethoxybenzyl)isoquinolin-8-ol hydrochloride 72 (39 mg, 9% yield) was obtained as a yellow solid.

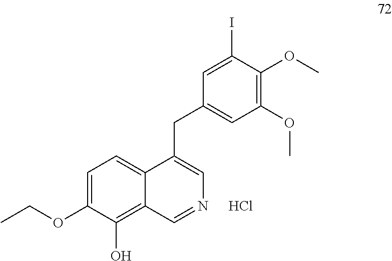 [SMILES: CCOc1ccc2c(Cc3cc(I)c(OC)c(OC)c3)cncc2c1O ; Cl]

MW: 501.75; Yield: 9%; Yellow Solid; Mp (° C.): 221.9

R_{f}: 0.40 (CH₂Cl₂:EtOAc=50:50, free base).

¹H-NMR (CD₃OD, δ): 1.50 (t, 3H, J=9 Hz, CH₃), 3.75 (s, 3H, OMe), 3.81 (s, 3H, OMe), 4.35 (q, 2H, J=9 Hz OCH₂), 4.47 (s, 2H, CH₂), 7.00 (s, 1H, ArH), 7.23 (s, 1H, ArH), 7.82 (d, 1H, J=9 Hz, ArH), 8.03 (d, 1H, J=9 Hz ArH), 8.09 (s, 1H, ArH), 9.66 (s, 1H, ArH).

¹³C-NMR (CD₃OD, δ): 15.0 (CH₃), 35.9 (CH₂), 56.5 (O—CH₃), 60.7 (O—CH₃), 66.9 (O—CH₂—CH₃), 93.1, 115.0 (ArH), 116.5 (ArH), 120.5, 126.8 (ArH), 128.4 (ArH), 131.6

(ArH), 132.6, 137.3, 137.4, 142.8 (ArH), 146.5, 147.0, 149.3, 154.2.

MS-ESI m/z (% rel. Int.): 466.0 ([MH]⁺, 100).

HPLC: Method A, XBridge™ column, detection UV 254 nm, RT=4.68 min, peak area 99.0%.

### Preparation of 7-ethoxy-4-(4-ethyl-3,5-dimethoxybenzyl)isoquinolin-8-ol hydrochloride 73

### 5-(Dimethoxymethyl)-1,2,3-trimethoxybenzene RBO 40122

In a 50 mL round bottom flask under N₂, to a solution of ammonium chloride (80 mg, 1.5 mmol) in MeOH (10 mL) and trimethyl orthoformate (10 mL) was added 3,4,5-trimethoxybenzaldehyde (5.0 g, 25 mmol). The mixture was heated for 3 h at reflux. After cooling to RT, Et₃N (750 μL) was added and the resulting mixture was stirred for 5 min, then H₂O (25 mL) was added and the reaction mixture was extracted with Et₂O (3×15 mL). The combined organic layers were washed with a 10% aq. NaHCO₃ solution (15 mL), H₂O (15 mL), brine (15 mL), dried over Na₂SO₄, filtered and concentrated to dryness under reduced pressure to give 5-(dimethoxymethyl)-1,2,3-trimethoxybenzene RBO 40122 (6.0 g, 99% yield) as a white solid.

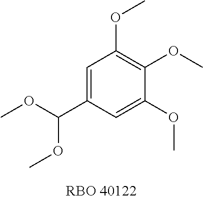

MW: 242.27; Yield: 99%; Off-white solid.

R_{f}: 0.84 (CH₂Cl₂:EtOAc=9:1).

¹H-NMR (CDCl₃, δ): 3.33 (s, 6H, 2×OMe), 3.83 (s, 3H, OMe), 3.87 (s, 6H, 2×OMe), 5.29 (s, 1H, CH), 6.68 (s, 2H, 2×ArH).

MS-ESI m/z (% rel. Int.): 197 ([MH]⁺ aldehyde, 100).

HPLC: Method A, XBridge™ column, detection UV 254 nm, RT=4.35 min, peak area 99.0%.

### 4-Ethyl-3,5-dimethoxybenzaldehyde RBO 40130

In a 50 mL round bottom flask, sodium (285 mg, 12.39 mmol) was placed in anhydrous THF (15 mL) at 0° C. under N₂. A solution of 5-dimethoxymethyl-1,2,3-trimethoxybenzene RBO 40122 (1.0 g, 4.13 mmol) in anhydrous THF (5 mL) was added carefully at 0° C. The resulting mixture was stirred for 24 h at RT.

Bromoethane (675 mg, 6.20 mmol) was added at 0° C. and the mixture was stirred for 2 days. H₂O (5 mL) and Et₂O (10 mL) were added. The aqueous layer was further extracted with Et₂O (3×10 mL). The combined organic layers were washed with brine (10 mL), dried over Na₂SO₄, filtered and concentrated to dryness to give a yellow oil. To the obtained yellow oil was added a mixture of THF:1 N HCl=1:1 solution and the resulting solution was stirred at RT for 5 min. After evaporation and drying, 4-ethyl-3,5-dimethoxybenzaldehyde RBO 40130 (733 mg, 91% crude yield) was obtained as a yellow solid. The resulting solid was engaged in the next step without purification.

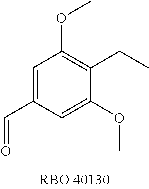

MW: 194.23; Yield: 91%; Yellow solid.

R_{f}: 0.55 (CH₂Cl₂).

¹H-NMR (CDCl₃, δ): 1.09 (t, 3H, J=3 Hz, CH₃), 2.72 (q, 2H, J=3 Hz, CH₂), 3.89 (s, 6H, 2×OMe), 7.06 (s, 2H, 2×ArH), 9.91 (s, 1H, CHO).

MS-ESI m/z (% rel. Int.): 195 ([MH]⁺, 18), 167.2 (100).

HPLC: Method A, detection UV 254 nm, RT=5.88 min, peak area 67%.

### 7-Ethoxy-4-(4-ethyl-3,5-dimethoxybenzyl)isoquinolin-8-ol hydrochloride 73

In an ace pressure tube (Aldrich, 20 mL), 2-((2,2-diethoxyethylamino)methyl)-6-ethoxyphenol SAO 33014 (365 mg, 1.29 mmol) and 4-ethyl-3,5-dimethoxybenzaldehyde RBO 40130 (250 mg, 1.29 mmol) were dissolved in EtOH:37% HCl solution=1:1 (10 mL) and the reaction mixture was stirred at 100° C. for 15 min, and cooled to RT. The volatiles were removed under vacuum and the residue was taken back in EtOAc (150 mL) and neutralized with a 10% NaHCO₃ aq. solution (15 mL). The separated organic layer was washed with water (3×30 mL), brine (30 mL), dried over Na₂SO₄, filtered and evaporated to give yellow solid. The above compound was purified by column chromatography (SiO₂, eluent CH₂Cl₂:EtOAc=1:1) to give, after evaporation and drying, 7-ethoxy-4-(4-ethyl-3,5-dimethoxybenzyl)isoquinolin-8-ol (76 mg). This solid was dissolved in MeOH (2 mL) and a 0.49 N HCl solution in MeOH (422 μL, 0.21 mmol) was slowly added. The reaction mixture was stirred at 4° C. for 15 min. After evaporation of solvent and drying under P₂O₅, 7-ethoxy-4-(4-ethyl-3,5-dimethoxybenzyl)-isoquinolin-8-ol hydrochloride 73 (82 mg, 16% yield) was obtained as a yellow solid.

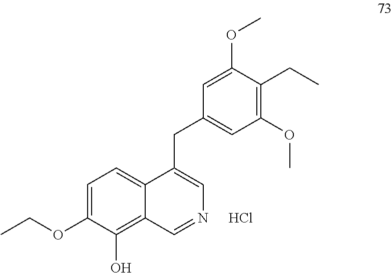 [SMILES: CCOc1ccc2c(Cc3cc(OC)c(CC)c(OC)c3)cncc2c1O ; Cl]

MW: 403.91; Yield: 16%; Yellow Solid; Mp (° C.): 227.5

R_{f}: 0.50 (CH₂Cl₂:EtOAc=1:1, free base).

¹H-NMR (MeOD, δ): 1.00 (t, 3H, J=9 Hz, CH₃), 1.47 (t, 3H, J=6 Hz, CH₃), 2.59 (q, 2H, J=6 Hz, CH₂), 3.73 (s, 6H, 2×OMe), 4.34 (q, 2H, J=6 Hz, OCH₂), 4.49 (s, 2H, CH₂), 6.52 (s, 2H, 2×ArH), 7.87 (m, 1H, ArH), 8.01 (m, 2H, 2×ArH), 9.64 (s, 1H, ArH).

¹³C-NMR (MeOD, δ): 14.2, 15.1, 17.0, 37.3, 56.2 (2×C), 66.9, 105.7 (2×C), 116.6, 120.5, 123.9, 126.7, 128.3, 132.9, 137.7, 138.2, 142.4, 146.4, 146.8, 159.8.

MS-ESI m/z (% rel. Int.): 368.0 [MH]⁺ (100).

HPLC: Method A, XBridge™ column, detection UV 254 nm, RT=4.96 min, peak area 99.0%.

### Preparation of 7-ethoxy-4-(4-ethoxy-3-methoxy-5-nitrobenzyl)isoquinolin-8-ol hydrochloride 74

### 4-Ethoxy-3-methoxy-5-nitrobenzaldehyde TTA 24158

5-Nitrovanilline (1.04 g, 5.27 mmol) was dissolved in DMF (10 mL) and K₂CO₃ (1.5 g, 10.85 mmol) was added at RT in ace pressure tube. Bromoethane (0.9 mL, 12.0 mmol) was added and the reaction mixture was stirred at 120° C. for 3 h. The cold reaction mixture was poured into water (150 mL) and extracted with Et₂O (120 mL). The organic layer was washed with water (2×50 mL), brine (30 mL), dried over MgSO₄, filtered and concentrated to dryness under reduced pressure to give yellow solid. After drying for 2 h under vacuum, 4-ethoxy-3-methoxy-5-nitrobenzaldehyde TTA 24158 (927 mg, 78% yield) was obtained as an off-white solid.

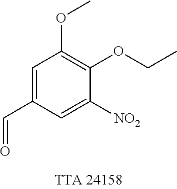 [SMILES: CCOc1c(OC)cc(C=O)cc1[N+](=O)[O-]]

MW: 225.20; Yield: 78%; Off-white solid.

¹H-NMR (CDCl₃, δ): 1.43 (t, 3H, J=7.0 Hz, CH₂CH₃), 3.99 (s, 3H, OCH₃), 4.33 (q, 2H, J=7.0 Hz, OCH₂), 7.61 (d, 1H, J=1.8 Hz, ArH), 7.82 (d, 1H, J=1.8 Hz, ArH), 9.92 (s, 1H, CHO).

¹³C-NMR (CDCl₃, δ): 15.4, 56.7, 71.2, 113.1, 119.7, 131.3, 145.1, 147.2, 154.9, 189.0.

MS-ESI m/z (% rel. Int.): 226.0 ([MH]⁺, 10), 153.0 (100).

HPLC: Method A, XBridge™ column, detection UV 254 nm, RT=5.37 min, peak area 97.0%.

### 7-Ethoxy-4-(4-ethoxy-3-methoxy-5-nitrobenzyl)isoquinolin-8-ol hydrochloride EHT 5220

In an ace pressure tube (Aldrich, 100 mL), 2-[(2,2-diethoxy-ethylamino)-methyl]-6-ethoxy-phenol SAO 33014 (142 mg, 0.51 mmol, 1.0 eq) and 4-ethoxy-3-methoxy-5-nitrobenzaldehyde TTA 24158 (115 mg, 0.51 mmol, 1.0 eq) were dissolved in EtOH (2 mL) and a 37% HCl solution (2 mL) was added. The reaction mixture was stirred at 100° C. for 15 min, cooled to 4° C. and concentrated. The residue was partitioned between EtOAc (510 mL) and K₂CO₃ (50 mL). The separated organic layer was washed with water (3×30 mL) and brine (30 mL), dried over MgSO₄, filtered and evaporated to give a yellow solid. This solid was purified by column chromatography (SiO₂, eluent cyclohexane:acetone=100:0 to 70:30) to give after evaporation and drying, 7-ethoxy-4-(4-ethoxy-3-methoxy-5-nitrobenzyl)isoquinolin-8-ol (57 mg). This solid was dissolved in MeOH (6 mL) and a 0.15 N HCl solution in MeOH (1 mL, 0.15 mmol) was slowly added and the reaction mixture was stirred for 15 min at 4° C. After evaporation and drying, 7-ethoxy-4-(4-ethoxy-3-methoxy-5-nitrobenzyl)isoquinolin-8-ol hydrochloride 74 (54.6 mg, 25% yield) was obtained as a yellow solid.

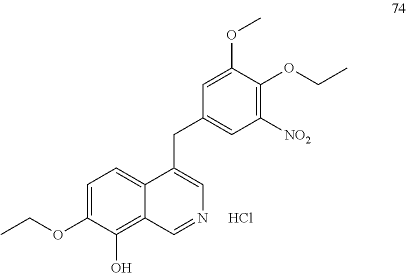 [SMILES: CCOc1ccc2c(Cc3cc(OC)c(OCC)c([N+](=O)[O-])c3)cncc2c1O ; Cl]

MW: 437.87; Yield: 25%; Yellow Solid; Mp (° C.): 218.4

R_{f}: 0.25 (cyclohexane:acetone=70:30, free base).

¹H-NMR (DMSO-d₆, δ): 1.23 (t, 3H, J=7 Hz, CH₃), 1.40 (t, 3H, J=7 Hz, CH₃), 3.87 (s, 3H, MeO), 4.08 (q, 2H, J=7 Hz, OCH₂), 4.28 (q, 2H, J=7 Hz, OCH₂), 4.53 (s, 2H, CH₂), 7.27 (s, 1H, ArH), 7.48 (s, 1H, ArH), 7.81 (d, 1H, J=9 Hz, ArH), 8.03 (d, 1H, J=9 Hz, ArH), 8.42 (s, 1H, ArH), 9.67 (s, 1H, ArH), 11.00 (broad s, 1H, OH).

¹³C-NMR (DMSO-d₆, δ): 14.6, 15.1, 34.1, 56.6, 63.4, 69.8, 114.8, 115.1, 117.3, 119.3, 125.7, 129.0, 130.9, 133.9, 135.3, 138.9, 141.8, 144.5, 144.7, 144.9, 153.4.

MS-ESI m/z (% rel. Int.): 399.1 ([MH]⁺, 100).

HPLC: Method A, XBridge™ column, detection UV 254 nm, RT=4.71 min, peak area 96.0%.

### Preparation of 4-(3-amino-4-ethoxy-5-methoxybenzyl)-7-ethoxyisoquinolin-8-ol hydrochloride 75

### 4-(3-Amino-4-ethoxy-5-methoxybenzyl)-7-ethoxyisoquinolin-8-ol hydrochloride 75

7-Ethoxy-4-(4-ethoxy-3-methoxy-5-nitrobenzyl)isoquinolin-8-ol (74 free base, 25.9 mg, 0.065 mmol, 1.0 eq) was dissolved in EtOH (518 μL), AcOH (518 μL), and water (259 μL). Fe (29.04 mg, 0.52 mmol, 8 eq) and a 37% HCl solution (10 μL, 0.044 mmol, 0.68 eq) were added. The reaction mixture was stirred at 100° C. for 20 min under a nitrogen atmosphere then cooled to RT and a 18 N NH₄OH aq. solution (0.6 mL) was added. H₂O (10 mL) was added and the solution was extracted with EtOAc (100 mL). The combined organic layers were washed with brine (10 mL), dried over MgSO₄, filtered and concentrated to give a pink solid. This solid was purified by column chromatography (SiO₂, eluent CH₂Cl₂:EtOAc=30:70) to give after evaporation and drying, an orange solid. This solid was dissolved in MeOH (5 mL) and a 0.14 N HCl solution in MeOH (363 μL, 0.051 mmol, 2.1 eq) was slowly added and the mixture stirred at RT for 3 min. After evaporation and drying, 4-(3-amino-4-ethoxy-5-methoxybenzyl)-7-ethoxyisoquinolin-8-ol hydrochloride 75 (21 mg, 73% yield) was obtained as a brown solid.

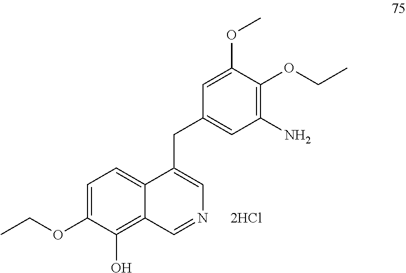 [SMILES: CCOc1ccc2c(Cc3cc(N)c(OCC)c(OC)c3)cncc2c1O ; "2HCl"]

MW: 441.34; Yield: 73%; Brown solid; Mp (° C.): 223.5

¹H-NMR (CD₃OD, δ): 1.17-1.25 (m, 3H, CH₃), 1.33-1.40 (m, 3H, CH₃), 3.65-3.68 (m, 3H, OMe), 3.87-3.93 (m, 2H, OCH₂), 4.18-4.26 (m, 2H, OCH₂), 4.79-4.81 (m, 2H, CH₂), 6.28-6.30 (m, 1H, ArH), 6.40-6.42 (m, 1H, ArH), 7.63-7.68 (m, 1H, ArH), 7.81-7.85 (m, 1H, ArH), 7.95-7.98 (m, 1H, ArH), 9.48-9.51 (m, 1H, ArH).

¹³C-NMR (CD₃OD, δ): 15.1, 15.9, 36.7, 56.4, 66.9, 69.3, 106.7, 112.0, 116.6, 120.5, 126.4, 129.0, 132.7, 135.3, 136.5, 137.6, 138.8, 142.6, 146.2, 146.6, 154.6

MS-ESI m/z (% rel. Int.): 369.1 ([MH]⁺, 100).

HPLC: Method A, XBridge™ column, detection UV 254 nm, RT=3.66 min, peak area 95.0%.

### Preparation of 4-(3,5-dimethoxy-4-(pyrrolidin-1-yl)benzyl)-7-ethoxyisoquinolin-8-ol hydrochloride 76

### 3,5-Dimethoxy-4-(pyrrolidin-1-yl)benzaldehyde RBO 40138

In a 20 mL microwave vial, 4-formyl-2,6-dimethoxyphenyl trifluoromethanesulfonate RBO 40040 (500 mg, 1.6 mmol), pyrrolidine (228 mg, 264 μL, 3.2 mmol), Pd(OAc)₂ (11 mg, 0.05 mmol), BINAP (44 mg, 0.07 mmol), and Cs₂CO₃ (730 mg, 2.24 mg) were dissolved in dioxan (16 mL). The reaction mixture was heated at 140° C. for 20 min under microwave irradiation. After filtration through Celite, dioxan was evaporated and EtOAc (30 mL) was added to the obtained residue. The organic layer was washed with water (2×30 mL), brine (30 mL), dried over Na₂SO₄, filtered and concentrated to dryness under reduced pressure to give a brown oil. This oil was purified by column chromatography (SiO₂, eluent CH₂Cl₂:EtOAc=1:1) to give, after evaporation and drying, 3,5-dimethoxy-4-(pyrrolidin-1-yl)benzaldehyde RBO 40138 (142 mg, 38% yield) as a pale yellow solid.

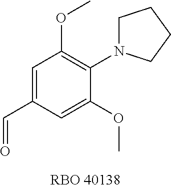 [SMILES: COc1cc(C=O)cc(OC)c1N1CCCC1]

MW: 235.29; Yield: 38%; Pale yellow solid.

R_{f}: 0.2 (CH₂Cl₂:EtOAc=9:1).

¹H-NMR (CDCl₃, δ): 1.88 (s, 4H, 2×CH₂), 3.56 (s, 4H, 2×CH₂), 3.85 (s, 6H, 2×OMe), 7.07 (s, 2H, 2×ArH), 9.75 (s, 1H, CHO).

MS-ESI m/z (% rel. Int.): 236.1 ([MH]⁺, 100).

HPLC: Method A (5 min), XBridge™ column, detection UV 254 nm, RT=1.92 min, peak area 99.0%.

### 4-(3,5-Dimethoxy-4-(pyrrolidin-1-yl)benzyl)-7-ethoxyisoquinolin-8-ol hydrochloride 76

In an ace pressure tube (20 mL, Aldrich), 2-((2,2-diethoxyethylamino)methyl)-6-ethoxyphenol SAO 33014 (171 mg, 0.60 mmol) and 3,5-dimethoxy-4-(pyrrolidin-1-yl)benzaldehyde RBO 40138 (142 mg, 0.60 mmol) were dissolved in EtOH:37% HCl solution=1:1 (4 mL) and the reaction mixture was stirred at 110° C. for 10 min then cooled to RT. The volatiles were removed under vacuum and EtOAc (100 mL) was added. This solution was neutralized with a 10% NaHCO₃ aq. solution (10 mL). The resulting solution was extracted with EtOAc (3×30 mL). The combined organic layers were washed with water (3×30 mL), brine (30 mL), dried over Na₂SO₄, filtered and evaporated to give yellow solid. This solid was purified by column chromatography, (SiO₂, eluent CH₂Cl₂:MeOH with a 7 N NH₃ aq. solution=100:0 to 90:10) to give, after evaporation and drying, 4-(3,5-dimethoxy-4-(pyrrolidin-1-yl)benzyl)-7-ethoxyisoquinolin-8-ol (142 mg). This solid was dissolved in MeOH (2 mL) and a 0.49 N HCl solution in MeOH (709 μL, 0.35 mmol) was slowly added and the reaction mixture was stirred at 4° C. for 15 min. After evaporation and drying under P₂O₅, 4-(3,5-dimethoxy-4-(pyrrolidin-1-yl)benzyl)-7-ethoxyisoquinolin-8-ol hydrochloride 76 (148 mg, 55% yield) was obtained as a yellow solid.

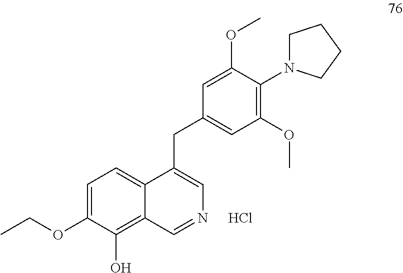 [SMILES: CCOc1ccc2c(Cc3cc(OC)c(N4CCCC4)c(OC)c3)cncc2c1O ; Cl]

MW: 444.96; Yield: 55%; Yellow Solid; Mp (° C.): 219.5

R_{f}: 0.30 (CH₂Cl₂:MeOH with 7 N NH₃ aq. solution=9:1, free base).

¹H-NMR (CD₃OD, δ): 1.50 (t, 3H, J=6 Hz, CH₃), 2.20 (m, 2H, CH₂CH₂N), 2.37 (m, 2H, CH₂CH₂N), 3.74 (m, 4H, 2×CH₂CH₂N), 3.96 (s, 6H, 2×OMe), 4.35 (q, 2H, OCH₂), 4.60 (s, 2H, CH₂), 6.87 (s, 2H, 2×ArH), 7.85 (d, 1H, J=9.0 Hz, ArH), 8.04 (d, 1H, J=9.0 Hz, ArH), 8.21 (s, 1H, ArH), 9.70 (s, 1H, ArH).

¹³C-NMR (CD₃OD, δ): 15.0, 26.7 (2×C), 37.0, 57.4 (2×C), 58.6 (2×C), 66.0, 107.3 (2×C), 116.5, 116.9, 120.5, 126.8, 128.7, 132.6, 143.0, 143.9, 146.5, 147.1, 154.3 (2×C).

MS-ESI m/z (% rel. Int.): 409.0 ([MH]⁺, 75), 205 (100).

HPLC: Method A, XBridge™ column, detection UV 254 nm, RT=3.42 min, peak area 99.0%.

### Preparation of 4-(3-bromo-4-ethoxy-5-methoxybenzyl)-7-ethoxyisoquinolin-8-ol hydrochloride 77

### 3-Bromo-4-ethoxy-5-methoxybenzaldehyde ANP 36050

In an ace pressure tube, 3-bromo-4-hydroxy-5-methoxybenzaldehyde (1.0 g, 4.33 mmol) was dissolved in anhydrous DMF (10 mL) and cesium carbonate (1.48 g, 4.54 mmol) was added at RT. Bromoethane (355 μL, 4.76 mmol) was added and the solution was stirred at 100° C. for 2 h. After cooling to RT, the reaction was poured into water (250 mL) and extracted with Et₂O (200 mL). The organic layer was washed with water (2×50 mL), brine (50 mL), dried over MgSO₄, and filtered. After evaporation and drying, 3-bromo-4-ethoxy-5-methoxybenzaldehyde ANP 36050 (1.09 g, 97% yield) was obtained as a white solid.

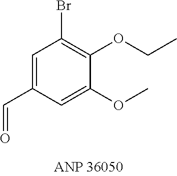

MW: 259.10; Yield: 97%; White solid; Mp (° C.): 55.9

R_{f}: 0.8 (CH₂Cl₂:EtOAc=8:2).

¹H-NMR (CD₃OD, δ): 1.44 (t, 3H, CH₃, J=7.04 Hz), 3.92 (s, 3H, OMe), 4.19 (q, 2H, CH₂, J=7.06 Hz), 7.38 (s, 1H, ArH), 7.65 (s, 1H, ArH), 9.84 (s, 1H, ArH).

¹³C-NMR (CD₃OD, δ): 15.7, 56.2, 69.6, 110.0, 118.4, 128.8, 132.9, 151.1, 154.3, 189.9.

MS-ESI m/z (% rel. Int.): 259.0/261.0 ([MH]⁺, 100/100).

HPLC: Method A, XBridge™ column, detection UV 254 nm, RT=5.81 min, peak area 99.9%.

### 4-(3-Bromo-4-ethoxy-5-methoxybenzyl)-7-ethoxyisoquinolin-8-ol hydrochloride 77

In an ace pressure tube, to a solution of 2-((2,2-diethoxyethylamino)methyl)-6-ethoxyphenol SAO 33014 (200 mg, 0.71 mmol) in EtOH (1.2 mL) was added at RT 3-bromo-4-ethoxy-5-methoxy-benzaldehyde ANP 36050 (183 mg, 0.71 mmol) and a 37% HCl solution (1.2 mL). The solution was stirred at 100° C. for 20 min then cooled to 4° C. The solvent was evaporated to give an orange solid (353 mg). This solid was dissolved in H₂O (5 mL) and a 18 N NH₄OH aq. solution (300 μL) was added (until basic pH). The resulting solution was extracted with EtOAc (80 mL). The combined organic layers were washed with brine (10 mL), dried over MgSO₄, filtered and evaporated to give red solid (295 mg). This crude solid was purified by column chromatography (SiO₂, cyclohexane:EtOAc=90:10 to 50:50) to give, after evaporation and drying, 4-(3-bromo-4-ethoxy-5-methoxybenzyl)-7-ethoxyisoquinolin-8-ol (50.4 mg) as a yellow oil. This oil was dissolved in a mixture of MeOH:CH₂Cl₂=1:1 (2 mL) and a 1.34 N HCl solution in MeOH (96 μL, 0.13 mmol, 1.05 eq) was added and the solution was stirred at 4° C. for 10 min. After evaporation and drying, 4-(3-bromo-4-ethoxy-5-methoxybenzyl)-7-ethoxyisoquinolin-8-ol hydrochloride 77 (38 mg, 11% yield) was obtained as a yellow solid.

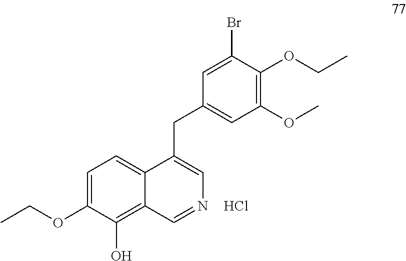 [SMILES: CCOc1ccc2c(Cc3cc(Br)c(OCC)c(OC)c3)cncc2c1O ; Cl]

MW: 468.77; Yield: 11%; Yellow solid; Mp (° C.): 229.6

R_{f}: 0.25 (cyclohexane:EtOAc=5:5, free base).

¹H-NMR (CD₃OD, δ): 1.35 (t, 3H, CH₃, J=7.04 Hz), 1.50 (t, 3H, CH₃, J=6.98 Hz), 3.81 (s, 3H, OMe), 3.99 (q, 2H, CH₂, J=7.02 Hz), 3.34 (q, 2H, CH₂, J=6.99 Hz), 4.48 (s, 2H, CH₂), 6.99 (s, 1H, ArH), 7.02 (s, 1H, ArH), 7.83 (d, 1H, ArH, J=9.02 Hz), 8.03 (d, 1H, ArH, J=9.08 Hz), 8.12 (s, 1H, ArH), 9.67 (s, 1H, ArH).

¹³C-NMR (CD₃OD, δ): 13.5, 14.3, 34.6, 55.2, 65.4, 68.5, 112.4, 115.0, 117.5, 119.0, 124.2, 125.3, 126.9, 131.1, 135.0, 135.7, 141.2, 144.3, 145.0, 145.5, 154.0.

MS-ESI m/z (% rel. Int.): 432.0/434.0 ([MH]⁺¹, 100/100).

HPLC: Method A, XBridge™ column, detection UV 254 nm, RT=4.86 min, peak area 99.9%.

### Preparation of 7-ethoxy-4-(4-isobutoxy-3,5-dimethoxybenzoyl)isoquinolin-8-yl acetate 78

### 7-Ethoxy-4-(4-isobutoxy-3,5-dimethoxybenzyl)isoquinolin-8-yl acetate ANP 361068

To a solution of 7-ethoxy-4-(4-isobutoxy-3,5-dimethoxybenzyl)-isoquinolin-8-ol 20 free base (3.6 g, 8.75 mmol) in anhydrous CH₂Cl₂ (150 mL) were added at RT successively DIEA (5.80 mL, 35.0 mmol), DMAP (246 mg, 2.01 mmol) and Ac₂O (3.31 mL, 35.0 mmol) under a nitrogen atmosphere. The reaction mixture was stirred overnight at RT then CH₂Cl₂ (500 mL) and water (50 mL) were added. The separated organic layer was washed with brine (50 mL), dried over MgSO₄, filtered and evaporated to give a brown solid (5.0 g). This crude solid was purified by column chromatography (SiO₂, cyclohexane:EtOAc=90:10 to 50:50) to yield, after evaporation and drying, 7-ethoxy-4-(4-isobutoxy-3,5-dimethoxybenzyl)isoquinolin-8-yl acetate ANP 36106B (3.60 g, 90% yield) as a yellow pale solid.

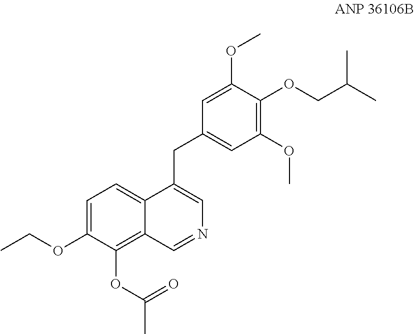 [SMILES: CCOc1ccc2c(Cc3cc(OC)c(OCC(C)C)c(OC)c3)cncc2c1OC(C)=O]

MW: 453.53; Yield: 90%; Yellow pale solid.

R_{f}: 0.35 (cyclohexane:EtOAc=5:5).

¹H-NMR (CDCl₃, δ): 0.98 (d, 6H, J=6.66 Hz, 2×CH₃), 1.41 (t, 3H, J=7 Hz, CH₃), 2.01 (m, 1H, CH), 2.47 (s, 3H, CH₃), 3.67 (d, 2H, J=6.72 Hz, OCH₂), 3.72 (s, 6H, 2×OMe), 4.18 (q, 2H, J=6.96 Hz, CH₂), 4.27 (s, 2H, CH₂), 6.37 (s, 2H, 2×ArH), 7.48 (d, 1H, J=9.27 Hz, ArH), 7.82 (d, 1H, J=9.25 Hz, ArH), 8.28 (s, 1H, ArH), 9.22 (s, 1H, ArH).

¹³C-NMR (CDCl₃, δ): 14.90, 19.15 (2×C), 20.45, 29.17, 56.39 (2×C), 65.48, 80.26, 108.24 (2×C), 120.64, 123.60, 124.07, 128.73, 128.88, 132.44, 134.22, 142.44, 143.27, 148.21, 148.62, 153.29, 168.54, 194.71.

MS-ESI m/z (% rel. Int.): 454.1 ([MH]⁺, 100), 455.1 (27), 456.1 (6).

HPLC: Method A, XBridge™ column, detection UV 254 nm, RT=5.26 min, peak area 99.9%.

### 7-Ethoxy-4-(4-isobutoxy-3,5-dimethoxybenzoyl)isoquinolin-8-yl acetate 78

To a solution of 7-ethoxy-4-(4-isobutoxy-3,5-dimethoxybenzyl)isoquinolin-8-yl acetate ANP 36106B (3.60 g, 7.94 mmol) in a mixture CH₃CN:H₂O=50:50 (160 mL) were added at RT N-hydroxyphthalimide (260 mg, 1.59 mmol) and NaClO₂ (1.26 g, 11.11 mmol). The reaction mixture was stirred at 85° C. for 4 h. After cooling to RT, Et₂O (2×300 mL) and a 10% aq. sodium sulfite solution were added (20 mL). The separated organic layer was washed with brine (100 mL), dried over MgSO₄, filtered, evaporated and to give a crude red solid (2.86 g). This crude product was purified by column chromatography (SiO₂, cyclohexane:EtOAc=100:0 to 70:30) to give, after evaporation and drying, 7-ethoxy-4-(4-isobutoxy-3,5-dimethoxybenzoyl)isoquinolin-8-yl acetate 78 (62.2 mg, 2% yield) as a yellow solid.

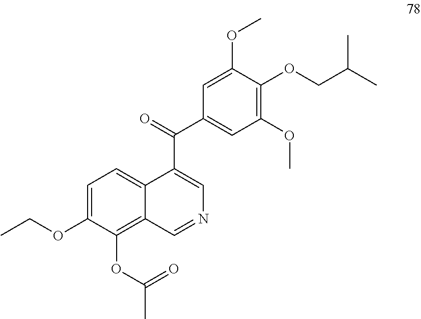 [SMILES: CCOc1ccc2c(C(=O)c3cc(OC)c(OCC(C)C)c(OC)c3)cncc2c1OC(C)=O]

MW: 467.51; Yield: 2%; Yellow solid; Mp (° C.):109.3

R_{f}: 0.25 (cyclohexane:EtOAc=70:30).

¹H-NMR (CDCl₃, δ): 1.03 (d, 6H, J=6.7 Hz, 2×CH₃), 1.45 (t, 3H, J=6.96 Hz, CH₃), 2.07 (m, 1H, J=6.69 Hz, CH), 2.52 (s, 3H, CH₃), 3.83 (s, 6H, 2×OMe), 3.86 (d, 2H, J=6.69 Hz, OCH₂), 4.24 (q, 2H, J=7 Hz, CH₂), 7.14 (s, 2H, ArH), 7.58 (d, 1H, J=9.37 Hz, ArH), 8.07 (d, 1H, J=9.33 Hz, ArH), 8.56 (s, 1H, ArH), 9.41 (s, 1H, ArH).

¹³C-NMR (CDCl₃, δ): 14.90, 19.15 (2×C), 20.45, 29.17, 56.39 (2×C), 65.48, 80.26, 108.24 (2×C), 120.64, 123.60, 124.07, 128.73, 128.88, 132.44, 134.22, 142.44, 143.27, 148.21, 148.62, 153.29, 168.54, 194.71.

MS-ESI m/z (% rel. Int.): 468.3 ([MH]⁺, 100), 469.3 (28), 470.3 (5).

HPLC: Method A, XBridge™ column, detection UV 254 nm, RT=6.02 min, peak area 99.9%.

### Preparation of (7-Ethoxy-8-hydroxyisoquinolin-4-yl)(4-isobutoxy-3,5-dimethoxyphenyl)methanone hydrochloride 79

### (7-Ethoxy-8-hydroxyisoquinolin-4-yl)(4-isobutoxy-3,5-dimethoxyphenyl)methanone hydrochloride 79

To a solution of 7-ethoxy-4-(4-isobutoxy-3,5-dimethoxybenzoyl)isoquinolin-8-yl acetate 78 (80 mg, 0.22 mmol) in anhydrous CH₂Cl₂ (6 mL) was added at RT a 7 N NH₃ solution in MeOH (1.86 mL, 13.05 mmol) under a nitrogen atmosphere and the reaction mixture was stirred at RT overnight. H₂O (20 mL) was added and the resulting solution extracted with CH₂Cl₂ (2×50 mL). The combined organic layers were washed with brine (20 mL), dried over MgSO₄, filtered and evaporated to give yellow solid (95.7 mg). This crude solid was purified by column chromatography (SiO₂, eluent CH₂Cl₂:MeOH=100:0 to 98:2) to yield, after evaporation and drying, (7-ethoxy-8-hydroxyisoquinolin-4-yl)(4-isobutoxy-3,5-dimethoxyphenyl)methanone (66.8 mg) as a yellow solid. This solid was dissolved in a mixture MeOH:CH₂Cl₂=1:1 (2 mL) and a 1.34 N HCl solution in MeOH (129 μL, 0.17 mmol) was added at 4° C. and the reaction mixture was stirred at 4° C. for 10 min. after evaporation and drying, (7-ethoxy-8-hydroxyisoquinolin-4-yl)(4-isobutoxy-3,5-dimethoxyphenyl)methanone hydrochloride 79 (70.8 mg, 70% yield) was obtained as an orange solid.

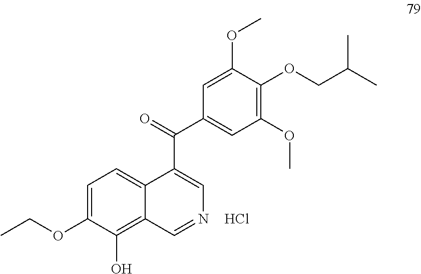 [SMILES: CCOc1ccc2c(C(=O)c3cc(OC)c(OCC(C)C)c(OC)c3)cncc2c1O ; Cl]

MW: 461.94; Yield: 70%; Orange solid; Mp (° C.): 234.3

R_{f}: 0.20 (CH₂Cl₂:MeOH=98:2, free base).

¹H-NMR (CD₃OD, δ): 1.0 (d, 6H, 2×CH₃, J=6.69 Hz), 1.50 (t, 3H, CH₃, J=7 Hz), 1.98 (m, 1H, CH, J=6.66 Hz), 3.78 (s, 6H, 2×OMe), 3.81 (d, 2H, OCH₂, J=6.55 Hz), 4.35 (q, 2H, CH₂, J=6.96 Hz), 7.18 (s, 2H, 2×ArH), 7.60 (d, 1H, ArH, J=9.03 Hz), 8.03 (d, 1H, ArH, J=9.1 Hz), 8.43 (s, 1H, ArH), 9.87 (s, 1H, ArH).

¹³C-NMR (CD₃OD, δ): 15.0, 19.47 (2×C), 30.42, 56.94 (2×C), 66.99, 81.26, 109.42 (2×C), 117.53, 120.95, 127.49, 129.21, 130.87, 132.65, 135.41, 145.33, 145.67, 146.88, 147.15, 154.96 (2×C), 192.55.

MS-ESI m/z (% rel. Int.): 426.3 ([MH]⁺, 100), 427.3 (26), 428.3 (4).

HPLC: Method A, XBridge™ column, detection UV 254 nm, RT=5.19 min, peak area 99.9%.

### Preparation of 6-(3,5-dimethoxy-4-propoxybenzyl)-3-ethyloxazolo[4,5-h]isoquinolin-2(3H)-one hydrochloride 80

### tert-Butyl (3-fluorophenyl)carbamate TTA 24190

3-Fluoroaniline (8.3 g, 74 mmol) was dissolved in di-tert-butyl dicarbonate (19.5 g, 90 mmol) at 30° C. The reaction mixture was stirred at RT for 15 h. H₂O (100 mL) was added and the precipitate was filtered and washed with water (3×20 mL). After drying under P₂O₅, tert-butyl (3-fluorophenyl)carbamate TTA 24190 (13.5 g, 86% yield) was obtained as a white solid.

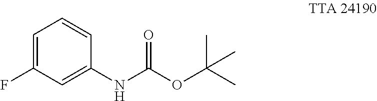

MW: 211.23; Yield: 86%; White Solid; Mp (° C.): 128.5

¹H-NMR (CDCl₃, δ): 1.52 (t, 9H, J=7.3 Hz, CH₃), 6.54 (broad s, 1H, NH), 6.69-6.75 (m, 1H, ArH), 6.96-9.99 (m, 1H, ArH), 7.17-7.22 (m, 1H, ArH), 7.29-7.33 (m, 1H, ArH).

MS-ESI m/z (% rel. Int.): 155.9 ([MH-(tBu)]⁺, 100).

HPLC: Method A, XBridge™ column, detection UV 254 nm, RT=5.90 min, peak area 98.0%.

### 2-Oxo-2,3-dihydro-benzooxazole-7-carbaldehyde TTA 391928

In a dry 50 mL round bottom flask, tert-butyl (3-fluorophenyl)carbamate TTA 24190 (1.5 g, 7.3 mmol) was dissolved in anhydrous THF (16 mL) under a nitrogen atmosphere. The reaction mixture was cooled at −70° C. and a 1.7 N ^{t}BuLi solution in pentane (13 mL, 22 mmol, 3 eq) was slowly added (temperature reached −62° C.). The mixture was stirred for 40 min at −62° C. At −60° C., anhydrous DMF (2 mL) was slowly added and the reaction mixture was stirred for 75 min at about −50° C. At −30° C., the reaction mixture was quenched with a 2 N HCl solution (15 mL) and the reaction mixture was abandoned overnight. At 9° C., the reaction mixture was partitioned between water (60 mL) and EtOAc (250 mL). The separated organic layer was washed with water (30 mL), brine (2×20 mL), dried over MgSO₄, filtered and evaporated to give a crude green solid. This crude solid was stirred in Et₂O (30 mL) for 1.5 h, filtered, and washed with ether (5 mL) to give after drying, 2-oxo-2,3-dihydro-benzooxazole-7-carbaldehyde TTA 39192B (1.14 g, 35% yield) as a beige solid.

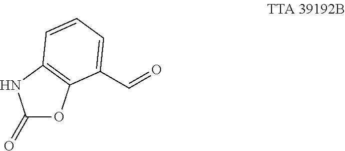 [SMILES: O=Cc1cccc2[nH]c(=O)oc12]

MW: 163.13, Yield: 31%, Beige solid; Mp (° C.): 217

R_{f}: 0.25 (CH₂Cl₂:EtOAc=70:30).

¹H-NMR (CDCl₃, δ): 7.33 (t, 1H, J=7.7 Hz, ArH), 7.40 (dd, 1H, J=7.7 Hz, J=1.2 Hz, ArH), 7.55 (dd, 1H, J=7.7 Hz, J=1.2 Hz, ArH), 12.01 (s, 1H, CHO).

¹³C-NMR (CDCl₃, δ): 115.07, 119.01, 121.92, 123.87, 131.56, 143.26, 154.13, 188.29.

MS-ESI m/z (% rel. Int.): 164.0 ([MH]⁺, 100), 205.0 (15).

HPLC: Method A, XBridge™ column, detection UV 254 nm, RT=3.41 min, peak area 98.0%.

### 3-Ethyl-2-oxo-2,3-dihydrobenzo[d]oxazole-7-carbaldehyde LPO 43046C

In an ace pressure tube, 2-oxo-2,3-dihydro-benzooxazole-7-carbaldehyde TTA 24192B (190 mg, 1.17 mmol) was dissolved in anhydrous DMF (4 mL). Bromoethane (96 μL, 1.28 mmol) and cesium carbonate (342 mg, 1.05 mmol) were slowly added. The reaction mixture was stirred at 100° C. for 1 h. Et₂O (170 mL) and water (30 mL) were added. The separated organic layer was washed with brine (30 mL), dried over MgSO₄, filtered and evaporated. The crude product was purified by column chromatography (SiO₂, eluent cyclohexane:EtOAc=100:0 to 70:30) to give after evaporation and drying, 3-ethyl-2-oxo-2,3-dihydrobenzo[d]oxazole-7-carbaldehyde LPO 43046C (131 mg, 59% yield) as a white solid.

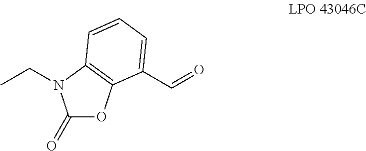

MW: 191.18; Yield: 59%; White Solid; Mp (° C.): 102.8

R_{f}: 0.3 (cyclohexane:EtOAc=70:30).

¹H-NMR (CDCl₃, δ): 1.42 (t, 3H, J=7.3 Hz, CH₃), 3.95 (q, 2H, J=7.3 Hz, NCH₂), 7.21-7.35 (m, 2H, 2×ArH), 7.61 (d, 1H, J=7.9 Hz, ArH), 10.37 (s, 1H, CHO).

¹³C-NMR (CDCl₃, δ): 12.9, 37.6, 113.1, 119.6, 120.9, 123.9, 131.9, 143.9, 153.6, 186.5.

MS-ESI m/z (% rel. Int.): 192.0 ([MH]⁺, 100).

HPLC: Method A, XBridge™ column, detection UV 254 nm, RT=4.22 min, peak area 98.0%.

### 7-(((2,2-Diethoxyethyl)amino)methyl)-3-ethylbenzo[d]oxazol-2(3H)-one SSA 39148

3-Ethyl-2-oxo-2,3-dihydrobenzo[d]oxazole-7-carbaldehyde LPO 43046C (130 mg, 0.68 mmol, 1.0 eq) was dissolved in CH₂Cl₂ (3.5 mL) under a nitrogen atmosphere at RT and aminoacetaldehyde diethyl acetal (119 μL, 0.82 mmol, 1.2 eq) was added. The reaction mixture was stirred at RT for 5 min. Acetic acid (8 μL, 0.136 mmol, 0.2 eq) and sodium cyanoborohydride (47 mg, 0.75 mmol, 1.1 eq) were slowly added. The reaction mixture was stirred at RT overnight then diluted with CH₂Cl₂ (80 mL) and water was added (50 mL). The separated organic layer was washed with water (50 mL), brine (20 mL), dried over MgSO₄, filtered and concentrated to give a yellow oil. The crude oil was purified by column chromatography (SiO₂, eluent CH₂Cl₂:MeOH=100:0 to 98:2) to give after evaporation and drying 7-(((2,2-diethoxyethyl)amino)methyl)-3-ethylbenzo[d]oxazol-2(3H)-one SSA 39148 (128 mg, 61% yield) as a pale yellow oil.

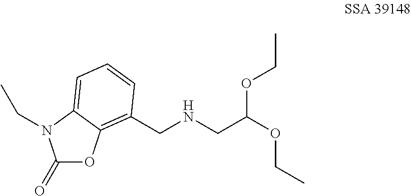 [SMILES: CCOC(CNCc1cccc2c1oc(=O)n2CC)OCC]

MW: 308.38; Yield: 61%; Pale yellow oil.

¹H-NMR (CDCl₃, δ): 1.28 (t, 6H, J=7 Hz, 2×CH₃), 1.37 (t, 3H, J=7 Hz, CH₃), 2.73 (d, 2H, J=6 Hz, NCH₂CH₃), 3.53 (q, 2H, J=6 Hz, OCH₂), 3.68 (q, 2H, J=6 Hz, OCH₂), 3.88 (q, 2H, J=7 Hz, OCH₂), 3.96 (s, 2H, CH₂), 4.61 (t, 1H, J=5 Hz, CH), 6.88 (d, 1H, J=7 Hz, ArH), 7.14-7.07 (m, 2H, 2×ArH).

¹³C-NMR (CDCl₃, δ): 154.2, 140.9, 130.7, 123.7, 123.2, 122.6, 106.8, 102.2, 62.5, 51.5, 47.20, 37.2, 15.4, 13.0

MS-ESI m/z (% rel. Int.): 309.1 ([M+H]⁺, 10), 263.1 (100).

HPLC: Method A, XBridge™ column, detection UV 230 nm, RT=3.69 min, peak area 96.0%.

### 6-(3,5-Dimethoxy-4-propoxybenzyl)-3-ethyloxazolo[4,5-h]isoquinolin-2(3H)-one hydrochloride 80

In an ace pressure tube, 7-(((2,2-diethoxyethyl)amino)methyl)-3-ethylbenzo[d]oxazol-2(3H)-one SSA 39148 (300 mg, 0.97 mmol, 1.0 eq) and 3,5-dimethoxy-4-propoxybenzaldehyde TTA 24142 (218 mg, 0.97 mmol, 1.0 eq) were dissolved in EtOH (2.5 mL) and a 37% HCl solution (2.5 mL) was added. The reaction mixture was stirred at 125° C. for 12 min, cooled to 4° C. and concentrated. A 1 N K₂CO₃ aq. solution (20 mL) and EtOAc (200 mL) were added to the obtained residue. The separated organic layer was washed with brine (10 mL), dried over MgSO₄, filtered and evaporated to give a brown oil. This oil was purified by column chromatography (SiO₂, eluent CH₂Cl₂:EtOAc=9:1 to 7:3) to give, after evaporation and drying, 6-(3,5-dimethoxy-4-propoxybenzyl)-3-ethyloxazolo[4,5-h]isoquinolin-2(3H)-one (39.2 mg) as a brown solid. This solid was dissolved in MeOH (1 mL) and a 1.34 N HCl solution in MeOH (72.7 μL, 0.097 mmol, 1.05 eq) was slowly added and the reaction mixture was stirred at 4° C. for 15 min. After evaporation and drying under P₂O₅, 6-(3,5-dimethoxy-4-propoxybenzyl)-3-ethyloxazolo[4,5-h]isoquinolin-2(3H)-one hydrochloride 80 (40.4 mg, 9% yield) was obtained as a yellow solid.

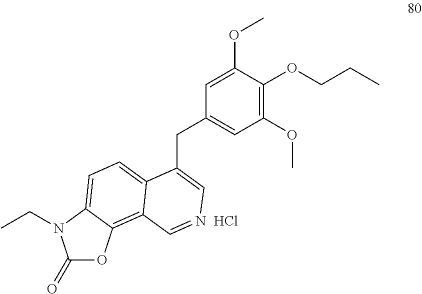 [SMILES: CCCOc1c(OC)cc(Cc2cncc3c2ccc2c3oc(=O)n2CC)cc1OC ; Cl]

MW: 458.93; Yield: 9%; Yellow solid; Mp (° C.): 85.8

R_{f}: 0.15 (CH₂Cl₂:EtOAc=9:1, free base).

¹H-NMR (CD₃OD, δ): 1.01 (t, 3H, J=7.4 Hz, CH₃), 1.45 (t, 3H, J=7.2 Hz, CH₃), 1.70 (q, 2H, J=7.1 Hz, CH₂), 3.78 (s, 6H, 2×OMe), 3.85 (t, 2H, J=6.6 Hz, OCH₂), 4.13 (q, 2H, J=7.3 Hz, NCH₂), 4.62 (s, 2H, CH₂), 6.65 (s, 2H, 2×ArH), 8.26 (d, 1H, J=9.0 Hz, ArH), 8.35 (s, 1H, ArH), 8.41 (d, 1H, J=9.0 Hz, ArH), 9.83 (s, 1H, ArH).

¹³C-NMR (CD₃OD, δ): 10.79, 13.39, 24.33, 37.30, 39.14, 56.75 (2×C), 76.18, 107.71 (2×C), 114.83, 121.08, 122.19, 130.07, 132.74, 133.53, 134.58, 137.58, 139.80, 140.18, 140.69, 155.09, 155.27 (2×C).

MS-ESI m/z (% rel. Int.): 423.1 ([MH]⁺, 100).

HPLC: Method A, XBridge™ column, detection UV 254 nm, RT=5.27 min, peak area 99.0%.

### Preparation of 6-(3,5-dimethoxy-4-propoxybenzoyl)-3-ethyloxazolo[4,5-h]isoquinolin-2(3H)-one hydrochloride 81

### 6-(3,5-Dimethoxy-4-propoxybenzoyl)-3-ethyloxazolo[4,5-h]isoquinolin-2(3H)-one hydrochloride 81

6-(3,5-Dimethoxy-4-propoxybenzyl)-3-ethyloxazolo[4,5-h]isoquinolin-2(3H)-one 80 free base (70.7 mg, 0.167 mmol), N-hydroxyphthalimide (5.4 mg, 0.033 mmol) and NaClO₂ (80% pure, 26.5 mg, 0.23 mmol) in CH₃CN:H₂O=2:1 (3 mL:1.5 mL) was stirred in a round bottom flask for 4.5 h at 85° C. After cooling to RT, the mixture was diluted with Et₂O (100 mL), washed with a 10% aq. sodium sulfite solution (10 mL), with brine (10 mL), dried over MgSO₄, filtered and concentrated. The obtained crude product was purified by column chromatography (SiO₂, eluent cyclohexane:acetone=100:0 to 75:25) to give, after evaporation and drying 6-(3,5-dimethoxy-4-propoxybenzoyl)-3-ethyloxazolo[4,5-h]isoquinolin-2(3H)-one (30.3 mg, 0.069 mmol). This material was dissolved in MeOH (1 mL), a 1.34 N HCl solution in MeOH (54.5 μL, 0.073 mmol, 1.05 eq) was slowly added and the reaction mixture was stirred at 4° C. for 15 min. After evaporation and drying under P₂O₅, 6-(3,5-dimethoxy-4-propoxybenzoyl)-3-ethyloxazolo[4,5-h]isoquinolin-2(3H)-one hydrochloride 81 (32.3 mg, 41% yield) was obtained as a yellow solid.

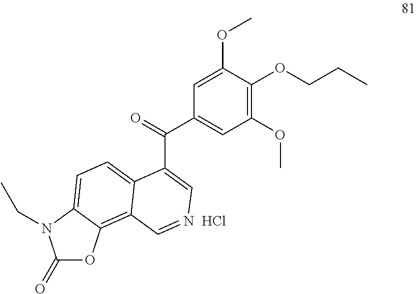 [SMILES: CCCOc1c(OC)cc(C(=O)c2cncc3c2ccc2c3oc(=O)n2CC)cc1OC ; Cl]

MW: 472.92; Yield: 41%; Yellow solid; Mp (° C.): 155.3

R_{f}: 0.25 (cyclohexane:acetone=75:25, free base).

¹H-NMR (CD₃OD, δ): 1.04 (t, 3H, J=7.4 Hz, CH₃), 1.46 (t, 3H, J=7.1 Hz, CH₃), 1.74 (q, 2H, J=7.1 Hz, CH₂), 3.81 (s, 6H, 2×OMe), 4.03 (t, 2H, J=6.5 Hz, OCH₂), 4.13 (q, 2H, J=7.2 Hz, NCH₂), 7.22 (s, 2H, 2×ArH), 8.08 (d, 1H, J=8.9 Hz, ArH), 8.25 (d, 1H, J=8.9 Hz, ArH), 8.69 (s, 1H, ArH), 10.04 (s, 1H, ArH).

¹³C-NMR (CD₃OD, δ): 10.73, 13.38, 24.48, 39.19, 56.99 (2×C), 76.39, 109.50 (2×C), 115.29, 121.39, 122.93, 131.58, 131.63, 132.64, 132.91, 136.40, 139.96, 144.37, 145.12, 155.01 (2×C), 155.07, 192.30.

MS-ESI m/z (% rel. Int.): 437.1 ([MH]⁺, 100).

HPLC: Method A, XBridge™ column, detection UV 254 nm, RT=6.27 min, peak area 97.0%.

### Preparation of S-(4-(3,5-dimethoxy-4-propoxybenzyl)-7-ethoxyisoquinolin-8-yl) dimethylcarbamothioate hydrochloride 82

### S-(2-Ethoxy-6-formylphenyl)dimethylcarbamothioate SSA 391828

3-Ethoxysalicylaldehyde (2.1 g, 12.6 mmol, 1.0 eq) was dissolved in H₂O (8 mL) and KOH (850 mg, 12.6 mmol, 1.0 eq) was added under a nitrogen atmosphere. The reaction mixture was cooled at 4° C. and a dimethylthiocarbamoyl chloride (1.6 g, 12.6 mmol, 1.0 eq) solution in THF (8 mL) was added dropwise for 20 min and the mixture was stirred 15 min to RT. A 0.5 N KOH aq. solution (8 mL) was added (until basic pH) and the reaction mixture was stirred for another 10 min. The reaction mixture was partitioned between a mixture ice-water (60 mL) and CH₂Cl₂ (150 mL). The separated organic layer was washed with water (60 mL), brine (35 mL), dried over MgSO₄, filtered and evaporated to give crude O-2-ethoxy-6-formylphenyl dimethylcarbamothioate SSA 39182A as a beige solid (2.76 g, 87% yield). SSA 39182A (1.2 g, 4.7 mmol) was dissolved in diphenylether (16 mL). This mixture was stirred at 240° C. for 20 min under a nitrogen atmosphere. After evaporation the obtained residue was purified by column chromatography (SiO₂, eluent cyclohexane:EtOAc=100:0 to 70:30) to give, after evaporation and drying, S-(2-ethoxy-6-formylphenyl)dimethylcarbamothioate SSA 39182B (950 mg, 80% yield) as a brown solid.

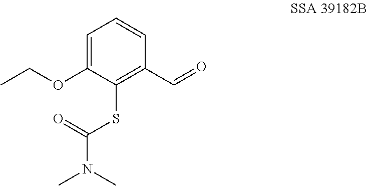 [SMILES: CCOc1cccc(C=O)c1SC(=O)N(C)C]

MW: 253.32, Yield: 80%, Brown Solid; Mp (° C.): 57.9

R_{f}: 0.15 (cyclohexane:EtOAc=80:20).

¹H-NMR (CDCl₃, δ): 1.44 (t, 3H, J=6.9 Hz, CH₃), 3.02 (s, 3H, NCH₃), 3.22 (s, 3H, NCH₃), 4.12 (q, 2H, J=6.9 Hz, OCH₂), 7.17 (d, 1H, J=8.1 Hz, ArH), 7.48 (t, 1H, J=8.0 Hz, ArH), 7.61 (d, 1H, J=8.1 Hz, ArH), 10.46 (s, 1H, CHO).

¹³C-NMR (CDCl₃, δ): 14.65, 37.20, 65.14, 117.49, 120.39, 121.45, 130.81, 139.09, 159.53, 165.00, 191.73.

MS-ESI m/z (% rel. Int.): 254.1 ([MH]⁺, 100).

HPLC: Method A, XBridge™ column, detection UV 254 nm, RT=5.60 min, peak area 89.0%.

### S-(2-(((2,2-Diethoxyethyl)amino)methyl)-6-ethoxyphenyl)dimethylcarbamothioate SSA 39184

S-(2-Ethoxy-6-formylphenyl)dimethylcarbamothioate SSA 39182B (950 mg, 3.75 mmol, 1.0 eq) was dissolved in CH₂Cl₂ (30 mL) under a nitrogen atmosphere at RT and aminoacetaldehyde diethyl acetal (736 μL, 5.06 mmol, 1.35 eq) was added. The reaction mixture was stirred at RT for 5 min. Acetic acid (94 μL) and sodium cyanoborohydride (371 mg, 5.63 mmol, 1.5 eq) were slowly added. The reaction mixture was stirred at RT overnight, then diluted with CH₂Cl₂ (180 mL) and H₂O (90 mL) and a 1 N K₂CO₃ aq. solution (5 mL) were added. The separated organic layer was washed with water (50 mL), brine (35 mL), dried over MgSO₄, filtered and concentrated to give a brown viscous oil (1.54 g). A small quantity (167 mg, 0.45 mmol) of this crude product was purified by column chromatography (SiO₂, eluent CH₂Cl₂:MeOH=100:0 to 95:5) to give, after evaporation and drying, S-(2-(((2,2-diethoxyethyl)amino)methyl)-6-ethoxyphenyl)dimethylcarbamothioate SSA 39184 (78 mg, 52% yield) as a colorless oil.

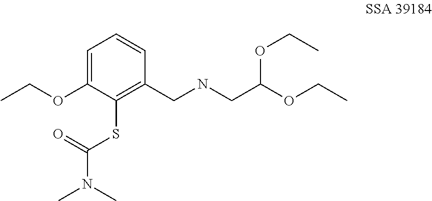 [SMILES: CCOc1cccc(CNCC(OCC)OCC)c1SC(=O)N(C)C]

MW: 370.51; Yield: 52%; Colorless oil.

R_{f}: 0.35 (CH₂Cl₂:MeOH=95:5).

¹H-NMR (CDCl₃, δ): 1.16-1.22 (m, 6H, 2×CH₃), 1.35-1.41 (m, 3H, CH₃), 2.73 (d, 2H, J=5 Hz, CH₂), 3.01-3.13 (m, 6H, N(CH₃)₂), 3.47-3.55 (m, 2H, OCH₂), 3.60-3.68 (m, 2H, OCH₂), 3.91 (s, 2H, PhCH₂), 4.02-4.09 (m, 2H, OCH₂), 6.83 (d, 1H, J=7 Hz, ArH), 7.05 (d, 1H, J=7 Hz, ArH), 7.31 (t, 1H, J=7 Hz, ArH).

¹³C-NMR (CDCl₃, δ): 14.7, 15.4, 29.6, 37.1, 51.4, 52.5, 62.2, 64.7, 102.3, 111.5, 117.1, 121.8, 130.7, 145.9, 159.8, 166.0.

MS-ESI m/z (% rel. Int.): 281.1 ([M+H]⁺, 10), 235.1 (100).

HPLC: Method A, XBridge™ column, detection UV 254 nm, RT=3.3 min, peak area 85%.

### S-(4-(3,5-Dimethoxy-4-propoxybenzyl)-7-ethoxyisoquinolin-8-yl) dimethylcarbamothioate hydrochloride 82

S-(2-(((2,2-Diethoxyethyl)amino)methyl)-6-ethoxyphenyl)dimethylcarbamothioate SSA 39184 (441 mg, 1.19 mmol) and 3,5-dimethoxy-4-propoxybenzaldehyde TTA 24142 (267 mg, 1.19 mmol) were dissolved in EtOH (3 mL) and a 37% HCl solution (3 mL). The reaction mixture was stirred at 100° C. for 20 min, cooled to RT and evaporated. EtOAc (500 mL) and a 1 N K₂CO₃ solution (20 mL) were added. The separated organic layer was washed with brine (20 mL), dried over MgSO₄, filtered and evaporated to give a brown oil. This oil was purified by column chromatography (SiO₂, eluent cyclohexane:EtOAc=50:50 to 0:100) to give after evaporation and drying S-(4-(3,5-dimethoxy-4-propoxybenzyl)-7-ethoxyisoquinolin-8-yl) dimethylcarbamothioate (75 mg, 0.154 mmol). This material was dissolved in MeOH (1 mL) and a 1.34 N HCl solution in MeOH (42.5 μL, 0.057 mmol) was slowly added and the reaction mixture was stirred at 4° C. for 15 min. After evaporation and drying under P₂O₅, S-(4-(3,5-dimethoxy-4-propoxybenzyl)-7-ethoxyisoquinolin-8-yl) dimethylcarbamothioate hydrochloride 82 (27.6 mg, 13% yield) was obtained as a yellow solid.

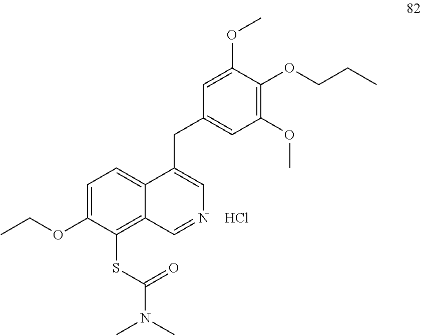 [SMILES: CCCOc1c(OC)cc(Cc2cncc3c(SC(=O)N(C)C)c(OCC)ccc23)cc1OC ; Cl]

MW: 521.07; Yield: 13%; Yellow solid; Mp (° C.): 75.1

R_{f}: 0.2 (cyclohexane:EtOAc=5:5, free base).

¹H-NMR (CD₃OD, δ): 1.02 (t, 3H, J=7.4 Hz, CH₃), 1.50 (t, 3H, J=7.0 Hz, CH₃), 1.71 (q, 2H, J=7.0 Hz, CH₂), 3.03 (s, 3H, NCH₃), 3.33 (s, 3H, NCH₃), 3.78 (s, 6H, 2×OMe), 3.86 (t, 2H, J=6.7 Hz, OCH₂), 4.41 (q, 2H, J=7.0 Hz, OCH₂), 4.60 (s, 2H, OCH₂), 6.63 (s, 2H, 2×ArH), 8.16 (d, 1H, J=9.5 Hz, ArH), 8.31 (s, 1H, ArH), 8.62 (d, 1H, J=9.5 Hz, ArH), 9.79 (s, 1H, ArH).

¹³C-NMR (CD₃OD, δ): 10.8, 15.0, 24.3, 36.9, 37.4, 37.8, 56.7 (2×C), 67.3, 76.2, 107.7 (2×C), 115.6, 125.9, 129.9, 130.1, 133.1, 134.8, 135.2, 137.5, 139.2, 144.4, 155.3 (2×C), 163.0, 166.2.

MS-ESI m/z (% rel. Int.): 485.2 ([MH]⁺, 100).

HPLC: Method A, XBridge™ column, detection UV 254 nm, RT=5.43 min, peak area 99.0%.

### Preparation of S-(4-(3,5-dimethoxy-4-propoxybenzoyl)-7-ethoxyisoquinolin-8-yl) dimethylcarbamothioate hydrochloride 83

### S-(4-(3,5-Dimethoxy-4-propoxybenzoyl)-7-ethoxyisoquinolin-8-yl) dimethylcarbamothioate hydrochloride 83

A mixture of S-(4-(3,5-dimethoxy-4-propoxybenzyl)-7-ethoxyisoquinolin-8-yl) dimethylcarbamothioate 82 free base (83 mg, 171 μmol), N-hydroxyphthalimide (5.6 mg, 34.2 μmol) and NaClO₂ (80% pure, 27.1 mg, 240 μmol) in CH₃CN:H₂O=2:1 (18 mL:9 mL) in a round bottom flask was stirred for 6 h at 85° C. After cooling to RT, the mixture was diluted with Et₂O (100 mL), washed with a 10% aq. sodium sulfite solution (10 mL), brine (10 mL), dried over MgSO₄, filtered and concentrated. The obtained crude product was purified by column chromatography, (SiO₂, eluent from cyclohexane:EtOAc=100:0 to 4:6) to give, after evaporation and drying, S-(4-(3,5-dimethoxy-4-propoxybenzoyl)-7-ethoxyisoquinolin-8-yl) dimethylcarbamothioate (33.6 mg). This material was dissolved in MeOH (1 mL) and a 1.34 HCl solution in MeOH (52.8 μL, 71 μmol) was slowly added and the reaction mixture was stirred at 4° C. for min. After evaporation and drying under P₂O₅, S-(4-(3,5-dimethoxy-4-propoxybenzoyl)-7-ethoxyisoquinolin-8-yl) dimethylcarbamothioate hydrochloride 83 (35.2 mg, 38% yield) was obtained as a yellow solid.

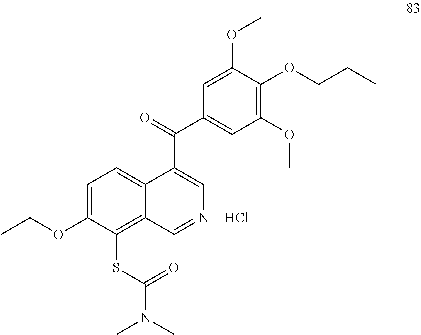 [SMILES: CCCOc1c(OC)cc(C(=O)c2cncc3c(SC(=O)N(C)C)c(OCC)ccc23)cc1OC ; Cl]

MW: 535.05; Yield: 38%; Yellow Solid; Mp (° C.): 133.2

R_{f}: 0.22 (cyclohexane:EtOAc=4:6, free base).

¹H-NMR (CD₃OD, δ): 1.05 (t, 3H, J=7.4 Hz, CH₃), 1.51 (t, 3H, J=7.0 Hz, CH₃), 1.75 (q, 2H, J=6.9 Hz, CH₂), 3.05 (s, 3H, NCH₃), 3.36 (s, 3H, NCH₃), 3.82 (s, 6H, 2×OMe), 4.05 (t, 2H, J=6.5 Hz, OCH₂), 4.42 (q, 2H, J=7.0 Hz, OCH₂), 7.23 (s, 2H, 2×ArH), 8.16 (d, 1H, J=9.5 Hz, ArH), 8.33 (d, 1H, J=9.5 Hz, ArH), 8.67 (s, 1H, ArH), 10.03 (s, 1H, ArH).

¹³C-NMR (CD₃OD, δ): 10.8, 15.1, 24.5, 37.4, 37.8, 57.0 (2×C), 67.3, 76.4, 109.5 (2×C), 115.3, 125.48, 130.49, 132.9, 133.0, 133.3, 133.4, 135.2, 145.0, 148.9, 155.1 (2×C), 162.8, 166.3, 193.0.

MS-ESI m/z (% rel. Int.): 499.1 ([MH]⁺, 100).

HPLC: Method A, XBridge™ column, detection UV 254 nm, RT=6.10 min, peak area 99.0%.

### Preparation of (3,5-dimethoxy-4-propoxyphenyl)(7-ethoxy-8-mercaptoisoquinolin-4-yl)methanone hydrochloride 84

### (3,5-Dimethoxy-4-propoxyphenyl)(7-ethoxy-8-mercaptoisoquinolin-4-yl)methanone hydrochloride 84

In an ace pressure tube, S-(4-(3,5-dimethoxy-4-propoxybenzoyl)-7-ethoxyisoquinolin-8-yl) dimethylcarbamothioate 83 free base (85 mg, 0.17 mmol, 1.0 eq) was dissolved in MeOH (2.1 mL). A 10% aq. NaOH solution (850 μL, 0.21 mmol, 1.25 eq) was added and the reaction mixture was stirred at 65° C. for 2 h. CH₂Cl₂ (200 mL) and water (15 mL) were added. The separated organic layer was washed with brine (10 mL), dried over MgSO₄, filtered and evaporated. The obtained crude product was purified by column chromatography (SiO₂, eluent CH₂Cl₂:EtOAc=95:15 to 85:15) to give, after evaporation and drying, (3,5-dimethoxy-4-propoxyphenyl)(7-ethoxy-8-mercaptoisoquinolin-4-yl)methanone (13.6 mg). This material was dissolved in MeOH (1 mL), a 1.34 N HCl solution in MeOH (25 μL, 33 μmol, 1.05 eq) was slowly added and the reaction mixture was stirred at 4° C. for 15 min. After evaporation and drying under P₂O₅, (3,5-dimethoxy-4-propoxyphenyl)(7-ethoxy-8-mercaptoisoquinolin-4-yl)methanone hydrochloride 84 (14.2 mg, 18% yield) was obtained as a yellow solid.

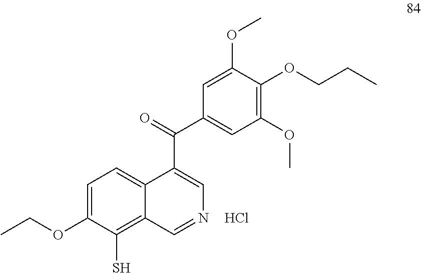 [SMILES: CCCOc1c(OC)cc(C(=O)c2cncc3c(S)c(OCC)ccc23)cc1OC ; Cl]

MW: 463.97; Yield: 18%; Yellow solid; Mp (° C.): 214.4

R_{f}: 0.4 (CH₂Cl₂:EtOAc=9:1, free base).

¹H-NMR (CD₃OD, δ): 1.01 (t, 3H, J=7.4 Hz, CH₃), 1.18 (t, 3H, J=6.8 Hz, CH₃), 1.73 (q, 2H, J=6.8 Hz, CH₂), 3.81 (s, 6H, 2×OMe), 4.04 (t, 2H, J=6.6 Hz, OCH₂), 4.13 (q, 2H, J=6.8 Hz, OCH₂), 7.18 (s, 2H, 2×ArH), 7.96 (d, 1H, J=9.5 Hz, ArH), 8.26 (d, 1H, J=9.4 Hz, ArH), 8.54 (s, 1H, ArH), 9.73 (s, 1H ArH).

¹³C-NMR (CD₃OD, δ): 14.43, 17.57, 27.07, 59.78 (2×C), 69.96, 79.07, 112.23 (2×C), 124.27, 128.12, 133.79, 135.07, 135.15, 135.55, 135.75, 137.98, 147.76, 150.48, 157.61 (2×C), 165.52, 195.08.

MS-ESI m/z (% rel. Int.): 427.1 ([MH]⁺, 100, SH), 853.3 ([MH]⁺, 20, S—S).

HPLC: Method A, XBridge™ column, detection UV 254 nm, 84 RT=6.38 min, peak area 10% (unstable), a disulfur derivative was observed at RT=7.72 min, peak area 90%.

### Preparation of 7-ethoxy-4-(3,4,5-trimethoxybenzyl)isoquinoline-8-carbonitrile hydrochloride 85

### 7-Ethoxy-4-(3,4,5-trimethoxybenzyl)isoquinoline-8-carbonitrile hydrochloride 85

A mixture of 7-ethoxy-4-(3,4,5-trimethoxybenzyl)isoquinolin-8-yl trifluoromethane-sulfonate CCH 34050 (150 mg, 299 μmol), Pd(PPh₃)₄ (35 mg, 30 μmol) and Zn(CN)₂ (40 mg, 341 μmol) in DMF (1 mL) in a 2.5 mL microwave vial equipped with a magnetic stirrer was heated for 15 min at 180° C. under microwave irradiation. After cooling to RT, water (2 mL) was added followed by a 2.0 M H₂SO₄ aqueous solution (0.6 mL) and the mixture was stirred for 20 min at RT before neutralisation with a saturated aq. NaHCO₃ solution (20 mL). The solution was extracted with CH₂Cl₂ (50 mL) and the organic phase was isolated, washed with brine (10 mL), dried over Na₂SO₄, filtered and concentrated at 40° C. under vacuum. Purification by column chromatography (SiO₂, eluent cyclohexane:EtOAc=100:0 to 50:50) gave an off-white solid (103 mg). A small portion of this solid (15 mg) was dissolved in MeOH (2 mL) in a 10 mL round-bottomed flask equipped with a magnetic stirrer before addition of 0.49 M HCl in MeOH (1 mL). The mixture was then stirred for 5 min at RT, and concentrated at 40° C. under vacuum to obtain 7-ethoxy-4-(3,4,5-trimethoxybenzyl)isoquinoline-8-carbonitrile hydrochloride 85 (16.5 mg, 91% yield) as a pale brown solid.

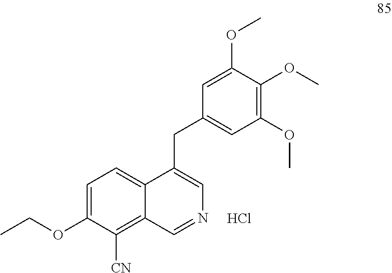 [SMILES: CCOc1ccc2c(Cc3cc(OC)c(OC)c(OC)c3)cncc2c1C#N ; Cl]

MW: 414.88; Yield: 91%; Pale brown solid; Mp (° C.): 180.5 (dec.)

R_{f}: 0.2 (cyclohexane:EtOAc=50:50, free base).

¹H-NMR (CD₃OD, δ): 1.32 (t, 3H, J=7.0 Hz, CH₂CH₃), 3.49 (s, 3H, OCH₃), 3.54 (s, 3H, OCH₃), 3.55 (s, 3H, OCH₃), 4.31 (q, 2H, J=7.0 Hz, CH₂CH₃), 4.38 (s, 2H, CH₂), 7.34-7.45 (m, 2H, 2×ArH), 7.99 (d, 1H, J=9.6 Hz, ArH), 8.22 (s, 1H, ArH), 8.52 (d, 1H, J=9.6 Hz, ArH), 9.34 (s, 1H, ArH).

¹³C-NMR (CD₃OD, δ): 14.9, 36.9, 56.7 (2×C), 61.1, 68.1, 97.7, 107.6 (2×C), 113.8, 125.5, 129.9, 130.1, 132.2, 132.9, 134.7, 138.3, 139.9, 142.8, 155.1 (2×C), 165.4.

MS-ESI m/z (rel. int.): 379 ([MH]⁺, 100).

HPLC: Method A (10 min), XBridge™ column, detection UV 254 nm, RT=4.38 min, peak area 98.8%.

### Preparation of 7-ethoxy-4-(3-isobutoxy-4,5-dimethoxybenzyl)isoquinolin-8-ol hydrochloride 86

### 3-Isobutoxy-4,5-dimethoxybenzaldehyde SIL 32172

To a solution of 3,4-dimethoxy-5-hydroxybenzaldehyde (250 mg, 1.37 mmol) in dry DMF (6 mL) in a 25 mL round-bottomed flask equipped with a magnetic stirrer was added cesium carbonate (492 mg, 1.51 mmol) and the mixture was stirred for 10 min under a nitrogen atmosphere. 1-Bromo-2-methylpropane (164 μl, 1.51 mmol) was added and the reaction mixture was stirred overnight at RT. Another portion of 1-bromo-2-methylpropane (224 μl, 2.06 mmol) was added and the mixture was stirred for 2 h at 80° C. After cooling to RT, the mixture was diluted with Et₂O (50 mL) and the organic solution was washed with water (3×5 mL), brine (10 mL), dried over Na₂SO₄, filtered and concentrated under vacuum. Purification by column chromatography (SiO₂, eluent cyclohexane:EtOAc=100:0 to 75:25) gave, after evaporation and drying, 3-isobutoxy-4,5-dimethoxybenzaldehyde SIL 32172 (320 mg, 98% yield) as a colorless oil.

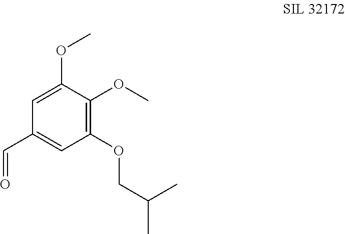 [SMILES: COc1cc(C=O)cc(OCC(C)C)c1OC]

MW: 238.28; Yield: 98%; Colorless oil.

R_{f}: 0.3 (cyclohexane: EtOAc=75:25).

¹H-NMR (CDCl₃, δ): 1.07 (d, 6H, J=6.7 Hz, 2×CH₃), 2.11-2.24 (m, 1H, CH), 3.83 (d, 2H, J=6.5 Hz, OCH₂), 3.92 (s, 3H, CH₃O), 3.95 (s, 3H, CH₃O), 7.11 (s, 2H, 2×ArH), 9.85 (s, 1H, CHO).

¹³C-NMR (CDCl₃, δ): 19.2 (2×C), 26.8, 56.2, 60.8, 75.6, 106.3, 108.1, 131.6, 143.9, 153.2, 153.7, 191.0.

MS-ESI m/z (rel. int.): 239 ([MH]⁺, 100).

HPLC: Method A (5 min), XBridge™ column, detection UV 254 nm, RT=3.30 min.

### 7-Ethoxy-4-(3-isobutoxy-4,5-dimethoxybenzyl)isoquinolin-8-ol hydrochloride CCH 34196B

A mixture of 2-((2,2-diethoxyethylamino)methyl)-6-ethoxyphenol SAO 33014 (343 mg, 1.34 mmol) and 3-isobutoxy-4,5-dimethoxybenzaldehyde SIL 32172 (320 mg, 1.34 mmol) in a mixture of EtOH:conc. HCl=1:1 (10 mL) in a 20 mL microwave vial equipped with a magnetic stirrer was heated for 20 min at 100° C. under microwave irradiation. The volatiles were then removed at 40° C. under vacuum and the residue was taken up in CH₂Cl₂ (50 mL) before neutralisation with a saturated aqueous NaHCO₃ solution (15 mL). The organic phase was isolated, washed with brine (10 mL), dried over Na₂SO₄, filtered and concentrated at 40° C. under vacuum. Purification by column chromatography (SiO₂, eluent cyclohexane:acetone=100:0 to 76:24), followed by a second purification by column chromatography (SiO₂, eluent cyclohexane:EtOAc=100:0 to 60:40), gave 7-ethoxy-4-(3-isobutoxy-4,5-dimethoxybenzyl)isoquinolin-8-ol. This solid was dissolved in MeOH (2 mL) in a 10 mL round-bottomed flask equipped with a magnetic stirrer before addition of 0.49 M HCl in MeOH (1 mL). The mixture was stirred for 5 min at RT, concentrated at 40° C. under vacuum to give 7-ethoxy-4-(3-isobutoxy-4,5-dimethoxybenzyl)isoquinolin-8-ol hydrochloride 86 (140 mg, 23% yield) as a pale brown solid.

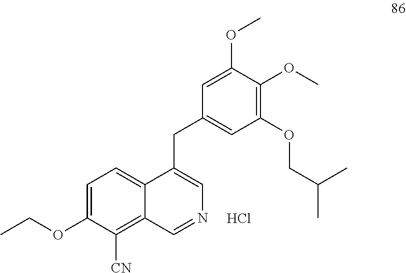 [SMILES: CCOc1ccc2c(Cc3cc(OC)c(OC)c(OCC(C)C)c3)cncc2c1C#N ; Cl]

MW: 447.95; Yield: 23%; Pale brown solid; Mp (° C.): 222.3 (dec.)

R_{f}: 0.2 (cyclohexane:acetone=60:40, free base).

¹H-NMR (CD₃OD, δ): 0.99-1.05 (m, 6H, 2×CH₃), 1.47-1.54 (m, 3H, CH₃), 2.00-2.05 (m, 1H, CH), 3.68-3.79 (m, 8H, 2×OCH₃ and OCH₂), 4.32-4.38 (m, 2H, OCH₂), 4.46 (s, 2H, CH₂), 6.59 (s, 2H, 2×ArH), 7.83-7.89 (m, 1H, ArH), 7.99-8.07 (m, 2H, 2×ArH), 9.63-9.65 (m, 1H, ArH).

¹³C-NMR (CD₃OD, δ): 15.0, 19.6 (2×C), 29.6, 37.0, 56.7, 61.2, 66.9, 76.6, 107.4, 108.7, 116.6, 120.5, 126.6, 128.5, 132.8, 135.0, 137.9, 138.4, 142.5, 146.4, 146.8, 154.5, 155.0.

MS-ESI m/z (rel. int.): 412 ([MH]⁺, 100).

HPLC: Method A (10 min), XBridge™ column, detection UV 254 nm, RT=4.82 min, peak area 98.1%.

### Preparation of 4-(3,4-dimethoxy-5-(2,2,2-trifluoroethoxy)benzyl)-7-ethoxyisoquinolin-8-ol hydrochloride 87

### 3,4-Dimethoxy-5-(2,2,2-trifluoroethoxy)benzaldehyde SIL 32170

To a solution of 3,4-dimethoxy-5-hydroxybenzaldehyde (200 mg, 1.10 mmol) in dry DMF (3 mL) in a 10 mL microwave vial equipped with a magnetic stirrer was added cesium carbonate (393 mg, 1.21 mmol) and the mixture was stirred for 10 min under a nitrogen atmosphere. 2,2,2-Trifluoroethyl-p-toluenesulfonate (0.307 mg, 1.21 mmol) was added and the reaction mixture was stirred for 10 min at 150° C. under microwave irradiation. After cooling to RT, the mixture was diluted with Et₂O (50 mL) and the organic solution was washed with water (3×5 mL), brine (10 mL), dried over Na₂SO₄, filtered and concentrated under vacuum. Purification by column chromatography (SiO₂, eluent cyclohexane:EtOAc=100:0 to 75:25) gave, after evaporation and drying, 3,4-dimethoxy-5-(2,2,2-trifluoroethoxy)benzaldehyde SIL 32170 (243 mg, 84% yield) as a yellow oil.

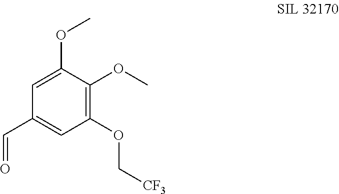 [SMILES: COc1cc(C=O)cc(OCC(F)(F)F)c1OC]

MW: 264.20; Yield: 84%; Yellow oil.

R_{f}: 0.45 (cyclohexane: EtOAc=75:25).

¹H-NMR (CDCl₃, δ): 3.94 (s, 3H, CH₃O), 3.96 (s, 3H, CH₃O), 4.48 (q, 2H, J=8.2 Hz, OCH₂), 7.15 (d, 1H, J=1.7 Hz, ArH), 7.22 (d, 1H, J=1.7 Hz, ArH), 9.84 (s, 1H, COH).

¹³C-NMR (CDCl₃, δ): 56.2, 60.3, 67.4 (q, J=35.2 Hz), 107.9, 110.7, 123.2 (q, J=257.5 Hz, CF₃), 131.3, 144.9, 151.3, 154.1, 190.4.

MS-ESI m/z (rel. int.): 265 ([MH]⁺, 100).

HPLC: Method A (5 min), XBridge™ column, detection UV 254 nm, RT=3.03 min.

4-(3,4-Dimethoxy-5-(2,2,2-trifluoroethoxy)benzyl)-7-ethoxyisoquinolin-8-ol hydrochloride 87

A mixture of 2-((2,2-diethoxyethylamino)methyl)-6-ethoxyphenol SAO 33014 (311 mg, 1.22 mmol) and 3,4-dimethoxy-5-(2,2,2-trifluoroethoxy)benzaldehyde SIL 32170 (321 mg, 1.31 mmol) in a mixture of EtOH:conc. HCl=1:1 (10 mL) in a 20 mL microwave vial equipped with a magnetic stirrer was stirred for 20 min at 100° C. under microwave irradiation. The volatiles were removed at 40° C. under vacuum and the residue was taken up in CH₂Cl₂ (50 mL) before neutralisation with a saturated aqueous NaHCO₃ solution (15 mL). The organic phase was isolated, washed with brine (10 mL), dried over Na₂SO₄, filtered and concentrated at 40° C. under vacuum. Purification by column chromatography (SiO₂, eluent cyclohexane:acetone=100:0 to 76:24), followed by a new purification by column chromatography (SiO₂, eluent cyclohexane:EtOAc=100:0 to 50:50) gave, after evaporation and drying, 4-(3,4-dimethoxy-5-(2,2,2-trifluoroethoxy)benzyl)-7-ethoxyisoquinolin-8-ol. This free base was dissolved in MeOH (2 mL) in a 10 mL round-bottomed flask equipped with a magnetic stirrer before addition of 0.49 M HCl in MeOH (1.5 mL). The mixture was then stirred for 5 min at RT and concentrated at 40° C. under vacuum to afford 4-(3,4-dimethoxy-5-(2,2,2-trifluoroethoxy)benzyl)-7-ethoxyisoquinolin-8-ol hydrochloride 87 (164 mg, 29% yield) as a pale brown solid.

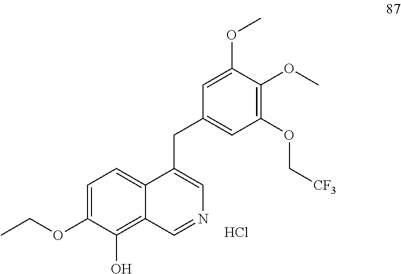 [SMILES: CCOc1ccc2c(Cc3cc(OC)c(OC)c(OCC(F)(F)F)c3)cncc2c1O ; Cl]

MW: 473.87; Yield: 29%; Pale brown solid; Mp (° C.): 213.5 (dec.)

R_{f}: 0.2 (cyclohexane:EtOAc=50:50, free base).

¹H-NMR (CD₃OD, δ): 1.49 (t, 3H, J=7.0 Hz, CH₂CH₃), 3.77 (s, 3H, CH₃O), 3.80 (s, 3H, CH₃O), 4.34 (q, 2H, J=7.0 Hz, CH₂CH₃), 4.48 (q, 2H, J=8.6 Hz, CH₂CF₃), 4.48 (s, 2H, CH₂), 6.64 (s, 1H, ArH), 6.74 (s, 1H, ArH), 7.85 (d, 1H, J=9.1 Hz, ArH), 8.03 (d, 1H, J=9.1 Hz, ArH), 8.07 (s, 1H, ArH), 9.66 (s, 1H, ArH).

¹³C-NMR (CD₃OD, δ): 15.0, 36.8, 56.8, 61.3, 66.9, 68.2 (q, J=35.2 Hz), 109.4, 110.5, 116.5, 120.5, 126.8, 128.4, 132.8, 135.3, 137.6, 139.4, 142.6, 146.4, 146.9, 152.8, 155.4, (CF₃ not seen).

MS-ESI m/z (rel. int.): 438 ([MH]⁺, 100).

HPLC: Method A (10 min), XBridge™ column, detection UV 254 nm, RT=4.65 min, peak area 98.2%.

### Preparation of 8-amino-7-ethoxy-2-methyl-4-(3,4,5-trimethoxybenzyl)isoquinolinium chloride 88

### 8-Amino-7-ethoxy-2-methyl-4-(3,4,5-trimethoxybenzyl)isoquinolinium chloride 88

A mixture of 7-ethoxy-4-(3,4,5-trimethoxybenzyl)isoquinolin-8-amine CCH 34058 (125 mg, 0.34 mmol), diisopropylaminomethyl-polystyrene (200-400 mesh particle size, ˜3 mmol/g, 0.23 g, 0.69 mmol) and MeI (85 μl, 1.37 mmol) in dry CH₂Cl₂ (5 mL) in a 10 mL microwave vial equipped with a magnetic stirrer was heated at 120° C. for 20 min under microwave irradiation. After cooling to RT, the mixture was filtered through cotton wool and the organic solution was washed with a 2 N aq. NaOH solution (3 mL), brine (3 mL), dried over Na₂SO₄, filtered and concentrated under vacuum. Purification by column chromatography (SiO₂, eluent CH₂Cl₂:MeOH=100:0 to 95:5) gave, after evaporation and drying, 28 mg of a solid that was taken up in MeOH (5 mL) and filtered through Amberlite IR-A 410 resin (Cl⁻ form, 4 mL) before concentration at 40° C. under vacuum. 8-Amino-7-ethoxy-2-methyl-4-(3,4,5-trimethoxybenzyl)isoquinolinium chloride 88 (17 mg, 12% yield) was obtained as a red solid.

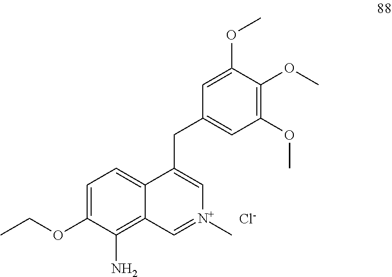 [SMILES: CCOc1ccc2c(Cc3cc(OC)c(OC)c(OC)c3)c[n+](C)cc2c1N ; [Cl-]]

MW: 418.91; Yield: 12%; Red solid; Mp (° C.): 207.6 (dec.)

¹H-NMR (CDCl₃, δ): 1.49 (t, 3H, J=6.9 Hz, CH₂CH₃), 3.81 (s, 6H, 2×OCH₃), 3.85 (s, 3H, OCH₃), 4.21 (q, 2H, J=6.9 Hz, CH₂CH₃), 4.25 (s, 2H, CH₂), 4.43 (s, 3H, CH₃), 6.40-6.41 (m, 2H, 2×ArH), 7.18-7.22 (m, 1H, ArH), 7.29-7.40 (m, 2H, ArH and NH), 7.47-7.50 (m, 1H, ArH), 11.39 (s, 1H, ArH), (1×NH not seen).

¹³C-NMR (CDCl₃, δ): 14.8, 36.5, 47.1, 56.3 (2×C), 60.9, 65.0, 106.1 (2×C), 109.2, 115.1, 121.5, 128.6, 128.8, 132.2, 135.9, 137.2, 140.6, 144.0, 147.6, 153.7 (2×C).

MS-ESI m/z (rel. int.): 383 ([M]⁺, 100).

HPLC: Method A (10 min), XBridge™ column, detection UV 254 nm, RT=4.22 min, peak area 98.1%.

### Preparation of 7-ethoxy-N-ethyl-4-(3,4,5-trimethoxybenzyl)isoquinolin-8-amine dihydrochloride 89

### 7-Ethoxy-N-ethyl-4-(3,4,5-trimethoxybenzyl)isoquinolin-8-amine dihydrochloride 89

A dry 5 mL microwave vial equipped with a magnetic stirrer was charged with a mixture of 7-ethoxy-4-(3,4,5-trimethoxybenzyl)isoquinolin-8-yl trifluoromethanesulfonate hydrochloride 2 (134 mg, 0.25 mmol), Pd(OAc)₂ (5 mg, 22 μmol), (±) BINAP (25 mg, 40 μmol) and Cs₂CO₃ (244 mg, 0.75 mmol). The vial was sealed and purged under vacuum then under N₂ (done 3 times). Ethylamine (2.0 N solution in THF, 1.00 mL, 2.00 mmol) then THF (1 mL) were added and the mixture was heated at 120° C. for 10 min then at 140° C. for 25 min under microwave irradiation. After cooling to RT, the volatiles were removed at 40° C. under vacuum and the residue was taken up in CH₂Cl₂ (50 mL) and filtered through celite. The organic solution was washed with brine (10 mL), dried over Na₂SO₄, filtered and concentrated at 40° C. under vacuum. Purification by column chromatography (SiO₂, eluent cyclohexane:EtOAc=100:0 to 40:60) gave 118 mg of 7-ethoxy-N-ethyl-4-(3,4,5-trimethoxybenzyl)isoquinolin-8-amine. This oil was dissolved in MeOH (5 mL) in a 25 mL round-bottomed flask equipped with a magnetic stirrer before addition of 0.49 M HCl in MeOH (2 mL). The mixture was then stirred for 5 min at RT, concentrated at 40° C. under vacuum to afford a solid that was washed with Et₂O (2×10 mL) and recrystallized from MeOH:EtOAc to give 7-ethoxy-N-ethyl-4-(3,4,5-trimethoxybenzyl)isoquinolin-8-amine dihydrochloride 89 (82 mg, 70% yield) as a red solid.

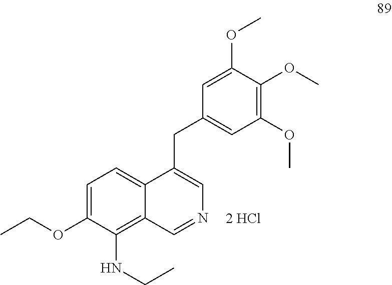 [SMILES: CCNc1c(OCC)ccc2c(Cc3cc(OC)c(OC)c(OC)c3)cncc12 ; "2 HCl"]

MW: 469.40; Yield: 70%; Red solid; Mp (° C.): 200.8 (dec.)

¹H-NMR (CDCl₃, δ): 1.39-1.44 (m, 3H, CH₃), 1.55-1.63 (m, 3H, CH₃), 3.69-3.80 (m, 2H, CH₂), 3.80 (s, 6H, 2×OCH₃), 3.84 (s, 3H, OCH₃), 4.38 (s, 2H, CH₂), 6.41 (s, 2H, 2×ArH), 7.77-7.84 (m, 1H, ArH), 7.91-7.94 (m, 1H, ArH), 8.05 (s, 1H, ArH), 10.09 (s, 1H, ArH).

¹³C-NMR (CDCl₃, δ): 14.3, 14.8, 36.9, 46.3, 56.3 (2×C), 60.9, 66.3, 106.2 (2×C), 119.7, 120.6, 123.0, 127.7, 132.2, 136.4, 137.4, 141.3, 149.9, 153.8 (2×C), (2×C not seen).

MS-ESI m/z (rel. int.): 397 ([MH]⁺, 100).

HPLC: Method A (10 min), XBridge™ column, detection UV 254 nm, RT=4.38 min, peak area 99.4%.

### Preparation of 7-ethoxy-N-(2,2,2-trifluoroethyl)-4-(3,4,5-trimethoxybenzyl)isoquinolin-8-amine dihydrochloride 90

### 7-Ethoxy-N-(2,2,2-trifluoroethyl)-4-(3,4,5-trimethoxybenzyl)isoquinolin-8-amine dihydrochloride 90

A dry 5 mL microwave vial equipped with a magnetic stirrer was charged with a mixture of 7-ethoxy-4-(3,4,5-trimethoxybenzyl)isoquinolin-8-yl trifluoromethanesulfonate hydrochloride 2 (134 mg, 0.25 mmol), Pd(OAc)₂ (10 mg, 44 μmol), (±) BINAP (25 mg, 40 μmol) and Cs₂CO₃ (244 mg, 0.75 mmol). The vial was sealed and purged under vacuum then under N₂ (done 3 times). 2,2,2-Trifluoroethylamine (80 μl, 1.00 mmol) and THF (2 mL) were added and the mixture was heated at 140° C. for 25 min under microwave irradiation. Another portion of 2,2,2-trifluoroethylamine (80 μl, 1.00 mmol) was then added and the mixture was stirred for 25 min at 140° C. under microwave irradiation. After cooling to RT, the volatiles were removed at 40° C. under vacuum and the residue was taken up in CH₂Cl₂ (50 mL) and filtered through celite. The organic solution was washed with brine (10 mL), dried over Na₂SO₄, filtered and concentrated at 40° C. under vacuum. Purification by column chromatography (SiO₂, eluent cyclohexane:EtOAc=100:0 to 60:40) gave 7-ethoxy-N-(2,2,2-trifluoroethyl)-4-(3,4,5-trimethoxybenzyl)isoquinolin-8-amine. This free base was dissolved in MeOH (5 mL) in a 25 mL round-bottomed flask equipped with a magnetic stirrer before addition of 0.49 M HCl in MeOH (2 mL). The mixture was then stirred for 5 min at RT and concentrated at 40° C. under vacuum to give 7-ethoxy-N-(2,2,2-trifluoroethyl)-4-(3,4,5-trimethoxybenzyl)isoquinolin-8-amine dihydrochloride 90 (92 mg, 70% yield) as an orange solid.

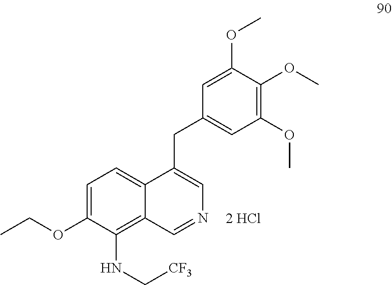 [SMILES: CCOc1ccc2c(Cc3cc(OC)c(OC)c(OC)c3)cncc2c1NCC(F)(F)F ; "2 HCl"]

MW: 523.37; Yield: 70%; Orange solid; Mp (° C.): 229.7 (dec.)

¹H-NMR (CDCl₃, δ): 1.53 (t, 3H, J=6.8 Hz, CH₂CH₃), 3.79 (s, 6H, 2×CH₃O), 3.85 (s, 3H, CH₃O), 4.22-4.33 (m, 6H, 3×CH₂), 6.38 (s, 2H, 2×ArH), 6.60 (br, s, 1H, NH), 7.64-7.71 (m, 2H, 2×ArH), 7.93 (s, 1H, ArH), 10.22 (s, 1H, ArH), (2×NH not seen).

¹³C-NMR (CDCl₃, δ): 14.8, 36.9, 48.1 (q, J=32.6 Hz, CH₂CF₃), 56.2 (2×C), 60.9, 65.5, 106.0 (2×C), 116.1, 121.2, 123.3, 124.1, 127.1, 132.2, 132.4, 135.4, 135.5, 137.3, 141.6, 148.7, 153.8 (2×C), (CF₃ not seen).

MS-ESI m/z (rel. int.): 451 ([MH]⁺, 100).

HPLC: Method A (10 min), XBridge™ column, detection UV 254 nm, RT=4.68 min, peak area 98.3%.

### Preparation of 7-ethoxy-N,N-dimethyl-4-(3,4,5-trimethoxybenzyl)isoquinolin-8-amine dihydrochloride 91

### 7-Ethoxy-N,N-dimethyl-4-(3,4,5-trimethoxybenzyl)isoquinolin-8-amine dihydrochloride 91

A dry 5 mL microwave vial equipped with a magnetic stirrer was charged with a mixture of 7-ethoxy-4-(3,4,5-trimethoxybenzyl)isoquinolin-8-yl trifluoromethanesulfonate hydrochloride 2 (134 mg, 0.25 mmol), Pd(OAc)₂ (8 mg, 35 μmol), (±) BINAP (25 mg, 40 μmol), dimethylamine hydrochloride (102 mg, 1.25 mmol) and Cs₂CO₃ (324 mg, 0.99 mmol). The vial was sealed and purged under vacuum then under a nitrogen atmosphere (done 3 times). THF (2 mL) was added and the mixture was heated at 140° C. for 25 min under microwave irradiation. Another portion of dimethylamine hydrochloride (84 mg, 1.03 mmol) and Cs₂CO₃ (340 mg, 1.04 mmol) were added and the mixture was heated at 140° C. for 20 min under microwave irradiation. After cooling to RT, the volatiles were removed at 40° C. under vacuum and the residue was taken up in CH₂Cl₂ (50 mL) and filtered through celite. The organic solution was washed with brine (10 mL), dried over Na₂SO₄, filtered and concentrated at 40° C. under vacuum. Purification by column chromatography (SiO₂, eluent cyclohexane:EtOAc=100:0 to 50:50) gave 7-ethoxy-N,N-dimethyl-4-(3,4,5-trimethoxybenzyl)isoquinolin-8-amine. This free base was dissolved in MeOH (2 mL) in a 25 mL round-bottomed flask equipped with a magnetic stirrer before addition of 0.49 M HCl in MeOH (0.2 mL). The mixture was then stirred for 5 min at RT and concentrated at 40° C. under vacuum to give 7-ethoxy-N,N-dimethyl-4-(3,4,5-trimethoxybenzyl)isoquinolin-8-amine dihydrochloride 91 (12 mg, 10% yield) as a pale brown solid.

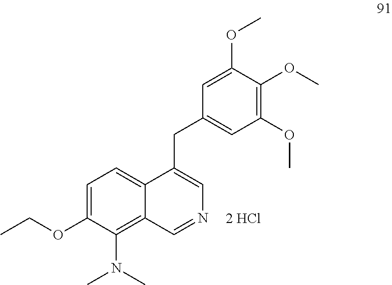 [SMILES: CCOc1ccc2c(Cc3cc(OC)c(OC)c(OC)c3)cncc2c1N(C)C ; "2 HCl"]

MW: 469.40; Yield: 10%; Pale brown solid; Mp (° C.): 189.8 (dec.)

¹H-NMR (CDCl₃, δ): 1.52-1.60 (m, 3H, CH₃), 2.92 (s, 6H, 2×NCH₃), 3.80 (s, 6H, 2×OCH₃), 3.85 (s, 3H, OCH₃), 4.25-4.33 (m, 2H, OCH₂), 4.37 (s, 2H, CH₂), 6.37 (s, 2H, 2×ArH), 7.78-7.85 (m, 1H, ArH), 7.96-8.06 (m, 2H, 2×ArH), 9.85 (s, 1H, ArH), (NH not seen).

¹³C-NMR (CDCl₃, δ): 15.0, 36.8, 44.0 (2×C), 56.3 (2×C), 60.9, 65.1, 106.0 (2×C), 122.1, 124.8, 127.9, 128.1, 132.0, 132.6, 135.9, 137.4, 139.0, 142.3, 153.9 (2×C), 157.0.

MS-ESI m/z (rel. int.): 397 ([MH]⁺, 100).

HPLC: Method A (10 min), XBridge™ column, detection UV 254 nm, RT=4.67 min, peak area 98.0%.

### Preparation of 7-ethoxy-N-methyl-4-(3,4,5-trimethoxybenzyl)isoquinolin-8-amine dihydrochloride 92

### 7-Ethoxy-N-methyl-4-(3,4,5-trimethoxybenzyl)isoquinolin-8-amine dihydrochloride 92

A dry 5 mL microwave vial equipped with a magnetic stirrer was charged with a mixture of 7-ethoxy-4-(3,4,5-trimethoxybenzyl)isoquinolin-8-yl trifluoromethanesulfonate hydrochloride 2 (134 mg, 0.25 mmol), Pd(OAc)₂ (10 mg, 44 μmol), (±) BINAP (25 mg, 40 μmol) and Cs₂CO₃ (244 mg, 0.75 mmol). The vial was sealed and purged under vacuum then under N₂ (done 3 times). Methylamine (2.0 N in THF, 1.00 mL, 2.00 mmol) and THF (1 mL) were added and the mixture was heated, at 120° C. for 10 min then at 140° C. for 25 min, under microwave irradiation. After cooling to RT, the volatiles were removed at 40° C. under vacuum and the residue was taken up in CH₂Cl₂ (50 mL) and filtered through celite. The organic solution was washed with brine (10 mL), dried over Na₂SO₄, filtered and concentrated at 40° C. under vacuum. Purification by column chromatography (SiO₂, eluent cyclohexane:EtOAc=100:0 to 0:100) gave 7-ethoxy-N-methyl-4-(3,4,5-trimethoxybenzyl)isoquinolin-8-amine. The free base was dissolved in MeOH (5 mL) in a 25 mL round-bottomed flask equipped with a magnetic stirrer before addition of 0.49 M HCl in MeOH (2 mL). The mixture was then stirred for 5 min at RT and concentrated at 40° C. under vacuum to give 7-ethoxy-N-methyl-4-(3,4,5-trimethoxybenzyl)isoquinolin-8-amine dihydrochloride 92 (94 mg, 83% yield) as a red solid.

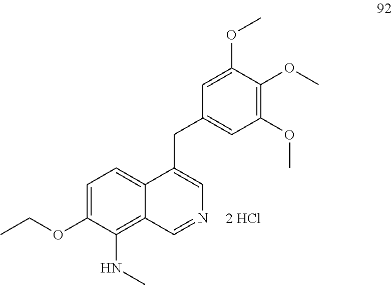 [SMILES: CCOc1ccc2c(Cc3cc(OC)c(OC)c(OC)c3)cncc2c1NC ; "2 HCl"]

MW: 455.37; Yield: 83%; Red solid; Mp (° C.): 168.3 (dec.)

¹H-NMR (CD₃OD, δ): 1.58 (t, 3H, J=7.0 Hz, CH₂CH₃), 3.29 (s, 3H, NCH₃), 3.74 (s, 3H, CH₃O), 3.79 (s, 6H, 2×CH₃O), 4.56 (q, 2H, J=7.0 Hz, CH₂CH₃), 4.61 (s, 2H, CH₂), 6.65 (s, 2H, 2×ArH), 8.26 (d, 1H, J=9.4 Hz, ArH), 8.41 (s, 1H, ArH), 8.54 (d, 1H, J=9.4 Hz, ArH), 10.03 (s, 1H, ArH).

¹³C-NMR (CD₃OD, δ): 14.9, 37.1, 37.7, 56.8 (2×C), 61.1, 68.0, 107.6 (2×C), 122.9, 124.9, 125.8, 127.2, 130.6, 134.1, 134.8, 138.3, 139.5, 141.2, 153.4, 155.0 (2×C).

MS-ESI m/z (rel. int.): 383 ([MH]⁺, 100).

HPLC: Method A (10 min), XBridge™ column, detection UV 254 nm, RT=4.27 min, peak area 98.0%.

### Preparation of methyl 2-(7-ethoxy-4-(3,4,5-trimethoxybenzyl)isoquinolin-8-ylamino)acetate dihydrochloride 93

### Methyl 2-(7-ethoxy-4-(3,4,5-trimethoxybenzyl)isoquinolin-8-ylamino)acetate dihydrochloride 93

A dry 10 mL microwave vial equipped with a magnetic stirrer was charged with a mixture of 7-ethoxy-4-(3,4,5-trimethoxybenzyl)isoquinolin-8-yl trifluoromethanesulfonate hydrochloride 2 (134 mg, 0.25 mmol), Pd(OAc)₂ (5 mg, 22 μmol), (±) BINAP (25 mg, 40 μmol), glycine methyl ester hydrochloride (70 mg, 0.56 mmol) and Cs₂CO₃ (320 mg, 0.98 mmol). The vial was sealed and purged under vacuum then under N₂ (done 3 times). THF (5 mL) was then added and the mixture was stirred for 4.5 h at 150° C. under microwave irradiation. After cooling to RT, THF was removed at 40° C. under vacuum and the residue was taken up in CH₂Cl₂ (50 mL) and filtered through celite. The organic solution was washed with brine (10 mL), dried over Na₂SO₄, filtered and concentrated at 40° C. under vacuum. Purification by column chromatography (SiO₂, eluent cyclohexane:EtOAc=100:0 to 0:100) gave, after evaporation and drying, methyl 2-(7-ethoxy-4-(3,4,5-trimethoxybenzyl)isoquinolin-8-ylamino)acetate. The free base was dissolved in MeOH (2 mL) in a 10 mL round-bottomed flask equipped with a magnetic stirrer before addition of a 1 N aq. NaOH solution (0.5 mL). The mixture was stirred overnight at RT, acidified with a 1N aq. HCl solution (1 mL) before to be concentrated under vacuum. The residue was purified by prep. HPLC (eluent H₂O:CH₃CN:TFA=100:0:0.05 to 50:50:0.05 in 10 min, Prep C18 Xterra 19×50 mm column 186001108) and concentrated under vacuum. The acid was finally dissolved in 0.4 N HCl in MeOH (5 mL) in a 10 mL round-bottomed flask equipped with a magnetic stirrer and the solution was stirred for 1 h under reflux. After evaporation to dryness, methyl 2-(7-ethoxy-4-(3,4,5-trimethoxybenzyl)isoquinolin-8-ylamino)acetate dihydrochloride 93 (5 mg, 4% yield) was obtained as a red solid.

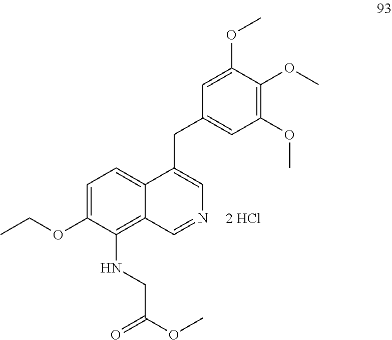 [SMILES: CCOc1ccc2c(Cc3cc(OC)c(OC)c(OC)c3)cncc2c1NCC(=O)OC ; "2 HCl"]

MW: 513.41; Yield: 4%; Red solid.

¹H-NMR (CD₃OD, δ): 1.42-1.46 (m, 3H, CH₃), 3.67-3.75 (m, 12H, 4×OCH₃), 4.19-4.23 (m, 2H, CH₂), 4.39-4.40 (m, 4H, 2×CH₂), 6.57 (s, 2H, 2×ArH), 7.75-7.76 (m, 1H, ArH), 7.85-7.88 (m, 1H, ArH), 7.98 (s, 1H, ArH), 9.67 (s, 1H, ArH).

¹³C-NMR (CD₃OD, δ): 15.0, 37.2, 52.6, 56.7 (2×C), 61.1, 66.8, 107.5 (2×C), 116.5, 121.2, 126.0, 127.9, 133.5, 135.2, 137.7, 138.1, 142.8, 149.0, 154.9 (2×C), 173.8.

MS-ESI m/z (rel. int.): 441 ([MH]⁺, 100).

HPLC: Method A (10 min), XBridge™ column, detection UV 254 nm, RT=4.32 min, peak area 98.3%.

### Preparation of 7-ethoxy-8-iodo-4-(3,4,5-trimethoxybenzyl)isoquinoline hydrochloride 94

### 7-Ethoxy-8-iodo-4-(3,4,5-trimethoxybenzyl)isoquinoline hydrochloride 94

To a solution of 7-ethoxy-4-(3,4,5-trimethoxybenzyl)isoquinolin-8-amine dihydrochloride 3 (212 mg, 480 μmol) in a mixture of conc. HCl (1 mL) and AcOH (2 mL) at +1° C. in a 10 mL round-bottomed flask equipped with a magnetic stirrer was added a solution of sodium nitrite (41 mg, 594 μmol) in H₂O (1 mL). The reaction mixture was stirred for 2 h at RT and cold H₂O (2 mL) was added followed by a solution of KI (115 mg, 693 μmol) and I₂ (88 mg, 347 μmol) in H₂O (1 mL). The reaction mixture was stirred for 3 h, allowing the medium to warm up to RT. Another portion of KI (115 mg, 693 μmol) and I₂ (88 mg, 347 μmol) in H₂O (1 mL) were added and stirring was continued overnight at RT. The reaction mixture was then neutralized with a careful addition of a saturated NaHCO₃ aq. solution (20 mL) before extraction with CH₂Cl₂ (70 mL). The organic solution was washed with brine (15 mL), dried over Na₂SO₄, filtered and concentrated at 40° C. under vacuum. Purification by column chromatography (SiO₂, eluent cyclohexane:EtOAc=100:0 to 66:34 to 0:100) gave, after evaporation and drying, 7-ethoxy-8-iodo-4-(3,4,5-trimethoxybenzyl)isoquinoline. This free base was dissolved in MeOH (2 mL) in a 10 mL round-bottomed flask equipped with a magnetic stirrer before addition of 0.49 M HCl in MeOH (1 mL). The mixture was then stirred for 5 min at RT, and concentrated at 40° C. under vacuum to give 7-ethoxy-8-iodo-4-(3,4,5-trimethoxybenzyl)isoquinoline hydrochloride 94 (19 mg, 8% yield) as a brown solid.

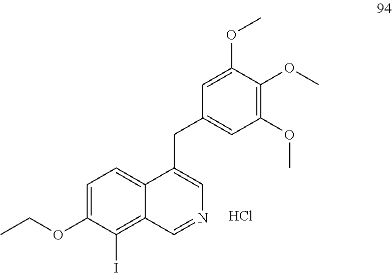 [SMILES: CCOc1ccc2c(Cc3cc(OC)c(OC)c(OC)c3)cncc2c1I ; Cl]

MW: 515.77; Yield: 8%; Brown solid; Mp (° C.): 160.4 (dec.)

R_{f}: 0.2 (cyclohexane:EtOAc=66:34, free base).

¹H-NMR (CDCl₃, δ): 1.54-1.62 (m, 3H, CH₃), 3.80 (s, 3H, OCH₃), 3.84 (s, 6H, 2×OCH₃), 4.32-4.52 (m, 4H, 2×CH₂), 6.40 (s, 2H, 2×ArH), 7.68-7.82 (m, 1H, ArH), 8.13-8.42 (m, 2H, 2×ArH), 9.66 (s, 1H, ArH).

¹³C-NMR (CDCl₃, δ): 14.8, 36.9, 56.4 (2×C), 60.9, 66.7, 106.1 (2×C), 123.1, 126.5, 129.3, 130.9, 131.9, 134.7, 137.0, 137.4, 148.1, 153.9 (2×C), 159.7, (1×C not observed).

MS-ESI m/z (rel. int.): 480 ([MH]⁺, 100).

HPLC: Method A (10 min), XBridge™ column, detection UV 254 nm, RT=4.53 min, peak area 95.7%.

### Preparation of 7-ethoxy-4-(1-(3,4,5-trimethoxyphenyl)vinyl)isoquinolin-8-ol hydrochloride 95

### 8-(Benzyloxy)-7-ethoxy-4-(3,4,5-trimethoxybenzyl)isoquinoline CCH 42024-1

To a solution of 7-ethoxy-4-(3,4,5-trimethoxybenzyl)isoquinolin-8-ol CCH 34046-2 (1.00 g, 2.71 mmol) in dry DMF (10 mL) in a 25 mL round-bottomed flask equipped with a magnetic stirrer was added Cs₂CO₃ (0.97 g, 2.98 mmol) followed by benzyl bromide (0.35 mL, 2.93 mmol) and the reaction mixture was stirred for 1 h at 60° C. After cooling to RT, the mixture was diluted with Et₂O (50 mL), washed with brine (10 mL), dried over Na₂SO₄, filtered and concentrated under vacuum. The residue was finally purified by column chromatography (SiO₂, eluent cyclohexane:EtOAc=100:0 to 60:40) to give 8-(benzyloxy)-7-ethoxy-4-(3,4,5-trimethoxybenzyl)isoquinoline CCH 42024-1 as a brown oil (290 mg, 23% yield).

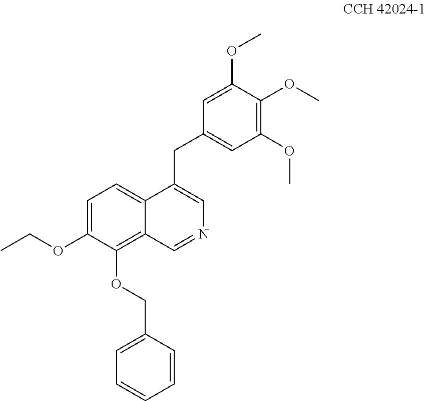 [SMILES: CCOc1ccc2c(Cc3cc(OC)c(OC)c(OC)c3)cncc2c1OCc1ccccc1]

MW: 459.53; Yield: 23%; Brown oil.

R_{f}: 0.2 (cyclohexane: EtOAc=60:40)

### (8-(Benzyloxy)-7-ethoxyisoquinolin-4-yl)(3,4,5-trimethoxyphenyl)methanone CCH 42024-2

A mixture of 8-(benzyloxy)-7-ethoxy-4-(3,4,5-trimethoxybenzyl)isoquinoline CCH 42024-1 (270 mg, 588 μmol), N-hydroxyphthalimide (20 mg, 123 μmol) and NaClO₂ (80% pure, 100 mg, 884 μmol) in CH₃CN:H₂O=1:1 (10 mL) in a 20 mL microwave vial equipped with a magnetic stirrer was stirred for 30 min at 100° C. under microwave irradiation. After cooling to RT, CH₃CN was removed at 40° C. under vacuum and the residue was extracted with CH₂Cl₂ (50 mL). The organic layer was washed with brine (10 mL), dried over Na₂SO₄, filtered and concentrated at 40° C. under vacuum. Purification by column chromatography (SiO₂, eluent cyclohexane:EtOAc=100:0 to 60:40) gave (8-(benzyloxy)-7-ethoxyisoquinolin-4-yl)(3,4,5-trimethoxyphenyl)methanone CCH 42024-2 (105 mg, 38% yield) as a yellow oil.

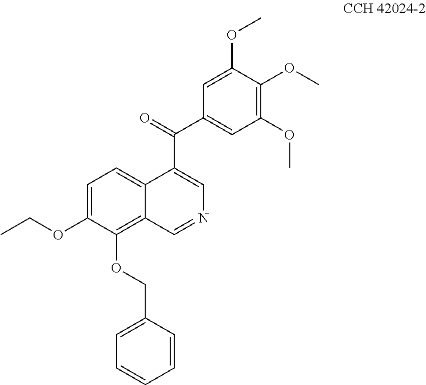 [SMILES: CCOc1ccc2c(C(=O)c3cc(OC)c(OC)c(OC)c3)cncc2c1OCc1ccccc1]

MW: 473.52; Yield: 38%; Yellow oil.

R_{f}: 0.3 (cyclohexane: EtOAc=50:50)

### 7-Ethoxy-4-(1-(3,4,5-trimethoxyphenyl)vinyl)isoquinolin-8-ol hydrochloride 95

To a solution of (8-(benzyloxy)-7-ethoxyisoquinolin-4-yl)(3,4,5-trimethoxyphenyl)methanone CCH 42024-2 (40 mg, 87 μmol) in dry THF (2 mL) in a 5 mL microwave vial equipped with a magnetic stirrer was added MeLi (1.6 M in Et₂O, 0.20 mL, 0.32 mmol) and the reaction mixture was stirred for 2 h at RT, then quenched by a careful addition of conc. HCl (0.5 mL). The mixture was stirred for 1.5 h at RT, then for 40 min at 110° C. under microwave irradiation. After cooling to RT, the mixture was concentrated to dryness at 40° C. and the residue was partitioned between CH₂Cl₂ (50 mL) and a saturated aqueous NaHCO₃ solution (10 mL). The organic phase was isolated, washed with brine (10 mL), dried over Na₂SO₄, filtered and concentrated under vacuum. Purification by column chromatography (SiO₂, eluent cyclohexane:EtOAc=100:0 to 0:100) gave, after evaporation and drying, 7-ethoxy-4-(1-(3,4,5-trimethoxyphenyl)vinyl)isoquinolin-8-ol. This free base was dissolved in MeOH (2 mL) in a 10 mL round-bottomed flask equipped with a magnetic stirrer before addition of a 0.49 M HCl solution in MeOH (1 mL). The reaction mixture was stirred for 5 min at RT and concentrated at 40° C. under vacuum to give 7-ethoxy-4-(1-(3,4,5-trimethoxyphenyl)vinyl)isoquinolin-8-ol hydrochloride 95 (8 mg, 22% yield) as a brown solid.

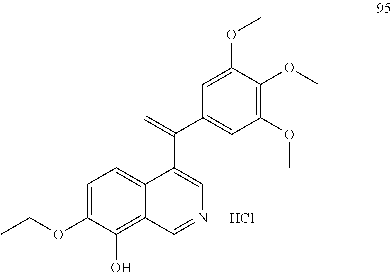 [SMILES: C=C(c1cc(OC)c(OC)c(OC)c1)c1cncc2c(O)c(OCC)ccc12 ; Cl]

MW: 417.88; Yield: 22%; Brown solid; Mp (° C.): 210.7 (dec.)

¹H-NMR (CD₃OD, δ): 1.45-1.51 (m, 3H, CH₂CH₃), 3.70 (s, 6H, 2×OCH₃), 3.74 (s, 3H, CH₃O), 4.25-4.33 (m, 2H, CH₂CH₃), 5.56-5.58 (m, 1H, CH), 6.14-6.17 (m, 1H, CH), 6.60 (s, 2H, 2×ArH), 7.39-7.44 (m, 1H, ArH), 7.88-7.93 (m, 1H, ArH), 8.28 (s, 1H, ArH), 9.75 (s, 1H, ArH).

¹³C-NMR (CD₃OD, δ): 15.0, 56.7 (2×C), 61.1, 66.9, 105.6 (2×C), 118.2, 120.3, 120.6, 126.7, 128.5, 132.5, 136.5, 139.1, 140.0, 143.6, 144.3, 146.4, 146.6, 154.9 (2×C).

MS-ESI m/z (rel. int.): 382 ([MH]⁺, 100).

HPLC: Method A (10 min), XBridge™ column, detection UV 254 nm, RT=4.16 min, peak area 95.1%.

### Preparation of 7-ethoxy-4-(3,4,5-trimethoxybenzoyl)isoquinolin-8-yl acetate 96

### 7-Ethoxy-4-(3,4,5-trimethoxybenzyl)isoquinolin-8-yl acetate CCH 42032-1

To a solution of 7-ethoxy-4-(3,4,5-trimethoxybenzyl)isoquinolin-8-ol CCH 34046-2 (400 mg, 1.08 mmol) in dry CH₂Cl₂ (20 mL) in a 100 mL round-bottomed flask equipped with a magnetic stirrer was added diisopropylaminomethyl-polystyrene (200-400 mesh particle size, ˜3 mmol/g, 1.43 g, 4.29 mmol), DMAP (30 mg, 0.25 mmol) and acetic anhydride (0.58 mL, 6.18 mmol) and the reaction mixture was stirred overnight at RT. The organic solution was then filtered, washed with brine (10 mL), dried over Na₂SO₄, filtered and concentrated under vacuum. The residue was purified by column chromatography (SiO₂, eluent cyclohexane:EtOAc=100:0 to 50:50) to give, after evaporation and drying, 7-ethoxy-4-(3,4,5-trimethoxybenzyl)isoquinolin-8-yl acetate CCH 42032-1 (398 mg, 89% yield) as a pale brown solid.

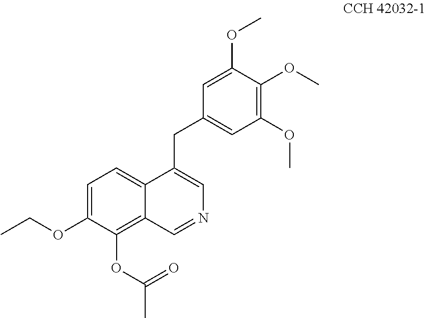 [SMILES: CCOc1ccc2c(Cc3cc(OC)c(OC)c(OC)c3)cncc2c1OC(C)=O]

MW: 411.45; Yield: 89%; Pale brown solid.

R_{f}: 0.25 (cyclohexane: EtOAc=50:50).

¹H-NMR (CDCl₃, δ): 1.42 (t, 3H, J=7.0 Hz, CH₂CH₃), 2.48 (s, 3H, CH₃), 3.76 (s, 6H, 2×OCH₃), 3.81 (s, 3H, CH₃O), 4.19 (q, 2H, J=7.0 Hz, CH₂CH₃), 4.28 (s, 2H, CH₂), 6.39 (s, 2H, 2×ArH), 7.48 (d, 1H, J=8.8 Hz), 7.81 (d, 1H, J=8.8 Hz, ArH), 8.30 (s, 1H, ArH), 9.22 (s, 1H, ArH).

¹³C-NMR (CDCl₃, δ): 15.0, 20.5, 36.7, 56.1 (2×C), 60.9, 65.4, 105.7 (2×C), 119.8, 122.5, 123.4, 129.2, 130.2, 134.4, 135.2, 136.6, 141.9, 145.4, 147.6, 153.4 (2×C), 168.6.

MS-ESI m/z (rel. int.): 412 ([MH]⁺, 100).

HPLC: Method A (10 min), XBridge™ column, detection UV 254 nm, RT=4.26 min.

### 7-Ethoxy-4-(3,4,5-trimethoxybenzoyl)isoquinolin-8-yl acetate 96

A mixture of 7-ethoxy-4-(3,4,5-trimethoxybenzyl)isoquinolin-8-yl acetate CCH 42032-1 (360 mg, 875 μmol), N-hydroxyphthalimide (30 mg, 184 μmol) and NaClO₂ (80% pure, 150 mg, 1.33 mmol) in CH₃CN:H₂O=1:1 (10 mL) in a 20 mL microwave vial equipped with a magnetic stirrer was stirred for 30 min at 100° C. under microwave irradiation. After cooling to RT, the mixture was concentrated to dryness at 40° C. under vacuum then taken up in CH₂Cl₂ (50 mL) and the organic solution was washed with a 10% aq. sodium sulfite solution (10 mL), brine (10 mL), dried over Na₂SO₄, filtered and concentrated at 40° C. under vacuum. Purification by column chromatography (SiO₂, eluent cyclohexane:EtOAc=100:0 to 50:50) followed by a second purification by column chromatography (SiO₂, eluent CH₂Cl₂:EtOAc=100:0 to 80:20) gave, after evaporation and drying, 7-ethoxy-4-(3,4,5-trimethoxybenzoyl)isoquinolin-8-yl acetate 96 (105 mg, 28% yield) as an off-white solid.

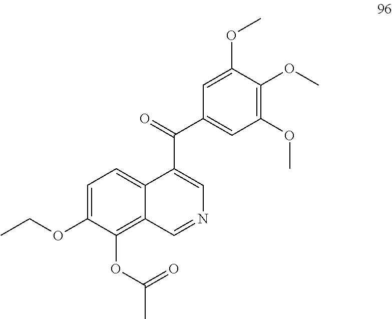 [SMILES: CCOc1ccc2c(C(=O)c3cc(OC)c(OC)c(OC)c3)cncc2c1OC(C)=O]

MW: 425.43; Yield: 28%; Off-white solid; Mp (° C.): 159.0 (dec.)

R_{f}: 0.2 (cyclohexane: EtOAc=60:40).

¹H-NMR (CDCl₃, δ): 1.46 (t, 3H, J=7.0 Hz, CH₂CH₃), 2.52 (s, 3H, CH₃), 3.85 (s, 6H, 2×OCH₃), 3.96 (s, 3H, CH₃O), 4.25 (q, 2H, J=7.0 Hz, CH₂CH₃), 7.15 (s, 2H, 2×ArH), 7.59 (d, 1H, J=9.3 Hz), 8.08 (d, 1H, J=9.3 Hz, ArH), 8.56 (s, 1H, ArH), 9.41 (s, 1H, ArH).

¹³C-NMR (CDCl₃, δ): 14.9, 20.5, 56.4 (2×C), 61.0, 65.5, 107.9 (2×C), 120.9, 123.6, 124.0, 128.7, 128.8, 132.8, 134.2, 142.3, 143.2, 148.2, 148.7, 153.1 (2×C), 168.6, 194.7.

MS-ESI m/z (rel. int.): 426 ([MH]⁺, 100).

HPLC: Method A (10 min), XBridge™ column, detection UV 254 nm, RT=4.88 min, peak area 99.7%.

### Preparation of (7-ethoxy-8-hydroxyisoquinolin-4-yl)(3,4,5-trimethoxyphenyl)methanone hydrochloride 97

### (7-Ethoxy-8-hydroxyisoquinolin-4-yl)(3,4,5-trimethoxyphenyl)methanone hydrochloride 97

To a solution of 7-ethoxy-4-(3,4,5-trimethoxybenzoyl)isoquinolin-8-yl acetate 96 (50 mg, 118 μmol) in CH₂Cl₂ (5 mL) in a 25 mL round-bottomed flask equipped with a magnetic stirrer was added a 7 N NH₃ solution in MeOH (1 mL) and the reaction mixture was stirred overnight at RT. The organic solution was then diluted with more CH₂Cl₂ (45 mL), washed with brine (10 mL), dried over Na₂SO₄, filtered and concentrated under vacuum. The residue was purified by column chromatography (SiO₂, eluent CH₂Cl₂:MeOH=100:0 to 95:5) to give, after evaporation and drying, 7-ethoxy-8-hydroxyisoquinolin-4-yl)(3,4,5-trimethoxyphenyl)methanone. This free base was dissolved in MeOH (3 mL) in a 10 mL round-bottomed flask equipped with a magnetic stirrer before addition of a 0.49 M HCl solution in MeOH (0.5 mL). The reaction mixture was stirred for 5 min at RT and concentrated at 40° C. under vacuum to afford after drying 7-ethoxy-8-hydroxyisoquinolin-4-yl)(3,4,5-trimethoxyphenyl)methanone hydrochloride 97 (42 mg, 85% yield) as an orange solid.

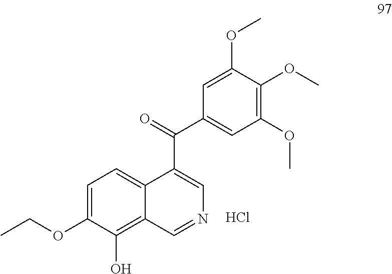 [SMILES: CCOc1ccc2c(C(=O)c3cc(OC)c(OC)c(OC)c3)cncc2c1O ; Cl]

MW: 419.86; Yield: 85%; Orange solid; Mp (° C.): 222.3 (dec.)

R_{f}: 0.15 (cyclohexane:acetone=75:25, free base).

¹H-NMR (CD₃OD:CDCl₃=1:1, 6): 1.54 (t, 3H, J=7.0 Hz, CH₂CH₃), 3.85 (s, 6H, 2×OCH₃), 3.96 (s, 3H, CH₃O), 4.35 (q, 2H, J=7.0 Hz, CH₂CH₃), 7.12 (s, 2H, 2×ArH), 7.63 (d, 1H, J=9.0 Hz), 7.92 (d, 1H, J=9.0 Hz, ArH), 8.31 (s, 1H, ArH), 9.86 (s, 1H, ArH).

¹³C-NMR (CD₃OD:CDCl₃=1:1, 6): 15.5, 57.5 (2×C), 62.0, 67.1, 109.4 (2×C), 117.7, 120.9, 127.6, 128.6, 130.7, 132.6, 135.6, 145.5, 145.7, 147.2, 147.5, 154.8 (2×C), 192.4.

MS-ESI m/z (rel. int.): 384 ([MH]⁺, 100).

HPLC: Method A (10 min), XBridge™ column, detection UV 254 nm, RT=4.12 min, peak area 98.3%.

### Preparation of 7-ethoxy-4-(3,4,5-trimethoxybenzoyl)isoquinolin-8-yl disodium phosphate 98

### 7-ethoxy-4-(3,4,5-trimethoxybenzoyl)isoquinolin-8-yl disodium phosphate 98

To a mixture of phosphoryl chloride (0.35 mL, 3.75 mmol) and NEt₃ (0.16 mL, 1.15 mmol) in dry CH₂Cl₂ (5 mL) at 1° C. in a 10 mL round-bottomed flask equipped with a magnetic stirrer was added dropwise a suspension of 7-ethoxy-8-hydroxyisoquinolin-4-yl)(3,4,5-trimethoxyphenyl)methanone (97 free base, 291 mg, 759 μmol) in dry CH₂Cl₂ (7 mL). The mixture was stirred for 30 min at 1° C., and concentrated to dryness under vacuum. The residue was then taken up in a 5 N aq. NaOH solution (2.5 mL) and stirred for 15 min at RT before concentration to a volume of around 1 mL. This residue was purified by reversed phase flash chromatography (RP18, 11.0 g, 25-40 μm, eluent H₂O:CH₃CN=100:0 to 85:15) to give, after concentration and drying under vacuum, 7-ethoxy-4-(3,4,5-trimethoxybenzoyl)isoquinolin-8-yl disodium phosphate 98 (216 mg, 56% yield) as a brown solid.

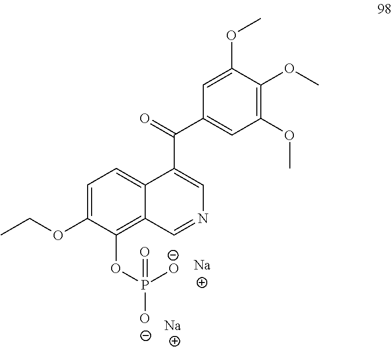 [SMILES: CCOc1ccc2c(C(=O)c3cc(OC)c(OC)c(OC)c3)cncc2c1OP(=O)([O-])[O-] ; [Na+] ; [Na+]]

MW: 507.34; Yield: 56%; Brown solid; Mp (° C.): 154.8 (dec.)

R_{f}: 0.25 (H₂O:CH₃CN=85:15).

¹H-NMR (CD₃OD, δ): 1.37 (t, 3H, J=6.5 Hz, CH₂CH₃), 3.73 (s, 6H, 2×OCH₃), 3.80 (s, 3H, OCH₃), 4.31 (q, 2H, J=6.5 Hz, CH₂CH₃), 7.08 (s, 2H, 2×ArH), 7.55-7.61 (m, 2H, 2×ArH), 8.24 (s, 1H, ArH), 10.07 (s, 1H, ArH).

¹³C-NMR (CD₃OD, δ): 15.4, 56.8 (2×C), 61.2, 67.6, 109.1 (2×C), 120.2, 125.8, 127.3 (d, J=2.2 Hz), 130.5, 130.7, 134.6, 141.4, 142.9 (d, J=8.0 Hz), 144.5, 150.1 (d, J=8.0 Hz), 154.2, 154.6 (2×C), 196.7.

MS-ESI m/z (rel. int.): 464 ([M+H]⁺, 100, OPO(OH)₂ form).

HPLC: Method A, XBridge™ column, detection UV 254 nm, RT=3.47 min, peak area 95.6%.

### Preparation of 7-(2,2,2-trifluoroethoxy)-4-(3,4,5-trimethoxybenzyl)isoquinolin-8-ol hydrochloride 99

### 3-Hydroxy-2-methoxymethoxy-benzaldehyde SIL 32152

To a solution of 2,3-dihydroxybenzaldehyde (1.00 g, 7.24 mmol) in dry DMF (36 mL) in a 100 mL round-bottomed flask equipped with a magnetic stirrer was added cesium carbonate (2.36 g, 7.24 mmol) and the mixture was stirred for 10 min under N₂. Chloromethyl methyl ether (0.55 mL, 7.24 mmol) was added and stirring was continued overnight at RT. DMF was then evaporated under vacuum and the residue was diluted with CH₂Cl₂ (200 mL) and H₂O (50 mL). The mixture was acidified with a 5% aqueous solution of citric acid (1 mL) to pH=7. The organic layer was isolated and the aqueous layer was further extracted with CH₂Cl₂ (2×100 mL). The organic layers were combined, washed with brine (25 mL), dried over Na₂SO₄, filtered and concentrated under vacuum. Purification by column chromatography (SiO₂, eluent cyclohexane:acetone=100:0 to 60:40) gave, after evaporation and drying, 3-hydroxy-2-methoxymethoxy-benzaldehyde (0.55 g, 42% yield) as a white solid.

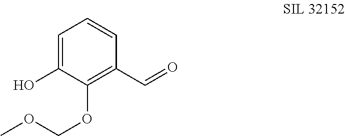

MW: 182.18; Yield: 42%; White solid; Mp (° C.): 53.2

R_{f}: 0.2 (cyclohexane: EtOAc=3:1).

¹H-NMR (CDCl₃, δ): 3.69 (s, 3H, CH₃O), 5.17 (s, 2H, OCH₂O), 7.17 (dd, 1H, J=7.8 Hz, ArH), 7.25 (dd, 1H, J=1.9 and 7.8 Hz, ArH), 7.36 (dd, 1H, J=1.9 and 7.8 Hz, ArH), 7.54 (br, s, 1H, OH), 10.31 (s, 1H, CHO).

¹³C-NMR (CDCl₃, δ): 57.7, 100.4, 120.6, 123.3, 125.5, 129.4, 147.7, 149.3, 189.8.

MS-ESI m/z (rel. int.): 151 (100).

HPLC: Method A (5 min), XBridge™ column, detection UV 254 nm, RT=2.38 min, peak area 77%.

### 2-Methoxymethoxy-3-(2,2,2-trifluoro-ethoxy)-benzaldehyde SIL 32120

To a solution of 3-hydroxy-2-methoxymethoxy-benzaldehyde SIL 32152 (850 mg, 4.66 mmol) in dry DMF (15 mL) in a 50 mL round-bottomed flask equipped with a magnetic stirrer was added cesium carbonate (1.67 g, 5.13 mmol) and the mixture was stirred at RT for 10 min under N₂. 2,2,2-Trifluoroethyl iodide (0.51 mL, 5.13 mmol) was added and the mixture was stirred overnight at RT. Another portion of 2,2,2-trifluoroethyl iodide (0.51 mL, 5.13 mmol) was added and the mixture was stirred for another 2 h at 50° C. then for 8 h at 100° C. The reaction mixture was diluted with Et₂O (100 mL) and H₂O (50 mL). The organic layer was isolated and the aqueous layer was further extracted with Et₂O (2×100 mL). The organic layers were combined, washed with brine (30 mL), dried over Na₂SO₄, filtered and concentrated under vacuum. Purification by column chromatography (SiO₂, eluent cyclohexane:EtOAc=100:0 to 90:10) gave, after evaporation and drying, 2-methoxymethoxy-3-(2,2,2-trifluoro-ethoxy)-benzaldehyde SIL 32120 (175 mg, 14% yield) as a yellow oil.

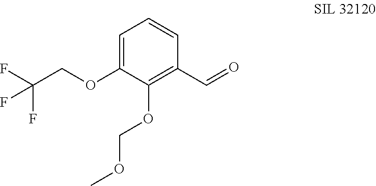 [SMILES: COCOc1c(C=O)cccc1OCC(F)(F)F]

MW: 264.20; Yield: 14%; Yellow oil.

R_{f}: 0.2 (cyclohexane:EtOAc=90:10).

¹H-NMR (CDCl₃, δ): 3.58 (s, 3H, CH₃O), 4.42 (q, 2H, J=8.0 Hz, CH₂CF₃), 5.24 (s, 2H, OCH₂O), 7.18 (d, 1H, J=3.6 Hz, ArH), 7.19 (d, 1H, J=5.8 Hz, ArH), 7.57 (dd, 1H, J=3.6 and 5.8 Hz, ArH), 10.46 (s, 1H, CHO).

¹³C-NMR (CDCl₃, δ): 58.0, 67.2 (q, J=35.8 Hz, CH₂CF₃), 100.0, 120.9, 122.2, 124.6, 131.1, 150.1, 150.4, 189.8, CF₃ not seen.

### (E)-2,2-Diethoxy-N-(2-(methoxymethoxy)-3-(2,2,2-trifluoroethoxy)benzylidene) ethanamine SIL 32134

A mixture of 2-methoxymethoxy-3-(2,2,2-trifluoro-ethoxy)-benzaldehyde SIL 32120 (168 mg, 0.636 mmol) and aminoacetaldehyde diethyl acetal (138 μL, 0.954 mmol) in toluene (25 mL) in a 100 mL round-bottomed flask equipped with a magnetic stirrer was stirred for 4 h under reflux using a Dean Stark apparatus. The mixture was then concentrated to dryness under vacuum to give 2-methoxymethoxy-3-(2,2,2-trifluoro-ethoxy)-benzaldehyde SIL 32134 as a yellow oil (265 mg, 100% yield). The crude product was used in the next step without any further purification.

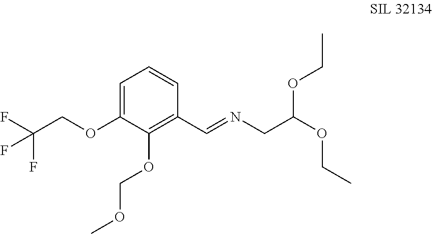 [SMILES: CCOC(C/N=C/c1cccc(OCC(F)(F)F)c1OCOC)OCC]

MW: 379.38; Yield: 100%; Yellow oil.

¹H-NMR (CDCl₃, δ): 1.20-1.25 (m, 6H, 2×OCH₂CH₃), 3.52-3.61 (m, 2H, CH₂N), 3.57 (s, 3H, CH₃O), 3.69-3.82 (m, 4H, 2×OCH₂CH₃), 4.38 (q, 2H, J=8.1 Hz, CH₂CF₃), 4.81 (t, 1H, J=5.3 Hz, OCHO), 5.15 (s, 2H, OCH₂O), 6.98 (dd, 1H, J=1.5 and 7.9 Hz, ArH), 7.10 (t, 1H, J=7.9 Hz, ArH), 7.10 (dd, 1H, J=1.5 and 7.9 Hz, ArH), 8.69 (s, 1H, CHN).

### (2,2-Diethoxy-ethyl)-[2-methoxymethoxy-3-(2,2,2-trifluoro-ethoxy)-benzyl]-amine SIL 32140

A mixture of the crude 2-methoxymethoxy-3-(2,2,2-trifluoro-ethoxy)-benzaldehyde SIL 32134 (243 mg, 0.64 mmol) and NaBH₄ (46 mg, 1.21 mmol) in EtOH (6 mL) in a 25 mL round-bottomed flask equipped with a magnetic stirrer was stirred under reflux for 2.5 h then cooled to RT. Water (2 mL) was added and the solvents were removed under vacuum. The residue was partitioned between water (50 mL) and CH₂Cl₂ (100 mL). The organic layer was washed with brine (15 mL), dried over Na₂SO₄, filtered and concentrated to dryness under vacuum. After purification by column chromatography (SiO₂, eluent cyclohexane:EtOAc=100:0 to 50:50), (2,2-diethoxy-ethyl)-[2-methoxymethoxy-3-(2,2,2-trifluoro-ethoxy)-benzyl]-amine (49 mg, 20% yield) was isolated as a yellow oil.

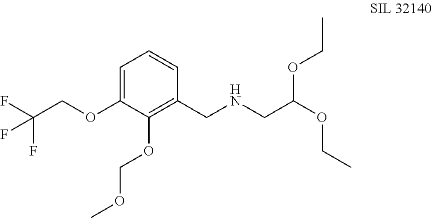 [SMILES: CCOC(CNCc1cccc(OCC(F)(F)F)c1OCOC)OCC]

MW: 381.40; Yield: 20%; Yellow oil.

R_{f}: 0.2 (cyclohexane:EtOAc=50:50).

¹H-NMR (CDCl₃, δ): 1.20 (t, 6H, J=7.0 Hz, 2×CH₃CH₂), 1.88 (br, s, 1H, NH), 2.74 (d, 2H, J=5.6 Hz, CHCH₂N), 3.50-3.58 (m, 2H, OCH₂), 3.59 (s, 3H, OCH₃), 3.65-3.71 (m, 2H, OCH₂), 3.86 (s, 2H, ArCH₂N), 4.35 (q, 2H, J=8.2 Hz, OCH₂CF₃), 4.61 (t, 1H, J=5.6 Hz, CH₂CHO), 5.12 (s, 2H, OCH₂O), 6.83 (dd, 1H, J=3.7 and 6.1 Hz, ArH), 7.02-7.05 (m, 2H, 2×ArH).

¹³C-NMR (CDCl₃, δ): 15.2, 48.6, 51.5, 57.3, 62.2, 66.7 (q, J=35.2 Hz, CH₂CF₃), 99.2, 102.2, 113.8, 123.5 (J=278 Hz, CF₃), 124.1 (2×C), 135.0, 145.3, 149.8.

### 7-(2,2,2-Trifluoroethoxy)-4-(3,4,5-trimethoxybenzyl)isoquinolin-8-ol hydrochloride 99

A mixture of (2,2-diethoxy-ethyl)-[2-methoxymethoxy-3-(2,2,2-trifluoro-ethoxy)-benzyl]-amine SIL 32140 (45 mg, 118 μmol) and 3,4,5-trimethoxybenzaldehyde (40 mg, 204 μmol) in a mixture of EtOH:conc. HCl=1:1 (5 mL) in a 10 mL microwave vial equipped with a magnetic stirrer was stirred for 20 min at 100° C. under microwave irradiation. The volatiles were then removed at 40° C. under vacuum and the residue was taken up in CH₂Cl₂ (30 mL) before neutralisation with a saturated aqueous NaHCO₃ solution (10 mL). The organic layer was isolated, washed with brine (10 mL), dried over Na₂SO₄, filtered and concentrated at 40° C. under vacuum. Purification by column chromatography (SiO₂, eluent cyclohexane:acetone=100:0 to 60:40) gave a brown solid that was dissolved in MeOH (2 mL) in a 10 mL round-bottomed flask equipped with a magnetic stirrer before addition of 0.49 M HCl in MeOH (1 mL). The mixture was then stirred for 5 min at RT, and concentrated at 40° C. under vacuum to afford 7-(2,2,2-trifluoroethoxy)-4-(3,4,5-trimethoxybenzyl)isoquinolin-8-ol hydrochloride 99 (16 mg, 29% yield) as a brown solid.

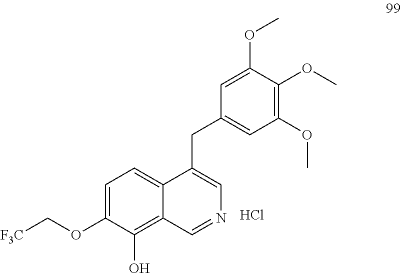 [SMILES: COc1cc(Cc2cncc3c(O)c(OCC(F)(F)F)ccc23)cc(OC)c1OC ; Cl]

MW: 459.84; Yield: 29%; Brown solid; Mp (° C.): 88.1 (dec.)

R_{f}: 0.2 (cyclohexane:acetone=60:40, free base).

¹H-NMR (CD₃OD, δ): 3.73 (s, 3H, OCH₃), 3.77 (s, 6H, 2×OCH₃), 4.51 (s, 2H, CH₂), 4.82-4.87 (m, 2H, OCH₂), 6.62 (s, 2H, 2×ArH), 7.91 (d, 1H, J=9.2 Hz, ArH), 8.09 (d, 1H, J=9.2 Hz, ArH), 8.16 (s, 1H, ArH), 9.72 (s, 1H, ArH).

¹³C-NMR (CD₃OD, δ): 37.0, 56.7 (2×C), 61.1, 68.2 (CH₂CF₃, J=35.6 Hz), 107.5 (2×C), 116.7, 121.4, 125.5 (CF₃, J=271.8 Hz), 127.8, 129.6, 134.7, 134.9, 138.1, 138.2, 142.8, 145.0, 147.4, 155.0 (2×C).

MS-ESI m/z (rel. int.): 424 ([MH]⁺, 100).

HPLC: Method A (5 min), XBridge™ column, detection UV 254 nm, RT=2.36 min, peak area 95.7%.

### Preparation of 4-(3,4,5-trimethoxybenzyl)isoquinoline-7,8-diol hydrochloride 100

### (E)-3-((2,2-diethoxyethylimino)methyl)benzene-1,2-diol SLA 41050

A mixture of 2,3-dihydroxybenzaldehyde (5.00 g, 36.2 mmol) and 2,2-diethoxyethanamine (7.9 mL, 54.3 mmol) in toluene (100 mL) in a 500 mL round-bottomed flask equipped with a magnetic stirrer was stirred for 4 h under reflux using a Dean Stark apparatus. After cooling to RT, toluene was removed at 60° C. under vacuum to give (E)-3-((2,2-diethoxyethylimino)methyl)benzene-1,2-diol SLA 41050 (10.76 g, >100%) as a crude brown oil.

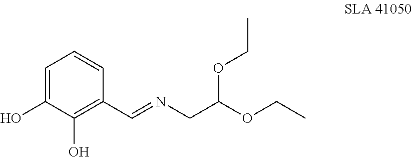 [SMILES: CCOC(C/N=C/c1cccc(O)c1O)OCC]

MW: 253.29; Yield: >100%; Crude brown oil.

R_{f}: 0.4 (CH₂Cl₂:MeOH=97:3).

¹H-NMR (CDCl₃ exchanged with CD₃OD, δ): 1.21 (t, 6H, J=7.2 Hz, 2×CH₃), 3.51-3.63 (m, 2H, CH₂), 3.67-3.80 (m, 4H, 2×CH₂), 4.69 (t, 1H, J=5.4 Hz, CH), 6.63 (dd, 1H, J=7.8 Hz, ArH), 6.75 (d, 1H, J=7.8 Hz, ArH), 6.92 (d, 1H, J=7.8 Hz, ArH), 8.20 (s, 1H, CHN).

¹³C-NMR (CDCl₃, δ): 15.4 (2×C), 59.2, 62.5, 63.1 (2×C), 101.4, 116.4, 116.9, 122.1, 146.4, 155.4, 166.6.

MS-ESI m/z (% rel. Int.): 208 (100).

HPLC: Method A (5 min), XBridge™ column, detection UV 254 nm, RT=2.92 min.

### 3-((2,2-Diethoxyethylamino)methyl)benzene-1,2-diol SLA 41054

To a stirred solution of (E)-3-((2,2-diethoxyethylimino)methyl)benzene-1,2-diol SLA 41050 (9.17 g, 36.2 mmol) in EtOH (100 mL) in a 250 mL round-bottomed flask equipped with a magnetic stirrer was added NaBH₄ (2.74 g, 72.4 mmol) and the mixture was stirred for 1 h at RT and cooled in an ice bath before addition of H₂O (2 mL). After stirring for 5 min at +4° C., EtOH was removed at 40° C. under vacuum and the solution was extracted with CH₂Cl₂ (200 mL). The organic layer was washed with brine (20 mL), dried over Na₂SO₄, filtered, and concentrated under vacuum to give 3-((2,2-diethoxyethylamino)methyl)benzene-1,2-diol SLA 41054 (5.30 g, 57% yield) as a brown oil.

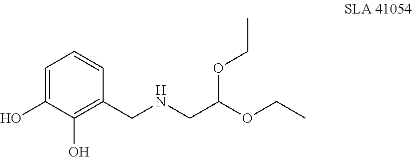 [SMILES: CCOC(CNCc1cccc(O)c1O)OCC]

MW: 255.31; Yield: 57%; Brown oil.

¹H NMR (CDCl₃, δ): 1.10-1.15 (m, 1H, NH), 1.18-1.24 (m, 6H, 2×CH₃), 2.78 (d, 2H, J=5.0 Hz, CH₂), 3.46-3.58 (m, 2H, OCH₂), 3.63-3.78 (m, 2H, OCH₂), 3.99 (s, 2H, CH₂), 4.60 (t, 1H, J=5.0 Hz, CH), 4.93 (broad s, 2H, 2×OH), 6.52 (d, 1H, J=7.5 Hz, ArH), 6.64-6.69 (m, 1H, ArH), 6.80 (d, 1H, J=8.1 Hz, ArH).

¹³C-NMR (CDCl₃, δ): 15.3 (2×C), 44.3, 50.6, 52.1, 62.6, 62.7, 101.2, 103.4, 113.9, 119.3, 122.2, 145.0.

MS-ESI m/z (% rel. Int.): 210 (100), 256 ([MH]⁺, 6).

HPLC: Method A (5 min), XBridge™ column, detection UV 254 nm, RT=2.02 min.

### 4-(3,4,5-Trimethoxybenzyl)isoquinoline-7,8-diol hydrochloride 100

To a stirred solution of 3-((2,2-diethoxyethylamino)methyl)benzene-1,2-diol SLA 41054 (1.0 g, 3.92 mmol) in EtOH:conc. HCl=1:1 (10 mL) in a 20 mL microwave vial equipped with a magnetic stirrer was added 3,4,5-trimethoxybenzaldehyde (0.77 g, 3.92 mmol) and the mixture was stirred at 100° C. for 20 min under microwave irradiation. After cooling to RT, the volatiles were removed at 40° C. under vacuum and the residue was diluted with CH₂Cl₂ (100 mL) and neutralized with a saturated NaHCO₃ aq. solution (20 mL). The organic layer was isolated, washed with brine (20 mL), dried over Na₂SO₄, filtered, and concentrated at 40° C. under vacuum. The crude product was purified by prep. HPLC (Prep C18Xterra 19×50 mm column 186001108 A, eluent H₂O:CH₃CN:TFA=100:0:0.05 to 70:30:0.05 in 10 min) to give a yellow residue. This residue was dissolved in CH₂Cl₂ (2 mL) in a 10 mL round-bottomed flask equipped with a magnetic stirrer before addition of 0.49 M HCl in MeOH (0.40 mL) and the solution was stirred for 5 min at RT then concentrated at 40° C. under vacuum, to afford 4-(3,4,5-trimethoxybenzyl)isoquinoline-7,8-diol hydrochloride 100 (24 mg, 2% yield) as a yellow solid.

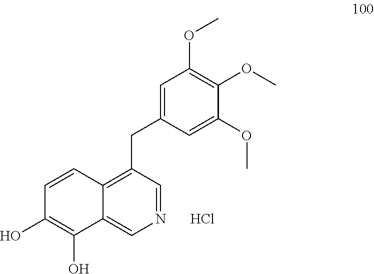 [SMILES: COc1cc(Cc2cncc3c(O)c(O)ccc23)cc(OC)c1OC ; Cl]

MW: 377.82; Yield: 2%; Yellow solid; Mp (° C.): 225.0

¹H NMR (CD₃OD, δ): 3.73 (s, 3H, OCH₃), 3.77 (s, 6H, 2×OCH₃), 4.46 (s, 2H, CH₂), 6.60 (s, 2H, 2×ArH), 7.78-7.86 (m, 2H, 2×ArH), 8.01 (s, 1H, ArH), 9.59 (s, 1H, ArH).

¹³C-NMR (CD₃OD, δ): 37.0, 56.7 (2×C), 61.1, 107.5 (2×C), 116.6, 121.0, 127.8, 129.5, 132.5, 135.1, 137.9, 138.1, 141.7, 144.6, 145.5, 154.9 (2×C).

MS-ESI m/z (rel. int.): 342 ([MH]⁺, 100).

HPLC: Method A (10 min), XBridge™ column, detection UV 254 nm, RT=3.62 min, peak area 99.8%.

### Preparation of 4-(3,4,5-trimethoxybenzyl)isoquinolin-7-ol hydrochloride 101

### 4-(3,4,5-Trimethoxybenzyl)isoquinolin-7-ol hydrochloride 101

To a solution of 7-ethoxy-4-(3,4,5-trimethoxybenzyl)isoquinolin-8-amine hydrochloride 3 (150 mg, 0.358 mmol) in conc. HCl (1.3 mL) at 5° C. in a 10 mL round-bottomed flask equipped with a magnetic stirrer was added a solution of sodium nitrite (18 mg, 261 μmol) in H₂O (2.7 mL). The reaction mixture was stirred for 1 h at 5° C. before adding AcOH (1 mL), THF (2 mL) and another portion of sodium nitrite (12 mg, 174 μmol). After stirring for 30 min at 5° C., HBF₄ (60 μl, 0.96 mmol) was added and the reaction mixture was stirred overnight at RT. The reaction mixture was basified to pH=9 with a 2 N aq. NaOH solution before to be extracted with CH₂Cl₂ (70 mL). The organic layer was washed with brine (15 mL), dried over Na₂SO₄, filtered and concentrated at 40° C. under vacuum. Purification by column chromatography (SiO₂, eluent CH₂Cl₂:MeOH=100:0 to 90:10) gave, after evaporation and drying, 4-(3,4,5-trimethoxybenzyl)isoquinolin-7-ol as a brown solid. This product was dissolved in CH₂Cl₂ (2 mL) in a 10 mL round-bottomed flask equipped with a magnetic stirrer before addition of 0.49 M HCl in MeOH (0.40 mL) and the solution was stirred for 5 min at RT and concentrated at 40° C. under vacuum to afford 4-(3,4,5-trimethoxybenzyl)isoquinolin-7-ol hydrochloride 101 (15 mg, 12% yield) as a yellow solid.

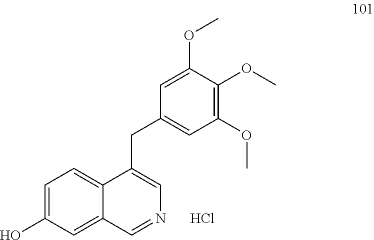 [SMILES: COc1cc(Cc2cncc3cc(O)ccc23)cc(OC)c1OC ; Cl]

MW: 361.82; Yield: 12%; Yellow solid; Mp (° C.): 98.6 (dec.)

¹H NMR (CD₃OD, δ): 3.75 (s, 3H, OCH₃), 3.84 (s, 6H, 2×OCH₃), 4.54 (s, 2H, CH₂), 6.63 (s, 2H, 2×ArH), 7.69 (d, 1H, J=2.7 Hz, ArH), 7.79 (dd, 1H, J=2.7 and 9.3 Hz, ArH), 8.15 (s, 1H, ArH), 8.38 (d, 1H, J=9.3 Hz, ArH), 9.44 (s, 1H, ArH).

¹³C-NMR (CD₃OD, δ): 36.8, 56.7 (2×C), 61.1, 107.6 (2×C), 112.2, 127.6, 128.9, 130.7, 131.1, 133.8, 135.1, 138.3, 138.7, 144.7, 155.0 (2×C), 160.9.

MS-ESI m/z (% rel. Int.): 326 ([MH]⁺, 100).

HPLC: Method A (10 min), XBridge™ column, detection UV 254 nm, RT=3.68 min, peak area 98.0%.

### Preparation of 7-ethoxy-4-(4-hydroxy-3-methoxy-5-nitrobenzyl)isoquinolin-8-ol hydrochloride 102

### 7-Ethoxy-4-(4-hydroxy-3-methoxy-5-nitrobenzyl)isoquinolin-8-ol hydrochloride 102

A mixture of 2-((2,2-diethoxyethylamino)methyl)-6-ethoxyphenol SAO 33014 (204 mg, 799 μmol) and 5-nitrovanillin (158 mg, 801 μmol) in a mixture of EtOH:conc. HCl=1:1 (2 mL) in a 5 mL microwave vial equipped with a magnetic stirrer was stirred for 20 min at 100° C. under microwave irradiation. The volatiles were removed at 40° C. under vacuum and the residue was taken up in a mixture of CH₂Cl₂:MeOH=9:1 (100 mL) before neutralisation with a saturated aqueous NaHCO₃ solution (15 mL). The separated organic layer was washed with brine (20 mL), dried over Na₂SO₄, filtered and concentrated at 40° C. under vacuum. Purification by column chromatography (SiO₂, eluent: CH₂Cl₂:MeOH=100:0 to 97:3) gave 7-ethoxy-4-(4-hydroxy-3-methoxy-5-nitrobenzyl)isoquinolin-8-ol. This free base was dissolved in MeOH (2 mL) in a 10 mL round-bottomed flask equipped with a magnetic stirrer before addition of a 0.4 M HCl solution in MeOH (1 mL). The reaction mixture was stirred for 5 min at RT and concentrated at 40° C. under vacuum to give 7-ethoxy-4-(4-hydroxy-3-methoxy-5-nitrobenzyl)isoquinolin-8-ol hydrochloride 102 (95 mg, 29% yield) as a pale brown solid.

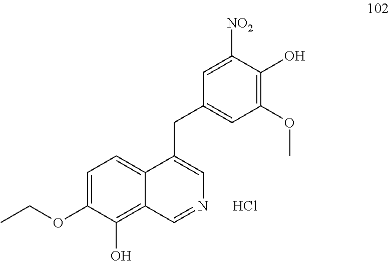 [SMILES: CCOc1ccc2c(Cc3cc(OC)c(O)c([N+](=O)[O-])c3)cncc2c1O ; Cl]

MW: 406.82; Yield: 29%; Pale brown solid; Mp (° C.): 250.1 (dec.)

R_{f}: 0.2 (CH₂Cl₂:MeOH=97:3, free base).

¹H-NMR (DMSO, δ): 1.40 (t, 3H, J=6.8 Hz, CH₂CH₃), 3.84 (s, 3H, OCH₃), 4.29 (q, 2H, J=6.8 Hz, CH₂CH₃), 4.49 (s, 2H, CH₂), 7.36 (s, 2H, 2×ArH), 7.82 (d, 1H, J=9.0 Hz), 8.05 (d, 1H, J=9.0 Hz), 8.39 (s, 1H, ArH), 9.66 (s, 1H, ArH), 10.34 (broad s, 1H, NH or OH), 11.03 (br, s, 1H, NH or OH) (1×NH or OH not seen).

¹³C-NMR (DMSO, δ): 14.6, 56.6, 65.3, 115.2, 115.5, 117.0, 119.2, 125.7, 128.3, 129.2, 130.9, 134.6, 137.0, 141.1, 141.3, 144.5, 144.8, 149.5.

MS-ESI m/z (rel. int.): 371 ([MH]⁺, 100).

HPLC: Method A (10 min), XBridge™ column, detection UV 254 nm, RT=4.02 min, peak area 96.3%.

### Preparation of 4-(3,4-dimethoxy-5-nitrobenzyl)-7-ethoxyisoquinolin-8-ol hydrochloride 103

### 3,4-Dimethoxy-5-nitrobenzaldehyde CCH 42028-2

To a solution of 5-nitrovanillin (300 mg, 1.52 mmol) in dry DMF (5 mL) in a 10 mL microwave vial equipped with a magnetic stirrer was added cesium carbonate (600 mg, 1.84 mmol) and the mixture was stirred for 10 min under N₂. Iodomethane (130 μl, 2.09 mmol) was added and the reaction mixture was stirred overnight at RT. Another portion of iodomethane (500 μl, 8.03 mmol) was then added and the reaction mixture was stirred for 1 h at 100° C. under microwave irradiation. After cooling to RT, the mixture was diluted with Et₂O (50 mL) and the organic solution was washed with water (3×5 mL), with brine (10 mL), dried over Na₂SO₄, filtered and concentrated under vacuum. Purification by column chromatography (SiO₂, eluent cyclohexane:EtOAc=100:0 to 75:25) gave, after evaporation and drying, 3,4-dimethoxy-5-nitrobenzaldehyde CCH42028-2 (128 mg, 40% yield) as an off-white solid.

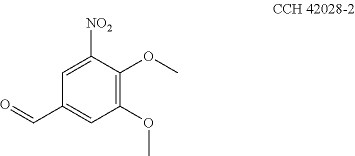

MW: 211.17; Yield: 40%; Off-white solid.

¹H-NMR (CDCl₃, δ): 4.01 (s, 3H, CH₃O), 4.08 (s, 3H, CH₃O), 7.63 (s, 1H, ArH), 7.84 (s, 1H, ArH), 9.92 (s, 1H, CHO).

¹³C-NMR (CDCl₃, δ): 56.7, 62.3, 113.4, 119.5, 131.4, 144.8, 147.8, 154.7, 189.0.

MS-ESI m/z (rel. int.): 212 ([MH]⁺, 100).

HPLC: Method A (10 min), XBridge™ column, detection UV 254 nm, RT=4.98 min.

### Preparation of 4-(3,4-dimethoxy-5-nitrobenzyl)-7-ethoxyisoquinolin-8-ol hydrochloride CCH 42028C

### 4-(3,4-Dimethoxy-5-nitrobenzyl)-7-ethoxyisoquinolin-8-ol hydrochloride 103

A mixture of 2-((2,2-diethoxyethylamino)methyl)-6-ethoxyphenol SAO 33014 (165 mg, 646 μmol) and 3,4-dimethoxy-5-nitrobenzaldehyde CCH 42028-2 (126 mg, 597 μmol) in a mixture of EtOH:conc. HCl=1:1 (5 mL) in a 10 mL microwave vial equipped with a magnetic stirrer was stirred for 20 min at 100° C. under microwave irradiation. The volatiles were removed at 40° C. under vacuum and the residue was neutralized with a saturated aqueous NaHCO₃ solution (20 mL) before extraction with CH₂Cl₂ (100 mL). The separated organic layer was washed with brine (20 mL), dried over Na₂SO₄, filtered and concentrated at 40° C. under vacuum. Purification by column chromatography (SiO₂, eluent cyclohexane:EtOAc=100:0 to 50:50) gave, after evaporation and drying, 4-(3,4-dimethoxy-5-nitrobenzyl)-7-ethoxyisoquinolin-8-ol. This free base was dissolved in MeOH (2 mL) in a 10 mL round-bottomed flask equipped with a magnetic stirrer before addition of a 0.4 M HCl solution in MeOH (2 mL). The reaction mixture was stirred for 5 min at RT and concentrated at 40° C. under vacuum to give 4-(3,4-dimethoxy-5-nitrobenzyl)-7-ethoxyisoquinolin-8-ol hydrochloride 103 (130 mg, 52% yield) as a yellow solid.

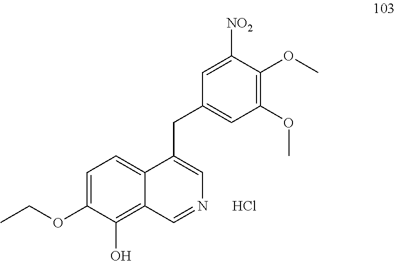 [SMILES: CCOc1ccc2c(Cc3cc(OC)c(OC)c([N+](=O)[O-])c3)cncc2c1O ; Cl]

MW: 420.84; Yield: 52%; Yellow solid; Mp (° C.): 168.3 (dec.)

¹H-NMR (CD₃OD, δ): 1.34-1.43 (m, 3H, CH₂CH₃), 3.76-3.81 (m, 6H, 2×OCH₃), 4.18-4.27 (m, 2H, CH₂CH₃), 4.46 (s, 2H, CH₂), 7.05 (s, 1H, ArH), 7.18 (s, 1H, ArH), 7.67-7.70 (m, 1H, ArH), 7.89-7.95 (m, 1H, ArH), 8.04 (s, 1H, ArH), 9.55 (s, 1H, ArH).

¹³C-NMR (CD₃OD, δ): 15.0, 36.1, 57.1, 62.3, 66.9, 116.4, 116.5, 118.0, 120.5, 126.8, 128.6, 132.5, 136.0, 136.6, 142.4, 143.0, 146.5, 146.5, 147.1, 155.7.

MS-ESI m/z (rel. int.): 385 ([MH]⁺, 100).

HPLC: Method A (10 min), XBridge™ column, detection UV 254 nm, RT=4.39 min, peak area 98.3%.

### Preparation of tert-butyl N-(7-ethoxy-4-(3,4,5-trimethoxybenzoyl)isoquinolin-8-yl)sulfamoylcarbamate 104

### tert-Butyl N-(7-ethoxy-4-(3,4,5-trimethoxybenzoyl)isoquinolin-8-yl)sulfamoylcarbamate 104

To a solution of chlorosulfonyl isocyanate (40 μl, 460 μmol) in dry THF (5 mL) at 0° C. in a 25 mL round-bottomed flask equipped with a magnetic stirrer was added dropwise tert-BuOH (46 μl, 484 μmol) and the mixture was stirred for 2 h at RT. (8-Amino-7-ethoxyisoquinolin-4-yl)(3,4,5-trimethoxyphenyl)methanone 29 free base (124 mg, 324 μmol) was added to the reaction mixture and stirring was continued for 1 h at RT. NEt₃ (67 μl, 482 μmol) was added and stirring was continued overnight at RT. The volatiles were removed at 40° C. under vacuum and the residue was taken up in CH₂Cl₂ (30 mL). The organic layer was washed with water (5 mL), brine (5 mL), dried over Na₂SO₄, filtered and concentrated at 40° C. under vacuum. Purification by column chromatography (SiO₂, eluent: cyclohexane:EtOAc=100:0 to 55:45) gave, after evaporation and drying, tert-butyl N-(7-ethoxy-4-(3,4,5-trimethoxybenzoyl)isoquinolin-8-yl)sulfamoylcarbamate 104 (61 mg, 34% yield) as a beige solid.

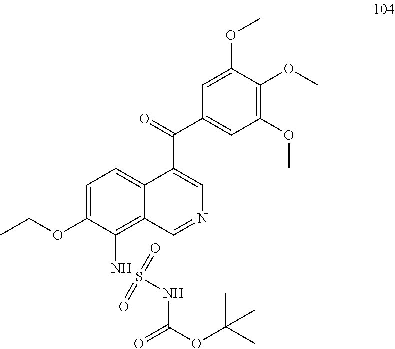

MW: 561.60; Yield: 34%; Beige solid; Mp (° C.): 221.0 (dec.).

R_{f}: 0.2 (cyclohexane: EtOAc=55:45).

¹H-NMR (CDCl₃, δ): 1.50-1.53 (m, 3H, CH₂CH₃), 1.51 (s, 9H, C(CH₃)₃), 3.83 (s, 6H, 2×OCH₃), 3.96 (s, 3H, OCH₃), 4.31 (q, 2H, J=7.0 Hz, CH₂CH₃), 7.13 (s, 2H, 2×ArH), 7.52 (d, 1H, J=9.4 Hz), 8.17 (d, 1H, J=9.4 Hz), 8.50 (s, 1H, ArH), 9.75 (s, 1H, ArH).

¹³C-NMR (CDCl₃, δ): 14.8, 28.1 (3×C), 56.4 (2×C), 61.0, 65.5, 83.9, 108.0 (2×CH), 118.5, 119.1, 126.8, 127.4, 128.4, 129.1, 132.7, 142.2, 143.3, 149.9, 151.7, 152.4, 153.1 (2×C), 194.7.

MS-ESI m/z (rel. int.): 562 ([MH]⁺, 100).

HPLC: Method A (10 min), XBridge™ column, detection UV 254 nm, RT=4.71 min, peak area 99.4%.

### Preparation of N-(7-ethoxy-4-(3,4,5-trimethoxybenzoyl)isoquinolin-8-yl)sulfamide hydrochloride 105

### N-(7-Ethoxy-4-(3,4,5-trimethoxybenzoyl)isoquinolin-8-yl)sulfamide hydrochloride 105

A solution of tert-butyl N-(7-ethoxy-4-(3,4,5-trimethoxybenzoyl)isoquinolin-8-yl)sulfamoylcarbamate 104 (42 mg, 77 μmol) in a mixture of CH₂Cl₂:TFA=5:1 (6 mL) in a 10 mL round-bottomed flask equipped with a magnetic stirrer was stirred overnight at RT. The volatiles were removed at 40° C. under vacuum and the residue was neutralized with a saturated aqueous NaHCO₃ solution (15 mL) and extracted with CH₂Cl₂ (2×25 mL). The combined organic layers were washed with brine (5 mL), dried over Na₂SO₄, filtered and concentrated at 40° C. under vacuum. Purification by column chromatography (SiO₂, eluent: cyclohexane:EtOAc=100:0 to 40:60) gave N-(7-ethoxy-4-(3,4,5-trimethoxybenzoyl)isoquinolin-8-yl)sulfamide. This free base was dissolved in MeOH (2 mL) in a 10 mL round-bottomed flask equipped with a magnetic stirrer before addition of a 0.4 M HCl solution in MeOH (1 mL). The reaction mixture was stirred for 5 min at RT and concentrated at 40° C. under vacuum to give N-(7-ethoxy-4-(3,4,5-trimethoxybenzoyl)isoquinolin-8-yl)sulfamide hydrochloride 105 (11 mg, 29% yield) as a red solid.

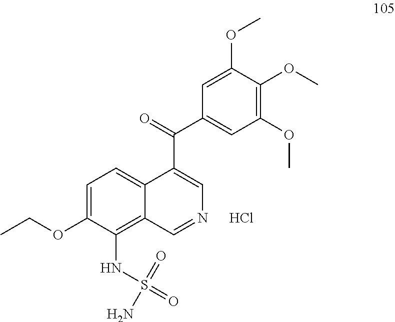 [SMILES: CCOc1ccc2c(C(=O)c3cc(OC)c(OC)c(OC)c3)cncc2c1NS(N)(=O)=O ; Cl]

MW: 497.95; Yield: 29%; Red solid.

¹H-NMR (CD₃OD, δ): 1.58 (t, 3H, J=6.9 Hz, CH₂CH₃), 3.85 (s, 6H, 2×OCH₃), 3.92 (s, 3H, OCH₃), 4.47 (q, 2H, J=6.9 Hz, CH₂CH₃), 7.25 (s, 2H, 2×ArH), 8.15-8.25 (m, 2H, 2×ArH), 8.62 (s, 1H, ArH), 9.99 (s, 1H, ArH).

¹³C-NMR (CD₃OD, δ): 14.9, 57.0 (2×C), 61.3, 67.0, 109.4 (2×C), 124.0, 126.8, 127.3, 129.3, 130.3, 132.2, 132.9, 135.7, 145.6, 148.0, 154.9 (2×C), 156.9, 192.3.

MS-ESI m/z (rel. int.): 462 ([MH]⁺, 100).

HPLC: Method A, detection UV 254 nm, XBridge™ column, RT=3.92 min, peak area 95.2%.

### Preparation of 8-chloro-7-ethoxy-4-(3,4,5-trimethoxybenzyl)isoquinoline hydrochloride 106

### 8-Chloro-7-ethoxy-4-(3,4,5-trimethoxybenzyl)isoquinoline hydrochloride 106

To a solution of 7-ethoxy-4-(3,4,5-trimethoxybenzyl)isoquinolin-8-amine dihydrochloride 3 (150 mg, 0.34 mmol) in a mixture of conc. HCl (1 mL) and AcOH (2 mL) at 1° C. in a 10 mL round-bottomed flask equipped with a magnetic stirrer was added a solution of sodium nitrite (24 mg, 0.35 mmol) in H₂O (1 mL). The reaction mixture was stirred for 2 h at RT and cold H₂O (2 mL) was added followed by a solution of CuCl (67 mg, 0.68 mmol) in a 6N aq. HCl solution (0.5 mL). The reaction mixture was stirred for 4 h, allowing the medium to warm up to RT. The reaction mixture was basified to pH=9 with a 2 N aq. NaOH solution before extraction with CH₂Cl₂ (70 mL). The organic layer was washed with brine (15 mL), dried over Na₂SO₄, filtered and concentrated at 40° C. under vacuum. Purification by column chromatography (SiO₂, eluent cyclohexane:EtOAc=100:0 to 60:40) gave 8-chloro-7-ethoxy-4-(3,4,5-trimethoxybenzyl)isoquinoline. This free base was dissolved in MeOH (2 mL) in a 10 mL round-bottomed flask equipped with a magnetic stirrer before addition of 0.40 M HCl in MeOH (1 mL). The mixture was then stirred for 5 min at RT, and concentrated at 40° C. under vacuum to give 8-chloro-7-ethoxy-4-(3,4,5-trimethoxybenzyl)isoquinoline hydrochloride 106 (95 mg, 66% yield) as a pale yellow solid.

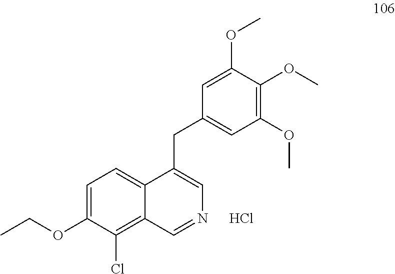 [SMILES: CCOc1ccc2c(Cc3cc(OC)c(OC)c(OC)c3)cncc2c1Cl ; Cl]

MW: 424.32; Yield: 66%; Pale yellow solid; Mp (° C.): 198.2 (dec.)

R_{f}: 0.2 (cyclohexane: EtOAc=60:40, free base).

¹H-NMR (CD₃OD, δ): 1.51-1.55 (m, 3H, CH₃), 3.72 (s, 3H, OCH₃), 3.77 (s, 6H, 2×OCH₃), 4.40-4.46 (m, 2H, CH₂), 4.59 (s, 2H, CH₂), 6.62 (s, 2H, 2×ArH), 8.15-8.19 (m, 1H, ArH), 8.33 (s, 1H, ArH), 8.42-8.48 (m, 1H, ArH), 9.77 (s, 1H, ArH).

¹³C-NMR (CD₃OD, δ): 15.0, 36.9, 56.7 (2×C), 61.1, 67.4, 107.5 (2×C), 120.1, 126.3, 126.4, 128.4, 130.4, 134.9, 138.2, 139.2, 143.0, 155.0 (2×C), 157.0, 1×C not observed.

MS-ESI m/z (rel. int.): 388 ([MH]⁺, ³⁵Cl, 100), 390 ([MH]⁺, ³⁷Cl, 36).

HPLC: Method A (10 min), XBridge™ column, detection UV 254 nm, RT=4.53 min, peak area 98.7%.

### Preparation of 8-azido-7-ethoxy-4-(3,4,5-trimethoxybenzyl)isoquinoline 107

### 8-Azido-7-ethoxy-4-(3,4,5-trimethoxybenzyl)isoquinoline 107

To a solution of 7-ethoxy-4-(3,4,5-trimethoxybenzyl)isoquinolin-8-amine dihydrochloride 3 (150 mg, 0.34 mmol) in a mixture of conc. HCl (1 mL) and AcOH (2 mL) at 1° C. in a 10 mL round-bottomed flask equipped with a magnetic stirrer was added a solution of sodium nitrite (24 mg, 0.35 mmol) in H₂O (1 mL). The reaction mixture was stirred for 2 h at RT and cold H₂O (2 mL) was added followed by a solution of NaN₃ (27 mg, 0.42 mmol) in H₂O (1 mL). The reaction mixture was stirred for 4 h, allowing the medium to warm up to RT. The reaction mixture was basified to pH=9 with a 2 N aq. NaOH solution before extraction with CH₂Cl₂ (70 mL). The organic layer was washed with brine (15 mL), dried over Na₂SO₄, filtered and concentrated at 40° C. under vacuum. Purification by column chromatography (SiO₂, eluent cyclohexane:EtOAc=100:0 to 65:35) gave 8-azido-7-ethoxy-4-(3,4,5-trimethoxybenzyl)isoquinoline 107 (16 mg, 12% yield) as a pale brown solid.

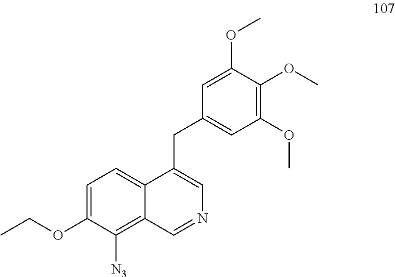 [SMILES: CCOc1ccc2c(Cc3cc(OC)c(OC)c(OC)c3)cncc2c1N=[N+]=[N-]]

MW: 394.42; Yield: 12%; Pale brown solid; Mp (° C.): 147.6 (dec.)

R_{f}: 0.2 (cyclohexane: EtOAc=65:35).

¹H-NMR (CDCl₃, δ): 1.51 (t, 3H, J=7.0 Hz, CH₂CH₃), 3.75 (s, 6H, 2×OCH₃), 3.81 (s, 3H, OCH₃), 4.24 (q, 2H, J=7.0 Hz, CH₂CH₃), 4.26 (s, 2H, CH₂), 6.36 (s, 2H, 2×ArH), 7.37 (d, 1H, J=9.2 Hz, ArH), 7.64 (d, 1H, J=9.2 Hz, ArH), 8.29 (s, 1H, ArH), 9.45 (s, 1H, ArH).

¹³C-NMR (CDCl₃, δ): 14.5, 36.7, 56.1 (2×C), 60.8, 66.0, 105.6 (2×C), 118.8, 120.8, 122.7, 122.8, 128.5, 130.3, 135.3, 136.6, 142.3, 147.3, 149.9, 153.3 (2×C).

MS-ESI m/z (rel. int.): 341 (12), 367 (100), 395 ([MH]⁺, 18).

HPLC: Method A (10 min), XBridge™ column, detection UV 254 nm, RT=4.48 min, peak area 96.7%.

### Preparation of 8-azido-7-ethoxy-4-(3,4,5-trimethoxybenzoyl)isoquinoline 108

### 8-Azido-7-ethoxy-4-(3,4,5-trimethoxybenzoyl)isoquinoline 108

To a solution of (8-amino-7-ethoxyisoquinolin-4-yl)(3,4,5-trimethoxyphenyl)methanone hydrochloride 29 (213 mg, 509 μmol) in a mixture of conc. HCl (1.5 mL) and AcOH (3 mL) at 5° C. in a 10 mL round-bottomed flask equipped with a magnetic stirrer was added a solution of sodium nitrite (36 mg, 522 μmol) in H₂O (1.5 mL). The reaction mixture was stirred for 1 h at 5° C. before adding NaN₃ (40 mg, 615 μmol) and stirring was then continued for 2.5 h, allowing the medium to warm up to RT. Another portion of NaN₃ (24 mg, 369 μmol) was added and stirring was continued for 1 h at RT. The reaction mixture was then basified to pH=9 with a 2 N aq. NaOH solution before extraction with CH₂Cl₂ (70 mL). The organic layer was washed with brine (15 mL), dried over Na₂SO₄, filtered and concentrated at 40° C. under vacuum. Purification by column chromatography (SiO₂, eluent cyclohexane:EtOAc=100:0 to 65:35) gave 8-azido-7-ethoxy-4-(3,4,5-trimethoxybenzoyl)isoquinoline 108 (12 mg, 6% yield) as a pale yellow solid.

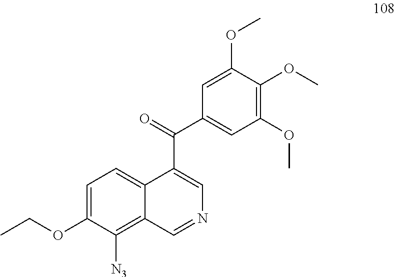 [SMILES: CCOc1ccc2c(C(=O)c3cc(OC)c(OC)c(OC)c3)cncc2c1N=[N+]=[N-]]

MW: 408.41; Yield: 6%; Pale yellow solid; Mp (° C.): 148.6 (dec.)

R_{f}: 0.15 (cyclohexane: EtOAc=65:35).

¹H-NMR (CDCl₃, δ): 1.54 (t, 3H, J=7.0 Hz, CH₂CH₃), 3.83 (s, 6H, 2×OCH₃), 3.95 (s, 3H, OCH₃), 4.29 (q, 2H, J=7.0 Hz, CH₂CH₃), 7.12 (s, 2H, 2×ArH), 7.47 (d, 1H, J=9.3 Hz, ArH), 7.88 (d, 1H, J=9.3 Hz, ArH), 8.53 (s, 1H, ArH), 9.65 (s, 1H, ArH).

¹³C-NMR (CDCl₃, δ): 14.5, 56.3 (2×C), 61.0, 66.0, 107.9 (2×C), 119.9, 122.2, 122.7, 128.5, 128.9, 132.8, 142.4, 143.3, 150.4, 150.5, 153.1 (2×C), 194.7, (1×C not observed).

MS-ESI m/z (rel. int.): 409 ([MH]⁺, 16), 381 (100), 355 (8).

HPLC: Method A (10 min), XBridge™ column, detection UV 254 nm, RT=5.22 min, peak area 97.4%.

### Preparation of 7-ethoxy-4-(3,4,5-trimethoxybenzoyl)isoquinolin-8-yl pivalate 109

### 7-Ethoxy-4-(3,4,5-trimethoxybenzoyl)isoquinolin-8-yl pivalate 109

To a solution of 7-ethoxy-8-hydroxyisoquinolin-4-yl)(3,4,5-trimethoxyphenyl)methanone (97 free base, 70 mg, 183 μmol) in dry CH₂Cl₂ (10 mL) in a 50 mL round-bottomed flask equipped with a magnetic stirrer was added diisopropylaminomethyl-polystyrene (200-400 mesh particle size, ˜3 mmol/g, 243 mg, 729 μmol), DMAP (10 mg, 82 μmol) and trimethylacetyl chloride (27 μL, 219 μmol) and the reaction mixture was stirred overnight at RT, diluted with CH₂Cl₂ (30 mL). This organic solution was then filtered, washed with brine (10 mL), dried over Na₂SO₄, filtered and concentrated under vacuum. Purification by column chromatography (SiO₂, eluent CH₂Cl₂:EtOAc=100:0 to 90:10) gave 7-ethoxy-4-(3,4,5-trimethoxybenzoyl)isoquinolin-8-yl pivalate 109 (48 mg, 56% yield) as a yellow solid.

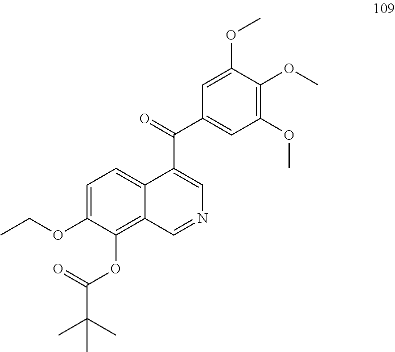 [SMILES: CCOc1ccc2c(C(=O)c3cc(OC)c(OC)c(OC)c3)cncc2c1OC(=O)C(C)(C)C]

MW: 467.51; Yield: 56%; Yellow solid; Mp (° C.): 134.3 (dec.)

¹H-NMR (CDCl₃, δ): 1.44 (t, 3H, J=6.9 Hz, CH₂CH₃), 1.50 (s, 9H, C(CH₃)₃), 3.85 (s, 6H, 2×OCH₃), 3.96 (s, 3H, OCH₃), 4.22 (q, 2H, J=6.9 Hz, CH₂CH₃), 7.13 (s, 2H, 2×ArH), 7.64 (d, 1H, J=9.2 Hz, ArH), 8.06 (d, 1H, J=9.2 Hz, ArH), 8.57 (s, 1H, ArH), 9.60 (s, 1H, ArH).

¹³C-NMR (CDCl₃, δ): 14.8, 27.3 (3×C), 39.4, 56.4 (2×C), 61.0, 65.4, 107.9 (2×C), 122.1, 123.8, 124.0, 128.9, 130.1, 132.0, 134.8, 140.0, 143.6, 149.0, 149.1, 153.2 (2×C), 175.9, 193.3.

MS-ESI m/z (rel. int.): 468 ([MH]⁺, 100).

HPLC: Method A (10 min), XBridge™ column, detection UV 254 nm, RT=6.56 min, peak area 99.3%.

### Preparation of 4-(2-chloro-3,4,5-trimethoxybenzyl)-7-ethoxyisoquinolin-8-ol hydrochloride 110

### 4-(2-Chloro-3,4,5-trimethoxybenzyl)-7-ethoxyisoquinolin-8-ol hydrochloride 110

Two 20 mL microwave vials equipped with a magnetic stirrer were charged with a mixture of 7-ethoxy-4-(3,4,5-trimethoxybenzyl)isoquinolin-8-yl acetate CCH 42032-1 (1.48 g, 3.60 mmol), N-hydroxyphthalimide (65 mg, 0.40 mmol) and NaClO₂ (80% pure, 0.63 g, 5.57 mmol) in CH₃CN:H₂O=5:4 (9 mL) and each vial was heated with stirring for 30 min at 110° C. under microwave irradiation. After cooling to RT, the mixtures were combined and CH₃CN was removed at 40° C. under vacuum. The residue was taken up in CH₂Cl₂ (100 mL) and the organic solution was washed with a 1 M Na₂S₂O₃ aq. solution (15 mL), brine (10 mL), dried over Na₂SO₄, filtered and concentrated at 40° C. under vacuum. Purification by column chromatography (SiO₂, eluent cyclohexane:EtOAc=100:0 to 50:50) gave 7-ethoxy-4-(3,4,5-trimethoxybenzoyl)isoquinolin-8-yl acetate 96 (0.52 g, 17%) and an other fraction of unpure 96 (0.70 g). This unpure fraction was dissolved in CH₂Cl₂ (100 mL) in a 250 mL round-bottomed flask equipped with a magnetic stirrer before addition of a 7 N NH₃ solution in MeOH (10 mL) and the reaction mixture was stirred overnight at RT. The organic solution was washed with brine (10 mL), dried over Na₂SO₄, filtered and concentrated under vacuum. Purification by column chromatography (SiO₂, eluent cyclohexane:acetone=100:0 to 75:25) gave 7-ethoxy-8-hydroxyisoquinolin-4-yl)(3,4,5-trimethoxyphenyl)methanone 97 free base (0.32 g) and another compound identified as 4-(2-chloro-3,4,5-trimethoxybenzyl)-7-ethoxyisoquinolin-8-ol. This free base was dissolved in MeOH (4 mL) in a 10 mL round-bottomed flask equipped with a magnetic stirrer before addition of a 0.4 M HCl solution in MeOH (2 mL). The reaction mixture was stirred for 5 min at RT and concentrated at 40° C. under vacuum to afford after drying 4-(2-chloro-3,4,5-trimethoxybenzyl)-7-ethoxyisoquinolin-8-ol hydrochloride 110 (0.18 g, 11% yield over 2 steps) as a yellow solid.

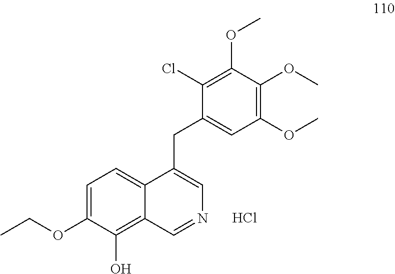 [SMILES: CCOc1ccc2c(Cc3cc(OC)c(OC)c(OC)c3Cl)cncc2c1O ; Cl]

MW: 454.30; Yield: 11%; Yellow solid; Mp (° C.): 215.0 (dec.)

¹H-NMR (CD₃OD:CDCl₃=1:1, 6): 1.53 (t, 3H, J=7.0 Hz, CH₂CH₃), 3.77 (s, 3H, OCH₃), 3.88 (s, 3H, OCH₃), 3.92 (s, 3H, OCH₃), 4.37 (q, 2H, J=7.0 Hz, CH₂CH₃), 4.58 (s, 2H, CH₂), 6.70 (s, 1H, ArH), 7.72 (s, 1H, ArH), 7.85 (d, 1H, J=9.0 Hz, ArH), 8.05 (d, 1H, J=9.0 Hz, ArH), 9.65 (s, 1H, ArH).

¹³C-NMR (CD₃OD:CDCl₃=1:1, 6): 15.3, 34.9, 57.1, 61.8, 61.9, 67.0, 111.3, 116.0, 120.4, 121.4, 126.9, 127.6, 131.5, 132.6, 136.7, 142.5, 144.1, 146.6, 147.1, 151.7, 154.0.

MS-ESI m/z (rel. int.): 406 ([MH]⁺, ³⁷Cl, 35), 404 ([MH]⁺, ³⁵Cl, 100).

HPLC: Method A (10 min), XBridge™ column, detection UV 254 nm, RT=4.50 min, peak area 99.1%.

### Preparation of N-(7-ethoxy-4-(3,4,5-trimethoxybenzoyl)isoquinolin-8-yl)acetamide hydrochloride 111

### N-(7-Ethoxy-4-(3,4,5-trimethoxybenzoyl)isoquinolin-8-yl)acetamide hydrochloride 111

To a solution of (8-amino-7-ethoxyisoquinolin-4-yl)(3,4,5-trimethoxyphenyl)methanone hydrochloride 29 (150 mg, 0.36 mmol) in THF (5 mL) at 5° C. was added N-methylmorpholine (79 μL, 0.72 mmol) followed by acetic anhydride (0.17 mL, 1.8 mmol). The mixture was stirred overnight at 25° C. then diluted in CH₂Cl₂ (20 mL), washed with a 0.1 M Na₂HPO₄ aq. solution (3×20 mL), brine (20 mL), dried over Na₂SO₄, filtered and concentrated at 40° C. under vacuum and purified by column chromatography (SiO₂, eluent EtOAc) to obtain a mixture of mono and bis acetamides. The obtained residue was treated for 1 h with a solution of THF:1 N HCl=1:1 (50 mL), and the volatiles were removed at 40° C. under vacuum to give a yellow solid (33 mg, 22% yield) that was partitioned between CH₂Cl₂ (15 mL) and a saturated NaHCO₃ solution (15 mL). The separated organic layer was washed with water (3×15 mL), brine (15 mL), dried over Na₂SO₄, filtered and concentrated at 40° C. under vacuum to give a yellow solid (33 mg). This solid was diluted in MeOH (5 mL), cooled at 4° C. and treated with a 0.49 N HCl solution in MeOH (0.16 mL). The solution was stirred for 15 min at 4° C. before concentration to dryness to yield N-(7-ethoxy-4-(3,4,5-trimethoxybenzoyl)isoquinolin-8-yl)acetamide hydrochloride 111 (38 mg, 25% yield) as a yellow solid.

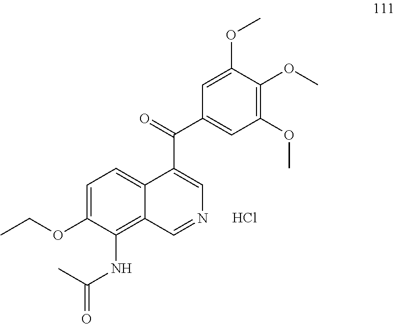 [SMILES: CCOc1ccc2c(C(=O)c3cc(OC)c(OC)c(OC)c3)cncc2c1NC(C)=O ; Cl]

MW: 460.91; Yield: 25%; Yellow solid; Mp (° C.): 221.8

R_{f}: 0.25 (EtOAc, free base).

¹H-NMR (CD₃OD, δ): 1.50 (t, 3H, J=6.9 Hz, CH₃), 2.37 (s, 3H, CH₃), 3.82 (s, 6H, 2×OMe), 3.89 (s, 3H, OMe), 4.39 (q, 2H, J=6.9 Hz, OCH₂), 7.22 (s, 2H, 2×ArH), 8.17 (s, 2H, 2×ArH), 8.58 (s, 1H, ArH), 9.65 (s, 1H, ArH).

¹³C-NMR (CD₃OD, δ): 15.0, 22.9, 30.4, 57.0 (2×C), 61.3, 67.0, 109.5 (2×C), 123.9, 127.1 (2×C), 130.2, 131.9, 132.9, 136.1, 145.7, 146.9, 154.9 (2×C), 156.0, 173.8, 192.2.

MS-ESI m/z (% rel. Int.): 425.1 ([MH]⁺, 100).

HPLC: Method A, XBridge™ column, detection UV 254 nm, RT=3.97 min, peak area 95.8%.

### Preparation of 2-amino-N-(7-ethoxy-4-(3,4,5-trimethoxybenzoyl)isoquinolin-8-yl)acetamide 112

### 2-Amino-N-(7-ethoxy-4-(3,4,5-trimethoxybenzoyl)isoquinolin-8-yl)acetamide 112

To a solution of Boc-Gly-OH (63 mg, 0.36 mmol) in THF (5 mL) was added at RT N-methylmorpholine (73 mg, 0.72 mmol). The reaction mixture was cooled down at −15° C. with an ice-methanol bath and isobutylchloroformate (49 mg, 0.36 mmol) and (8-amino-7-ethoxyisoquinolin-4-yl)(3,4,5-trimethoxyphenyl)methanone hydrochloride 29 (150 mg, 0.36 mmol) were successively added. The reaction mixture was heated at 70° C. for 48 h. After cooling, CH₂Cl₂ (20 mL) was added and the resulting organic solution was washed with a 0.1 M Na₂HPO₄ aq. solution (3×20 mL), brine (20 mL), dried over Na₂SO₄, filtered and concentrated. Purification by column chromatography (SiO₂, eluent CH₂Cl₂:EtOAc=50:50) gave a yellow solid (28 mg) that was treated with a 0.5 N HCl solution in MeOH (5 mL) for 2 days, and solvents were removed at 40° C. under vacuum to give a yellow solid. The solid was treated on resin Amberlite 400 OH⁻ form, purified by column chromatography (SiO₂, eluent CH₂Cl₂ to CH₂Cl₂:MeOH=100:0 to 90:10) to give, after evaporation, a yellow solid (14 mg). This solid was diluted in MeOH (5 mL) and treated at 4° C. with a 0.5 N HCl solution in MeOH (127 μL). The solution was stirred for 15 min at 0° C. before concentration to dryness at RT under vacuum, to yield 2-amino-N-(7-ethoxy-4-(3,4,5-trimethoxybenzoyl)isoquinolin-8-yl)acetamide dihydrochloride 112 (17 mg, 9% yield) as a yellow solid.

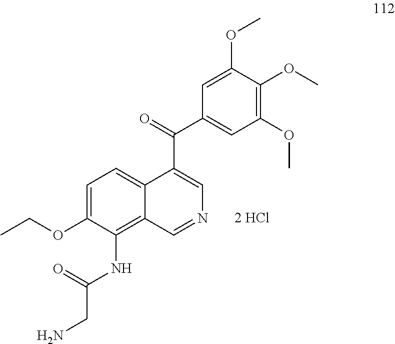 [SMILES: CCOc1ccc2c(C(=O)c3cc(OC)c(OC)c(OC)c3)cncc2c1NC(=O)CN ; "2 HCl"]

MW: 512.39; Yield: 9%; Yellow solid; Mp (° C.): 217.4

R_{f}: 0.20 (CH₂Cl₂: EtOAc=50:50, free base).

¹H-NMR (CD₃OD, δ): 1.50 (t, 3H, J=6.9 Hz, CH₃), 3.81 (s, 6H, 2×OMe), 3.88 (s, 3H, OMe), 4.21 (s, 2H, CH₂), 4.39 (q, 2H, J=6.9 Hz, OCH₂), 7.22 (s, 2H, 2×ArH), 8.14-8.22 (m, 2H, 2×ArH), 8.61 (s, 1H, ArH), 9.77 (s, 1H, ArH).

¹³C-NMR (CD₃OD, δ): 15.0, 42.3, 57.0 (2×C), 61.3, 67.1, 109.5 (2×C), 122.4, 126.8 (2×C), 127.6, 131.1, 131.9, 133.0, 135.9, 145.7, 147.1, 154.9 (2×C), 156.1, 168.1, 192.4.

MS-ESI m/z (% rel. Int.): 440.0 ([MH]⁺, 100).

HPLC: Method A, detection UV 254 nm, RT=3.63 min, peak area 99.0%.

### Preparation of isobutyl (7-ethoxy-4-(3,4,5-trimethoxybenzoyl)isoquinolin-8-yl)carbamate hydrochloride 113

### Isobutyl (7-ethoxy-4-(3,4,5-trimethoxybenzoyl)isoquinolin-8-yl)carbamate hydrochloride 113

To a solution of BOC-Val-OH (78 mg, 0.36 mmol) in THF (5 mL) at RT was added N-methylmorpholine (73 mg, 79 μL, 0.72 mmol). The reaction mixture was cooled down at −15° C. with an ice-methanol bath, treated with isobutylchloroformate (47 μL, 0.36 mmol) and (8-amino-7-ethoxyisoquinolin-4-yl)(3,4,5-trimethoxyphenyl)methanone hydrochloride 29 (150 mg, 0.36 mmol). The mixture was stirred overnight at RT, diluted in CH₂Cl₂ (20 mL) and the resulting organic solution was washed with a 0.1 M Na₂HPO₄ aq. solution (3×20 mL), brine (20 mL), dried over Na₂SO₄, filtered and concentrated. The crude product was purified by column chromatography (SiO₂, eluent CH₂Cl₂:EtOAc=100:0 to 90:10) to give, after evaporation of solvents, a yellow solid (72 mg). This solid was diluted in a 0.49 N HCl solution in MeOH (5 mL), and the solution was stirred for 2 h at RT. After concentration to dryness, isobutyl (7-ethoxy-4-(3,4,5-trimethoxybenzoyl)isoquinolin-8-yl)carbamate hydrochloride 113 (48 mg, 26% yield) was obtained as an orange solid.

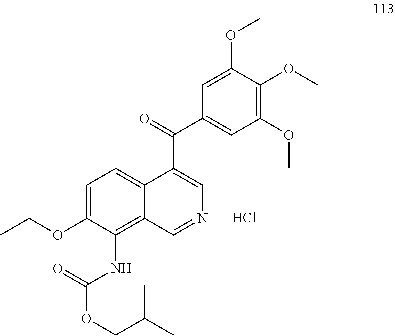 [SMILES: CCOc1ccc2c(C(=O)c3cc(OC)c(OC)c(OC)c3)cncc2c1NC(=O)OCC(C)C ; Cl]

MW: 518.99; Yield: 26%; Orange solid; Mp (° C.): 199.8

R_{f}: 0.20 (CH₂Cl₂:EtOAc=90:10, free base).

¹H-NMR (CD₃OD, δ): 0.9-1.05 (m, 6H, 2×CH₃), 1.50 (t, 3H J=7.2 Hz, CH₃), 1.95-2.10 (m, 1H, CH), 3.81 (s, 6H, 2×OMe), 3.89 (s, 3H, OMe), 4.01 (d, 2H J=6.6 Hz, CH₂), 4.38 (q, 2H, J=6.9 Hz, OCH₂), 7.22 (s, 2H, 2×ArH), 8.16 (s, 2H, 2×ArH), 8.58 (s, 1H, ArH), 9.72 (s, 1H, ArH).

¹³C-NMR (CD₃OD, δ): 15.04, 19.33 (2×C), 29.35, 56.99 (2×C), 61.33, 67.00, 73.23, 109.46 (2×C), 124.33, 126.83, 127.03, 128.03, 130.40, 131.92, 132.95, 136.06, 145.71, 146.67, 154.91 (2×C), 156.34, 158.02, 192.29.

MS-ESI m/z (% rel. Int.): 483.0 ([MH]⁺, 100).

HPLC: Method A, detection UV 254 nm, XBridge™ column, RT=5.06 min, peak area 99.0%.

### Preparation of (S)-2-amino-N-(7-ethoxy-4-(3,4,5-trimethoxybenzoyl)isoquinolin-8-yl)-3-phenylpropanamide dihydrochloride 114

### (S)-2-Amino-N-(7-ethoxy-4-(3,4,5-trimethoxybenzoyl)isoquinolin-8-yl)-3-phenylpropanamide dihydrochloride 114

To a solution of (8-amino-7-ethoxyisoquinolin-4-yl)(3,4,5-trimethoxyphenyl)methanone hydrochloride 29 (150 mg, 0.36 mmol) in CH₂Cl₂ (10 mL) was added Boc-Phe-OH (96 mg, 0.36 mmol), N-methylmorpholine (79 μL, 0.72 mmol) and EDCI (138 mg, 0.36 mmol). The mixture was stirred for 3 days at RT then diluted with CH₂Cl₂ (20 mL) and the resulting organic solution was washed with water (3×10 mL), brine (10 mL), dried over Na₂SO₄, filtered and concentrated. The crude product was purified by column chromatography (SiO₂, eluent CH₂Cl₂:EtOAc=1:1) to give, after evaporation of solvents, a yellow solid (17 mg). The above solid was treated with a 0.49 N HCl solution in MeOH (5 mL) for 2 days at RT. After concentration to dryness, ((S)-2-amino-N-(7-ethoxy-4-(3,4,5-trimethoxybenzoyl)isoquinolin-8-yl)-3-phenylpropanamide dihydrochloride 114 (15 mg, 8% yield) was obtained as an orange solid.

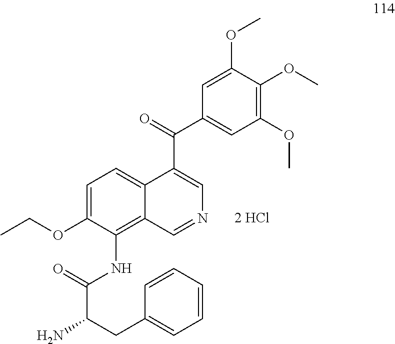 [SMILES: CCOc1ccc2c(C(=O)c3cc(OC)c(OC)c(OC)c3)cncc2c1NC(=O)[C@@H](N)Cc1ccccc1 ; "2 HCl"]

MW: 602.52; Yield: 8%; Orange Solid; Mp (° C.): 227.5

R_{f}: 0.55 (CH₂Cl₂:EtOAc=1:1, free base).

¹H-NMR (MeOD, δ): 1.52 (t, 3H, J=6.6 Hz, CH₃), 3.62 (m, 1H, 0.5×CH₂), 3.84 (s, 3H, OMe), 3.91 (s, 6H, 2×OMe), 4.42 (q, 2H, J=6.6 Hz, OCH₂), 4.72 (broad s, 1H, CH), 7.25 (s, 2H, 2×ArH), 7.30-7.53 (m, 5H, 5×ArH), 8.24 (s, 2H, 2×ArH), 8.65 (s, 1H, ArH), 9.74 (s, 1H, ArH).

¹³C-NMR (MeOD, δ): 15.23, 38.74, 56.33, 57.26 (2×C), 61.46, 67.31, 109.29 (2×C), 122.30, 127.38, 127.77, 127.96, 129.18 (2×C), 130.36 (2×C), 130.80 (2×C), 132.21, 132.88, 135.47, 136.32, 145.74, 146.61, 154.91 (2×C), 156.41, 170.32, 192.02.

MS-ESI m/z (% rel. Int.): 530.0 ([MH]⁺, 100).

HPLC: Method A, detection UV 254 nm, XBridge™ column, RT=4.18 min, peak area 99.0%.

### Preparation of 4-(3-bromo-4,5-dimethoxybenzoyl)-7-ethoxyisoquinolin-8-yl acetate 115

### 4-(3-Bromo-4,5-dimethoxybenzyl)-7-ethoxyisoquinolin-8-yl acetate RBO 45020

To a solution of 4-(3-bromo-4,5-dimethoxybenzyl)-7-ethoxyisoquinolin-8-ol 71 free base (377 mg, 0.89 mmol) in CH₂Cl₂ (10 mL), in a 25 mL round bottom flask equipped with a magnetic stirrer, was added at RT, DIEA (621 μL, 3.57 mmol), DMAP (22 mg, 0.18 mmol), and acetic anhydride (0.505 mL, 5.34 mmol). The reaction was stirred at RT for 16 h, then diluted with CH₂Cl₂ (15 mL) and the resulting organic solution was washed with water (3×10 mL), brine (10 mL), dried over Na₂SO₄, filtered and evaporated to give a brown solid (mg). This crude solid was purified by column chromatography (SiO₂, eluent CH₂Cl₂:EtOAc=50:50) to give, after evaporation and drying, 4-(3-bromo-4,5-dimethoxybenzyl)-7-ethoxyisoquinolin-8-yl acetate RBO 45020 (293 mg, 71% yield) as white solid.

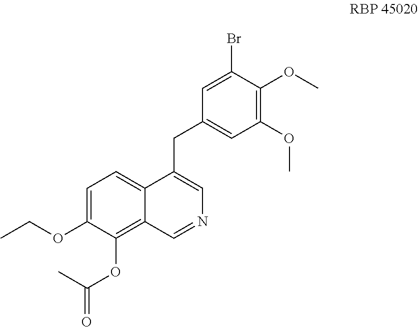 [SMILES: CCOc1ccc2c(Cc3cc(Br)c(OC)c(OC)c3)cncc2c1OC(C)=O]

MW: 460.33; Yield: 71%; Off-white solid.

R_{f}: 0.5 (CH₂Cl₂: EtOAc=1:1).

¹H-NMR (CD₃OD, δ): 1.41 (t, 3H, J=7 Hz, CH₃), 2.48 (s, 3H, CH₃), 3.76 (s, 3H, OMe), 3.78 (s, 3H, OMe), 4.23 (q, 2H, J=7 Hz OCH₂), 4.37 (s, 2H, CH₂), 6.90 (s, 1H, ArH), 6.95 (s, 1H, ArH), 7.72 (d, 1H, J=9.6 Hz, ArH), 7.97 (d, 1H, J=9 Hz, ArH), 8.26 (s, 1H, ArH), 9.16 (s, 1H, ArH).

MS-ESI m/z (% rel. Int.): 461.9 ([MH]⁺, 100), 459.9 ([MH]⁺, 96).

HPLC: Method A, detection UV 254 nm, XBridge™ column, RT=4.77 min, peak area 99.0%.

### 4-(3-Bromo-4,5-dimethoxybenzoyl)-7-ethoxyisoquinolin-8-yl acetate 115

To a solution of 4-(3-bromo-4,5-dimethoxybenzyl)-7-ethoxyisoquinolin-8-yl acetate RBO 45020 (293 mg, 0.64 mmol) in a mixture of CH₃CN:H₂O=1:1 (10 mL), was successively added at RT, sodium chlorite (108 mg, (108 mg, 0.96 mmol), N-hydroxyphtalimide (20.7 mg, 0.13 mmol) and the reaction mixture was stirred at 100° C. for 1 h. After evaporation of solvents at 40° C. under vacuum, the residue was diluted with CH₂Cl₂ (15 mL) and the resulting organic solution was washed with a 10% aq. solution of sodium thiosulfate (3×10 mL), brine (10 mL), dried over Na₂SO₄, filtered and evaporated to give 4-(3-bromo-4,5-dimethoxybenzoyl)-7-ethoxyisoquinolin-8-yl acetate 115 (77 mg, 25% yield) as a yellow solid.

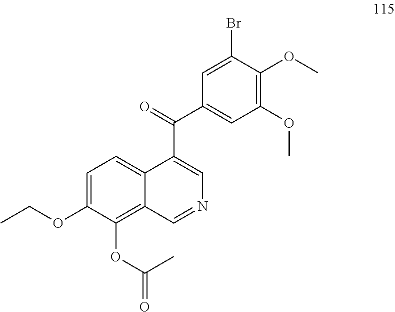 [SMILES: CCOc1ccc2c(C(=O)c3cc(Br)c(OC)c(OC)c3)cncc2c1OC(C)=O]

MW: 474.30; Yield: 25%; Yellow Solid; Mp (° C.): 177.7

R_{f}: 0.7 (CH₂Cl₂: EtOAc=1:1).

¹H-NMR (CDCl₃, δ): 1.46 (t, 3H, J=6.9 Hz, CH₃), 2.52 (s, 3H, CH₃), 3.92 (s, 3H, OMe), 3.97 (s, 3H, OMe), 4.24 (q, 2H, J=6.9 Hz, OCH₂), 7.51 (s, 1H, ArH), 7.58 (m, 2H, 2×ArH), 8.04 (d, 1H, J=9.3 Hz, ArH), 8.54 (s, 1H, ArH), 9.43 (s, 1H, ArH).

¹³C-NMR (CDCl₃, δ): 14.89, 20.45, 56.35, 60.82, 65.48, 112.60 (2×C), 117.53, 121.02, 123.82 (2×C), 128.32, 128.47, 134.30 (2×C), 142.42, 148.35, 149.02, 151.30, 153.70, 168.46, 193.72.

MS-ESI m/z (% rel. Int.): 476.2 ([MH]⁺, 100), 474.2 ([MH]⁺, 96).

HPLC: Method A, XBridge™ column, detection UV 254 nm, RT=5.67 min, peak area 98.0%.

### Preparation of (3-bromo-4,5-dimethoxyphenyl)(7-ethoxy-8-hydroxyisoquinolin-4-yl)methanone hydrochloride 116

### (3-Bromo-4,5-dimethoxyphenyl)(7-ethoxy-8-hydroxyisoquinolin-4-yl)methanone hydrochloride 116

To a solution of 4-(3-bromo-4,5-dimethoxybenzoyl)-7-ethoxyisoquinolin-8-yl acetate 115 (49 mg, 0.10 mmol) in CH₂Cl₂ (5 mL) was added a 7 N ammonia solution in MeOH (886 μL). The reaction was stirred overnight at RT, then diluted with CH₂Cl₂ (10 mL). The resulting organic solution was washed with brine (3×10 mL), dried over Na₂SO₄, filtered and evaporated to give (3-bromo-4,5-dimethoxyphenyl)(7-ethoxy-8-hydroxyisoquinolin-4-yl)methanone as an orange solid (37 mg). This solid was diluted in MeOH (5 mL) and treated at 0° C. by a 0.18 N HCl solution in MeOH (0.574 mL, 0.103 mmol) and the reaction mixture was stirred for 15 min. After concentration to dryness, (3-bromo-4,5-dimethoxyphenyl)(7-ethoxy-8-hydroxyisoquinolin-4-yl)methanone hydrochloride 116 (38 mg, 48% yield) was obtained as a yellow solid.

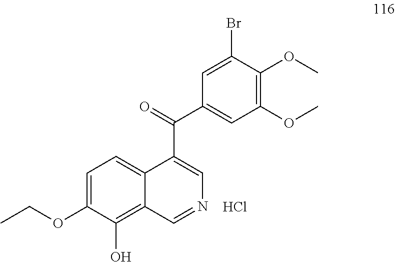 [SMILES: CCOc1ccc2c(C(=O)c3cc(Br)c(OC)c(OC)c3)cncc2c1O ; Cl]

MW: 468.72; Yield: 48%; Yellow solid; Mp (° C.): 224.7

R_{f}: 0.6 (CH₂Cl₂:EtOAc=1:1, free base).

¹H-NMR (CD₃OH, δ): 1.51 (t, 3H, J=6.9 Hz, CH₃), 3.92 (s, 3H, OMe), 3.94 (s, 3H, OMe), 4.35 (q, 2H, J=6.9 Hz, OCH₂), 7.62 (m, 3H, 3×ArH), 8.04 (d, 1H, J=9.0 Hz, ArH), 8.45 (s, 1H, ArH), 9.88 (s, 1H, ArH).

¹³C-NMR (CD₃OD, δ): 14.98, 56.94, 61.28, 66.98, 113.87, 117.38, 118.48, 120.94, 127.56, 129.19, 129.52, 130.66, 134.48, 134.72, 146.04, 146.95, 147.27, 153.37, 155.39, 191.63.

MS-ESI m/z (% rel. Int.): 434.1 ([MH]⁺, 100), 432.0 ([MH]⁺, 98).

HPLC: Method A, XBridge™ column, detection UV 254 nm, RT=4.67 min, peak area 99.0%.

### In Vitro Biological Data for Compounds:

1) MTT Assay:

Assessment of Cell Viability via MTT Assay: Cytotoxicity of compounds against the K562 cell line was assessed using the [3-(4,5-dimethylthiazol-2-yl)-2,5-diphenyl tetrazolium bromide] (MTT) assay. Fifteen thousand tumor cells were seeded into wells of flat-bottomed 96-well plates. An amount of 2 μL of a dilution series of each compound dissolved in DMSO was added to 198 μL of appropriate growth medium at three replicates per concentration and the plates were incubated in a 5% CO₂ incubator at 37° C. After 72 h, the number of metabolically active cells was determined by MTT assay. Absorbance of each well was measured at 550 nm on a Tecan Sunrise reader. Dose-response curves for growth inhibition were generated as a percentage of untreated control. IC₅₀ was determined by nonlinear regression (Prism, Graph Pad Software, Inc.).

| Compound | IC₅₀ (nM) |
|---|---|
| BNC105 | 0.55 |
| 58 | 1.23 |
| 29 | 1.64 |
| Combretastatin | 2.07 |
| Azixa | 2.15 |
| 57 | 2.69 |
| AVE8063 | 2.71 |
| Vinblastine | 3.66 |
| 65 | 3.81 |
| 105 | 4.18 |
| 66 | 4.27 |
| 56 | 4.39 |
| 67 | 4.64 |
| 97 | 4.86 |
| 96 | 5.85 |
| Taxol | 6.90 |
| 78 | 7.02 |
| 59 | 7.04 |
| 98 | 7.57 |
| AVE8062 | 7.78 |
| 112 | 7.90 |
| Colchicine | 8.52 |
| 3 | 10.73 |
| 64 | 11.20 |
| 79 | 12.05 |
| 18 | 13.18 |
| BNC105P | 17.29 |
| 72 | 18.32 |
| 40 | 19.65 |
| 20 | 20.81 |
| 115 | 20.81 |
| 32 | 21.52 |
| 19 | 21.52 |
| 71 | 25.97 |
| 1 | 29.13 |
| 14 | 30.31 |
| 38 | 30.38 |
| 21 | 36.86 |
| 31 | 37.03 |
| 104 | 38.81 |
| 28 | 42.15 |
| 77 | 48.63 |
| 95 | 49.71 |
| 8 | 53.36 |
| 42 | 60.71 |
| 30 | 63.82 |
| 34 | 64.99 |
| 109 | 67.86 |
| 110 | 68.01 |
| 50 | 69.83 |
| 108 | 74.83 |
| 116 | 86.91 |
| 114 | 92.60 |
| 70 | 92.85 |
| 111 | 96.82 |
| 15 | 104.79 |
| 52 | 118.25 |
| 41 | 123.00 |
| 47 | 129.00 |
| 103 | 138.35 |
| 22 | 142.40 |
| 7 | 147.40 |
| 68 | 152.05 |
| 33 | 159.85 |
| Indibulin | 196.80 |
| 63 | 213.30 |
| 113 | 230.80 |
| 75 | 236.50 |
| 69 | 237.90 |
| 74 | 238.17 |
| 87 | 253.85 |
| 10 | 283.05 |
| 76 | 312.50 |
| 93 | 334.35 |
| 73 | 334.80 |
| 13 | 349.15 |
| 9 | 375.60 |
| 39 | 400.05 |
| 55 | 422.20 |
| 16 | 434.65 |
| 107 | 575.40 |
| 46 | 687.57 |
| 51 | 741.15 |
| 48 | 811.65 |
| 27 | 864.00 |
| 106 | 885.40 |
| 37 | 899.45 |
| 92 | 943.45 |

2) Tubulin Polymerisation Assay:

The polymerization of purified tubulin was monitored spectrophotometrically by the change in absorbance at 340 nm using the CytoDYNAMIX Screen kit (#BK006P, Cytoskeleton) according to the instructions of the manufacturer. This assay is based on an adaptation of the original method of Shelanski et al. (1973) and Lee et al. (1977), which demonstrated that light is scattered by microtubules to an extent that is proportional to the concentration of microtubule polymer. The absorbance was measured at 1 minute intervals for 60 minutes. The test compound was added to a 4 mg/ml tubulin solution containing 10% glycerol and 1 mM GTP in Buffer 1 (Buffer 1: 80 mM PIPES; 2 mM MgCl₂; 0.5 mM EGTA, pH 6.9). Increased absorbance indicates increase in microtubule formation from tubulin heterodimers.

| | % Inhibition of tubulin | |
|---|---|---|
| Compound | polymerization at 5 μM | IC₅₀ (μM) |
| 29 | 71.29 | 0.88 |
| 64 | 79.50 | 0.90 |
| Combretastatin | 68.90 | 0.90 |
| 15 | 69.00 | 0.97 |
| Colchicine | 84.33 | 0.98 |
| 18 | 61.44 | 0.99 |
| 3 | 76.64 | 1.04 |
| 14 | ND | 1.05 |
| Vinblastine | 77.67 | 1.05 |
| AVE8063 | 71.29 | 1.07 |
| 21 | 65.75 | 1.09 |
| 38 | 67.40 | 1.11 |
| 40 | 58.83 | 1.13 |
| 1 | 85.00 | 1.19 |
| 33 | 59.00 | 1.31 |
| 32 | 63.40 | 1.50 |
| 20 | 56.86 | 1.52 |
| 31 | 55.40 | 1.54 |
| BNC105 | 64.75 | 1.78 |
| AVE8062 | 59.77 | 1.79 |
| 19 | 61.86 | 1.85 |
| 56 | 65.13 | 1.94 |
| Azixa | 65.67 | 2.52 |
| 10 | ND | 3.44 |
| 58 | 38.25 | 3.49 |
| BNC105P | 39.00 | 4.10 |
| 28 | 44.50 | ND |
| 30 | 21.00 | ND |
| 57 | 17.00 | ND |
| 59 | 21.50 | ND |
| 65 | 13.00 | ND |
| 67 | 10.00 | ND |
| 96 | 54.50 | ND |
| 97 | 4.00 | ND |
| 98 | 49.00 | ND |
| 105 | 35.00 | ND |

3) Tubulin Displacement Assay:

A radioligand binding assay for the colchicine site on tubulin was developed to measure the ability of drugs to compete with ³H-colchicine. The assay was developped following the method published by Tahir et al. (2001) and others using biotinylated porcine tubulin captured to streptavidin coated yttrium silicate scintillation proximity assay (YSi SPA) beads in a PIPES/EGTA/MgCl₂/GTP buffer. The assay was run with 50 nM [³H]-colchicine for 4 h at 37° C. IC₅₀s were determined using GraphPad Prism Version 5.01 and the sigmoidal dose response with variable slope curve fit.

| Compound | IC₅₀ (nM) |
|---|---|
| 67 | 38.7 |
| 97 | 41.35 |
| 57 | 91.89 |
| Combretastatin | 120.0 |
| 98 | 120.7 |
| 3 | 170 |
| 1 | 220 |
| 59 | 744.8 |
| Colchicine | 825.0 |
| 10 | 1600 |
| 96 | 3303 |

4) Activity on Neovessels Formation and on Pre-Established Vessels In Vitro:

The capability of compounds to inhibit the formation of capillaries was evaluated in vitro. Twenty thousand Endothelial cells (HUVEC) were placed on a Matrigel matrix and allowed to form capillary tubes in the presence or absence of the compounds overnight. The cells were stained with calcein AM, and the effect of the compounds on capillary tube formation was assessed using an inverted fluorescent microscope (Nikon). The reference compound used in this assay AVE-8063 is 3-amino-deoxycombretastatin A-4.

| | Neovessels Formation | |
|---|---|---|
| | Inhibition | |
| | | Number of |
| | Total Tubule | Branch |
| | Length | Junctions |
| Compound | IC₅₀ (nM) | IC₅₀ (nM) |
| 58 | 0.59 | 0.53 |
| 29 | 1.47 | 1.04 |
| BNC105P | 1.53 | 0.44 |
| Azixa | 2.16 | 1.38 |
| BNC105 | 2.49 | 1.76 |
| Vinblastine | 3.44 | 1.55 |
| 66 | 3.58 | 2.23 |
| 57 | 3.97 | 2.65 |
| 65 | 4.23 | 2.88 |
| 67 | 4.80 | 3.84 |
| AVE8063 | 5.33 | 3.12 |
| 97 | 5.52 | 4.04 |
| 98 | 6.50 | 3.68 |
| Taxol | 7.16 | 0.79 |
| 105 | 8.06 | 5.71 |
| 78 | 8.07 | — |
| 96 | 8.10 | 5.07 |
| 64 | 9.54 | 7.02 |
| 56 | 12.18 | 10.67 |
| 40 | 12.88 | 8.20 |
| AVE8062 | 15.36 | 13.89 |
| 18 | 18.66 | 13.01 |
| 3 | 21.82 | 15.27 |
| 32 | 21.87 | 12.41 |
| Colchicine | 22.12 | 15.48 |
| 19 | 23.01 | 14.44 |
| 59 | 25.17 | 8.32 |
| 112 | 31.30 | 16.39 |
| 20 | 31.91 | 24.92 |
| 14 | 39.50 | 31.05 |
| 21 | 52.76 | 47.33 |
| 1 | 54.49 | 38.52 |
| 8 | 137.90 | 100.00 |

To explore the effect of the compounds on pre-established vasculature, twenty thousand cells (HUVEC) were seeded and incubated over Matrigel for 5 hours to allow the capillary tubes to form. Then, compounds were added and incubated overnight. The disappearance of existing vasculature was monitored with fluorescent microscopy.

| | Pre-established Vessels | |
|---|---|---|
| | Disruption | |
| | | Number of |
| | Total Tubule | Branch |
| | Length | Junctions |
| Compound | IC₅₀ (nM) | IC₅₀ (nM) |
| 58 | 1.36 | 0.99 |
| 29 | 2.26 | 1.78 |
| Vinblastine | 5.39 | 2.09 |
| 66 | 5.47 | 3.76 |
| Azixa | 6.01 | 3.75 |
| BNC105 | 6.37 | 5.20 |
| 57 | 6.72 | 4.37 |
| BNC105P | 7.37 | 5.83 |
| AVE8063 | 7.74 | 5.72 |
| 97 | 8.82 | 6.65 |
| 105 | 9.30 | 9.27 |
| 65 | 9.92 | 3.36 |
| 67 | 12.59 | 9.18 |
| Colchicine | 12.72 | 4.93 |
| 64 | 14.74 | 7.41 |
| 98 | 15.08 | 9.22 |
| 96 | 15.28 | 9.14 |
| 19 | 15.87 | 6.33 |
| 56 | 16.10 | 14.45 |
| 78 | 19.41 | 11.14 |
| 20 | 31.68 | 22.34 |
| 18 | 32.10 | 17.79 |
| 59 | 38.25 | 27.15 |
| 3 | 45.60 | 14.88 |
| 32 | 48.84 | 20.01 |
| AVE8062 | 52.93 | 32.53 |
| 40 | 53.40 | 32.76 |
| 21 | 60.45 | 26.69 |
| 112 | 62.77 | 68.55 |

These assays characterized the antiangiogenic and vascular disrupting effects of the compounds of the invention.

1.-17. (canceled)

18. A compound of formula (I): 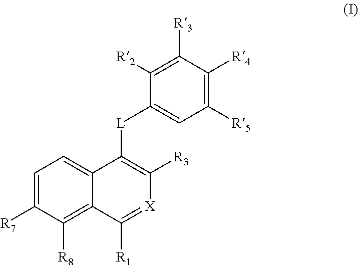
wherein:
X represents N or N⁺—Z, wherein Z is selected in the group consisting of a (C₁-C₆)alkyl, an aryl and an acyl;
R₁ represents H, CN, a CORa or a (C₁-C₅)alkyl;
wherein Ra represents a NRa′Ra″ or ORa′″;
wherein Ra′ and Ra″, independently from each other, are selected from the group consisting of H and (C₁-C₅)alkyl;
wherein Ra′″ represents H or (C₁-C₅)alkyl;
R₃ represents H, CN, OH, a CORb, NH₂ or a (C₁-C₅)alkyl group;
wherein Rb represents a NRb′Rb″ or ORb′″ group;
wherein Rb′ and Rb′, independently from each other, are selected from the group consisting of H and (C₁-C₅)alkyl;
wherein Rb′″ represents H or (C₁-C₅)alkyl;
R₇ represents a (C₁-C₅)alkyl, a (C₁-C₅)alkoxy, a (C₁-C₅)alkylthio, a (C₁-C₆)alkylamino, a (C₁-C₅)dialkylamino;
R₈ represents H, halogen, nitro, CN, N₃, a diarylmethylenehydrazinyl, a di(C₁-C₆)alkyl-N—C═S(O)— group or a di(C₁-C₆)alkyl-N—C═O(S)— group, a (C₁-C₆)alkyl-SO₂, SR, OR or NRR′ group;
wherein R and R′, independently from each other, are selected from the group consisting of H, a (C₁-C₆)alkyl, a (C₂-C₆)alkenyl, an acyl, an aryl, a heterocyclyl, an amino acid, a Y—SO₂ group, a —P(O)(OG)(OG′) group,
wherein Y is selected in the group consisting of a hydrogen atom, (C₁-C₆)alkyl, NH₂, (C₁-C₆)alkylamino, di(C₁-C₆)alkylamino and (C₁-C₆)alkoxycarbonylamino;
wherein G and G′, independently from each other, are selected in the group consisting of H, (C₁-C₆)alkyl and aryl;
R₇ and R₈, taken together with the carbon atom to which they are attached, may form a 5- to 6-membered ring which may contain one or more heteroatom(s) selected from N, O and S;
L represents CH₂, C═O, CF₂, CHF, CHOZ′, O, S, SO, SO₂, C═NZ′, or NR″;
wherein R″ is selected from H, a (C₁-C₆)alkyl and an acyl group;
wherein Z′ is H, a (C₁-C₆)alkyl, an aryl, amino or OR′″;
wherein R′″ is H or a (C₁-C₆)alkyl;
R′₂ represents H, halogen, a (C₁-C₅)alkyl, a (C₁-C₃)alkylthio, a nitro group or OZ″;
wherein Z″ is selected from H, a (C₁-C₅)alkyl, a (C₂-C₅)alkenyl or a (C₁-C₅)alkylsulfonyl;
R′₃ and R′₅ independently represent halogen, an amino, a nitro group, a (C₁-C₅)alkyl, a (C₁-C₃)alkylthio, a (C₁-C₃)alkylamino, or a di(C₁-C₃)alkylamino, SR* or OR*;
wherein R* is selected from H, a (C₁-C₆)alkyl, an acyl, a (C₁-C₆)alkenyl or a (C₁-C₅)alkylsulfonyl;
R′₄ represents H, halogen, an amino, (C₁-C₅)alkyl, (C₁-C₃)alkylthio, (C₁-C₃)alkylamino, a di(C₁-C₃)alkylamino, heterocyclyl, aryl, heteroaryl, SR** or OR**;
wherein R** is selected from H, (C₁-C₆)alkyl, an acyl, a (C₁-C₆)alkenyl, an aryl, a cycloalkenyl, a cycloalkyl, a heterocyclyl, or a (C₁-C₅)alkylsulfonyl
R′₄ and R′₅, taken together with the carbon atom to which they are attached, may form a 5- to 6-membered ring which may contain one or more heteroatom(s) selected from N, O and S.

19. The compound according to claim 18 wherein:
X represents N or N⁺—Z, wherein Z is selected in the group consisting of a (C₁-C₆)alkyl, an aryl and an acyl;
R₁ represents H, CN, a CORa or a (C₁-C₅)alkyl;
wherein Ra represents a NRa′Ra″ or ORa′″ group;
wherein Ra′ and Ra″, independently from each other, are selected from the group consisting of H and (C₁-C₅)alkyl;
wherein Ra′″ represents H or (C₁-C₅)alkyl;
R₃ represents H, CN, OH, a CORb, NH₂ or a (C₁-C₅)alkyl;
wherein Rb represents a NRb′Rb″ or ORb′″ group;
wherein Rb′ and Rb′, independently from each other, are selected from the group consisting of H and (C₁-C₅)alkyl;
wherein Rb′″ represents H or (C₁-C₅)alkyl;
R₇ represents a (C₁-C₅)alkyl, a (C₁-C₅)alkoxy, a (C₁-C₅)alkylthio, a (C₁-C₅)alkylamino, a (C₁-C₅)dialkylamino;
R₈ represents H, halogen, nitro, a (C₁-C₆)alkyl-SO₂, SR, OR or NRR′ group;
wherein R and R′, independently from each other, are selected from the group consisting of H, a (C₁-C₆)alkyl, a (C₂-C₆)alkenyl, an acyl, an aryl, a heterocyclyl, amino acid, a Y—SO₂ group, a P(O)(OG)(OG′) group,
wherein Y is selected in the group consisting of a hydrogen atom, (C₁-C₆)alkyl, NH₂, (C₁-C₆)alkylamino, di(C₁-C₆)alkylamino and (C₁-C₆)alkoxycarbonylamino;
wherein G and G′, independently from each other, are selected in the group consisting of H, (C₁-C₆)alkyl and aryl;
L represents CH₂, C═O, CF₂, CHF, CHOZ′, O, S, SO, SO₂, C═NZ′, or NR″;
wherein R″ is selected from H, a (C₁-C₆)alkyl and an acyl group;
wherein Z′ is H, a (C₁-C₆)alkyl, an aryl, amino or OR′″;
wherein R′″ is H or a (C₁-C₆)alkyl;
R′₂ represents H, halogen, a (C₁-C₅)alkyl, a (C₁-C₃)alkylthio, or OZ″;
wherein Z″ is selected from H, a (C₁-C₅)alkyl, a (C₂-C₅)alkenyl or a (C₁-C₅)alkylsulfonyl;
R′₃ and R′₅ independently represent halogen, an amino, a (C₁-C₅)alkyl, a (C₁-C₃)alkylthio, a (C₁-C₃)alkylamino, or a di(C₁-C₃)alkylamino, SR* or OR*;
wherein R* is selected from H, a (C₁-C₆)alkyl, an acyl, a (C₁-C₆)alkenyl or a (C₁-C₅)alkylsulfonyl;
R′₄ represents H, halogen, an amino, (C₁-C₅)alkyl, (C₁-C₃)alkylthio, (C₁-C₃)alkylamino, or a di(C₁-C₃)alkylamino, SR** or OR**;
wherein R** is selected from H, (C₁-C₆)alkyl, an acyl, a (C₁-C₆)alkenyl, an aryl, a cycloalkenyl, a cycloalkyl, a heterocyclyl, or a (C₁-C₅)alkylsulfonyl.

20. The compound according to claim 18, wherein
X represents N and/or
R₇ represents an alkoxy, and/or
L represents a CH₂ or CO group, and/or
R₈ represents H, (C₁-C₆)alkyl-SO₂, OR or NRR′ group;
wherein R and R′, independently from each other, are selected from the group consisting of H, a (C₁-C₆)alkyl group, an acyl group, a (C₁-C₆)alkyl-SO₂ group, SO₂NH₂ group, a (C₁-C₆)alkyl-NH—SO₂ group, an alkoxycarbonylamino-SO₂ group, and an amino acid.

21. The compound according to claim 18, wherein
X represents N;
R₁ represents H, CN or CNCH₂;
R₃ represents H;
R₇ represents an ethoxy or methoxy;
R₈ represents H, CH₃SO₂, OH, NH₂, CH₃O, CNCH₃O, a CF₃SO₃, NH₂SO₃, CH₃CONH, CH₃SO₂NH, NH₂SO₂NH, serine-NH, glycine-NH, phenylalanine-NH, or a tert-butyloxycarbonylaminosulfonylamino group;
L represents a CH₂ or CO group;
R′₂ represents H or bromine;
R′₃ and R′₅ independently from each other represent F, a methoxy or an ethoxy group; and
R′₄ represents H, F, a hydroxyl group, a methoxy, ethoxy, n-propyloxy, sec-butyloxy or CH₃OCH₂CH₂O group.

22. The compound according to claim 18, wherein R′₃ and R′₅ are identical.

23. The compound according to claim 18, wherein R₈ represent a NRR″ group.

24. The compound according to claim 18, wherein R′₄ is an alkoxy group.

25. The compound according to claim 18, wherein the compound has at least one of the following features:
X represents N;
R₁ represents H;
R₃ represents H;
R₇ represents a methoxy or an ethoxy;
R₈ represents H, OH, NH₂, methoxy, NH₂SO₃, NH₂SO₂NH, CH₃COO, OPO₃²⁻, NH₂CH₂CONH;
L represents a CH₂ or C═O group;
R′₂ represents H;
R′₃ represents a methoxy or halogen atom;
R′₄ represents a methoxy, ethoxy, n-propyloxy, isobutyloxy or allyloxy group;
R′₅ represents a methoxy group.

26. The compound according to claim 18, selected in the group consisting of:
4-(3,4,5-Trimethoxybenzyl)-7-ethoxyisoquinolin-8-ol hydrochloride 1,
7-ethoxy-4-(3,4,5-trimethoxybenzyl)isoquinolin-8-yl trifluoromethanesulfonate hydrochloride 2,
7-ethoxy-4-(3,4,5-trimethoxybenzyl)isoquinolin-8-amine dihydrochloride 3,
N-(7-ethoxy-4-(3,4,5-trimethoxybenzyl)isoquinolin-8-yl)acetamide hydrochloride 4,
N-(7-ethoxy-4-(3,4,5-trimethoxybenzyl)isoquinolin-8-yl)methanesulfonamide hydrochloride 5,
7-ethoxy-8-(methylsulfonyl)-4-(3,4,5-trimethoxybenzyl)isoquinoline hydrochloride 6,
tert-butyl N-(7-ethoxy-4-(3,4,5-trimethoxybenzyl)isoquinolin-8-yl)sulfamoylcarbamate 7,
7-ethoxy-4-(3,4,5-trimethoxy-benzyl)isoquinolin-8-yl]-sulfamide hydrochloride 8,
4-(4-ethoxy-3,5-dimethoxybenzyl)-7,8-dimethoxyisoquinoline hydrochloride 9,
4-(3,4,5-trimethoxybenzyl)-7,8-dimethoxyisoquinoline hydrochloride 10,
2,6-dimethoxy-4-((7,8-dimethoxyisoquinolin-4-yl)methyl)phenol hydrochloride 11,
7-ethoxy-4-(3,4,5-trifluorobenzyl)isoquinolin-8-ol hydrochloride 12,
7-ethoxy-4-(4-ethoxy-3,5-dimethoxybenzyl)isoquinoline hydrochloride 13,
4-(4-ethoxy-3,5-dimethoxybenzyl)-7-ethoxyisoquinolin-8-ol hydrochloride 14,
4-(3-ethoxy-4,5-dimethoxybenzyl)-7-ethoxyisoquinolin-8-ol hydrochloride 15,
4-(3,4,5-trimethoxybenzyl)-7-ethoxyisoquinoline hydrochloride 16,
4-(2-bromo-3,5-dimethoxybenzyl)-7-ethoxyisoquinolin-8-ol hydrochloride 17,
4-(4-ethoxy-3,5-dimethoxybenzyl)-7-ethoxyisoquinolin-8-amine dihydrochloride 18,
4-(3,5-dimethoxy-4-propoxybenzyl)-7-ethoxyisoquinolin-8-ol hydrochloride 19,
4-(4-isobutoxy-3,5-dimethoxybenzyl)-7-ethoxyisoquinolin-8-ol hydrochloride 20,
4-(3,4,5-trimethoxybenzyl)-7-ethoxyisoquinolin-8-yl sulfamate hydrochloride 21,
4-(4-(2-methoxyethoxy)-3,5-dimethoxybenzyl)-7-ethoxyisoquinolin-8-ol hydrochloride 22,
7-ethoxy-4-(4-ethoxy-3,5-dimethoxybenzyl)isoquinoline-1-carbonitrile 23,
2-(4-(3,4,5-trimethoxybenzyl)-7-ethoxyisoquinolin-8-yloxy)acetonitrile 24,
(4-(4-ethoxy-3,5-dimethoxybenzyl)-7-ethoxyisoquinolin-1-yl)methanamine dihydrochloride 25,
(S)-2-amino-N-(7-ethoxy-4-(3,4,5-trimethoxybenzyl)isoquinolin-8-yl)-3-hydroxypropanamide dihydrochloride 26,
4-(3,4,5-trimethoxybenzyl)-7-methoxyisoquinoline hydrochloride 27,
(7-methoxyisoquinolin-4-yl)(3,4,5-trimethoxyphenyl)methanone hydrochloride 28,
(8-amino-7-ethoxyisoquinolin-4-yl)(3,4,5-trimethoxyphenyl)methanone dihydrochloride 29,
4-(3,5-dimethoxy-4-(2,2,2-trifluoroethoxy)benzyl)-7-ethoxyisoquinolin-8-ol hydrochloride 30,
4-(4-isobutoxy-3,5-dimethoxybenzyl)-7-ethoxyisoquinolin-8-amine dihydrochloride 31,
4-(4-(allyloxy)-3,5-dimethoxybenzyl)-7-ethoxyisoquinolin-8-ol hydrochloride 32,
7-ethoxy-4-(4-(isopentyloxy)-3,5-dimethoxybenzyl)isoquinolin-8-ol hydrochloride 33,
4-(4-(cyclobutylmethoxy)-3,5-dimethoxybenzyl)-7-ethoxyisoquinolin-8-ol hydrochloride 34,
4-(4-(2-cyclohexylethoxy)-3,5-dimethoxybenzyl)-7-ethoxyisoquinolin-8-ol hydrochloride 35,
4-(4-(cyclohexylmethoxy)-3,5-dimethoxybenzyl)-7-ethoxyisoquinolin-8-ol hydrochloride 36,
7-ethoxy-4-(4-(2-ethylbutoxy)-3,5-dimethoxybenzyl)isoquinolin-8-ol hydrochloride 37,
4-(4-butoxy-3,5-dimethoxybenzyl)-7-ethoxyisoquinolin-8-ol hydrochloride 38,
4-(3,5-dimethoxy-4-(neopentyloxy)benzyl)-7-ethoxyisoquinolin-8-ol hydrochloride 39,
(7-ethoxyisoquinolin-4-yl)(3,4,5-trimethoxyphenyl)methanone hydrochloride 40,
(7-ethoxyisoquinolin-4-yl)(3,4,5-trimethoxyphenyl)methanone oxime hydrochloride 41,
(7-ethoxyisoquinolin-4-yl)(3,4,5-trimethoxyphenyl)methanethione hydrochloride 42,
(7-ethoxyisoquinolin-4-yl)(3,4,5-trimethoxyphenyl)methanol hydrochloride hydrochloride 43,
4-(3,5-dimethoxy-4-(3-phenylpropoxy)benzyl)-7-ethoxyisoquinolin-8-ol hydrochloride 44,
4-(3,5-dimethoxy-4-phenethoxybenzyl)-7-ethoxyisoquinolin-8-ol hydrochloride 45,
7-ethoxy-4-(4-ethoxy-3,5-dimethoxybenzyl)-8-methoxyisoquinoline hydrochloride 46,
(4-ethoxy-3,5-dimethoxyphenyl)-(7-ethoxy-8-methoxyisoquinolin-4-yl)-methanone hydrochloride 47,
methyl 2-(4-((7-ethoxy-8-hydroxyisoquinolin-4-yl)methyl)-2,6-dimethoxyphenoxy)acetate hydrochloride 48,
2-(4-((7-ethoxy-8-hydroxyisoquinolin-4-yl)methyl)-2,6-dimethoxyphenoxy)acetamide hydrochloride 49,
2-(4-((7-ethoxy-8-hydroxyisoquinolin-4-yl)methyl)-2,6-dimethoxyphenoxy)acetonitrile hydrochloride 50,
4-(4-(benzylamino)-3,5-dimethoxybenzyl)-7-ethoxyisoquinolin-8-ol hydrochloride 51,
4-(3,5-dimethoxy-4-(phenylamino)benzyl)-7-ethoxyisoquinolin-8-ol hydrochloride 52,
4-((2,6-dimethoxy-[1,1′-biphenyl]-4-yl)methyl)-7-ethoxyisoquinolin-8-ol hydrochloride 53,
(7-methoxyisoquinolin-4-yl)(3,4,5-trimethoxy-2-nitrophenyl)methanone hydrochloride 54,
(8-(benzyloxy)-7-ethoxyisoquinolin-4-yl)(4-ethoxy-3,5-dimethoxyphenyl)methanone 55,
(4-ethoxy-3,5-dimethoxyphenyl)(7-ethoxy-8-hydroxyisoquinolin-4-yl)methanone hydrochloride 56,
7-ethoxy-4-(4-ethoxy-3,5-dimethoxybenzoyl)isoquinolin-8-yl sulfamate 57,
(8-amino-7-ethoxyisoquinolin-4-yl)(4-ethoxy-3,5-dimethoxyphenyl)methanone hydrochloride 58,
7-ethoxy-4-(4-ethoxy-3,5-dimethoxybenzoyl)isoquinolin-8-yl sulfamide 59,
(8-(2-(diphenyl methylene)hydrazinyl)-7-ethoxy-4-(4-ethoxy-3,5-dimethoxybenzyl)isoquinoline hydrochloride 60,
7-ethoxy-4-((8-methoxy-2,3-dihydrobenzo[b][1,4]dioxin-6-yl)methyl)isoquinolin-8-ol hydrochloride 61,
7-ethoxy-4-((7-methoxybenzo[d][1,3]dioxol-5-yl)methyl)isoquinolin-8-ol hydrochloride 62,
4-(3,5-dimethoxy-4-(pentyloxy)benzyl)-7-ethoxyisoquinolin-8-ol hydrochloride 63,
4-(3,5-dimethoxy-4-propoxybenzyl)-7-ethoxyisoquinolin-8-amine dihydrochloride 64,
4-(3,5-Dimethoxy-4-propoxybenzoyl)-7-ethoxyisoquinolin-8-yl acetate 65,
(8-amino-7-ethoxyisoquinolin-4-yl)(3,5-dimethoxy-4-propoxyphenyl)methanone hydrochloride 66,
(3,5-dimethoxy-4-propoxyphenyl)(7-ethoxy-8-hydroxyisoquinolin-4-yl)methanone hydrochloride 67,
O-(4-(3,5-dimethoxy-4-propoxybenzyl)-7-ethoxyisoquinolin-8-yl) dimethylcarbamothioate hydrochloride 68,
4-(3,5-dichloro-4-methoxybenzyl)-7-ethoxyisoquinolin-8-ol hydrochloride 69,
4-(3,5-diiodo-4-methoxybenzyl)-7-ethoxyisoquinolin-8-ol hydrochloride 70,
4-(3-bromo-4,5-dimethoxybenzyl)-7-ethoxyisoquinolin-8-ol hydrochloride 71,
7-ethoxy-4-(3-iodo-4,5-dimethoxybenzyl)isoquinolin-8-ol hydrochloride 72,
7-ethoxy-4-(4-ethyl-3,5-dimethoxybenzyl)isoquinolin-8-ol hydrochloride 73,
7-ethoxy-4-(4-ethoxy-3-methoxy-5-nitrobenzyl)isoquinolin-8-ol hydrochloride 74,
4-(3-amino-4-ethoxy-5-methoxybenzyl)-7-ethoxyisoquinolin-8-ol hydrochloride 75,
4-(3,5-dimethoxy-4-(pyrrolidin-1-yl)benzyl)-7-ethoxyisoquinolin-8-ol hydrochloride 76,
4-(3-bromo-4-ethoxy-5-methoxybenzyl)-7-ethoxyisoquinolin-8-ol hydrochloride 77,
7-ethoxy-4-(4-isobutoxy-3,5-dimethoxybenzoyl)isoquinolin-8-yl acetate 78,
(7-ethoxy-8-hydroxyisoquinolin-4-yl)(4-isobutoxy-3,5-dimethoxyphenyl)methanone hydrochloride 79,
6-(3,5-dimethoxy-4-propoxybenzyl)-3-ethyloxazolo[4,5-h]isoquinolin-2(3H)-one hydrochloride 80,
6-(3,5-dimethoxy-4-propoxybenzoyl)-3-ethyloxazolo[4,5-h]isoquinolin-2(3H)-one hydrochloride 81,
S-(4-(3,5-dimethoxy-4-propoxybenzyl)-7-ethoxyisoquinolin-8-yl) dimethylcarbamothioate hydrochloride 82,
S-(4-(3,5-dimethoxy-4-propoxybenzoyl)-7-ethoxyisoquinolin-8-yl) dimethylcarbamothioate hydrochloride 83,
(3,5-dimethoxy-4-propoxyphenyl)(7-ethoxy-8-mercaptoisoquinolin-4-yl)methanone hydrochloride 84,
7-ethoxy-4-(3,4,5-trimethoxybenzyl)isoquinoline-8-carbonitrile hydrochloride 85,
7-ethoxy-4-(3-isobutoxy-4,5-dimethoxybenzyl)isoquinolin-8-ol hydrochloride 86,
4-(3,4-dimethoxy-5-(2,2,2-trifluoroethoxy)benzyl)-7-ethoxyisoquinolin-8-ol hydrochloride 87,
8-amino-7-ethoxy-2-methyl-4-(3,4,5-trimethoxybenzyl)isoquinolinium chloride 88,
7-ethoxy-N-ethyl-4-(3,4,5-trimethoxybenzyl)isoquinolin-8-amine dihydrochloride 89,
7-ethoxy-N-(2,2,2-trifluoroethyl)-4-(3,4,5-trimethoxybenzyl)isoquinolin-8-amine dihydrochloride 90,
7-ethoxy-N,N-dimethyl-4-(3,4,5-trimethoxybenzyl)isoquinolin-8-amine dihydrochloride 91,
7-ethoxy-N-methyl-4-(3,4,5-trimethoxybenzyl)isoquinolin-8-amine dihydrochloride 92,
methyl 2-(7-ethoxy-4-(3,4,5-trimethoxybenzyl)isoquinolin-8-ylamino)acetate dihydrochloride 93,
7-ethoxy-8-iodo-4-(3,4,5-trimethoxybenzyl)isoquinoline hydrochloride 94,
7-ethoxy-4-(1-(3,4,5-trimethoxyphenyl)vinyl)isoquinolin-8-ol hydrochloride 95,
7-ethoxy-4-(3,4,5-trimethoxybenzoyl)isoquinolin-8-yl acetate 96,
(7-ethoxy-8-hydroxyisoquinolin-4-yl)(3,4,5-trimethoxyphenyl)methanone hydrochloride 97,
7-ethoxy-4-(3,4,5-trimethoxybenzoyl)isoquinolin-8-yl disodium phosphate 98,
7-(2,2,2-trifluoroethoxy)-4-(3,4,5-trimethoxybenzyl)isoquinolin-8-ol hydrochloride 99,
4-(3,4,5-trimethoxybenzyl)isoquinoline-7,8-diol hydrochloride 100,
4-(3,4,5-trimethoxybenzyl)isoquinolin-7-ol hydrochloride 101,
7-ethoxy-4-(4-hydroxy-3-methoxy-5-nitrobenzyl)isoquinolin-8-ol hydrochloride 102,
4-(3,4-dimethoxy-5-nitrobenzyl)-7-ethoxyisoquinolin-8-ol hydrochloride 103,
tert-butyl N-(7-ethoxy-4-(3,4,5-trimethoxybenzoyl)isoquinolin-8-yl)sulfamoylcarbamate 104,
N-(7-ethoxy-4-(3,4,5-trimethoxybenzoyl)isoquinolin-8-yl)sulfamide hydrochloride 105,
8-chloro-7-ethoxy-4-(3,4,5-trimethoxybenzyl)isoquinoline hydrochloride 106,
8-azido-7-ethoxy-4-(3,4,5-trimethoxybenzyl)isoquinoline 107,
8-azido-7-ethoxy-4-(3,4,5-trimethoxybenzoyl)isoquinoline 108,
7-ethoxy-4-(3,4,5-trimethoxybenzoyl)isoquinolin-8-yl pivalate 109,
4-(2-chloro-3,4,5-trimethoxybenzyl)-7-ethoxyisoquinolin-8-ol hydrochloride 110,
N-(7-ethoxy-4-(3,4,5-trimethoxybenzoyl)isoquinolin-8-yl)acetamide hydrochloride 111,
2-amino-N-(7-ethoxy-4-(3,4,5-trimethoxybenzoyl)isoquinolin-8-yl)acetamide 112,
isobutyl (7-ethoxy-4-(3,4,5-trimethoxybenzoyl)isoquinolin-8-yl)carbamate hydrochloride 113,
(S)-2-amino-N-(7-ethoxy-4-(3,4,5-trimethoxybenzoyl)isoquinolin-8-yl)-3-phenylpropanamide dihydrochloride 114,
4-(3-bromo-4,5-dimethoxybenzoyl)-7-ethoxyisoquinolin-8-yl acetate 115,
(3-bromo-4,5-dimethoxyphenyl)(7-ethoxy-8-hydroxyisoquinolin-4-yl)methanone hydrochloride 116.

27. A pharmaceutical composition comprising a compound according to claim 18, in a pharmaceutically acceptable carrier, alone or in combination with another therapeutic agent.

28. A method for the treatment of a disease state in need thereof, comprising administering to said subject a compound according to claim 18, thereby inhibiting tubulin polymerization or unwanted neovascularisation.

29. The method according to claim 28, wherein the disease state is a cancer, inflammation or a disorder caused by unwanted neovascularisation.

30. The method according to claim 29, wherein the cancer is selected in the group consisting of sarcomas, leukemias, melanomas, glioblastomas, oligodendroglioma, astrocytic glioma, thyroid, colon, ovarian, skin, breast, prostate, CNS, renal and lung cancers, liver neoplasms, meningeoma, testis cancer, uterine cancer, cervical neoplasm, bladder cancer, neuroblastoma, retinoblastoma, embryonal carcinoma, Wilm's tumors or Ewing's tumor.

31. The method according to claim 28, wherein said method is for the treatment of a cancer by combination therapy with other chemotherapeutic or radiation treatments, or with anti-angiogenic therapies.

32. The method according to claim 28, wherein the method is for the treatment of a disease caused by abnormal angiogenesis.

33. The method according to claim 32, wherein the disease caused by abnormal angiogenesis is age-related macular degeneration, neovascular glaucoma, retinal vein obstruction, myopic macular degeneration, retinopathy of prematurity, proliferative diabetic retinopathy, posterior capsular opacification (PCO) or pediatric hemangiomas.

34. The method according to claim 28, wherein the method is for the treatment of acne rosacea, atopic keratitis, epidemic keratoconjunctivitis, bacterial ulcers, fungal ulcers, Herpes simplex infections, Herpes zoster infections, protozoan infections, Mycobacterium infections, polyarteritis, sarcoidosis, scleritis, flush, Sjogren's disease, systemic lupus, Acquired Immune Deficiency Syndrome (AIDS), syphilis or infection with Treponema pallidum or related parasites.
